(19) United States
(12) Patent Application Publication
(10) Pub. No.: US 2007/0213274 A1
(43) Pub. Date: Sep. 13, 2007

(54) Novel genes and markers associated with high-density lipoprotein-cholesterol (HDL-C)

(75) Inventor: Jukka T. Salonen, Kuopio (FI)

Correspondence Address: BIRCH STEWART KOLASCH & BIRCH, PO BOX 747, FALLS CHURCH, VA, 22040-0747, US

(73) Assignee: Oy Jurilab Ltd, Kuopio (FI)

(21) Appl. No.: 11/640,897

(22) Filed: Dec. 19, 2006

Related U.S. Application Data
(60) Provisional application No. 60/751,624, filed on Dec. 20, 2005.

(51) Int. Cl. A61K 48/00; A61K 38/17; G06F 19/00; C12Q 1/68

(52) U.S. Cl. 514/12; 514/44; 435/6; 705/3

(57) ABSTRACT

This invention relates to the therapeutic, diagnostic and pharmacogenetic use of nucleic acids and proteins involved in the regulation of human high density lipoprotein (HDL) and pharmaceutical agents and other therapies affecting this. This invention discloses methods for the treatment and prevention of low HDL states and diseases to prevent cardiovascular diseases such as coronary heart disease (CHD), acute myocardial infarction (AMI), chronic CHD and cerebrovascular stroke and for selecting treatment in a subject and for selecting subjects for studies testing HDL elevating agents, as well as to transgenic animals.

### FIELD OF THE INVENTION

The present invention relates generally to the field of treatment and prevention of low high-density lipoprotein-cholesterol (HDL-C) states, as it provides novel methods for prevention and treatment of low HDL-C status. The invention also relates to the field of prevention and treatment of conditions characterised by low HDL-C, such as cardiovascular diseases (CVD), type 2 diabetes (MD), the metabolic syndrome (MBO) and obesity, via diagnosis and treatment of low HDL-C. In addition, the invention relates to methods for screening new chemical entities for elevating HDL.

Low HDL-C and Disease

There are several main classes of plasma transporters, which carry and enhance the exchange of lipids in the circulation and between plasma and cells. These include the chylomicrons (CM), the very low-density lipoproteins (VLDL), the intermediate lipoproteins (ILP), the low-density lipoproteins (LDL) and HDL. A number of others exist (lipoprotein a, subtypes of the main classes), though not routinely measured.

Low HDL-cholesterol (HDL-C), high LDL-C and high plasma triglycerides (Tg) embody a dyslipidemia, common for atherosclerosis, T2D, obesity and MBO.

HDL represents one of the main lipoprotein carriers of cholesterol. Low HDL-C levels characterize about 10% of the general population (Sampietro T et al, 2005). Furthermore, low HDL concentration represents the most frequent dyslipidemia in patients with coronary artery disease (CAD) (Sampietro T, et al, 2005).

Despite of the existence of a number of drugs successfully reducing LDL plasma availability, the following reduction of cardiovascular risk does not prove to be enough sufficient. A number of clinical studies have been aiming to determine whether aggressive lowering of LDL-C beyond the currently accepted guidelines would result in further reduction of cardiovascular events (Cannon C P, et al, 2004; Waters D D, et al,2004). The results from some of those studies are still pending, while others such as the PROVE IT-TIMI 22 (Cannon, C. P., et al., 2004) have shown certain benefits of aggressive lowering of LDL, which, however, leave remarkably high residual cardiovascular disease (CVD) occurrence.

HDL is an independent predictor of the risk of CHD or CAD (Castelli W P et al, 1986, Salonen J T et al, 1991). Already in 1977 it was shown that CAD patients have 35% lower HDL-C levels than controls and those with lowered HDL have been exposed to three times higher likelihood of developing CAD than those with elevated LDL-C (Miller N E et al, 1977). Low HDL-C was observed to be the most common lipid abnormality in men with coronary artery disease (Genest J J et al, 1991). According to the first large-scale prospective trial to study the effect of raising HDL-C on CAD incidence (the Helsinki Heart Study), 11% increase in HDL-C levels was independently associated with a 34% reduction in CAD events (Manninen V et al, 1992). A number of other clinical studies have confirmed a significantly reduced incidence of coronary events after an increase in HDL-C concentration (Alberti K G 1998; Frick M H et al, 1987; Rubins H B et al, 1999). Thus elevating the low HDL-C levels independently or in combination with a decreasing of the high LDL-C state represents a frontier in the treatment and prevention of CVD.

Besides characterising the dyslipidemia related to T2D, low HDL-C has been related to a higher conversion rate from impaired glucose tolerance (IGT) to T2D (Todorova B et al, 2004). Subjects with type 2 diabetes generally carry an array of risk factors for cardiovascular disease (CVD), including hyperglycaemia, dyslipidaemia (high Tg, high LDL-C and low HDLC), alterations in inflammatory mediators and coagulation/thrombolytic parameters, as well as other ‘non-traditional’ risk factors, many of which may be closely associated with insulin resistance (Erdmann E, 2005). Consequently, rates of CVD mortality and morbidity are particularly high in this population (Erdmann E, 2005). Targeting hyperglycaemia alone does not reduce the excess cardiovascular risk in diabetic patients, highlighting the need for aggressive treatment of other risk factors and in that sense the low HDL-C levels.

Not lastly, low HDL-C is one of the hallmarks of the metabolic/insulin resistance syndrome (MS, IRS, MBO)—a concurrence of disturbed glucose and insulin metabolism, overweight and abdominal fat distribution, mild dyslipidemia and hypertension. The syndrome is characterized by insulin resistance, and is also known as the insulin resistance syndrome. An elevation of the decreased HDL-C levels yet again implies for a rationale drug target in the prevention and treatment of MS.

Atheroprotective Effect of HDL

At a molecular level, atherosclerosis is a time dependent, multistep process involving the interaction of many different key pathways, including lipoprotein metabolism (Chisolm G M and D Steinberg, 2000), lipoprotein oxidation (Salonen J T et al, 1992), coagulation (Tremoli E et al, 1999) and inflammation (Ross R, 1999). Gene mutations in any of these pathways will only provide a partial contribution to risk. Intermediate phenotypes such as hypertension, diabetes, smoking and obesity interact to modulate risk as will do gene-gene and gene-environment interactions (Stephens J W and Humphries S E, 2003).

The atheroprotective role of HDL particles has been widely studied though still to be elucidated. A proposed mechanism leading to the formation of the foam cells and thus to the formation of the atherosclerotic plaque is the imbalance between the uptake of lipoproteins and cholesterol efflux from Mf (Linsel-Nitschke P and Tall A R, 2005). HDL mediated efflux of cholesterol from cholesterol loaded macrophages, other cells and LDL particles takes place in the following described RCT pathway. The lipid content of the LDL particles is known to be more prone to oxidation than the one in the HDL (Navab M et al, 2004). Furthermore, the LDL particles have the characteristic to remain longer in the subendothelial space compared to the HDLs.

HDL is considered to expand its protective role further than only in promoting the efflux of cholesterol from lipid-loaded cells. HDL particles show anti-inflammatory activity and are effective antioxidants via suppressing the induction of cell-adhesion molecules in endothelial cells, mediated by tumour necrosis factor α (TNFα) (Cockerill G W et al, 1995) and C-reactive protein (CRP) (Wadham C et al, 2004). Thus they have a role to lessen the recruitment of blood monocytes into the arterial wall. Further on, by means of the ApoA1 and ApoA2, which are known to have antioxidant characteristics, as well as via the cotransport of paraoxonase, HDL particles expand their antioxidant functions (Barter P J et al, 2004).

It has been shown that oxidized LDL (oxLDL) depletes caveolae of cholesterol, which on turn results in the displacement of endothelial nitric-oxide synthase (eNOS) from caveolae with impairement of the eNOS activation (Uittenbogaard A et al, 2000). HDL binding to the scavenger receptor BI (SR-BI) maintains the concentration of caveola-associated cholesterol by promoting the uptake of cholesterol esters, thereby preventing oxLDL-induced depletion of caveola cholesterol (Uittenbogaard A et al, 2000). Furthermore, HDL maintains the subcellular location of eNOS which decreases the capacity for eNOS activation (Uittenbogaard A et al, 2000). Additionally, HDL activates eNOS (Yuhanna I. S et al, 2001) and accounts for increased myocardial perfusion via NO-dependent mechanisms (Levkau B et al, 2004).

The Reverse Cholesterol Transport

Several aspects engage HDL particles as modulators of the formation and progression of the atherosclerotic plaque, the main of which involves the promotion of cholesterol efflux in the reverse cholesterol transport pathway (RCT). RCT on the other hand encompasses the main pathway of metabolism, regulation, transformation and degradation of the HDL particle.

The primary site for nascent HDL formation is liver, whereas peripheral tissues and liver both are involved in further lipidation of the HDL particles. Apolipoprotein A 1 (ApoA1) is the main structural component of the HDL particles. Secreted by the liver ApoA1 becomes associated with phospholipids and shapes the discoidal nascent pre-βHDL particle.

The initial step of the RCT is the transfer of cholesterol and phospholipids to the lipid-poor ApoA1. It is mediated by the membrane ATP-binding cassette transporter 1 (ABCA1) protein. ABCA1 belongs to the ATP-binding cassette transporter superfamily which is known to carry a large number of molecules, such as proteins, ions and lipids across plasma membranes. ABCA1 deficiency results in little or no plasma HDL in human or animals (Attie A D, 2001), while its overexpression has been related to increased cholesterol and phospholipid efflux, accompanied by increased HDL levels (Singaraja R R, et al, 2001; Vaisman B L et al, 2001).

The free cholesterol on the surface of the small pre-βHDL particles undergoes esterification in order to be stored in the core of the particles. In this process the small discoidal particle is transformed to a spherical and larger one. The cholesterol esterification is mediated by the lecithin cholesterol acyl transferase (LCAT). LCAT is an enzyme, secreted by the liver and circulating in blood. It binds reversibly to the surface of the HDL particles. The process of cholesterol esterification and accumulation of the cholesterol esters in the particle core promotes further cholesterol efflux from cells to the HDL particles. Thus LCAT plays a pivotal role in promoting of the cholesterol uptake from the lipid loaded cells (Wang, M. and M. R. Briggs, 2004).

The spherical and smaller HDL particle (HDL₃) becomes larger HDL₂ as it accepts more free cholesterol from cells. This stage of cholesterol transfer from cells to HDL₃ is mediated by the scavenger receptor B1 (SR-B1) or a passive diffusion, both distinct from the one mediated by the ABCA1 (Wang M and Briggs M R, 2004). The scavenger receptors are cell surface membrane proteins that bind chemically modified lipoproteins such as acetylated LDL and oxidised LDL (Krieger M, 1997). SR-B1 binds HDL particles with high affinity and represents a mediator of the selective cholesterol uptake. Furthermore, it is as well the HDL receptor responsible for the selective HDL uptake in the liver (Wang M and Briggs M R, 2004).

A central element in the RCT is the interaction between the LDL, VLDL and HDL particles and particularly the exchange of cholesterol esters, phospholipids and triglycerides. As a result the excess cholesterol is transported from the periphery to a metabolic disposal or recycling processes. The cholesterol ester transfer protein (CETP) mediates the exchange of lipids from the large HDL₂ and LDL particles to the VLDL particles (Wang M and Briggs M R, 2004). CETP is associated with the HDL particles in plasma and its activity is reversely correlated to the HDL-C levels. The phospholipids transfer protein (PLTP) is another transferring protein, which mediates the transport of lipids from the VLDL to the HDL₃ particles (Wang M and Briggs M R, 2004).

The degradation of large cholesterol-rich HDL₂ particles follows the cholesterol ester selective uptake, mediated by the SR-B1 (Wang M and Briggs M R, 2004). In the liver the cholesterol molecules are excreted via the bile or further utilized in body systems, while the ApoA1 is used in a new cycle of RCT.

An alternative way for HDL degradation is present in kidneys where the ApoA1 undergoes a renal clearance via interaction with a receptor, known as cubulin. Cubulin is expressed in a various number of tissues and shows a co-expression with megalin—a member of the LDL receptor family (Moestrup SK et al, 1998). Cubulin is a major ligand not only for ApoA1 but for HDL particles as well, as it efficiently mediates their endocytosis (Moestrup S K and Kozyraki R, 2000).

Necessary to be mentioned is a number of lipases, playing major roles in the HDL metabolism. The lipoprotein lipase (LPL) is bound to the surface of the endothelial cells and its activity correlates positively with the HDL concentration (Tornvall P et al, 1995). In the process of hydrolysing Tg rich lipoproteins (chylomicrons and VLDL), redundant surface lipids (free cholesterol and phospholipids) and apolipoproteins are transferred to HDL particles, contributing to the plasma HDL-C levels (Lewis G F and Rader D J, 2005).

The hepatic lipase (HL), another member of the lipases family, is situated on the surface of the sinusoidal capillaries in liver. In contrast to LPL, the HL has greater affinity to HDL particles than to VLDL or chylomicrons and converts larger HDL particles to smaller cholesterol-poor HDL remnants (Lewis G F and Rader D J, 2005). The endothelial lipase (EL) represents one more important HDL modulating lipase, which is located on the surface of the endothelium and is recognised to have a phospholipase A activity (Lewis G F and Rader D J, 2005).

Determinants of HDL

The main participants in the RCT pathway are the principal contributors to the HDL plasma levels. It has been shown that mutations in the genes encoding for ABCA1, LCAT, CETP, PLTP, HL, LPL, EL and SR-B1 have an impact on the HDL levels (Singaraja R R et al, 2001; Miltiadous G et al, 2005; Thompson J F et al, 2005; Cohen J C et al, 2004; Brousseau M E et al, 2004; Mank-Seymour A R et al, 2004; Morabia A et al, 2004). However, a number of other receptors, transporters and enzymes have influence on the HDL metabolism and plasma concentration. In that order, ABCG1 and ABCG4 have been reported to mediate the efflux of cellular cholesterol to mature HDL particles (but not to lipid poor ApoA1) (Wang N et al, 2004). Polymorphisms in the genes encoding for the ABCG5 and ABCG8 transporter proteins, which are related to the sitosterolemia (an inherited disorder, characterised by high absorption and low biliary secretion of cholesterol and plant sterols) have been associated with low HDL-C (Gylling H et al, 2004). To be mentioned further are members of the secretory phospholipase A2 family, which are plausibly relevant to the physiology of the HDL metabolism, by influencing the size and catabolism of the HDL particles (Tietge U J et al, 2002).

ApoA1 accounts for up to 70% of apolipoprotein content of HDL particles (Lewis G F and Rader D J, 2005; Davidson W S and Silva R A, 2005). ApoA1 exists in three main plasma forms (Davidson W S and Silva R A, 2005). Approximately 5-10% of plasma ApoA1 is found in a lipoprotein-unassociated state (Davidson W S and Silva R A, 2005). The other two forms of ApoA1 are coupled with the state of HDL reshaping, varying between discoidal and spherical. It has been postulated that ApoA1 responds to changes of the HDL diameter by folding or unfolding its so-called “hinge” domains (Davidson WS and Silva RA, 2005).

Since ApoA1 represents the main structural component of the HDL particles, the regulation of the ApoA1 gene expression, mutations and ApoA1 synthesis would have a significant implication on the HDL plasma concentration. Among the factors influencing the ApoA1 synthesis and metabolism are some hormones, such as thyroid hormones, estrogens and glucocorticoids (Hargrove G M et al, 1999). Furthermore, glucose and insulin (Mooradian A D, 2004), as well as cellular acidity (ketoacidosis) (Mooradian A D, 2004) and insulin resistance (Lopez-Candales A, 2001; Vajo Z et al., 2002) have shown to be associated with ApoA1 and HDL-C levels as well.

ApoA2 is the second most abundant apolipoprotein on HDL particles. A variety of other proteins such as ApoA4, ApoC1, ApoC3, ApoD, ApoE, ApoJ, ApoL1, ApoM and others contribute additionally to the HDL structure and thus modulate HDL plasma levels (Singaraja R R et al., 2001).

Among the non-genetic factors with a major influence on the HDL-C levels are age, gender, smoking (Nash D T, 2004), diet (Nash D T, 2004), alcohol consumption (Nash D T, 2004), exercise and physical activity (Nash D T, 2004). Also body composition, and specifically fat distribution (Pi-Sunyer F X, 2004), which are only in part genetivcally determined, have a major influence on HDL levels.

Obesity and particularly visceral obesity is associated with low HDL-C concentration (Pi-Sunyer F X, 2004). Adipose tissue and precisely the visceral adipocytes are metabolically very active, expressing various secretory proteins such as adiponectine, angiotensinogen, tumour necrosis factor-α (TNF-α), interleukins (ILs), plasminogen activator inhibitor type 1 and others. The free fatty acids (FFA), the TNFα and the IL-1β have been shown to alter the ApoA1 activation and expression and thus encompass an additional weight on the HDL-C levels (Haas M J et al, 2003).

Public Health Significance of CVD, T2D and MBO

Cardiovascular Diseases (CVD) (ICD/10 codes I00-I99, Q20-Q28) include ischemic (coronary) heart disease (CHD), hypertensive diseases, cerebrovascular disease (stroke) and rheumatic fever/rheumatic heart disease, among others (AHA, 2004). In terms of morbidity, mortality and cost CHD is the most important disease group of CVD. CHD (ICD/10 codes I20-I25) includes acute myocardial infarction (AMI), other acute ischemic (coronary) heart disease, angina pectoris; atherosclerotic cardiovascular disease and all other forms of chronic ischemic heart disease (AHA, 2004). Dyslipidemia (low HDL-C, high LDL-C and high FFA levels) is among the major CVD risk factors (Stamler J et al, 1998).

In 2001 an estimated 16.6 million—or one-third of total global deaths—resulted from the various forms of CVD (7.2 million due to CHD, 5.5 million to cerebrovascular disease, and an additional 3.9 million to hypertensive and other heart conditions). At least 20 million people survive heart attacks and strokes every year, a significant proportion of them requiring costly clinical care, which puts a huge burden on long-term care resources. It is necessary to recognize that CVD are devastating to men, women and children (ADA, 2004).

The term diabetes mellitus (DM) (ICD/10 codes E10-E14) describes several syndromes of abnormal carbohydrate metabolism that are characterized by hyperglycaemia. According to the new etiologic classification of DM, four categories are differentiated: type 1 diabetes (T1D), type 2 diabetes (T2D), other specific types, and gestational diabetes mellitus (ADA, 2003). In the United States, Canada, and Europe, over 80% of cases of diabetes are due to T2D, 5 to 10% to T1D, and the remainder to other specific causes. T2D is associated with a relative or absolute impairment in insulin secretion, along with varying degrees of peripheral resistance to the action of insulin. The chronic hyperglycaemia of diabetes is associated with long-term damage, dysfunction, and failure of various organs, especially the eyes, kidneys, nerves, heart, and blood vessels (ADA, 2003). T2D is characterized by adult onset insulin resistance and a rise in blood sugar concentration. In T1D, formerly known as insulin-dependent (IDDM), the pancreas fails to produce the insulin which is essential for survival. This form develops most frequently in children and adolescents, but is being increasingly diagnosed later in life. T2D, formerly named non-insulin-dependent (NIDDM), results from the body's inability to respond properly to the action of insulin produced by the pancreas. T-2D occurs most frequently in adults, but is being noted increasingly in adolescents as well (WHO, 2004). The causes of T2D are multi-factorial and include both genetic and environmental elements that affect beta cell function and tissue insulin sensitivity (muscle, liver, adipose tissue, pancreas). Although there is considerable debate as to the relative contributions of beta-cell dysfunction and reduced insulin sensitivity to the pathogenesis of diabetes, it is generally agreed that both of these factors play important roles (Scheen A J, 2003).

In 2000, there were approximately 171 million people, worldwide, with diabetes. The number of people with diabetes will expectedly double over the next 25 years and reach a total of 366 million by 2030 (WHO/IDF, 2004). Most of this increase will occur as a result of a 150% rise in developing countries. This suggests the role of relatively modern environmental or behavioural risk factors such as high caloric intake or sedentary lifestyle. However, ethnic differences in the incidence and prevalence of T2D and the enrichment of T2D in families suggest heritable risk factors to play a major role. The two main contributors to the worldwide increase in prevalence of diabetes are population ageing and urbanization, especially in developing countries, with the consequent increase in the prevalence of obesity (WRO/IDF, 2004). Currently more than 1 billion adults are overweight—and at least 300 million of them are clinically obese. Current obesity levels range from below 5% in China, Japan and certain African nations, to over 75% in urban Samoa. The prevalence of obesity is 10-25% in Western Europe and 20-27% in the Americas (WHO, 2004).

A distinct increase in the occurrence of MS has been observed worldwide in both adults and adolescence (Williams C L et al, 2002), and it is considered as an epidemic affecting not only the industrialized countries but the developing world as well.

To conclude, in a view of the pandemic spread of obesity, MBO, CVD and T2D, all in which low HDL-C has an important role, there is a lack of treatment models and medications focusing on the elevation of HDL-C. HDL levels are largely genetically determined, with results from different studies ranging from 24% to 83%, depending on different twin or family studies. A cluster of genetic and environmental factors has been assigned to the origin and development of CVD, T2D and MBO, lipid genetic determinants included. Thus, the discovery of genes regulating HDL metabolism and HDL plasma concentrations offers novel therapeutic strategies and targets in the management of the mentioned conditions.

### SUMMARY OF THE INVENTION

This invention relates to genes and biomarkers associated with low HDL-C levels and their use in the treatment and prevention of diseases and traits associated with low HDL-C levels. As ApoA1 is the major lipoprotein present in HDL particles the genes associated with ApoA1 levels (table 9.) are associated with HDL-C levels as well. Thus, the terms “HDL” and “HDL-C” are used in this patent to denote both high density lipoprotein and apolipoprotein AI. The present invention provides novel low HDL-C plasma level associated genes and individual SNP markers and combinations of SNP markers (haplotypes). The invention further relates to physiological and biochemical routes and pathways related to these genes. These pathways provide a basis for further research and development of CVD, T2D, MBO and obesity predisposition, diagnosis and treatment.

One major object of this invention is to provide novel methods for the treatment of low HDL-C by modifying the expression of HDL-C associated genes, by modifying the activity or function of proteins and polypeptides encoded by said genes, or by modifying the activity or function of endogenous and exogenous modulators of said low HDL-C associated genes, proteins or polypeptides in the human or animal subject. Yet another aspect of the invention is methods for the treatment of diseases and conditions related to low HDL-C concentration, i.e. CVD, T2D, MBO and obesity.

Another major object of this invention is to provide novel methods for the prevention of low HDL-C by modifying the expression of HDL-C associated genes, by modifying the activity or function of proteins and polypeptides encoded by said genes, or by modifying the activity or function of endogenous and exogenous modulators of said low HDL-C associated genes, proteins or polypeptides in the human or animal subject. Yet another aspect of the invention is methods for the prevention of diseases and conditions related to low HDL-C concentration, i.e. CVD, T2D, MBO and obesity.

Still another object of the invention is to provide methods for prediction of clinical course and monitoring the efficacy of treatments for low HDL-C using biomarkers related to the low HDL-C associated genes of this invention. Yet another object of the invention is methods to targeting HDL elevating, anti-CHD or anti-diabetic treatments in subjects having low HDL-C level associated disease of trait by determining the presence of mutations and sequence variations effecting expression of one or more genes set forth in tables 1, 8 and 9.

Another object of the invention is providing novel pathways to elucidate the presently unknown modes of action of known drugs with impact on HDL-C levels.

The invention also provides methods for screening compounds for the treatment of the low HDL-C level associated diseases and traits. A further object of the invention is to provide a method for the selection of experimental animals and human subjects for studies testing HDL elevating effects of drugs. A further object of the invention is methods of using non-human transgenic and gene knock-out animals for screening agents targeted to a gene set forth in tables 1 to 11 for the treatment or prevention of the low HDL-C level associated diseases and traits.

In summary, the invention helps meet the unmet medical needs and promotes public health in at least two major ways: 1) it provides novel means to prevent and treat low HDL-C levels and reduce the risk of an individual having low HDL-C level associated diseases such as CVD, T2D, MBO and obesity and 2) it provides drug and other therapeutic targets that can be used further to screen and develop therapeutic agents and therapies that can be used to increase low HDL-C levels and consequently to prevent CVD, T2D, MBO, obesity and other conditions related to low HDL-C before they manifest clinically; to prevent complications, to treat clinical symptoms and/or to retard the progression of said diseases and conditions.

### DETAILED DESCRIPTION OF THE INVENTION

The present invention discloses methods for the prevention and treatment of low HDL-C levels. Furthermore, it includes methods for prevention and treatment of diseases and clinical conditions related to low HDL-C, i.e. CVD, T2D, MBO and obesity in a human or animal. In the following, the word treating shall also be understood to include preventing. In the present invention, an individual who has or is at risk of low HDL-C is an individual who has a risk-increasing allele in at least one of the HDL-C-associated genes set forth in tables 1, 8 and 9. The term “gene” as used herein, refers to an entirety containing all regulatory elements located both upstream and downstream as well as within of a polypeptide encoding sequence, 5′ and 3′ untranslated regions of mRNA and the entire polypeptide encoding sequence including all exon and intron sequences (also alternatively spliced exons and introns) of a gene.

Low levels of HDL-C relate significantly and independently to increased occurrence of atherosclerosis, CVD, T2D and metabolic syndrome. An increase in low HDL-C levels has been shown indisputably to relate to improved CVD survival.

Atherosclerosis is a continuous inflammatory process of lipid deposition in the arterial wall and further oxidation of the deposited lipids. Higher delivery of LDL particles to the endothelial intima and their longer deposition there predisposes to increased accumulation of subendothelial lipids and higher probability for developing atherosclerotic plaque or increasing/unstabilizing an existent one. HDL particles antagonize the oxidation of LDLU and decrease the availability of LDL lipid content prone to oxidation. Thus, decrease in plasma HDL-C (by increased HDL excretion or decreased production for instance) results in poorer protection of the endothelium against oxidative action, excess of prone to oxidation LDL particles, and increased risk for atherosclerosis.

Besides antagonising LDL oxidation and subendothelial accumulation HDL particles hold other anti-atherogenic properties, expressed in the RCT, possess antioxidative characteristics, and neutralise the effect of inflammatory markers on the endothelial cells.

ApoA1 is a main structural component of HDL particles, and alteration in its plasma level will reflect HDL-C concentration.

Therefore, we propose that genetic defects that modulate or alter HDL-C levels is a general mechanism in the body of a mammalian subject, such as human, which contributes to the development of common degenerative diseases and related traits, such as cardiovascular and metabolic diseases, and traits predisposing to them. Identification of novel genes and pathways responsible for the regulation of HDL-C concentration enables the development of new methods for improving/increasing HDL-C levels, and thus offers novel methods to treat and prevent said common degenerative diseases.

The present invention relates to the genes and the encoded proteins or polypeptides regulating HDL metabolism, and endogenous and exogenous modulators of said genes, proteins or polypeptides.

Methods of Therapy

The invention discloses novel methods for the treatment and prevention of low HDL-C levels based on modulation of polypeptides and related metabolic pathways regulating HDL-C levels. The invention further proposes methods of prevention, follow-up and treatment of conditions related to low HDL-C levels, i.e. CVD, T2D, MBO and obesity.

The term, “treatment” as used herein, refers not only to ameliorating symptoms associated with the trait or disease, but also preventing or delaying the onset of the disease, and also lessening the severity or frequency of symptoms of the disease, preventing or delaying the occurrence of a second episode of the disease or condition; and/or also lessening the severity or frequency of symptoms of the disease or condition.

In particular, the invention relates to methods of treatment for low HDL-C trait or susceptibility to low HDL-C (for example, for individuals in an at-risk population such as those described herein); as well as to methods of treatment for manifestations of low HDL-C related conditions including but not limited to atherosclerosis, CVD, T2D, MBO and obesity.

The present invention encompasses methods of treatment (prophylactic and/or therapeutic) for low HDL-C, such as individuals in the target populations described herein, using a low HDL-C level increasing therapeutic agent. A “low HDL-C level increasing therapeutic agent” is an agent that alters (e.g., enhances or inhibits) biological activity, function or concentration of a low HDL-C level affecting polypeptide and/or biological activity or function of low HDL-C level associated metabolic pathway as described herein. Useful therapeutic agents can alter a HDL-C associated polypeptide biological activity or function by a variety of means, such as, for example, by altering translation rate of a HDL-C associated polypeptide encoding mRNA; by altering the transcription rate of the HDL-C associated gene; by altering posttranslational processing of a HDL-C associated polypeptide; by interfering with a HDL-C associated polypeptide activity and/or function (e.g., by binding to a HDL-C associated polypeptide); by altering stability of a HDL-C associated polypeptide; by altering the transcription rate of splice variants of a HDL-C associated gene or by inhibiting or enhancing the elimination of a HDL-C associated polypeptide from target cells, organs and/or tissues.

Representative low HDL-C therapeutic agents comprise the following: (a) nucleic acids, fragments, variants or derivatives of HDL-C associated genes described in this invention, nucleic acids encoding a HDL-C associated polypeptide or an active fragment or a derivative thereof and nucleic acids modifying the expression of said low HDL-C associated genes (e.g. antisense polynucleotides, catalytically active polynucleotides (e.g. ribozymes and DNAzymes), molecules inducing RNA interference (RNAi and micro RNA), and vectors comprising said nucleic acids; (b) HDL-C associated polypeptides, active fragments, variants or derivatives thereof, binding agents of HDL-C associated polypeptides; peptidomimetics; fusion proteins or prodrugs thereof, antibodies (e.g., an antibody to a mutant HDL-C associated polypeptide, or an antibody to a non-mutant HDL-C associated polypeptide, or an antibody to a particular variant encoded by a HDL-C associated gene, as described above) and other polypeptides (e.g., HDL-C associated receptors, active fragments, variants or derivatives thereof); (c) metabolites of HDL-C associated polypeptides or derivatives thereof; (d) small molecules and compounds that alter (e.g., inhibit or antagonize, or activate or agonize) a HDL-C associated gene expression, activity and/or function of a HDL-C associated gene encoded polypeptide, or activity and/or function of a HDL-C associated gene related metabolic pathway and; (e) small molecules and compounds that alter (e.g. induce or agonize, or activate or antagonize) a HDL-C associated gene expression, activity and/or function of a HDL-C associated gene encoded polypeptide, or activity and/or function of a low HDL-C associated gene related metabolic pathway.

More than one low HDL-C therapeutic agent can be used concurrently, if desired. The therapy is designed to alter (e.g., inhibit or enhance), replace or supplement activity and/or function of a low HDL-C associated polypeptide or related metabolic pathway in an individual. For example, a low HDL-C therapeutic agent can be administered in order to upregulate or increase the expression or availability of a HDL-C associated gene or a specific variant of a HDL-C associated gene or, conversely, to downregulate or decrease the expression or availability of a HDL-C associated gene or a specific variant of a HDL-C associated gene. Upregulation or increasing expression or availability of a native HDL-C associated gene or a particular variant of a HDL-C associated gene could interfere with or compensate for the expression or activity of a defective gene or variant; downregulation or decreasing expression or availability of a native HDL-C associated gene or a particular splicing variant of a HDL-C associated gene could minimize the expression or activity of a defective gene or the particular variant and thereby minimize the impact of the defective gene or the particular variant.

The HDL-C increasing agent(s) are administered in a therapeutically effective amount (i.e., an amount that is sufficient to treat the low HDL-C trait or condition, such as by ameliorating symptoms associated with the low HDL-C trait or condition, preventing or delaying the onset of the low HDL-C trait or condition, and/or also lessening the severity or frequency of symptoms of the low HDL-C trait or condition). The amount which will be therapeutically effective in the treatment of a particular individual's disorder or condition will depend on the symptoms and severity of the low HDL-C trait or condition, and can be determined by standard clinical techniques. In addition, in vitro or in vivo assays may optionally be employed to help identify optimal dosage ranges. The precise dose to be employed in the formulation will also depend on the route of administration, and the seriousness of the trait or condition, and should be decided according to the judgment of a practitioner and each patient's circumstances. Effective doses may be extrapolated from dose-response curves derived from in vitro or animal model test systems.

In one embodiment, a nucleic acid of the invention (e.g., a nucleic acid encoding a HDL-C associated polypeptide, fragment, variant or derivative thereof), either by itself or included within a vector, can be introduced into cells of an individual affected by a low HDL-C using variety of experimental methods described in the art, so that the treated cells start to produce native HDL-C associated polypeptide. Thus, cells which, in nature, lack of a native HDL-C associated gene expression and activity, or have abnormal HDL-C associated gene expression and activity, can be engineered to express same HDL-C associated polypeptide or an active fragment or a different variant of said HDL-C associated polypeptide. Genetic engineering of cells may be done either “ex vivo” (i.e. suitable cells are isolated and purified from a patient and re-infused back to the patient after genetic engineering) or “in vivo” (i.e. genetic engineering is done directly to a tissue of a patient using a vehicle).

Alternatively, in another embodiment of the invention, a nucleic acid of the invention; a nucleic acid complementary to a nucleic acid of the invention; or a portion of such a nucleic acid (e.g., a polynucleotide), can be used in “antisense” therapy, in which a nucleic acid (e.g., a polynucleotide) which specifically hybridizes to the mRNA and/or genomic DNA of a HDL-C associated gene is administered in a pharmaceutical composition to the target cells or said nucleic acid is generated “in vivo”. The antisense nucleic acid that specifically hybridizes to the mRNA and/or DNA inhibits expression of the HDL-C associated polypeptide, e.g., by inhibiting translation and/or transcription. Binding of the antisense nucleic acid can be due to conventional base pairing, or, for example, in the case of binding to DNA duplexes, through specific interaction in the major groove of the double helix.

In a preferred embodiment nucleic acid therapeutic agents of the invention are delivered into cells that express a low HDL-C associated gene. A number of methods including, but not limited to, the methods known in the art can be used for delivering a nucleic acid to said cells. For example, a vector can be introduced in vivo such that it is taken up by a cell and directs the transcription of a RNA molecule, which induces RNA interference in the cell. Such a vector can remain episomal or become chromosomally integrated, and as long as it can be transcribed to produce the desired RNA molecules it will modify the expression of a HDL-C associated gene. Such vectors can be constructed by various recombinant DNA technology methods standard in the art.

The expression of an endogenous HDL-C associated gene can be also reduced by inactivating or “knocking out” a HDL-C associated gene or its promoter using targeted homologous recombination methods described in the art. Alternatively, expression of a functional, non-mutant HDL-C associated gene can be increased using a similar method: targeted homologous recombination can be used to replace a non-functional HDL-C associated gene with a functional form of the said gene in a cell.

In yet another embodiment of the invention, other low HDL-C therapeutic agents as described herein can also be used in the treatment or prevention of low HDL-C trait or condition. The therapeutic agents can be delivered in a pharmaceutical composition they can be administered systemically, or can be targeted to a particular tissue. The therapeutic agents can be produced by a variety of means, including chemical synthesis, cell culture and recombinant techniques (e.g. with transgenic cells and animals). Therapeutic agents can be isolated and purified to fulfil pharmaceutical requirements using standard methods described in the art.

A combination of any of the above methods of treatment (e.g., administration of non-mutant HDL-C associated polypeptide in conjunction with RNA molecules inducing RNA interference targeted to the mutant HDL-C associated mRNA) can also be used.

In the case of pharmaceutical therapy, the invention comprises compounds, which modulate the activity, function or concentration of one or more polypeptides encoded by HDL-C associated genes. The treatment may also enhance or reduce the expression of one or more genes selected from HDL-C associated genes set forth in tables 1, 8 and 9.

In another embodiment of the invention, pharmaceutical therapy of the invention comprises compounds, which enhance or reduce the activity and/or function of biological networks and/or metabolic pathways related to polypeptides encoded by HDL-C associated genes set forth in tables 1, 8 and 9. The treatment may also enhance or reduce the expression of one or several genes in biological networks and/or metabolic pathways related to said HDL-C associated genes set forth in tables 1, 8 and 9.

Furthermore, a disclosed method or a test based on HDL-C associated gene specific markers (e.g. polymorphic sites, expression or polypeptides) is useful in selecting drug therapy for patients with low HDL-C trait, and can be further used in the treatment of low HDL-C related diseases such as CVD, T2D, MBO and obesity. A gene test recognizing the low HDL-C associated allele homozygocity or carrier status of HDL-C associated genes set forth in tables 2 to 7 and 10 to 11 is useful in selecting prophylactic treatment for individuals having a high risk of a low HDL-C trait or condition.

Yet in another embodiment of the invention, a test or a method based on low HDL-C level associated gene specific biomarkers (e.g. polymorphic sites, expression products, polypeptides or metabolites) is useful in selecting subjects testing treatments for low HDL-C trait and/or conditions, such as CVD, T2D, MBO and obesity.

A test or a method of this invention based on low HDL-C level associated gene specific biomarkers (e.g. polymorphic sites, expression products, polypeptides or metabolites) is useful in selecting drug therapy for patients who might be at increased risk for adverse effects of drugs affecting HDL-C metabolism.

Pharmaceutical Compositions

The present invention also pertains to pharmaceutical compositions comprising agents described herein, particularly polynucleotides, polypeptides and any fractions, variants or derivatives of HDL-C associated genes set forth in tables 1, 8 and 9, and/or agents that alter (e.g., enhance or inhibit) expression of low HDL-C level associated gene or genes, or activity of one or more polypeptides encoded by HDL-C associated gene or genes as described herein. For instance, an agent that alters expression of HDL-C associated genes, or activity of one or more polypeptides encoded by low HDL-C associated genes or a low HDL-C associated polypeptide binding agent, binding partner, fragment, fusion protein or prodrug thereof, or polynucleotides of the present invention, can be formulated with a physiologically acceptable carrier or excipient to prepare a pharmaceutical composition. The carrier and composition can be sterile. The formulation should suit the mode of administration.

In a preferred embodiment pharmaceutical compositions comprise agent or agents reversing, at least partially, low HDL-C level associated changes in biological networks and/or metabolic pathways related to the HDL-C associated genes of this invention.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active agents.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrolidone, sodium saccharine, cellulose, magnesium carbonate, etc.

Methods of introduction of these compositions include, but are not limited to, intradermal, intramuscular, intraperitoneal, intraocular, intravenous, subcutaneous, topical, oral and intranasal. Other suitable methods of introduction can also include gene therapy (as described below), rechargeable or biodegradable devices, particle acceleration devises (“gene guns”) and slow release polymeric devices. The pharmaceutical compositions of this invention can also be administered as part of a combinatorial therapy with other agents.

The composition can be formulated in accordance with the routine procedures as a pharmaceutical composition adapted for administration to human beings. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., which are, if desired, sterilized or mixed with auxiliary agents, e.g., preservatives, stabilizers, wetting agents, buffers or salts for influencing osmotic pressure, etc. The agent may be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

Agents described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The agents are administered in a therapeutically effective amount. The amount of agents which will be therapeutically effective in the treatment of a particular disorder or condition will depend on the nature of the disorder or condition, and can be determined by standard clinical techniques. In addition, in vitro or in vivo assays may optionally be employed to help identify optimal dosage ranges. The precise dose to be employed in the formulation will also depend on the route of administration, and the seriousness of the symptoms of cardiovascular/metabolic disease, and should be decided according to the judgment of a practitioner and each patient's circumstances. Effective doses may be extrapolated from dose-response curves derived from in vitro or animal model test systems.

At-Risk Alleles and At-Risk Haplotypes

The genetic markers listed in tables 2 to 7 and 10 to 11 of this invention are particular “alleles” at “polymorphic sites” associated with low HDL-C (or low ApoA1). A nucleotide position, at which more than one sequence is possible in a population, is referred to herein as a “polymorphic site”. Where a polymorphic site is a single nucleotide in length, the site is referred to as a SNP. For example, if at a particular chromosomal location, one member of a population has an adenine and another member of the population has a thymine at the same position, then this position is a polymorphic site, and, more specifically, the polymorphic site is a SNP. Polymorphic sites may be several nucleotides in length due to insertions, deletions, conversions or translocations. Each version of the sequence with respect to the polymorphic site is referred to herein as an “allele” of the polymorphic site. Thus, in the previous example, the SNP allows for both an adenine allele and a thymine allele.

Typically, a reference nucleotide sequence is referred to for a particular gene. Alleles that differ from the reference are referred to as “variant” alleles. The polypeptide encoded by the reference nucleotide sequence is the “reference” polypeptide with a particular reference amino acid sequence, and polypeptides encoded by variant alleles are referred to as “variant” polypeptides with variant amino acid sequences.

Nucleotide sequence variants can result in changes affecting properties of a polypeptide. These sequence differences, when compared to a reference nucleotide sequence, include insertions, deletions, conversions and substitutions: e.g. an insertion, a deletion or a conversion may result in a frame shift generating an altered polypeptide; a substitution of at least one nucleotide may result in a premature stop codon, amino acid change or abnormal mRNA splicing; the deletion of several nucleotides, resulting in a deletion of one or more amino acids encoded by the nucleotides; the insertion of several nucleotides, such as by unequal recombination or gene conversion, resulting in an interruption of the coding sequence of a reading frame; duplication of all or a part of a sequence; transposition; or a rearrangement of a nucleotide sequence, as described in detail above. Such sequence changes alter the polypeptide encoded by a low HDL-C level associated gene described in this invention. For example, a nucleotide change resulting in a change in polypeptide sequence can alter the physiological properties of a polypeptide dramatically by resulting in altered activity, distribution and stability or otherwise affect on properties of a polypeptide.

Alternatively, nucleotide sequence variants can result in changes affecting transcription of a gene or translation of its mRNA. A polymorphic site located in a regulatory region of a gene may result in altered transcription of a gene e.g. due to altered tissue specificity, altered transcription rate or altered response to transcription factors. A polymorphic site located in a region corresponding to the mRNA of a gene may result in altered translation of the mRNA e.g. by inducing stable secondary structures to the mRNA and affecting the stability of the mRNA. Such sequence changes may alter the expression of a low HDL-C level associated gene of this invention.

A “haplotype”, as described herein, refers to any combination of genetic markers (“alleles”), such as those set forth in tables 4 and 7. A haplotype can comprise two or more alleles.

As it is recognized by those skilled in the art the same haplotype can be described differently by determining alleles from different strands e.g. the haplotype rs1872393, rs779744, rs779742, and rs3804900 (A A C C) is the same as haplotype rs1872393, rs779744, rs779742, and rs3804900 (T T G G) in which the alleles are determined from the other strand or haplotype rs1872393, rs779744, rs779742, and rs3804900 (T A C C), in which the first allele is determined from the other strand.

It is understood that the low HDL-C level associated alleles and haplotypes described in this invention may be associated with other “polymorphic sites” located in HDL-C associated genes of this invention. These other HDL-C associated polymorphic sites may be either equally useful as genetic markers or even more useful as causative variations explaining the observed association of at-risk alleles and at-risk haplotypes of this invention to low HDL-C.

In certain methods described herein, an individual who is at risk for low HDL-C is an individual in whom an at-risk allele or an at-risk haplotype is identified. In one embodiment, the at-risk allele or the at-risk haplotype is one that confers a significant risk of low HDL-C. In one embodiment, significance associated with an allele or a haplotype is measured by an odds ratio. In a further embodiment, the significance is measured by a percentage. In one embodiment, a significant risk is measured as odds ratio of at least about 1.2, including by not limited to: 0.1, 0.2, 0.3, 0.4, 0.5, 0.6, 0.7, 0.8, 0.9, 1.2, 1.3, 1.4, 1.5, 1.6, 1.7, 1.8, 1.9, 2.0, 2.5, 3.0, 4.0, 5.0, 10.0, 15.0, 20.0, 25.0, 30.0 and 40.0. In a further embodiment, a significant increase or reduction in risk is at least about 20%, including but not limited to about 25%, 30%, 35%, 40%, 45%, 50%, 55%, 60%, 65%, 70%, 75%, 80%, 85%, 90%, 95% and 98%. In a further embodiment, a significant increase in risk is at least about 50%. It is understood however, that identifying whether a risk is medically significant may also depend on a variety of factors, including the specific disease, the allele or the haplotype, and often, environmental factors.

An at-risk haplotype in, or comprising portions of, the low HDL-C associated gene, is one where the haplotype is more frequently present in an individual at risk for low HDL-C (affected), compared to the frequency of its presence in a healthy individual (control), and wherein the presence of the haplotype is indicative of low HDL-C or susceptibility to low HDL-C.

Primers, Probes and Nucleic Acid Molecules

“Probes” or “primers” are oligonucleotides that hybridize in a base-specific manner to a complementary strand of nucleic acid molecules. By “base specific manner” is meant that the two sequences must have a degree of nucleotide complementarity sufficient for the primer or probe to hybridize. Accordingly, the primer or probe sequence is not required to be perfectly complementary to the sequence of the template. Non-complementary bases or modified bases can be interspersed into the primer or probe, provided that base substitutions do not inhibit hybridization. The nucleic acid template may also include “non-specific priming sequences” or “nonspecific sequences” to which the primer or probe has varying degrees of complementarity. Such probes and primers include polypeptide nucleic acids (Nielsen P E et al, 1991).

A probe or a primer comprises a region of nucleic acid that hybridizes to at least about 15, for example about 20-25, and in certain embodiments about 40, 50 or 75, consecutive nucleotides of a nucleic acid of the invention, such as a nucleic acid comprising a contiguous nucleic acid sequence.

In preferred embodiments, a probe or primer comprises 100 or fewer nucleotides, in certain embodiments, from 6 to 50 nucleotides, for example, from 12 to 30 nucleotides. In other embodiments, the probe or primer is at least 70% identical to the contiguous nucleic acid sequence or to the complement of the contiguous nucleotide sequence, for example, at least 80% identical, in certain embodiments at least 90% identical, and in other embodiments at least 95% identical, or even capable of selectively hybridizing to the contiguous nucleic acid sequence or to the complement of the contiguous nucleotide sequence. Often, the probe or primer further comprises a label, e.g., radioisotope, fluorescent compound, enzyme, or enzyme co-factor.

Antisense nucleic acid molecules of the invention can be designed using the nucleotide sequences of low HDL-C level associated genes and/or their complementary sequences and constructed using chemical synthesis and enzymatic ligation reactions using procedures known in the art. For example, an antisense nucleic acid molecule (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Alternatively, the antisense nucleic acid molecule can be produced biologically using an expression vector into which a nucleic acid molecule encoding a HDL-C related gene, a fragment or a variant thereof has been cloned in antisense orientation (i.e., RNA transcribed from the expression vector will be complementary to the transcribed RNA of a cardiovascular/metabolic diseases risk gene of interest).

Portions or fragments of the nucleotide sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Additionally, the nucleotide sequences of the invention can be used to identify and express recombinant polypeptides for analysis, characterization or therapeutic use, or as markers for tissues in which the corresponding polypeptide is expressed, either constitutively, during tissue differentiation, or in diseased states. The nucleic acid sequences can additionally be used as reagents in the screening and/or diagnostic assays described herein, and can also be included as components of kits (e.g., reagent kits) for use in the screening and/or diagnostic assays described herein.

Polyclonal and Monoclonal Antibodies

The invention comprises polyclonal and monoclonal antibodies that bind to polypeptides of the invention. The term “antibody” as used herein refers to immunoglobulin molecules and immunologically active portions of immunoglobulin molecules, i.e., molecules that contain a binding site that specifically binds to an epitope (antigen, antigenic determinant). An antibody molecule that specifically binds to a polypeptide of the invention is a molecule that binds to an epitope present in said polypeptide or a fragment thereof, but does not substantially bind other molecules in a sample, e.g., a biological sample, which naturally contains the polypeptide. Examples of immunologically active portions of immunoglobulin molecules include F(ab) and F(ab′).sub.2 fragments which can be generated by treating the antibody with an enzyme such as pepsin. Polyclonal and/or monoclonal antibodies that specifically bind one form of the gene product but not to the other form of the gene product are also provided. Antibodies are also provided, that bind a portion of either the variant or the reference gene product that contains the polymorphic site or sites. The term “monoclonal antibody” or “monoclonal antibody composition”, as used herein refers to a population of antibody molecules that are directed against a specific epitope and are produced either by a single clone of B cells or a single hybridoma cell line. A monoclonal antibody composition thus typically displays a single binding affinity for a particular polypeptide of the invention with which it immunoreacts.

Polyclonal antibodies can be prepared as known by those skilled in the art by immunizing a suitable subject with a desired immunogen, e.g., polypeptide of the invention or fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique (Kohler G and Milstein C, 1975), the human B cell hybridoma technique (Kozbor D, 1982), the EBV-hybridoma technique (Cole S P et al, 1984), or trioma techniques (Hering S et al, 1988). To produce a hybridoma an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cell lines can be applied for the purpose of generating a monoclonal antibody to a polypeptide of the invention (Bierer B et al, 2002). Moreover, the ordinarily skilled worker will appreciate that there are many variations of such methods that also would be useful. Alternative to preparing monoclonal antibody-secreting hybridomas, a monoclonal antibody to a polypeptide of the invention can be identified and isolated by screening a recombinant combinatorial immunoglobulin library (e.g., an antibody phage display library) with the polypeptide to thereby isolate immunoglobulin library members that bind the polypeptide (Hay B N 1992; Hayashi N et al, 1995; Griffiths A D et al, 1993; Huse W D et al, 1989). Kits for generating and screening phage display libraries are commercially available.

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

In general, antibodies of the invention (e.g., a monoclonal antibody) can be used to isolate a polypeptide of the invention by standard techniques, such as affinity chromatography or immunoprecipitation. An antibody specific for a polypeptide of the invention can facilitate the purification of a native polypeptide of the invention from biological materials, as well as the purification of recombinant form of a polypeptide of the invention from cultured cells (culture media or cells). Moreover, an antibody specific for a polypeptide of the invention can be used to detect the polypeptide (e.g., in a cellular lysate, cell supernatant, or tissue sample) in order to evaluate the abundance and pattern of expression of the polypeptide. Antibodies can be used diagnostically to monitor protein levels in tissue such as blood as part of a test predicting the susceptibility to cardiovascular/metabolic diseases or as part of a clinical testing procedure, e.g., to, for example, determine the efficacy of a given treatment regimen. Antibodies can be coupled to various enzymes, prosthetic groups, fluorescent materials, luminescent materials, bioluminescent materials, and radioactive materials to enhance detection. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include .sup.125I, 131I, 35S or 3H.

Diagnostic and Screening Assays

The probes, primers and antibodies described herein can be used in methods of selecting efficient and safe therapy for increasing HDL-C levels of a subject. For example biomarkers associated to the low HDL-C level associated genes of this invention can be assessed from a subject and therapy can be focused to genes having altered activity.

Determination of the nucleotides present in one or more of the low HDL-C associated SNP markers of this invention, as well as polymorphic sites associated with low HDL-C associated SNP markers of this invention, in an individual's nucleic acid can be done by any method or technique which can accurately determine nucleotides present in a polymorphic site. Numerous suitable methods have been described in the art (Kwok P Y, 2001; Syvanen A C, 2001), these methods include, but are not limited to, hybridization assays, ligation assays, primer extension assays, enzymatic cleavage assays, chemical cleavage assays and any combinations of these assays. The assays may or may not include PCR, solid phase step, a microarray, modified oligonucleotides, labeled probes or labeled nucleotides and the assay may be multiplex or singleplex. As it is obvious in the art the nucleotides present in a polymorphic site can be determined from either nucleic acid strand or from both strands.

Diagnostically the most useful polymorphic sites are those altering the polypeptide biological activity, function or concentration of a low HDL-C associated gene due to a frame shift; due to a premature stop codon, due to an amino acid change or due to abnormal mRNA splicing. Nucleotide changes resulting in a change in polypeptide sequence in many cases alter the physiological properties of a polypeptide by resulting in altered activity, distribution and stability or otherwise affect on properties of a polypeptide. Other diagnostically useful polymorphic sites are those affecting transcription of a low HDL-C associated genes or translation of it's mRNA due to altered tissue specificity, due to altered transcription rate, due to altered response to physiological status, due to altered translation efficiency of the mRNA and due to altered stability of the mRNA. Alterations in transcription can be assessed by a variety of methods described in the art, including e.g. hybridization methods, enzymatic cleavage assays, RT-PCR assays and microarrays. A test sample from an individual is collected and the alterations in the transcription of low HDL-C associated genes are assessed from the RNA present in the sample.

A test sample from an individual may be assessed for presence of alterations in the biological activity, function, concentration and/or structure of polypeptides encoded by low HDL-C associated genes set forth in tables 1, 8 and 9 by various methods known in the art e.g. by assays based on chromatography, spectroscopy, colorimetry, electrophoresis, isoelectric focusing, specific cleavage, immunologic techniques and measurement of biological activity as well as combinations of different assays. An “alteration” as used herein, refers to an alteration in expression or composition of a polypeptide of the test sample, as compared with the expression or composition in a control sample. A control sample is a sample that corresponds to the test sample (e.g., is from the same type of cells), and is from an individual who is not affected by low HDL-C.

Western blotting analysis, using an antibody as described above that specifically binds to a polypeptide encoded by a mutant HDL-C associated gene or an antibody that specifically binds to a polypeptide encoded by a non-mutant gene, or an antibody that specifically binds to a particular splicing variant encoded by a HDL-C associated gene can be used to identify the presence or absence in a test sample of a particular polypeptide encoded by a polymorphic or mutant HDL-C associated gene.

Methods of selecting efficient and safe therapy of this invention may further comprise a step of combining information concerning age, gender, the family history of low HDL-C, as well as CVD, diabetes and hypercholesterolemia or/and a medical history for those, and the medical history concerning HDL-C, smoking status, and waist-to-hip circumference ratio (cm/cm) of the subject. The detection method of the invention may also further comprise a step determining blood, serum or plasma cholesterol, HDL cholesterol, LDL cholesterol, triglyceride, ApoA1 and apolipoprotein B, fibrinogen, ferritin, transferrin receptor, C-reactive protein, serum or plasma insulin concentration.

### EXAMPLE 1

### KIHD Cohort Genotyping Study

Study Design

This invention is based on whole-genome association study approach, in which distributions or means of the phenotypic measurement (HDL and ApoAI) are compared across genotypes or patterns of genetic markers. The study subjects were a subset of a population-based study in East Finland, the KIHD (Salonen 1988). This work is based on 246 male participants in the KIHD study.

Study Population

The subjects were participants of the Kuopio Ischaemic Heart Disease Risk Factor Study (KIHD), which is an ongoing prospective population-based study designed to investigate risk factors for chronic diseases, including AMI, CHD, HT, stroke, T2D, MBO and obesity, among middle-aged men from East Finland. The study population was a random age-stratified sample of men living in Eastern Finland who were 42, 48, 54 or 60 years old at baseline examinations in 1984-1989. A total of 2682 men were examined in the baseline examinations during 1984-89. Data used here concerning serum HDL and apolipoprotein Al concentrations are from measurements at this baseline examination. The recruitment and examination of the subjects has been described previously in detail (Salonen J T 1988, WO02074230, WO03089638). The University of Kuopio and Kuopio University Hospital Ethics Committee approved the study. All participants gave their written informed consent. For this study, 246 male KIHD baseline participants and four female KIHD 11-year examination participants were selected.

The Measurement of HDL and Apolipoprotein AI

HDL fractions were separated from fresh serum by combined ultracentrifugation and precipitation (Salonen et al 1991, W003052129). The cholesterol contents of lipoprotein fractions and serum triglycerides were measured enzymatically. The mean HDL-C was 1.29 mmol/L, minimum 0.76 mmol/L, maximum 2.77 mmol/L and standard deviation 0.30 mmol/L.

Serum apolipoprotein AI concentrations were measured for 241 male subjects as described previously (Salonen et al 1992). The mean ApoAI was 1.33 mg/L, minimum 0.85 mg/L, maximum 2.50 mg/L and the SD 0.25 mg/L.

Genomic DNA Isolation and Quality Testing

High molecular weight genomic DNA samples were extracted from frozen venous whole blood using standard methods and dissolved in standard TE buffer. The quantity and purity of each DNA sample was evaluated by measuring the absorbance at 260 and 280 nm and integrity of isolated DNA samples were evaluated with 0,9% agarose gel electrophoresis and Ethidiumbromide staining. A sample was qualified for genome wide scan (GWS) analysis if A260/A280 ratio was ≧1.7 and average size of isolated DNA was over 20 kb in agarose gel electrophoresis. Before GWS analysis samples were diluted to concentration of 50 ng/μl in reduced EDTA TE buffer (TEKnova).

Genome-Wide Scan

Genotyping of SNP markers was performed by using the technology access version of Affymetrix GeneChip® human mapping 100k system. The assay consisted of two arrays, Xba and Hind, which were used to genotype over 126,000 SNP markers from each DNA sample. The assays were performed according to the instructions provided by the manufacturer. A total of 250 ng of genomic DNA was used for each individual assay. DNA sample was digested with either Xba I or Hind III enzyme (New England Biolabs, NEB) in the mixture of NE Buffer 2 (1×; NEB), bovine serum albumin (1×; NEB), and either Xba I or Hind III (0,5 U/μl; NEB) for 2 h at +37° C. followed by enzyme inactivation for 20 min at +70° C. Xba I or Hind III adapters were then ligated to the digested DNA samples by adding Xba or Hind III adapter (0,25 μM, Affymetrix), T4 DNA ligase buffer (1×; NEB), and T4 DNA ligase (250 U; NEB). Ligation reactions were allowed to proceed for 2 h at +16° C. followed by 20 min incubation at +70° C. Each ligated DNA sample was diluted with 75 μl of molecular biology-grade water (BioWhittaker Molecular Applications/Cambrex).

Diluted ligated DNA samples were subjected to four identical 100 μl volume polymerase chain reactions (PCR) by implementing a 10 μl aliquot of DNA sample with Pfx Amplification Buffer (1×; Invitrogen), PCR Enhancer (1×; Invitrogen), MgSO₄ (1 mM; Invitrogen), dNTP (300 μM each; Takara), PCR primer (1 μM; Affymetrix), and Pfx Polymerase (0,05 U/μl; Invitrogen). The PCR was allowed to proceed for 3 min at +94° C., followed by 30 cycles of 15 sec at +94° C., 30 sec at +60° C., 60 sec at +68° C., and finally for the final extension for 7 min at +68° C. The performance of the PCR was checked by standard 2% agarose gel electrophoresis in 1×TBE buffer for 1 h at 120V.

PCR products were purified according to Affymetrix manual using MinElute 96 UF PCR Purification kit (Qiagen) by combining all four PCR products of an individual sample into same purification reaction. The purified PCR products were eluted with 40 μl of EB buffer (Qiagen), and the yields of the products were measured at the absorbance 260 nm. A total of 40 μg of each PCR product was then subjected to fragmentation reaction consisting of 0,2 U/μl fragmentation reagent (Affymetrix) in 1×Fragmentation Buffer. Fragmentation reaction was allowed to proceed for 35 min at +37° C. followed by 15 min incubation at +95° C. for enzyme inactivation. Completeness of fragmentation was checked by running an aliquot of each fragmented PCR product in 4% agarose 1×TBE (BMA Reliant precast) for 30-45 min at 120V.

Fragmented PCR products were then labeled using 1×Terminal Deoxinucleotidyl Transferase (TdT) buffer (Affymetrix), GeneChip DNA Labeling Reagent (0,214 mM; Affymetrix), and TdT (1,5 U/μl; Affymetrix) for 2 h at +37° C. followed by 15 min at +95° C. Labeled DNA samples were combined with hybridization buffer consisting of 0,056 M MES solution (Sigma), 5% DMSO (Sigma), 2,5×Denhardt's solution (Sigma), 5,77 mM EDTA (Ambion), 0,115 mg/ml Herring Sperm DNA (Promega), 1×Oligonucleotide Control reagent (Affymetrix), 11,5 μg/ml Human Cot-1 (Invitrogen), 0,0115% Tween-20 (Pierce), and 2,69 M Tetramethyl Ammonium Chloride (Sigma). DNA-hybridization buffer mix was denatured for 10 min at +95° C., cooled on ice for 10 sec and incubated for 2 min at +48° C. prior to hybridization onto corresponding Xba or Hind GeneChip® array. Hybridization was completed at +48° C. for 16-18 h at 60 rpm in an Affymetrix GeneChip Hybridization Oven. Following hybridization, the arrays were stained and washed in GeneChip Fluidics Station 450 according to fluidics station protocol Mapping10Kv1_{—}450 as recommended by the manufacturer. Arrays were scanned with GeneChip 3000 Scanner and the genotype calls for each of the SNP markers on the array were generated using Affymetrix Genotyping Tools (GTT) software. The confidence score in SNP calling algorithm was adjusted to 0.20.

Initial SNP Selection for Statistical Analysis

Prior to the statistical analysis, SNP quality was assessed on the basis of three values: the call rate (CR), minor allele frequency (MAF), and Hardy-Weinberg equilibrium (H-W). The CR is the proportion of samples with successful genotyping result. It does not take into account whether the genotypes are correct or not. The call rate was calculated as: CR=number of samples with successful genotype call/total number of samples. The MAF is the frequency of the allele that is less frequent in the study sample. MAF was calculated as: MAF=min(p, q), where p is frequency of the SNP allele ‘A’ and q is frequency of the SNP allele ‘B’; p=(number of samples with “AA”-genotype+0.5*number of samples with “AB”-genotype)/total number of samples with successful genotype call; q=1−p. SNPs that are homozygous (MAF=0) can not be used in genetic analysis and were thus discarded. H-W equilibrium is tested for controls. The test is based on the standard Chi-square test of goodness of fit. The observed genotype distribution is compared with the expected genotype distribution under H-W equilibrium. For two alleles this distribution is p2, 2pq, and q2 for genotypes ‘AA’, ‘AB’ and ‘BB’, respectively. If the SNP is not in H-W equilibrium it can be due to genotyping error or some unknown population dynamics (e.g. random drift, selection). Only the SNPs that had CR>50%, MAF>1%, and were in H-W equilibrium (Chi-square test statistic<23.93) were used in the statistical analysis.

Statistical Methods

Single SNP Analysis

For each SNP, differences in the mean HDL-C and ApoA1 levels for two allele groups were tested with t-test. Similarly differences in the mean HDL-C and ApoA1 levels for each genotype group were tested with one-way analysis of variance (ANOVA, F-test). For t-test either equal (ttest1) or unequal (ttest2) variances were assumed. Tests that gave P-value less than 0.005 were considered statistically significant.

Haplotype Region Analysis: HaploRec+HPMQ

The data set was analyzed with a haplotype pattern mining algorithm HPMQ software that is based on the HPM software (Toivonen H T et al, 2000). For HPMQ software genotypes must have phase known i.e. to determine which alleles are coming from the mother and which from the father. Without family data phases must be estimated based on population data. We used the HaploRec-program (Eronen L et al, 2004) to estimate the phases. HPMQ is very fast and can handle a large number of SNPs in a single run

For phase-known data HPMQ finds all haplotype patterns that are in concordance with the phase configuration. The length of the haplotype patterns can vary. As an example, if there are four SNPs and an individual has alleles A T for the SNP1, C C for the SNP2, C G for the SNP3, and A C for the SNP4 then HPMQ considers haplotype patterns that are in concordance with estimated phase (done by HaploRec). If the estimated phase is ACGA (from the mother/father) and TCCC (from the father/mother) then HPMQ considers two patterns (of length 4 SNPs): ACGA and TCCC. For each haplotype pattern, a Z-test statistic is computed based on the difference in the mean value of a continuous trait between a group that has the haplotype pattern and the group that does not have the haplotype pattern. A SNP is scored based on the number of times it is included in a haplotype pattern that passes the threshold value set for the Z-test. Significance of the score values is tested based on permutation tests.

Several parameters can be modified in the HPMQ program including the Z-test threshold value (-x), the maximum haplotype pattern length (-l), the maximum number of wildcards that can be included in a haplotype pattern (-w), and the number of permutation test in order to estimate the P-value (-p). Wildcards allow gaps in haplotypes. HPMQ was run with the following parameter settings: haplotype analysis with 5 SNPs (-x4-l5-w1-p10000). Haplotype genomic regions that gave P-value less than 0.005 were considered statistically significant.

Multivariate Analyses

Partial associations of SNPs with HDL-C and ApoAI, adjusted for all independent variables entering the model, were estimated by using the least squares regression analysis. SPSS for Windows 13.0 software was used.

Definition of Terms used in the Haplotype Analysis Results

The term “haplotype genomic region” or “haplotype region” refers to a genomic region that has been found significant in the haplotype analysis (HPMQ or similar statistical method/program). The haplotype region in this patent is defined as a sub-region of the pre-selected genomic region where for any SNP the permutated P-value is less or equal than 0.005.

Findings of the KIHD Cohort Study

Tables 2, 3 and 4 show the SNP markers with the strongest association with serum HDL-C concentration. SNP physical position is according to NCBI Human Genome Build 35.1. Gene locus is as reported by NCBI dbSNP database build 124.

Table 2 presents results from t-tests, in which equal variances between groups are assumed and p value is less than 0.005. The genes with intragenic markers with the strongest associations with serum HDL-C were ANGPT1, EFHA1, UNC13C, TULP4, ARFRP2, FLJ10099, CNNM2, DOK5L, SGCG, SNAP25, ZFPM2, SERPINA5, 13CDNA73, PHACTR1, NT5C2, DGKB, LOC283553, LTBP1, and MSR1.

In table 3 results from F-test (one-way ANOVA test) are summarized. P value of less than 0.005 is the significance limit.

Table 4 presents the most significant haplotype regions associated with HDL-C level based on HaploRec+HPMQ analysis. The strongest genes with an association with HDL-C P of less than 0.0005 were ANGPT1, HNRPD, LOC391672, CNGB3, MAPK8, LOC399763, LOC442115, GRIM1, ABCD3 AND SGCG.

Tables 5, 6, and 7 present corresponding results for SNP markers with the strongest association with serum ApoA1 levels. SNP physical position is according to NCBI Human Genome Build 35.1. Gene locus is as reported by NCBI dbSNP database build 124.

Table 5 presents results from t-tests, in which equal variances between groups are assumed and p value is less than 0.005.

In table 6 results from F-test (one-way ANOVA test) are summarized. P value of less than 0.005 is the significance limit.

Table 7 shows the most significant haplotype regions for ApoA1 based on HaploRec+HPM analysis.

Table 8 lists all genes, which were associated with HDL-C level in the pointwise and haplotype analyses (Tables 2, 3 and 4). Gene names are according to HUGO Gene Nomenclature Committee (HGNC).

Table 9 lists all genes, which were associated with ApoA1 level in pointwise and haplotype analyses (Tables 5, 6 and 7). Gene names are according to HUGO Gene Nomenclature Committee (HGNC).

Table 10 shows a linear regression model of the best HDL-C level predictive SNPs and genes.

Table 11 presents a linear regression model of the best ApoA1 level predictive SNPs and genes.

### EXAMPLE 2

### Low HDL-C Gene Replication Study

The replication study was based on HDL-C and genotype data of Jurilab's type 2 diabetes studies (SOHFA, GEDINO and DiaGen studies).

East Finnish (EF) Study Subjects

The study subjects (201 T2D cases and 200 healthy T2D-free controls) were participants of the SOHFA and GEDINO studies. SOHFA is a contractual study, in which the University of Kuopio is the contractee. “GEDINO” (Genetics of type 2 diabetes in North Savo) is a similar contractual project, in which the T2D cases and controls were collected by using a newspaper advertisement.

The cases had T2D and family history of T2D. All T2D cases (probands) had at least one additional affected relative, who was a parent, sibling or offspring of the proband. Most of them had more than one additional affected family member. The controls had neither T2D nor family history of T2D. The fasting blood glucose of the controls was 5.5 mmol/L or less and the glycated hemoglobin 5.5% or less.

Age and tobacco smoking were recorded on a self-administered questionnaire checked by an interviewer. HDL fractions were separated from fresh serum by combined ultracentrifugation and precipitation. The cholesterol contents of lipoprotein fractions and serum triglycerides were measured enzymatically. Both systolic and diastolic BPs were measured in the morning by a nurse with a mercury sphygmomanometer. The measuring protocol included three measurements in standing position with 5-minute intervals. The mean of all three measurements were used as SBP and DBP. Body mass index (BM) was computed as the ratio of weight to the square of height (kg/m²). Waist-to-hip ratio (WHR) was calculated as the ratio of waist circumference (average of one measure taken after inspiration and one taken after expiration at the midpoint between the lowest rib and the iliac crest) to hip circumference (measured at the level of the trochanter major).

The mean age of the cases was 64 years and that of the controls 67 years. Some cases had very low blood glucose, since they had hypoglycemic medication. In spite of this, the average blood glucose and glycated hemoglobin of the cases were higher than that of the controls. Since there was no matching according to obesity, the cases were on the average more obese than the controls.

Ashkenazi Jewish (AJ) Study Subjects

Subjects included in the study were collected in Israel by the physicians in charge in specialized clinics. Subjects were diagnosed with Type II Diabetes Mellitus according to the etiologic classification of Diabetes Mellitus proposed by the International Expert Committee under the sponsorship of the American Diabetes Association on May 1997. We included in the study 200 subjects (82 males and 118 females, mean age 64), each with 3 or more blood relatives of second degree or closer, suffering from T2D.

Matching 200 healthy control subjects (82 males and 118 females, mean age 74) were collected from the Israeli blood bank and elderly patients visiting general practitioners clinics. All subjects were of Ashkenazi Jewish origin. The study was approved by the appropriate ethics committees and participants had signed informed consent forms.

German (GE) and English (UK) Study Subjects

In Germany, cases were sampled from T2D patients from the Hospital of Diabetes and Metabolic Diseases (Karlsburg, Germany) and the diabetes dispensary unit of the Department of Endocrinology of the Ernst-Moritz-Arndt University (Greifswald, Germany). The controls were sampled from the non-diabetic examinees of the population based SHIP study cohort (Luedemann et al 2002). Total of 49 cases (24 females and 25 males) and 50 matched healthy controls (24 females and 26 males) from Germany were included in the study.

From England total of 50 cases (31 females and 19 males) and 50 matched healthy controls (31 females and 19 males) were included in the study. The controls were selected from the examinees of the Age and Cognitive Performance Research Centres (ACPRC) volunteer panel, a group of over 6000 older adults who have been previously described in detail (Rabbitt et al, 2004). A cohort of approximately 2000 of these individuals has DNA archived in the Dyne-Steel DNA bank. A group of 456 of these volunteers, residents of Greater Manchester, had previously taken part in a research study in 2001 which included medical history, including that of Diabetes Mellitus, and measurement of HbA_{1C}. From the original cohort of 456, a sample of 50 individuals was identified to sex match diabetic cases from Manchester. Each individual had an HbA_{1C} below 5.5% and at telephone interview of family diabetes mellitus history in 2006, reported no evidence of diabetes mellitus in parents or siblings. The University of Manchester research ethics committee approved the study and each individual completed an individual form of consent.

Study Subjects used to Replicate Low HDL-C Level Associated Genes

The replication was based on combined data set with 401 participants from the East Finland population, 98 participants from the German population and 85 participants from the UK population and using HDL as a quantitative trait. In addition to quantitative trait analysis, HDL was also categorized into two classes: normal: HDL>1.55 mmol/l and low: HDL<0.9 mmol/l. The combined data set of 292 participants included 145 participants from EF, 56 participants from Ashkenazi Jew population from Israel, 50 participants from GE, and 41 participants from UK.

Genotyping with Illumina's Sentrix HumanHap300

DNA isolation of cases and controls were done as described in example 1. The whole-genome genotyping of the DNA samples was performed by using Illumina's Sentrix HumanHap300 BeadChips and Infinium II genotyping assay. The HumanHap300 BeadChip contained over 317,000 tag SNPs markers derived from the International HapMap Project. TagSNPs are loci that can serve as proxies for many other SNPs. The use of tagSNPs greatly improves the power of association studies as only a subset of loci needs to be genotyped while maintaining the same information and power as if one had genotyped a larger number of SNPs.

The Infinium II genotyping with the HumanHap300 BeadChipassays was performed according to the “Single-Sample BeadChip Manual process” described in detail in “Infinium™ II Assay System Manual” provided by Illumina (San Diego, Calif., USA). Briefly, 750 ng of genomic DNA from a sample was subjected to whole-genome amplification. The amplified DNA was fragmented, precipitated and resuspended to hybridization buffer. The resuspended sample was heat denatured and then applied to one Sentrix HumanHap300 beadchip. After overnight hybridization mis- and non-hybridized DNA was washed away from the BeadChip and allele-specific single-base extension of the oligos on the BeadChip was performed in a Tecan GenePaint rack, using labeled deoxynucleotides and the captured DNA as a template. After staining of the extended DNA, the BeadChips were washed and scanned with the BeadArray Reader (Illumina) and genotypes from samples were called by using the BeadStudio software (Illumina).

Statistical Analyses

HDL as a quantitative trait was analysed from combined data set of 401 participants from the East Finland population, 98 participants from the German population and 85 participants from the UK population. The data set was analyzed with the R-programming language using a linear model of lm(z˜w+r+t+P), where for one individual z is a HDL measurement, w is a genotype, r is a T2D status, t is a gender, and P is indicating the population the individual is originating (either 1=EF, 2=AJ, 3=GE, 4=UK). Three different genotypic models were tested: an additive model where w can have three values e.g. 0=AA, 1=AG and 2=GG; a dominance model where w can have two values e.g. 0=AA, 1=AG or GG; and a recessive model where w can have two values e.g. 0=AA or AG and 1=GG.

In addition to quantitative trait analysis, HDL was also categorized into two classes: normal: HDL>1.55 mmol/l and low: HDL<0.9 mmol/l. The combined data set of 292 participants included 145 participants from EF, 56 participants from Ashkenazi Jew population (AJ) from Israel, 50 participants from GE, and 41 participants from UK. The statistical model used in the R-programming language was glm(z˜w+r+t+P,family=binomial(link=logit)), where z, w, r, t, and P are as above.

Replicating HDL-C Associated Genes

The HDL-C level associated genes which were discovered in Example 1 (fisted in Table 8.) and which replicated in combined SOHFA, GEDINO and DiaGen study data set are presented in table 1.

TABLE 1
List of genes that were first found with a data set of
246 male participants in the KIHD study and replicated with a
combined data set of 401 participants from the East Finland (EF)
population, 98 participants from the German (GE) population and
85 participants from the UK (UK) population and using HDL as a
quantitative trait or using a classified trait (class 1: HDL > 1.55
mmol/l, class 2: HDL < 0.9 mmol/l) and a combined data set of
292 paticipants: EF (145 participants), Ashkenazi Jews from Israel
(56 participants), GE (50 participants), and UK (41 participants).

| Gene | Gene_id | Chr | P-value1 | P-value2 |
|---|---|---|---|---|
| DLG2 | 1740 | 11 | 7.85E−04 | 7.39E−06 |
| SLC9A9 | 285195 | 3 | 3.45E−03 | 9.32E−06 |
| LRRN6C | 158038 | 9 | 3.36E−03 | 2.60E−05 |
| AMPH | 273 | 7 | 1.48E−03 | 5.15E−05 |
| CACNA1E | 777 | 1 | 7.09E−04 | 5.87E−05 |
| BCL2 | 596 | 18 | 2.06E−03 | 6.01E−05 |
| FMN2 | 56776 | 1 | 1.32E−03 | 7.80E−05 |
| GALNTL4 | 374378 | 11 | 1.38E−02 | 9.13E−05 |
| CSMD1 | 64478 | 8 | 2.07E−03 | 1.10E−04 |
| DMD | 1756 | X | 2.91E−04 | 1.18E−04 |
| PRKG1 | 5592 | 10 | 6.15E−04 | 1.56E−04 |
| HSPC117 | 51493 | 22 | 3.02E−03 | 1.57E−04 |
| AAA1 | 404744 | 7 | 1.41E−03 | 1.86E−04 |
| TEC | 7006 | 4 | 3.55E−03 | 2.21E−04 |
| CDH13 | 1012 | 16 | 4.17E−03 | 2.33E−04 |
| WWOX | 51741 | 16 | 4.03E−03 | 2.41E−04 |
| ANK3 | 288 | 10 | 1.15E−03 | 2.43E−04 |
| TMEFF2 | 23671 | 2 | 3.76E−03 | 2.60E−04 |
| TMCC2 | 9911 | 1 | 1.41E−02 | 2.76E−04 |
| CUBN | 8029 | 10 | 8.77E−04 | 2.77E−04 |
| KIAA0774 | 23281 | 13 | 4.74E−03 | 3.05E−04 |
| RAPGEF4 | 11069 | 2 | 1.85E−03 | 3.70E−04 |
| PTPRT | 11122 | 20 | 1.69E−03 | 3.85E−04 |
| DGKB | 1607 | 7 | 1.73E−03 | 4.49E−04 |
| POU6F2 | 11281 | 7 | 2.44E−03 | 4.70E−04 |
| GRM7 | 2917 | 3 | 3.81E−03 | 4.77E−04 |
| NAV3 | 89795 | 12 | 4.28E−03 | 4.79E−04 |
| C12orf26 | 84190 | 12 | 1.40E−03 | 4.92E−04 |
| FSTL4 | 23105 | 5 | 3.58E−03 | 5.00E−04 |
| MAGI2 | 9863 | 7 | 1.91E−06 | 5.42E−04 |
| LRP1B | 53353 | 2 | 1.25E−03 | 5.62E−04 |
| CNTN5 | 53942 | 11 | 1.91E−03 | 5.76E−04 |
| RORA | 6095 | 15 | 5.06E−04 | 6.04E−04 |
| NAALADL2 | 254827 | 3 | 4.08E−04 | 6.64E−04 |
| VGCNL1 | 259232 | 13 | 7.53E−04 | 7.22E−04 |
| NTN1 | 9423 | 17 | 3.31E−04 | 7.49E−04 |
| PAPPA2 | 60676 | 1 | 3.78E−03 | 7.84E−04 |
| NFIA | 4774 | 1 | 2.80E−03 | 7.89E−04 |
| COL25A1 | 84570 | 4 | 1.73E−03 | 7.90E−04 |
| C10orf107 | 219621 | 10 | 1.11E−03 | 8.01E−04 |
| NTRK2 | 4915 | 9 | 8.20E−03 | 8.42E−04 |
| KCNQ5 | 56479 | 6 | 2.20E−03 | 8.80E−04 |
| ARL15 | 54622 | 5 | 3.45E−05 | 9.40E−04 |
| TCBA1 | 154215 | 6 | 4.05E−03 | 9.59E−04 |
| FLJ33708 | 285780 | 6 | 4.08E−06 | Flanking |
| GALC | 2581 | 14 | 3.17E−05 | Flanking |
| LOC644954 | 644954 | 10 | 6.68E−05 | Flanking |
| SPATA4 | 132851 | 4 | 3.52E−04 | Flanking |
| APLP2 | 334 | 11 | 7.24E−04 | Flanking |
| E2F2 | 1870 | 1 | 8.90E−04 | Flanking |
| LOC653748 | 653748 | 7 | 9.00E−04 | Flanking |
| DKFZP566M1046 | 84067 | 11 | 1.16E−03 | Flanking |
| C10orf112 | 340895 | 10 | 1.21E−03 | Flanking |
| GPC6 | 10082 | 13 | 1.67E−03 | Flanking |
| PLEKHA7 | 144100 | 11 | 1.85E−03 | Flanking |
| RIPK5 | 25778 | 1 | 2.00E−03 | Flanking |
| CRSP8 | 9442 | 9 | 2.19E−03 | Flanking |
| LOC646697 | 646697 | 4 | 2.19E−03 | Flanking |
| DTNA | 1837 | 18 | 3.02E−03 | Flanking |
| C20orf133 | 140733 | 20 | 3.44E−03 | Flanking |
| KIAA0746 | 23231 | 4 | 3.55E−03 | Flanking |
| ZNF659 | 79750 | 3 | 3.77E−03 | Flanking |
| DDAH1 | 23576 | 1 | 4.02E−03 | Flanking |
| SYNE1 | 23345 | 6 | 4.05E−03 | Flanking |
| FBXO33 | 254170 | 14 | 4.40E−03 | Flanking |
| RGS3 | 5998 | 9 | 4.42E−03 | Flanking |
| PALLD | 23022 | 4 | 4.53E−03 | Flanking |
| ZBTB20 | 26137 | 3 | 4.98E−03 | Flanking |
| ROR1 | 4919 | 1 | 5.32E−03 | Flanking |

Flanking: gene is 100kb from the snp that has a P-value < 0.001 in the replication study.

TABLE 2
SNP markers with P-value less than 0.005 for HDL from t-test assuming
equal variaces between groups. Number of observations are marked by n1 and n2
and group avarages by avg1 and avg2.

| RS_id | SEQ_ID_no | Gene | Gene_id | Chr | Position | n1 | avg1 | n2 | avg2 | ttest | df | P-value |
|---|---|---|---|---|---|---|---|---|---|---|---|---|
| RS2195989 | 872 | ANGPT1 | 284 | 8 | 108555088 | 71 | 1.46 | 409 | 1.27 | 4.78 | 478 | 2.34E−06 |
| RS1480145 | 796 | | | 8 | 16181494 | 488 | 1.29 | 12 | 1.67 | 4.42 | 498 | 1.21E−05 |
| RS2029259 | 484 | | | 4 | 188422292 | 437 | 1.28 | 57 | 1.46 | 4.42 | 492 | 1.22E−05 |
| RS10503573 | 780 | MSR1 | 4481 | 8 | 16047855 | 486 | 1.29 | 14 | 1.63 | 4.25 | 498 | 2.55E−05 |
| RS4631686 | 3 | | | 1 | 14322780 | 482 | 1.29 | 8 | 1.74 | 4.22 | 488 | 2.91E−05 |
| RS250387 | 518 | ARL15 | 54622 | 5 | 53620394 | 411 | 1.27 | 83 | 1.42 | 4.18 | 492 | 3.45E−05 |
| RS1851006 | 1278 | UNC13C | 440279 | 15 | 52691684 | 156 | 1.22 | 212 | 1.35 | 4.09 | 366 | 5.31E−05 |
| RS10503586 | 803 | | | 8 | 16792998 | 476 | 1.29 | 12 | 1.65 | 4.05 | 486 | 5.96E−05 |
| RS397618 | 1063 | TMEM16C | 63982 | 11 | 26546754 | 486 | 1.29 | 14 | 1.61 | 4.01 | 498 | 7.00E−05 |
| RS285406 | 855 | | | 8 | 87094129 | 478 | 1.29 | 22 | 1.55 | 3.99 | 498 | 7.60E−05 |
| RS2854946 | 1259 | SERPINA5 | 5104 | 14 | 94118132 | 335 | 1.26 | 93 | 1.4 | 3.94 | 426 | 9.52E−05 |
| RS33375 | 598 | | | 5 | 170998718 | 407 | 1.28 | 61 | 1.44 | 3.92 | 466 | 1.02E−04 |
| RS10516673 | 370 | HNRPD | 3184 | 4 | 83635972 | 487 | 1.29 | 13 | 1.62 | 3.91 | 498 | 1.05E−04 |
| RS4770403 | 1173 | SGCG | 6445 | 13 | 22653127 | 448 | 1.28 | 52 | 1.45 | 3.88 | 498 | 1.18E−04 |
| RS9315157 | 1181 | 13CDNA73 | 10129 | 13 | 31642352 | 435 | 1.28 | 61 | 1.43 | 3.88 | 494 | 1.19E−04 |
| RS346421 | 527 | | | 5 | 62565739 | 447 | 1.28 | 47 | 1.46 | 3.86 | 492 | 1.29E−04 |
| RS6990997 | 867 | ZFPM2 | 23414 | 8 | 106881703 | 443 | 1.28 | 51 | 1.45 | 3.86 | 492 | 1.29E−04 |
| RS2170116 | 791 | | | 8 | 16157007 | 481 | 1.29 | 19 | 1.56 | 3.85 | 498 | 1.34E−04 |
| RS3770899 | 163 | CRIM1 | 51232 | 2 | 36566423 | 454 | 1.29 | 36 | 1.49 | 3.84 | 488 | 1.39E−04 |
| RS341148 | 696 | TULP4 | 56995 | 6 | 158810407 | 251 | 1.33 | 105 | 1.2 | 3.85 | 354 | 1.40E−04 |
| RS10517562 | 436 | | | 4 | 153393348 | 164 | 1.22 | 328 | 1.33 | 3.81 | 490 | 1.57E−04 |
| RS1226489 | 634 | GPR110 | 266977 | 6 | 47100017 | 478 | 1.29 | 10 | 1.65 | 3.81 | 486 | 1.57E−04 |
| RS10506790 | 1141 | | | 12 | 77762475 | 456 | 1.28 | 44 | 1.46 | 3.8 | 498 | 1.63E−04 |
| RS10482871 | 1439 | | | 21 | 15700259 | 495 | 1.29 | 5 | 1.8 | 3.8 | 498 | 1.63E−04 |
| RS10503572 | 789 | | | 8 | 16131101 | 491 | 1.29 | 5 | 1.81 | 3.8 | 494 | 1.63E−04 |
| RS9299445 | 993 | LOC644954 | 644954 | 10 | 63244794 | 446 | 1.28 | 40 | 1.47 | 3.8 | 484 | 1.63E−04 |
| RS10520350 | 472 | | | 4 | 177769342 | 478 | 1.28 | 20 | 1.54 | 3.79 | 496 | 1.69E−04 |
| RS965158 | 868 | | | 8 | 107564094 | 489 | 1.29 | 11 | 1.64 | 3.78 | 498 | 1.76E−04 |
| RS539254 | 836 | | | 8 | 80079448 | 343 | 1.28 | 91 | 1.41 | 3.74 | 432 | 2.09E−04 |
| RS10488888 | 395 | | | 4 | 111641312 | 490 | 1.29 | 8 | 1.69 | 3.72 | 496 | 2.22E−04 |
| RS2277456 | 1238 | PYGL | 5836 | 14 | 50447627 | 390 | 1.28 | 34 | 1.48 | 3.72 | 422 | 2.26E−04 |
| RS250388 | 519 | ARL15 | 54622 | 5 | 53621019 | 423 | 1.27 | 65 | 1.42 | 3.71 | 486 | 2.31E−04 |
| RS10485111 | 681 | | | 6 | 116299831 | 428 | 1.28 | 64 | 1.42 | 3.7 | 490 | 2.40E−04 |
| RS1577879 | 34 | | | 1 | 80924966 | 385 | 1.28 | 89 | 1.41 | 3.67 | 472 | 2.70E−04 |
| RS10512405 | 927 | SVEP1 | 79987 | 9 | 110316352 | 308 | 1.28 | 92 | 1.42 | 3.65 | 398 | 2.97E−04 |
| RS439269 | 897 | | | 9 | 14481347 | 440 | 1.28 | 58 | 1.43 | 3.63 | 496 | 3.13E−04 |
| RS1044045 | 1167 | EFHA1 | 221154 | 13 | 20965072 | 398 | 1.33 | 86 | 1.19 | 3.63 | 482 | 3.14E−04 |
| RS2056247 | 1092 | | | 11 | 93315872 | 406 | 1.27 | 94 | 1.4 | 3.62 | 498 | 3.25E−04 |
| RS9312592 | 462 | SPATA4 | 132851 | 4 | 177493018 | 60 | 1.43 | 408 | 1.28 | 3.6 | 466 | 3.52E−04 |
| RS9299446 | 994 | LOC644954 | 644954 | 10 | 63244886 | 457 | 1.28 | 43 | 1.45 | 3.59 | 498 | 3.63E−04 |
| RS1407466 | 889 | | | 9 | 7958548 | 403 | 1.27 | 93 | 1.4 | 3.58 | 494 | 3.78E−04 |
| RS9316436 | 1168 | EFHA1 | 221154 | 13 | 21000655 | 424 | 1.32 | 68 | 1.18 | 3.58 | 490 | 3.78E−04 |
| RS362988 | 1422 | SNAP25 | 6616 | 20 | 10229370 | 242 | 1.34 | 196 | 1.24 | 3.58 | 436 | 3.82E−04 |
| RS1942578 | 1402 | | | 18 | 73322001 | 279 | 1.33 | 211 | 1.24 | 3.57 | 488 | 3.92E−04 |
| RS4611835 | 313 | NAALADL2 | 254827 | 3 | 176610777 | 459 | 1.29 | 23 | 1.52 | 3.56 | 480 | 4.08E−04 |
| RS340686 | 500 | | | 5 | 8440200 | 107 | 1.21 | 391 | 1.32 | 3.54 | 496 | 4.38E−04 |
| RS1905155 | 556 | | | 5 | 113301097 | 313 | 1.32 | 141 | 1.21 | 3.54 | 452 | 4.42E−04 |
| RS10513319 | 952 | | | 9 | 117781003 | 482 | 1.29 | 16 | 1.56 | 3.53 | 496 | 4.54E−04 |
| RS1502887 | 358 | FRAS1 | 80144 | 4 | 79340562 | 455 | 1.29 | 41 | 1.46 | 3.52 | 494 | 4.71E−04 |
| RS723043 | 157 | LTBP1 | 4052 | 2 | 33453438 | 255 | 1.25 | 189 | 1.35 | 3.51 | 442 | 4.94E−04 |
| RS2414689 | 1285 | RORA | 6095 | 15 | 59051547 | 79 | 1.38 | 195 | 1.25 | 3.52 | 272 | 5.06E−04 |
| RS10509764 | 1013 | CNNM2 | 54805 | 10 | 104766465 | 348 | 1.33 | 150 | 1.23 | 3.5 | 496 | 5.07E−04 |
| RS8049647 | 1310 | | | 16 | 50695297 | 408 | 1.28 | 90 | 1.4 | 3.5 | 496 | 5.07E−04 |
| RS6954679 | 728 | | | 7 | 62940814 | 452 | 1.28 | 44 | 1.45 | 3.5 | 494 | 5.07E−04 |
| RS2222654 | 430 | | | 4 | 137363666 | 198 | 1.35 | 244 | 1.25 | 3.5 | 440 | 5.13E−04 |
| RS295525 | 525 | | | 5 | 60508623 | 487 | 1.29 | 13 | 1.58 | 3.49 | 498 | 5.26E−04 |
| RS10514844 | 1182 | | | 13 | 37808480 | 489 | 1.29 | 7 | 1.69 | 3.49 | 494 | 5.26E−04 |
| RS10504709 | 848 | | | 8 | 80781919 | 118 | 1.39 | 372 | 1.27 | 3.49 | 488 | 5.27E−04 |
| RS295577 | 524 | mimitin | 91942 | 5 | 60457592 | 472 | 1.29 | 14 | 1.57 | 3.48 | 484 | 5.47E−04 |
| RS340680 | 499 | | | 5 | 8438180 | 100 | 1.21 | 384 | 1.32 | 3.48 | 482 | 5.47E−04 |
| RS2034022 | 1396 | DOK6 | 220164 | 18 | 65341066 | 307 | 1.34 | 177 | 1.24 | 3.48 | 482 | 5.47E−04 |
| RS3218491 | 753 | XRCC2 | 7516 | 7 | 151791396 | 482 | 1.29 | 6 | 1.72 | 3.47 | 486 | 5.67E−04 |
| RS1433450 | 1293 | | | 15 | 85342066 | 450 | 1.31 | 46 | 1.15 | 3.46 | 494 | 5.87E−04 |
| RS6741304 | 175 | LOC400954 | 400954 | 2 | 55099846 | 336 | 1.26 | 152 | 1.37 | 3.46 | 486 | 5.88E−04 |
| RS745708 | 976 | FRMPD2 | 143162 | 10 | 49115681 | 363 | 1.27 | 79 | 1.39 | 3.46 | 440 | 5.93E−04 |
| RS2254162 | 774 | | | 8 | 14632707 | 278 | 1.27 | 76 | 1.39 | 3.46 | 352 | 6.07E−04 |
| RS1490125 | 651 | | | 6 | 66451580 | 428 | 1.32 | 66 | 1.18 | 3.45 | 492 | 6.09E−04 |
| RS1452528 | 670 | | | 6 | 79155079 | 247 | 1.34 | 241 | 1.25 | 3.45 | 486 | 6.09E−04 |
| RS7738834 | 671 | | | 6 | 79215706 | 260 | 1.33 | 132 | 1.22 | 3.45 | 390 | 6.22E−04 |
| RS2333821 | 170 | LOC646790 | 646790 | 2 | 44288177 | 330 | 1.27 | 146 | 1.37 | 3.44 | 474 | 6.33E−04 |
| RS2785949 | 1195 | | | 13 | 53806676 | 327 | 1.27 | 141 | 1.37 | 3.44 | 466 | 6.34E−04 |
| RS2110556 | 704 | | | 7 | 7556827 | 160 | 1.36 | 230 | 1.26 | 3.44 | 388 | 6.45E−04 |
| RS4733224 | 817 | WRN | 7486 | 8 | 31086911 | 280 | 1.25 | 182 | 1.35 | 3.42 | 460 | 6.82E−04 |
| RS10500783 | 1049 | | | 11 | 13530190 | 444 | 1.28 | 52 | 1.43 | 3.41 | 494 | 7.03E−04 |
| RS1607664 | 1410 | ZNF429 | 353088 | 19 | 21497820 | 396 | 1.27 | 90 | 1.39 | 3.41 | 484 | 7.04E−04 |
| RS1935229 | 1 | CAMTA1 | 23261 | 1 | 7695267 | 463 | 1.29 | 33 | 1.47 | 3.4 | 494 | 7.28E−04 |
| RS2127258 | 566 | | | 5 | 117512842 | 451 | 1.28 | 45 | 1.44 | 3.4 | 494 | 7.28E−04 |
| RS6699989 | 44 | | | 1 | 87784403 | 80 | 1.4 | 412 | 1.28 | 3.4 | 490 | 7.29E−04 |
| RS2357344 | 1242 | SYNE2 | 23224 | 14 | 63578006 | 404 | 1.27 | 36 | 1.45 | 3.4 | 438 | 7.36E−04 |
| RS2863171 | 1074 | | | 11 | 45207308 | 51 | 1.44 | 385 | 1.29 | 3.4 | 434 | 7.36E−04 |
| RS2405708 | 428 | | | 4 | 137339327 | 169 | 1.36 | 231 | 1.25 | 3.4 | 398 | 7.42E−04 |
| RS979323 | 1248 | | | 14 | 84118710 | 275 | 1.26 | 185 | 1.36 | 3.39 | 458 | 7.59E−04 |
| RS1025927 | 831 | TRPA1 | 8989 | 8 | 73125689 | 469 | 1.29 | 29 | 1.48 | 3.38 | 496 | 7.82E−04 |
| RS10509161 | 996 | LOC644954 | 644954 | 10 | 63245347 | 454 | 1.28 | 38 | 1.46 | 3.38 | 490 | 7.83E−04 |
| RS4146127 | 350 | | | 4 | 59260295 | 408 | 1.28 | 80 | 1.4 | 3.38 | 486 | 7.83E−04 |
| RS4510935 | 945 | | | 9 | 117364905 | 424 | 1.28 | 74 | 1.41 | 3.37 | 496 | 8.10E−04 |
| RS10490025 | 235 | KIAA1843 | 84540 | 2 | 210558906 | 456 | 1.29 | 36 | 1.46 | 3.37 | 490 | 8.11E−04 |
| RS10517618 | 439 | | | 4 | 156628360 | 327 | 1.26 | 131 | 1.36 | 3.37 | 456 | 8.16E−04 |
| RS1226491 | 633 | GPR110 | 266977 | 6 | 47097807 | 486 | 1.29 | 14 | 1.56 | 3.36 | 498 | 8.39E−04 |
| RS1544589 | 733 | PTPN12 | 5782 | 7 | 76874004 | 226 | 1.35 | 274 | 1.26 | 3.36 | 498 | 8.39E−04 |
| RS10513301 | 943 | ASTN2 | 23245 | 9 | 117241331 | 414 | 1.28 | 82 | 1.4 | 3.35 | 494 | 8.70E−04 |
| RS719484 | 1228 | | | 14 | 26547587 | 408 | 1.28 | 74 | 1.41 | 3.35 | 480 | 8.72E−04 |
| RS198278 | 711 | | | 7 | 24021433 | 233 | 1.24 | 243 | 1.33 | 3.35 | 474 | 8.73E−04 |
| RS3800818 | 730 | FLJ10099 | 55069 | 7 | 65865293 | 408 | 1.32 | 58 | 1.17 | 3.35 | 464 | 8.74E−04 |
| RS7087360 | 966 | CUBN | 8029 | 10 | 16912343 | 426 | 1.28 | 22 | 1.5 | 3.35 | 446 | 8.77E−04 |
| RS967220 | 264 | | | 3 | 29275054 | 72 | 1.19 | 348 | 1.33 | 3.35 | 418 | 8.81E−04 |
| RS596255 | 837 | | | 8 | 80164544 | 355 | 1.27 | 141 | 1.37 | 3.34 | 494 | 9.01E−04 |
| RS717806 | 160 | | | 2 | 36487186 | 358 | 1.27 | 122 | 1.37 | 3.34 | 478 | 9.03E−04 |
| RS868691 | 971 | | | 10 | 25945882 | 448 | 1.28 | 32 | 1.47 | 3.33 | 478 | 9.36E−04 |
| RS1424963 | 174 | | | 2 | 53360168 | 56 | 1.44 | 378 | 1.29 | 3.33 | 432 | 9.43E−04 |
| RS4890429 | 1387 | RIT2 | 6014 | 18 | 38951479 | 352 | 1.27 | 138 | 1.37 | 3.32 | 488 | 9.68E−04 |
| RS1541503 | 703 | LOC285929 | 285929 | 7 | 7173834 | 440 | 1.28 | 42 | 1.44 | 3.32 | 480 | 9.69E−04 |
| RS1867584 | 985 | | | 10 | 49321355 | 196 | 1.36 | 254 | 1.26 | 3.32 | 448 | 9.74E−04 |
| RS10500986 | 1053 | LUZP2 | 338645 | 11 | 24592114 | 363 | 1.28 | 95 | 1.39 | 3.31 | 456 | 1.01E−03 |
| RS10505540 | 882 | CCDC23 | 137196 | 8 | 130592619 | 479 | 1.29 | 21 | 1.51 | 3.3 | 498 | 1.04E−03 |
| RS4646981 | 1106 | NCAM1 | 4684 | 11 | 112596015 | 488 | 1.29 | 10 | 1.61 | 3.3 | 496 | 1.04E−03 |
| RS10507082 | 1148 | | | 12 | 96255332 | 107 | 1.21 | 371 | 1.32 | 3.3 | 476 | 1.04E−03 |
| RS10495202 | 115 | LOC644053 | 644053 | 1 | 219385670 | 382 | 1.27 | 118 | 1.38 | 3.29 | 498 | 1.07E−03 |
| RS171785 | 596 | | | 5 | 170994432 | 174 | 1.35 | 322 | 1.26 | 3.29 | 494 | 1.07E−03 |
| RS725500 | 364 | | | 4 | 80236951 | 384 | 1.31 | 106 | 1.21 | 3.29 | 488 | 1.07E−03 |
| RS1565741 | 804 | | | 8 | 16799856 | 431 | 1.28 | 59 | 1.42 | 3.29 | 488 | 1.07E−03 |
| RS717694 | 442 | | | 4 | 156772800 | 104 | 1.21 | 380 | 1.32 | 3.29 | 482 | 1.08E−03 |
| RS9312013 | 260 | | | 3 | 8299589 | 457 | 1.29 | 11 | 1.6 | 3.29 | 466 | 1.08E−03 |
| RS10509158 | 990 | C10orf107 | 219621 | 10 | 63177675 | 396 | 1.28 | 88 | 1.4 | 3.28 | 482 | 1.11E−03 |
| RS1813404 | 1246 | | | 14 | 84116590 | 329 | 1.26 | 147 | 1.36 | 3.28 | 474 | 1.11E−03 |
| RS1252225 | 854 | LOC138046 | 138046 | 8 | 85677664 | 292 | 1.27 | 102 | 1.38 | 3.28 | 392 | 1.13E−03 |
| RS10516142 | 601 | FLT4 | 2324 | 5 | 180003769 | 492 | 1.29 | 8 | 1.64 | 3.27 | 498 | 1.15E−03 |
| RS9284617 | 361 | FRAS1 | 80144 | 4 | 79349747 | 457 | 1.28 | 41 | 1.45 | 3.27 | 496 | 1.15E−03 |
| RS10517616 | 441 | | | 4 | 156733549 | 335 | 1.33 | 163 | 1.23 | 3.27 | 496 | 1.15E−03 |
| RS7706518 | 576 | | | 5 | 127308921 | 450 | 1.28 | 48 | 1.43 | 3.27 | 496 | 1.15E−03 |
| RS7718000 | 551 | | | 5 | 112998258 | 487 | 1.29 | 5 | 1.74 | 3.27 | 490 | 1.15E−03 |
| RS3890717 | 114 | | | 1 | 217860308 | 214 | 1.35 | 276 | 1.26 | 3.27 | 488 | 1.15E−03 |
| RS896990 | 251 | MGC42174 | 129563 | 2 | 232714361 | 437 | 1.29 | 47 | 1.44 | 3.27 | 482 | 1.15E−03 |
| RS10498121 | 242 | | | 2 | 222353045 | 460 | 1.29 | 22 | 1.5 | 3.27 | 480 | 1.15E−03 |
| RS10500660 | 1038 | DKFZP566M1046 | 84067 | 11 | 6199970 | 363 | 1.27 | 103 | 1.38 | 3.27 | 464 | 1.16E−03 |
| RS10489027 | 392 | | | 4 | 109645336 | 322 | 1.27 | 136 | 1.38 | 3.27 | 456 | 1.16E−03 |
| RS989177 | 748 | | | 7 | 124006837 | 70 | 1.41 | 358 | 1.27 | 3.26 | 426 | 1.20E−03 |
| RS2362932 | 120 | SMYD3 | 64754 | 1 | 242240325 | 141 | 1.38 | 273 | 1.28 | 3.26 | 412 | 1.21E−03 |
| RS4934799 | 972 | | | 10 | 36577492 | 378 | 1.32 | 122 | 1.22 | 3.25 | 498 | 1.23E−03 |
| RS10513299 | 941 | ASTN2 | 23245 | 9 | 117184531 | 428 | 1.28 | 64 | 1.41 | 3.25 | 490 | 1.23E−03 |
| RS6856098 | 331 | PCDH7 | 5099 | 4 | 30797074 | 393 | 1.28 | 97 | 1.38 | 3.25 | 488 | 1.23E−03 |
| RS722569 | 963 | | | 10 | 10538667 | 201 | 1.25 | 289 | 1.34 | 3.25 | 488 | 1.23E−03 |
| RS1385775 | 206 | LRP1B | 53353 | 2 | 141112833 | 195 | 1.33 | 223 | 1.25 | 3.25 | 416 | 1.25E−03 |
| RS1156968 | 1233 | | | 14 | 38232538 | 439 | 1.28 | 57 | 1.42 | 3.24 | 494 | 1.28E−03 |
| RS10509448 | 1005 | NRG3 | 10718 | 10 | 83972610 | 449 | 1.29 | 21 | 1.51 | 3.24 | 468 | 1.28E−03 |
| RS666941 | 240 | | | 2 | 221567963 | 262 | 1.33 | 206 | 1.24 | 3.24 | 466 | 1.28E−03 |
| RS1428250 | 508 | | | 5 | 38252169 | 450 | 1.29 | 14 | 1.55 | 3.24 | 462 | 1.28E−03 |
| RS10509160 | 995 | LOC644954 | 644954 | 10 | 63245218 | 457 | 1.28 | 43 | 1.44 | 3.23 | 498 | 1.32E−03 |
| RS10495466 | 118 | FMN2 | 56776 | 1 | 236834608 | 474 | 1.29 | 22 | 1.5 | 3.23 | 494 | 1.32E−03 |
| RS7148403 | 1234 | | | 14 | 38248309 | 441 | 1.28 | 55 | 1.42 | 3.23 | 494 | 1.32E−03 |
| RS1936671 | 97 | | | 1 | 189055410 | 373 | 1.27 | 121 | 1.37 | 3.23 | 492 | 1.32E−03 |
| RS10485148 | 619 | | | 6 | 23080312 | 340 | 1.27 | 150 | 1.37 | 3.23 | 488 | 1.32E−03 |
| RS1792369 | 1090 | FAT3 | 120114 | 11 | 92092081 | 203 | 1.25 | 287 | 1.34 | 3.23 | 488 | 1.32E−03 |
| RS1604613 | 1197 | | | 13 | 69021604 | 217 | 1.33 | 255 | 1.25 | 3.23 | 470 | 1.32E−03 |
| RS4668806 | 139 | | | 2 | 13630775 | 263 | 1.34 | 207 | 1.25 | 3.23 | 468 | 1.33E−03 |
| RS10518089 | 357 | SLC4A4 | 8671 | 4 | 72794260 | 367 | 1.27 | 131 | 1.37 | 3.22 | 496 | 1.37E−03 |
| RS9285122 | 1183 | | | 13 | 37809901 | 490 | 1.29 | 8 | 1.64 | 3.22 | 496 | 1.37E−03 |
| RS2150468 | 1184 | UFM1 | 51569 | 13 | 37825888 | 490 | 1.29 | 8 | 1.64 | 3.22 | 496 | 1.37E−03 |
| RS2217486 | 489 | | | 4 | 188707345 | 103 | 1.38 | 393 | 1.27 | 3.22 | 494 | 1.37E−03 |
| RS7897663 | 1015 | NT5C2 | 22978 | 10 | 104895594 | 343 | 1.33 | 153 | 1.23 | 3.21 | 494 | 1.41E−03 |
| RS2278016 | 1308 | | | 16 | 50674510 | 402 | 1.28 | 86 | 1.39 | 3.21 | 486 | 1.42E−03 |
| RS2969004 | 422 | | | 4 | 131139275 | 182 | 1.36 | 276 | 1.27 | 3.21 | 456 | 1.42E−03 |
| RS1016579 | 881 | | | 8 | 129269143 | 412 | 1.28 | 28 | 1.46 | 3.21 | 438 | 1.42E−03 |
| RS2323936 | 1186 | | | 13 | 38001773 | 492 | 1.29 | 8 | 1.64 | 3.2 | 498 | 1.46E−03 |
| RS2323937 | 1187 | | | 13 | 38001927 | 492 | 1.29 | 8 | 1.64 | 3.2 | 498 | 1.46E−03 |
| RS2409037 | 577 | | | 5 | 127314089 | 448 | 1.28 | 48 | 1.43 | 3.2 | 494 | 1.46E−03 |
| RS3815035 | 1355 | MAP2K6 | 5608 | 17 | 65043983 | 438 | 1.28 | 58 | 1.42 | 3.2 | 494 | 1.46E−03 |
| RS10517222 | 332 | | | 4 | 30831187 | 383 | 1.27 | 111 | 1.37 | 3.2 | 492 | 1.46E−03 |
| RS1861373 | 1150 | | | 12 | 96275336 | 212 | 1.25 | 282 | 1.34 | 3.2 | 492 | 1.46E−03 |
| RS10511857 | 904 | | | 9 | 30276600 | 408 | 1.28 | 82 | 1.4 | 3.2 | 488 | 1.46E−03 |
| RS1916043 | 1050 | PSMA1 | 5682 | 11 | 14593532 | 456 | 1.29 | 34 | 1.46 | 3.2 | 488 | 1.46E−03 |
| RS1927914 | 951 | TLR4 | 7099 | 9 | 117544279 | 220 | 1.34 | 266 | 1.25 | 3.2 | 484 | 1.46E−03 |
| RS741651 | 1243 | | | 14 | 80689322 | 443 | 1.28 | 39 | 1.45 | 3.2 | 480 | 1.47E−03 |
| RS2477035 | 965 | | | 10 | 10571483 | 193 | 1.25 | 287 | 1.34 | 3.2 | 478 | 1.47E−03 |
| RS631807 | 613 | LOC442161 | 442161 | 6 | 10475686 | 338 | 1.27 | 132 | 1.37 | 3.2 | 468 | 1.47E−03 |
| RS3805134 | 295 | SLC15A2 | 6565 | 3 | 123100746 | 189 | 1.35 | 277 | 1.26 | 3.2 | 464 | 1.47E−03 |
| RS1517290 | 440 | | | 4 | 156679488 | 219 | 1.26 | 199 | 1.35 | 3.2 | 416 | 1.48E−03 |
| RS10516865 | 379 | | | 4 | 91462479 | 495 | 1.29 | 5 | 1.72 | 3.19 | 498 | 1.51E−03 |
| RS10514845 | 1185 | | | 13 | 37910815 | 488 | 1.29 | 8 | 1.64 | 3.19 | 494 | 1.51E−03 |
| RS1886452 | 1200 | | | 13 | 72884855 | 395 | 1.28 | 95 | 1.39 | 3.19 | 488 | 1.51E−03 |
| RS2834370 | 1454 | | | 21 | 34346680 | 460 | 1.29 | 16 | 1.54 | 3.19 | 474 | 1.52E−03 |
| RS2811930 | 909 | UBQLN1 | 29979 | 9 | 83513404 | 247 | 1.33 | 157 | 1.23 | 3.19 | 402 | 1.53E−03 |
| RS10492335 | 1156 | | | 12 | 113042283 | 474 | 1.31 | 26 | 1.11 | 3.18 | 498 | 1.56E−03 |
| RS10502320 | 1361 | LOC388458 | 388458 | 18 | 4137742 | 267 | 1.34 | 231 | 1.25 | 3.18 | 496 | 1.56E−03 |
| RS3912653 | 1091 | FAT3 | 120114 | 11 | 92135172 | 204 | 1.25 | 290 | 1.33 | 3.18 | 492 | 1.57E−03 |
| RS652238 | 1395 | | | 18 | 64109605 | 417 | 1.31 | 77 | 1.19 | 3.18 | 492 | 1.57E−03 |
| RS2169942 | 565 | | | 5 | 117512644 | 449 | 1.28 | 43 | 1.43 | 3.18 | 490 | 1.57E−03 |
| RS10510809 | 274 | | | 3 | 59414093 | 402 | 1.28 | 80 | 1.4 | 3.18 | 480 | 1.57E−03 |
| RS7963889 | 1134 | | | 12 | 73136950 | 156 | 1.35 | 284 | 1.25 | 3.18 | 438 | 1.58E−03 |
| RS10517442 | 351 | | | 4 | 59273158 | 88 | 1.39 | 412 | 1.28 | 3.17 | 498 | 1.62E−03 |
| RS7002137 | 769 | | | 8 | 10653610 | 425 | 1.28 | 73 | 1.4 | 3.17 | 496 | 1.62E−03 |
| RS10508392 | 960 | | | 10 | 9722419 | 466 | 1.29 | 30 | 1.47 | 3.17 | 494 | 1.62E−03 |
| RS10512935 | 304 | EPHB1 | 2047 | 3 | 136107953 | 381 | 1.27 | 109 | 1.38 | 3.17 | 488 | 1.62E−03 |
| RS10516655 | 366 | | | 4 | 82801964 | 372 | 1.28 | 118 | 1.38 | 3.17 | 488 | 1.62E−03 |
| RS10500474 | 1318 | | | 16 | 62073509 | 401 | 1.28 | 73 | 1.4 | 3.17 | 472 | 1.62E−03 |
| RS1556082 | 1215 | GPC6 | 10082 | 13 | 93502465 | 95 | 1.38 | 401 | 1.27 | 3.16 | 494 | 1.67E−03 |
| RS3897748 | 113 | | | 1 | 214677604 | 407 | 1.31 | 79 | 1.19 | 3.16 | 484 | 1.68E−03 |
| RS10488642 | 751 | TRIM24 | 8805 | 7 | 137646187 | 373 | 1.27 | 109 | 1.38 | 3.16 | 480 | 1.68E−03 |
| RS1407473 | 890 | | | 9 | 7989681 | 357 | 1.27 | 123 | 1.36 | 3.16 | 478 | 1.68E−03 |
| RS7453920 | 631 | HLA-DQB2 | 3120 | 6 | 32837990 | 187 | 1.35 | 291 | 1.26 | 3.16 | 476 | 1.68E−03 |
| RS10510155 | 1037 | | | 10 | 128313990 | 424 | 1.28 | 50 | 1.42 | 3.16 | 472 | 1.68E−03 |
| RS989766 | 1431 | PTPRT | 11122 | 20 | 40443639 | 320 | 1.27 | 116 | 1.38 | 3.16 | 434 | 1.69E−03 |
| RS6852538 | 427 | | | 4 | 137338578 | 221 | 1.34 | 275 | 1.26 | 3.15 | 494 | 1.73E−03 |
| RS1639239 | 707 | DGKB | 1607 | 7 | 14564608 | 91 | 1.21 | 403 | 1.32 | 3.15 | 492 | 1.73E−03 |
| RS2881290 | 394 | COL25A1 | 84570 | 4 | 110563718 | 432 | 1.31 | 60 | 1.18 | 3.15 | 490 | 1.73E−03 |
| RS10495618 | 140 | | | 2 | 13631037 | 284 | 1.33 | 206 | 1.25 | 3.15 | 488 | 1.73E−03 |
| RS10507083 | 1149 | | | 12 | 96255384 | 114 | 1.22 | 372 | 1.32 | 3.15 | 484 | 1.73E−03 |
| RS1926029 | 1014 | NT5C2 | 22978 | 10 | 104845660 | 314 | 1.33 | 80 | 1.21 | 3.15 | 392 | 1.76E−03 |
| RS591576 | 1203 | FBXL3 | 26224 | 13 | 76488937 | 81 | 1.39 | 419 | 1.28 | 3.14 | 498 | 1.79E−03 |
| RS10506626 | 1128 | TSPAN8 | 7103 | 12 | 69815758 | 341 | 1.27 | 153 | 1.36 | 3.14 | 492 | 1.79E−03 |
| RS1050692 | 970 | GPR158 | 57512 | 10 | 25677276 | 487 | 1.29 | 5 | 1.72 | 3.14 | 490 | 1.79E−03 |
| RS10519371 | 426 | | | 4 | 137332410 | 222 | 1.34 | 256 | 1.26 | 3.14 | 476 | 1.79E−03 |
| RS2043179 | 1273 | | | 15 | 44563353 | 239 | 1.25 | 239 | 1.33 | 3.14 | 476 | 1.79E−03 |
| RS2121956 | 79 | | | 1 | 104801417 | 196 | 1.35 | 278 | 1.26 | 3.14 | 472 | 1.80E−03 |
| RS7445088 | 526 | | | 5 | 62306508 | 404 | 1.28 | 68 | 1.4 | 3.14 | 470 | 1.80E−03 |
| RS1917952 | 429 | | | 4 | 137355240 | 205 | 1.35 | 251 | 1.26 | 3.14 | 454 | 1.80E−03 |
| RS3751462 | 1239 | LOC283553 | 283553 | 14 | 50897971 | 346 | 1.32 | 66 | 1.2 | 3.14 | 410 | 1.81E−03 |
| RS4757441 | 1051 | PLEKHA7 | 144100 | 11 | 16859250 | 440 | 1.31 | 60 | 1.18 | 3.13 | 498 | 1.85E−03 |
| RS10521210 | 1335 | | | 17 | 12966928 | 233 | 1.34 | 261 | 1.26 | 3.13 | 492 | 1.85E−03 |
| RS10514632 | 212 | RAPGEF4 | 11069 | 2 | 173530419 | 391 | 1.32 | 95 | 1.21 | 3.13 | 484 | 1.85E−03 |
| RS2296697 | 40 | LPHN2 | 23266 | 1 | 82149240 | 400 | 1.28 | 80 | 1.39 | 3.13 | 478 | 1.86E−03 |
| RS6978964 | 701 | SDK1 | 221935 | 7 | 3646860 | 437 | 1.28 | 61 | 1.41 | 3.12 | 496 | 1.91E−03 |
| RS10487779 | 709 | DGKB | 1607 | 7 | 14617576 | 105 | 1.21 | 393 | 1.32 | 3.12 | 496 | 1.91E−03 |
| RS4102401 | 866 | RRM2B | 50484 | 8 | 103288416 | 477 | 1.29 | 21 | 1.5 | 3.12 | 496 | 1.91E−03 |
| RS323416 | 1359 | | | 17 | 69240908 | 476 | 1.29 | 22 | 1.49 | 3.12 | 496 | 1.91E−03 |
| RS7794555 | 754 | LOC653748 | 653748 | 7 | 153481879 | 462 | 1.28 | 34 | 1.45 | 3.12 | 494 | 1.91E−03 |
| RS3118650 | 1194 | | | 13 | 49927325 | 482 | 1.29 | 14 | 1.54 | 3.12 | 494 | 1.91E−03 |
| RS10501933 | 1100 | CNTN5 | 53942 | 11 | 99284564 | 403 | 1.28 | 91 | 1.38 | 3.12 | 492 | 1.91E−03 |
| RS7768538 | 630 | HLA-DQB2 | 3120 | 6 | 32837799 | 299 | 1.26 | 193 | 1.35 | 3.12 | 490 | 1.92E−03 |
| RS10498836 | 654 | | | 6 | 66585591 | 422 | 1.32 | 64 | 1.19 | 3.12 | 484 | 1.92E−03 |
| RS9284616 | 360 | FRAS1 | 80144 | 4 | 79349710 | 448 | 1.28 | 32 | 1.45 | 3.12 | 478 | 1.92E−03 |
| RS10226709 | 708 | DGKB | 1607 | 7 | 14570874 | 382 | 1.32 | 68 | 1.19 | 3.12 | 448 | 1.93E−03 |
| RS10497689 | 224 | | | 2 | 188708247 | 495 | 1.29 | 5 | 1.71 | 3.11 | 498 | 1.98E−03 |
| RS2969001 | 423 | | | 4 | 131140397 | 305 | 1.26 | 195 | 1.35 | 3.11 | 498 | 1.98E−03 |
| RS10492135 | 1114 | GRIN2B | 2904 | 12 | 13777535 | 110 | 1.38 | 388 | 1.27 | 3.11 | 496 | 1.98E−03 |
| RS715428 | 1365 | KIAA1772 | 80000 | 18 | 17131449 | 486 | 1.29 | 12 | 1.57 | 3.11 | 496 | 1.98E−03 |
| RS10511852 | 903 | | | 9 | 30227951 | 477 | 1.29 | 15 | 1.54 | 3.11 | 490 | 1.98E−03 |
| RS10509863 | 1020 | | | 10 | 109828476 | 439 | 1.28 | 35 | 1.45 | 3.11 | 472 | 1.98E−03 |
| RS1598045 | 171 | LOC646790 | 646790 | 2 | 44288776 | 261 | 1.25 | 209 | 1.34 | 3.11 | 468 | 1.99E−03 |
| RS2089907 | 122 | | | 2 | 2454467 | 342 | 1.27 | 112 | 1.37 | 3.11 | 452 | 1.99E−03 |
| RS923544 | 45 | | | 1 | 87792449 | 246 | 1.34 | 246 | 1.26 | 3.1 | 490 | 2.05E−03 |
| RS2881836 | 234 | AOX2 | 344454 | 2 | 201469088 | 326 | 1.26 | 148 | 1.36 | 3.1 | 472 | 2.05E−03 |
| RS10504722 | 853 | ZBTB10 | 65986 | 8 | 81592447 | 60 | 1.41 | 396 | 1.28 | 3.1 | 454 | 2.06E−03 |
| RS2849382 | 1394 | BCL2 | 596 | 18 | 59127173 | 408 | 1.28 | 48 | 1.43 | 3.1 | 454 | 2.06E−03 |
| RS10490026 | 236 | KIAA1843 | 84540 | 2 | 210558951 | 460 | 1.29 | 40 | 1.44 | 3.09 | 498 | 2.11E−03 |
| RS10490028 | 238 | KIAA1843 | 84540 | 2 | 210560666 | 460 | 1.29 | 40 | 1.44 | 3.09 | 498 | 2.11E−03 |
| RS10517787 | 444 | | | 4 | 165039745 | 454 | 1.29 | 42 | 1.43 | 3.09 | 494 | 2.11E−03 |
| RS3886738 | 992 | LOC644954 | 644954 | 10 | 63234254 | 451 | 1.28 | 39 | 1.44 | 3.09 | 488 | 2.12E−03 |
| RS1293700 | 1416 | | | 19 | 58837765 | 65 | 1.41 | 417 | 1.29 | 3.09 | 480 | 2.12E−03 |
| RS1484317 | 76 | | | 1 | 101802287 | 342 | 1.28 | 110 | 1.38 | 3.09 | 450 | 2.13E−03 |
| RS10485880 | 736 | MAGI2 | 9863 | 7 | 77560883 | 376 | 1.28 | 56 | 1.41 | 3.09 | 430 | 2.13E−03 |
| RS10507926 | 1211 | | | 13 | 81335314 | 456 | 1.31 | 44 | 1.16 | 3.08 | 498 | 2.18E−03 |
| RS2480010 | 617 | | | 6 | 23014923 | 371 | 1.27 | 121 | 1.36 | 3.08 | 490 | 2.19E−03 |
| RS2072506 | 722 | AMPH | 273 | 7 | 38275635 | 459 | 1.29 | 31 | 1.46 | 3.08 | 488 | 2.19E−03 |
| RS10518211 | 365 | | | 4 | 80294244 | 265 | 1.33 | 223 | 1.25 | 3.08 | 486 | 2.19E−03 |
| RS10517561 | 434 | LOC646697 | 646697 | 4 | 153190724 | 296 | 1.26 | 192 | 1.35 | 3.08 | 486 | 2.19E−03 |
| RS4131874 | 819 | | | 8 | 42051123 | 124 | 1.22 | 364 | 1.32 | 3.08 | 486 | 2.19E−03 |
| RS9328534 | 954 | CRSP8 | 9442 | 9 | 131904359 | 335 | 1.32 | 153 | 1.23 | 3.08 | 486 | 2.19E−03 |
| RS9292042 | 517 | ARL15 | 54622 | 5 | 53572755 | 377 | 1.27 | 105 | 1.38 | 3.08 | 480 | 2.19E−03 |
| RS1429130 | 432 | | | 4 | 148581368 | 443 | 1.29 | 25 | 1.48 | 3.08 | 466 | 2.19E−03 |
| RS10505777 | 1113 | | | 12 | 13378527 | 354 | 1.3 | 4 | 1.77 | 3.08 | 356 | 2.23E−03 |
| RS10503626 | 808 | PSD3 | 23362 | 8 | 18591364 | 475 | 1.29 | 23 | 1.49 | 3.07 | 496 | 2.26E−03 |
| RS4440168 | 475 | | | 4 | 178345257 | 424 | 1.28 | 72 | 1.4 | 3.07 | 494 | 2.26E−03 |
| RS724334 | 273 | CAST1 | 26059 | 3 | 55933122 | 411 | 1.28 | 79 | 1.39 | 3.07 | 488 | 2.26E−03 |
| RS10497650 | 223 | | | 2 | 185048541 | 469 | 1.29 | 5 | 1.71 | 3.07 | 472 | 2.26E−03 |
| RS9318612 | 1208 | | | 13 | 78663468 | 239 | 1.26 | 235 | 1.34 | 3.07 | 472 | 2.26E−03 |
| RS4876853 | 878 | | | 8 | 119765786 | 448 | 1.29 | 10 | 1.59 | 3.07 | 456 | 2.27E−03 |
| RS4418368 | 758 | DLGAP2 | 9228 | 8 | 1585785 | 264 | 1.28 | 148 | 1.37 | 3.07 | 410 | 2.28E−03 |
| RS10514831 | 907 | | | 9 | 82681244 | 439 | 1.28 | 61 | 1.41 | 3.06 | 498 | 2.33E−03 |
| RS10495390 | 116 | RYR2 | 6262 | 1 | 233639054 | 448 | 1.28 | 46 | 1.43 | 3.06 | 492 | 2.33E−03 |
| RS10513792 | 317 | MCF2L2 | 23101 | 3 | 184517877 | 416 | 1.28 | 52 | 1.42 | 3.06 | 466 | 2.34E−03 |
| RS9327177 | 567 | | | 5 | 120387365 | 184 | 1.35 | 272 | 1.26 | 3.06 | 454 | 2.34E−03 |
| RS10489945 | 46 | | | 1 | 88125610 | 469 | 1.31 | 27 | 1.12 | 3.05 | 494 | 2.41E−03 |
| RS1121640 | 1099 | | | 11 | 96694636 | 464 | 1.29 | 32 | 1.46 | 3.05 | 494 | 2.41E−03 |
| RS3770186 | 241 | EPHA4 | 2043 | 2 | 222137844 | 385 | 1.28 | 109 | 1.38 | 3.05 | 492 | 2.41E−03 |
| RS979322 | 1247 | | | 14 | 84118568 | 288 | 1.26 | 206 | 1.34 | 3.05 | 492 | 2.41E−03 |
| RS1112809 | 210 | | | 2 | 148314459 | 436 | 1.31 | 52 | 1.18 | 3.05 | 486 | 2.41E−03 |
| RS1525647 | 725 | | | 7 | 45289320 | 106 | 1.38 | 374 | 1.28 | 3.05 | 478 | 2.42E−03 |
| RS2451947 | 1138 | | | 12 | 76451623 | 278 | 1.34 | 184 | 1.25 | 3.05 | 460 | 2.42E−03 |
| RS360559 | 644 | CRISP2 | 7180 | 6 | 49767830 | 275 | 1.34 | 173 | 1.25 | 3.05 | 446 | 2.42E−03 |
| RS997132 | 1204 | | | 13 | 76829538 | 274 | 1.25 | 164 | 1.34 | 3.05 | 436 | 2.43E−03 |
| RS2503102 | 679 | LOC442238 | 442238 | 6 | 100650488 | 282 | 1.33 | 144 | 1.24 | 3.05 | 424 | 2.43E−03 |
| RS10492047 | 1158 | LOC144678 | 144678 | 12 | 125460210 | 475 | 1.29 | 25 | 1.48 | 3.04 | 498 | 2.49E−03 |
| RS10495667 | 141 | VSNL1 | 7447 | 2 | 17745120 | 493 | 1.29 | 5 | 1.7 | 3.04 | 496 | 2.49E−03 |
| RS6650321 | 1214 | GPC6 | 10082 | 13 | 93419035 | 388 | 1.28 | 110 | 1.37 | 3.04 | 496 | 2.49E−03 |
| RS1818711 | 1279 | UNC13C | 440279 | 15 | 52692074 | 288 | 1.26 | 210 | 1.35 | 3.04 | 496 | 2.49E−03 |
| RS10489026 | 391 | | | 4 | 109640337 | 358 | 1.27 | 134 | 1.36 | 3.04 | 490 | 2.49E−03 |
| RS1322137 | 893 | | | 9 | 10220744 | 330 | 1.27 | 162 | 1.35 | 3.04 | 490 | 2.49E−03 |
| RS1749541 | 57 | ABCD3 | 5825 | 1 | 94670399 | 338 | 1.27 | 148 | 1.36 | 3.04 | 484 | 2.49E−03 |
| RS10489036 | 320 | KIAA1276 | 27146 | 4 | 17373956 | 350 | 1.27 | 130 | 1.36 | 3.04 | 478 | 2.50E−03 |
| RS10518502 | 419 | | | 4 | 127915337 | 349 | 1.32 | 121 | 1.22 | 3.04 | 468 | 2.50E−03 |
| RS2900932 | 1022 | | | 10 | 113232182 | 351 | 1.28 | 119 | 1.37 | 3.04 | 468 | 2.50E−03 |
| RS2304376 | 727 | GBAS | 2631 | 7 | 55823228 | 389 | 1.32 | 77 | 1.21 | 3.04 | 464 | 2.50E−03 |
| RS953273 | 92 | NMNAT2 | 23057 | 1 | 180031268 | 259 | 1.26 | 171 | 1.35 | 3.04 | 428 | 2.51E−03 |
| RS10495767 | 149 | FLJ34931 | 388939 | 2 | 29198843 | 357 | 1.27 | 47 | 1.42 | 3.04 | 402 | 2.52E−03 |
| RS878016 | 757 | DLGAP2 | 9228 | 8 | 1585693 | 355 | 1.27 | 143 | 1.36 | 3.03 | 496 | 2.57E−03 |
| RS2825652 | 1441 | | | 21 | 19865219 | 488 | 1.29 | 10 | 1.58 | 3.03 | 496 | 2.57E−03 |
| RS1487377 | 1375 | OSBPLIA | 114876 | 18 | 20137550 | 99 | 1.22 | 397 | 1.32 | 3.03 | 494 | 2.57E−03 |
| RS10484693 | 603 | GMDS | 2762 | 6 | 1855107 | 482 | 1.29 | 12 | 1.56 | 3.03 | 492 | 2.57E−03 |
| RS150344 | 1302 | | | 16 | 22728560 | 414 | 1.28 | 76 | 1.39 | 3.03 | 488 | 2.58E−03 |
| RS1886484 | 1199 | | | 13 | 71945230 | 178 | 1.35 | 308 | 1.27 | 3.03 | 484 | 2.58E−03 |
| RS10489925 | 67 | PAP2D | 163404 | 1 | 99173344 | 361 | 1.27 | 107 | 1.37 | 3.03 | 466 | 2.58E−03 |
| RS10486012 | 742 | | | 7 | 105824729 | 491 | 1.29 | 9 | 1.6 | 3.02 | 498 | 2.66E−03 |
| RS2161413 | 589 | | | 5 | 165940868 | 452 | 1.31 | 44 | 1.17 | 3.02 | 494 | 2.66E−03 |
| RS2162654 | 1155 | | | 12 | 113038233 | 467 | 1.3 | 29 | 1.13 | 3.02 | 494 | 2.66E−03 |
| RS7694034 | 377 | MAPK10 | 5602 | 4 | 87647635 | 402 | 1.31 | 84 | 1.2 | 3.02 | 484 | 2.66E−03 |
| RS7844425 | 818 | | | 8 | 32495159 | 321 | 1.27 | 155 | 1.36 | 3.02 | 474 | 2.66E−03 |
| RS2893569 | 723 | POU6F2 | 11281 | 7 | 38839984 | 461 | 1.29 | 7 | 1.64 | 3.02 | 466 | 2.67E−03 |
| RS4628499 | 119 | LOC645939 | 645939 | 1 | 237260520 | 379 | 1.27 | 87 | 1.38 | 3.02 | 464 | 2.67E−03 |
| RS2105233 | 117 | | | 1 | 235710329 | 375 | 1.32 | 81 | 1.2 | 3.02 | 454 | 2.67E−03 |
| RS10507553 | 1190 | | | 13 | 46687101 | 495 | 1.29 | 5 | 1.7 | 3.01 | 498 | 2.74E−03 |
| RS1852561 | 731 | | | 7 | 69770184 | 15 | 1.53 | 483 | 1.29 | 3.01 | 496 | 2.75E−03 |
| RS1460174 | 845 | | | 8 | 80266395 | 396 | 1.28 | 102 | 1.38 | 3.01 | 496 | 2.75E−03 |
| RS1938105 | 652 | | | 6 | 66567647 | 427 | 1.32 | 69 | 1.2 | 3.01 | 494 | 2.75E−03 |
| RS964453 | 1326 | USP10 | 9100 | 16 | 83325736 | 372 | 1.32 | 108 | 1.22 | 3.01 | 478 | 2.75E−03 |
| RS10509153 | 989 | | | 10 | 63041318 | 416 | 1.28 | 42 | 1.42 | 3.01 | 456 | 2.76E−03 |
| RS1582165 | 121 | | | 1 | 244927430 | 40 | 1.16 | 362 | 1.31 | 3.01 | 400 | 2.78E−03 |
| RS7574093 | 176 | | | 2 | 56818904 | 490 | 1.29 | 10 | 1.58 | 3 | 498 | 2.84E−03 |
| RS4684321 | 256 | | | 3 | 1842614 | 477 | 1.29 | 23 | 1.48 | 3 | 498 | 2.84E−03 |
| RS10491543 | 908 | | | 9 | 82683005 | 438 | 1.28 | 60 | 1.41 | 3 | 496 | 2.84E−03 |
| RS10483457 | 1232 | GARNL1 | 253959 | 14 | 35211683 | 464 | 1.29 | 34 | 1.45 | 3 | 496 | 2.84E−03 |
| RS2474263 | 678 | LOC442238 | 442238 | 6 | 100641976 | 315 | 1.32 | 177 | 1.24 | 3 | 490 | 2.84E−03 |
| RS10488693 | 1083 | C11orf11 | 747 | 11 | 61241732 | 467 | 1.29 | 23 | 1.48 | 3 | 488 | 2.84E−03 |
| RS1275637 | 1135 | | | 12 | 74445115 | 423 | 1.28 | 67 | 1.4 | 3 | 488 | 2.84E−03 |
| RS10488865 | 388 | | | 4 | 105801941 | 441 | 1.3 | 31 | 1.13 | 3 | 470 | 2.84E−03 |
| RS1537656 | 607 | | | 6 | 8060391 | 343 | 1.27 | 119 | 1.37 | 3 | 460 | 2.85E−03 |
| RS2248815 | 1453 | ITSN1 | 6453 | 21 | 34060196 | 386 | 1.32 | 59 | 1.19 | 3 | 446 | 2.85E−03 |
| RS10497776 | 228 | | | 2 | 196795652 | 493 | 1.29 | 5 | 1.7 | 2.99 | 496 | 2.93E−03 |
| RS10501410 | 1085 | CLPB | 81570 | 11 | 71766454 | 438 | 1.28 | 58 | 1.41 | 2.99 | 494 | 2.93E−03 |
| RS1513104 | 1127 | PTPRR | 5801 | 12 | 69387170 | 419 | 1.28 | 75 | 1.39 | 2.99 | 492 | 2.93E−03 |
| RS10223625 | 656 | | | 6 | 67183675 | 420 | 1.28 | 72 | 1.4 | 2.99 | 490 | 2.93E−03 |
| RS6987277 | 766 | | | 8 | 8977241 | 453 | 1.29 | 39 | 1.44 | 2.99 | 490 | 2.93E−03 |
| RS1964670 | 362 | FRAS1 | 80144 | 4 | 79351645 | 163 | 1.24 | 321 | 1.33 | 2.99 | 482 | 2.93E−03 |
| RS3124806 | 2 | CAMTA1 | 23261 | 1 | 7703744 | 456 | 1.28 | 26 | 1.45 | 2.99 | 480 | 2.93E−03 |
| RS219664 | 1445 | | | 21 | 26728062 | 376 | 1.32 | 104 | 1.22 | 2.99 | 478 | 2.93E−03 |
| RS1430593 | 739 | | | 7 | 84135009 | 400 | 1.32 | 70 | 1.2 | 2.99 | 468 | 2.94E−03 |
| RS10493179 | 5 | USP24 | 23358 | 1 | 55317664 | 413 | 1.29 | 43 | 1.43 | 2.99 | 454 | 2.94E−03 |
| RS4131826 | 87 | SDHC | 6391 | 1 | 158095457 | 349 | 1.27 | 147 | 1.36 | 2.98 | 494 | 3.02E−03 |
| RS7997251 | 1201 | | | 13 | 75508128 | 399 | 1.28 | 97 | 1.38 | 2.98 | 494 | 3.02E−03 |
| RS1786606 | 1384 | DTNA | 1837 | 18 | 30686115 | 417 | 1.32 | 79 | 1.21 | 2.98 | 494 | 3.02E−03 |
| RS2294311 | 1457 | HSPC117 | 51493 | 22 | 31117823 | 369 | 1.27 | 127 | 1.37 | 2.98 | 494 | 3.02E−03 |
| RS1343725 | 825 | | | 8 | 70070765 | 426 | 1.28 | 68 | 1.4 | 2.98 | 492 | 3.03E−03 |
| RS10509766 | 1016 | NT5C2 | 22978 | 10 | 104916324 | 339 | 1.33 | 151 | 1.24 | 2.98 | 488 | 3.03E−03 |
| RS2413809 | 1270 | | | 15 | 44497210 | 322 | 1.27 | 168 | 1.35 | 2.98 | 488 | 3.03E−03 |
| RS7164579 | 1288 | LCTL | 197021 | 15 | 64629063 | 26 | 1.47 | 456 | 1.29 | 2.98 | 480 | 3.03E−03 |
| RS6449326 | 321 | KIAA1276 | 27146 | 4 | 17376059 | 338 | 1.27 | 136 | 1.36 | 2.98 | 472 | 3.03E−03 |
| RS4955346 | 265 | | | 3 | 30879119 | 299 | 1.27 | 159 | 1.36 | 2.98 | 456 | 3.04E−03 |
| RS522071 | 1177 | MGC9850 | 219404 | 13 | 27109580 | 276 | 1.26 | 160 | 1.35 | 2.98 | 434 | 3.04E−03 |
| RS10519370 | 425 | | | 4 | 137332061 | 221 | 1.34 | 275 | 1.26 | 2.97 | 494 | 3.12E−03 |
| RS7467596 | 955 | CRSP8 | 9442 | 9 | 131942232 | 349 | 1.32 | 147 | 1.24 | 2.97 | 494 | 3.12E−03 |
| RS10492139 | 1115 | GRIN2B | 2904 | 12 | 13877862 | 113 | 1.37 | 383 | 1.27 | 2.97 | 494 | 3.12E−03 |
| RS2014357 | 883 | | | 8 | 133172472 | 441 | 1.28 | 53 | 1.41 | 2.97 | 492 | 3.12E−03 |
| RS10512680 | 509 | FLJ39155 | 133584 | 5 | 38307189 | 226 | 1.26 | 232 | 1.35 | 2.97 | 456 | 3.14E−03 |
| RS157819 | 1320 | | | 16 | 75582042 | 252 | 1.27 | 164 | 1.36 | 2.97 | 414 | 3.15E−03 |
| RS2248518 | 991 | C10orf107 | 219621 | 10 | 63181795 | 333 | 1.27 | 167 | 1.35 | 2.96 | 498 | 3.22E−03 |
| RS7827548 | 765 | CSMD1 | 64478 | 8 | 4593724 | 331 | 1.27 | 167 | 1.35 | 2.96 | 496 | 3.22E−03 |
| RS6954168 | 755 | LOC653748 | 653748 | 7 | 153533800 | 489 | 1.29 | 7 | 1.63 | 2.96 | 494 | 3.22E−03 |
| RS1385080 | 1271 | | | 15 | 44513999 | 470 | 1.29 | 20 | 1.5 | 2.96 | 488 | 3.23E−03 |
| RS1443786 | 367 | | | 4 | 83267782 | 338 | 1.28 | 146 | 1.37 | 2.96 | 482 | 3.23E−03 |
| RS10484230 | 1236 | | | 14 | 43700947 | 379 | 1.27 | 105 | 1.37 | 2.96 | 482 | 3.23E−03 |
| RS223128 | 1337 | | | 17 | 26933541 | 320 | 1.27 | 164 | 1.35 | 2.96 | 482 | 3.23E−03 |
| RS7779879 | 743 | | | 7 | 114677634 | 95 | 1.22 | 319 | 1.32 | 2.96 | 412 | 3.25E−03 |
| RS10502185 | 1107 | | | 11 | 113786625 | 468 | 1.29 | 32 | 1.45 | 2.95 | 498 | 3.33E−03 |
| RS10490838 | 263 | | | 3 | 24574382 | 483 | 1.29 | 15 | 1.52 | 2.95 | 496 | 3.33E−03 |
| RS10519443 | 564 | COMMD10 | 51397 | 5 | 115500392 | 470 | 1.28 | 28 | 1.46 | 2.95 | 496 | 3.33E−03 |
| RS10503272 | 763 | CSMD1 | 64478 | 8 | 4592774 | 332 | 1.27 | 166 | 1.35 | 2.95 | 496 | 3.33E−03 |
| RS7675645 | 352 | | | 4 | 59309086 | 410 | 1.28 | 86 | 1.38 | 2.95 | 494 | 3.33E−03 |
| RS950016 | 1244 | | | 14 | 81514393 | 467 | 1.29 | 27 | 1.46 | 2.95 | 492 | 3.33E−03 |
| RS2396821 | 635 | | | 6 | 47183758 | 401 | 1.28 | 89 | 1.38 | 2.95 | 488 | 3.33E−03 |
| RS2981069 | 826 | | | 8 | 70100501 | 426 | 1.28 | 64 | 1.4 | 2.95 | 488 | 3.33E−03 |
| RS9296486 | 616 | PHACTR1 | 221693 | 6 | 12956955 | 401 | 1.32 | 87 | 1.21 | 2.95 | 486 | 3.33E−03 |
| RS10484416 | 657 | | | 6 | 67185561 | 417 | 1.28 | 71 | 1.4 | 2.95 | 486 | 3.33E−03 |
| RS10500585 | 1319 | | | 16 | 72882363 | 347 | 1.32 | 139 | 1.23 | 2.95 | 484 | 3.33E−03 |
| RS7220359 | 1354 | RGS9 | 8787 | 17 | 60614155 | 472 | 1.29 | 14 | 1.53 | 2.95 | 484 | 3.33E−03 |
| RS1037364 | 1344 | YPEL2 | 388403 | 17 | 54792947 | 405 | 1.31 | 71 | 1.2 | 2.95 | 474 | 3.33E−03 |
| RS725882 | 409 | | | 4 | 122925927 | 417 | 1.31 | 51 | 1.18 | 2.95 | 466 | 3.34E−03 |
| RS10488493 | 726 | FLJ45974 | 401337 | 7 | 53543405 | 376 | 1.27 | 124 | 1.37 | 2.94 | 498 | 3.43E−03 |
| RS9298324 | 843 | | | 8 | 80208439 | 417 | 1.28 | 83 | 1.39 | 2.94 | 498 | 3.43E−03 |
| RS9325158 | 1123 | FLJ32942 | 121355 | 12 | 53146448 | 474 | 1.29 | 26 | 1.47 | 2.94 | 498 | 3.43E−03 |
| RS352427 | 816 | | | 8 | 28571996 | 482 | 1.29 | 16 | 1.52 | 2.94 | 496 | 3.44E−03 |
| RS4741021 | 894 | | | 9 | 10237653 | 165 | 1.35 | 333 | 1.27 | 2.94 | 496 | 3.44E−03 |
| RS10505768 | 1112 | | | 12 | 12790297 | 114 | 1.22 | 382 | 1.32 | 2.94 | 494 | 3.44E−03 |
| RS1233775 | 1425 | C20orf133 | 140733 | 20 | 15451543 | 379 | 1.32 | 117 | 1.22 | 2.94 | 494 | 3.44E-03 |
| RS10517281 | 348 | SCFD2 | 152579 | 4 | 53868523 | 366 | 1.27 | 128 | 1.36 | 2.94 | 492 | 3.44E−03 |
| RS10498321 | 1231 | NPAS3 | 64067 | 14 | 33263982 | 433 | 1.28 | 61 | 1.4 | 2.94 | 492 | 3.44E−03 |
| RS2831853 | 1449 | | | 21 | 28781867 | 482 | 1.3 | 10 | 1.02 | 2.94 | 490 | 3.44E−03 |
| RS503699 | 1178 | MGC9850 | 219404 | 13 | 27112734 | 184 | 1.35 | 304 | 1.27 | 2.94 | 486 | 3.44E−03 |
| RS8137239 | 1456 | LOC644802 | 644802 | 22 | 15504963 | 461 | 1.29 | 21 | 1.49 | 2.94 | 480 | 3.44E−03 |
| RS10517799 | 445 | | | 4 | 165398678 | 379 | 1.32 | 101 | 1.22 | 2.94 | 478 | 3.44E−03 |
| RS1154307 | 550 | | | 5 | 111049411 | 424 | 1.29 | 44 | 1.43 | 2.94 | 466 | 3.45E−03 |
| RS10513217 | 305 | SLC9A9 | 285195 | 3 | 144888396 | 163 | 1.36 | 301 | 1.27 | 2.94 | 462 | 3.45E−03 |
| RS10518928 | 1269 | MEIS2 | 4212 | 15 | 35140532 | 367 | 1.32 | 97 | 1.21 | 2.94 | 462 | 3.45E−03 |
| RS1993468 | 1413 | | | 19 | 33195886 | 167 | 1.35 | 249 | 1.26 | 2.94 | 414 | 3.47E−03 |
| RS9283695 | 323 | KIAA0746 | 23231 | 4 | 25462687 | 327 | 1.33 | 173 | 1.24 | 2.93 | 498 | 3.55E−03 |
| RS6829390 | 338 | TEC | 7006 | 4 | 48039914 | 484 | 1.29 | 16 | 1.51 | 2.93 | 498 | 3.55E−03 |
| RS10498681 | 615 | PHACTRI | 221692 | 6 | 12916270 | 464 | 1.31 | 34 | 1.15 | 2.93 | 496 | 3.55E−03 |
| RS1150030 | 959 | | | 10 | 9405319 | 467 | 1.29 | 31 | 1.45 | 2.93 | 496 | 3.55E−03 |
| RS1938887 | 1101 | | | 11 | 99767680 | 71 | 1.2 | 425 | 1.31 | 2.93 | 494 | 3.55E−03 |
| RS979022 | 387 | | | 4 | 105311555 | 260 | 1.26 | 232 | 1.34 | 2.93 | 490 | 3.55E−03 |
| RS1485870 | 453 | LOC442117 | 442117 | 4 | 173125585 | 386 | 1.32 | 100 | 1.22 | 2.93 | 484 | 3.55E−03 |
| RS4696124 | 435 | | | 4 | 153293160 | 233 | 1.26 | 251 | 1.34 | 2.93 | 482 | 3.55E−03 |
| RS2721304 | 559 | | | 5 | 113462331 | 428 | 1.29 | 56 | 1.41 | 2.93 | 482 | 3.55E−03 |
| RS1943752 | 1102 | | | 11 | 100601234 | 354 | 1.27 | 128 | 1.36 | 2.93 | 480 | 3.55E−03 |
| RS7328960 | 1225 | FLJ26443 | 400165 | 13 | 112376717 | 248 | 1.26 | 234 | 1.34 | 2.93 | 480 | 3.55E−03 |
| RS10509209 | 1001 | | | 10 | 66120490 | 243 | 1.26 | 233 | 1.34 | 2.93 | 474 | 3.55E−03 |
| RS10491082 | 1327 | LOC342531 | 342531 | 17 | 6498311 | 279 | 1.27 | 197 | 1.35 | 2.93 | 474 | 3.55E−03 |
| RS10490027 | 237 | KIAA1843 | 84540 | 2 | 210559968 | 373 | 1.29 | 27 | 1.46 | 2.93 | 398 | 3.58E−03 |
| RS1528842 | 209 | | | 2 | 148291308 | 442 | 1.31 | 58 | 1.19 | 2.92 | 498 | 3.66E−03 |
| RS2991463 | 1209 | | | 13 | 78682643 | 134 | 1.36 | 362 | 1.27 | 2.92 | 494 | 3.66E−03 |
| RS1158815 | 482 | | | 4 | 188232341 | 136 | 1.23 | 356 | 1.32 | 2.92 | 490 | 3.66E−03 |
| RS10484317 | 604 | FARS2 | 10667 | 6 | 5614669 | 469 | 1.28 | 23 | 1.47 | 2.92 | 490 | 3.66E−03 |
| RS665889 | 915 | | | 9 | 85011765 | 277 | 1.26 | 213 | 1.34 | 2.92 | 488 | 3.66E−03 |
| RS709228 | 1147 | | | 12 | 91074897 | 343 | 1.27 | 147 | 1.36 | 2.92 | 488 | 3.66E−03 |
| RS10508059 | 1223 | VGCNL1 | 259232 | 13 | 1007320832 | 407 | 1.28 | 81 | 1.38 | 2.92 | 486 | 3.66E−03 |
| RS2103391 | 1154 | CHST11 | 50515 | 12 | 103381740 | 317 | 1.33 | 169 | 1.24 | 2.92 | 484 | 3.66E−03 |
| RS7576837 | 215 | | | 2 | 180945207 | 90 | 1.37 | 394 | 1.27 | 2.92 | 482 | 3.66E−03 |
| RS6974799 | 737 | MAGI2 | 9863 | 7 | 77761741 | 390 | 1.28 | 90 | 1.38 | 2.92 | 478 | 3.67E−03 |
| RS1480757 | 356 | | | 4 | 71398388 | 300 | 1.27 | 172 | 1.36 | 2.92 | 470 | 3.67E−03 |
| RS10502909 | 1393 | | | 18 | 46384530 | 378 | 1.27 | 92 | 1.36 | 2.92 | 468 | 3.67E−03 |
| RS1456196 | 293 | | | 3 | 119137243 | 374 | 1.28 | 86 | 1.38 | 2.92 | 458 | 3.67E−03 |
| RS6537736 | 84 | | | 1 | 112623366 | 395 | 1.32 | 57 | 1.19 | 2.92 | 450 | 3.68E−03 |
| RS959902 | 574 | | | 5 | 121872175 | 304 | 1.32 | 92 | 1.22 | 2.92 | 394 | 3.70E−03 |
| RS2033874 | 138 | | | 2 | 12053486 | 345 | 1.27 | 155 | 1.36 | 2.91 | 498 | 3.78E−03 |
| RS899557 | 595 | | | 5 | 170994216 | 277 | 1.33 | 223 | 1.25 | 2.91 | 498 | 3.78E−03 |
| RS2480014 | 618 | | | 6 | 23021718 | 355 | 1.32 | 145 | 1.24 | 2.91 | 498 | 3.78E−03 |
| RS2166890 | 80 | | | 1 | 104824377 | 354 | 1.27 | 144 | 1.36 | 2.91 | 496 | 3.78E−03 |
| RS6801518 | 307 | | | 3 | 145604317 | 402 | 1.32 | 96 | 1.22 | 2.91 | 496 | 3.78E−03 |
| RS9312699 | 349 | | | 4 | 59216510 | 421 | 1.28 | 77 | 1.39 | 2.91 | 496 | 3.78E−03 |
| RS6956726 | 735 | RSBN1L | 222194 | 7 | 77019497 | 196 | 1.25 | 302 | 1.33 | 2.91 | 496 | 3.78E−03 |
| RS7654796 | 375 | MAPK10 | 5602 | 4 | 87588743 | 200 | 1.34 | 296 | 1.26 | 2.91 | 494 | 3.78E−03 |
| RS10509702 | 1009 | | | 10 | 97914086 | 484 | 1.29 | 12 | 1.55 | 2.91 | 494 | 3.78E−03 |
| RS10512573 | 1356 | | | 17 | 65056574 | 438 | 1.28 | 58 | 1.4 | 2.91 | 494 | 3.78E−03 |
| RS744832 | 191 | | | 2 | 117564871 | 437 | 1.28 | 55 | 1.41 | 2.91 | 490 | 3.78E−03 |
| RS6935765 | 655 | | | 6 | 67179216 | 421 | 1.29 | 71 | 1.4 | 2.91 | 490 | 3.78E−03 |
| RS10518774 | 1282 | UNC13C | 440279 | 15 | 52700466 | 249 | 1.34 | 237 | 1.26 | 2.91 | 484 | 3.78E−03 |
| RS2069837 | 710 | IL6 | 3569 | 7 | 22541267 | 458 | 1.29 | 24 | 1.47 | 2.91 | 480 | 3.78E−03 |
| RS726252 | 90 | PAPPA2 | 60676 | 1 | 173391904 | 303 | 1.27 | 177 | 1.35 | 2.91 | 478 | 3.78E−03 |
| RS717595 | 767 | LOC645986 | 645986 | 8 | 9234995 | 343 | 1.32 | 123 | 1.23 | 2.91 | 464 | 3.79E−03 |
| RS1881014 | 732 | | | 7 | 69770606 | 441 | 1.3 | 17 | 1.52 | 2.91 | 456 | 3.79E−03 |
| RS2458349 | 1105 | | | 11 | 105805096 | 210 | 1.34 | 248 | 1.26 | 2.91 | 456 | 3.79E−03 |
| RS10500985 | 1052 | LUZP2 | 338645 | 11 | 24590527 | 87 | 1.37 | 367 | 1.27 | 2.91 | 452 | 3.79E−03 |
| RS722708 | 1363 | SPIRE1 | 56907 | 18 | 12535581 | 185 | 1.32 | 209 | 1.24 | 2.91 | 392 | 3.82E−03 |
| RS10516773 | 376 | MAPK10 | 5602 | 4 | 87589325 | 202 | 1.34 | 298 | 1.27 | 2.9 | 498 | 3.90E−03 |
| RS6976144 | 734 | RSBN1L | 222194 | 7 | 77019455 | 197 | 1.25 | 303 | 1.33 | 2.9 | 498 | 3.90E−03 |
| RS711088 | 1137 | | | 12 | 76434536 | 292 | 1.33 | 202 | 1.25 | 2.9 | 492 | 3.90E−03 |
| RS2147085 | 37 | | | 1 | 80971158 | 232 | 1.33 | 258 | 1.25 | 2.9 | 488 | 3.90E−03 |
| RS2250756 | 477 | | | 4 | 182095665 | 262 | 1.26 | 188 | 1.35 | 2.9 | 448 | 3.92E−03 |
| RS9310281 | 282 | | | 3 | 73964504 | 339 | 1.27 | 103 | 1.36 | 2.9 | 440 | 3.92E−03 |
| RS10485485 | 1419 | GPR73L1 | 128674 | 20 | 5232380 | 102 | 1.37 | 398 | 1.28 | 2.89 | 498 | 4.02E−03 |
| RS233051 | 42 | DDAH1 | 23576 | 1 | 85550434 | 364 | 1.32 | 132 | 1.23 | 2.89 | 494 | 4.02E−03 |
| RS2725010 | 762 | CSMD1 | 64478 | 8 | 4542585 | 287 | 1.26 | 209 | 1.34 | 2.89 | 494 | 4.02E−03 |
| RS953177 | 823 | DNAJC5B | 85479 | 8 | 67096174 | 470 | 1.29 | 26 | 1.47 | 2.89 | 494 | 4.02E−03 |
| RS10503166 | 756 | DLGAP2 | 9228 | 8 | 1477325 | 60 | 1.4 | 434 | 1.28 | 2.89 | 492 | 4.02E−03 |
| RS7263438 | 1418 | GPR73L1 | 128674 | 20 | 5232251 | 391 | 1.28 | 101 | 1.37 | 2.89 | 490 | 4.02E−03 |
| RS10498835 | 653 | | | 6 | 66572761 | 423 | 1.32 | 65 | 1.2 | 2.89 | 486 | 4.02E−03 |
| RS9319534 | 1321 | WWOX | 51741 | 16 | 77648659 | 309 | 1.33 | 171 | 1.24 | 2.89 | 478 | 4.03E−03 |
| RS9297874 | 884 | | | 8 | 134984532 | 399 | 1.32 | 73 | 1.21 | 2.89 | 470 | 4.03E−03 |
| RS6837953 | 437 | | | 4 | 154288568 | 432 | 1.28 | 36 | 1.42 | 2.89 | 466 | 4.03E−03 |
| RS720613 | 749 | | | 7 | 124046621 | 366 | 1.28 | 98 | 1.38 | 2.89 | 462 | 4.03E−03 |
| RS1830826 | 41 | | | 1 | 83991668 | 443 | 1.31 | 15 | 1.08 | 2.89 | 456 | 4.04E−03 |
| RS802279 | 684 | TCBA1 | 154215 | 6 | 124562146 | 336 | 1.31 | 90 | 1.21 | 2.89 | 424 | 4.05E−03 |
| RS2163120 | 792 | | | 8 | 16165538 | 488 | 1.29 | 12 | 1.54 | 2.88 | 498 | 4.15E−03 |
| RS730837 | 306 | | | 3 | 145584497 | 399 | 1.32 | 93 | 1.22 | 2.88 | 490 | 4.15E−03 |
| RS2138992 | 105 | IL19 | 29949 | 1 | 203382799 | 121 | 1.23 | 363 | 1.32 | 2.88 | 482 | 4.15E−03 |
| RS10501826 | 1097 | | | 11 | 94739512 | 286 | 1.33 | 192 | 1.25 | 2.88 | 476 | 4.16E−03 |
| RS7609812 | 267 | | | 3 | 30885478 | 320 | 1.27 | 156 | 1.36 | 2.88 | 474 | 4.16E−03 |
| RS1298362 | 1136 | | | 12 | 74493708 | 225 | 1.25 | 251 | 1.33 | 2.88 | 474 | 4.16E−03 |
| RS1485871 | 454 | LOC442117 | 442117 | 4 | 173125753 | 377 | 1.32 | 91 | 1.22 | 2.88 | 466 | 4.16E−03 |
| RS10509757 | 1012 | CNNM2 | 54805 | 10 | 104742950 | 302 | 1.33 | 164 | 1.25 | 2.88 | 464 | 4.16E−03 |
| RS10497017 | 208 | | | 2 | 148275544 | 418 | 1.32 | 46 | 1.18 | 2.88 | 462 | 4.16E−03 |
| RS9284299 | 1277 | UNC13C | 440279 | 15 | 52685923 | 115 | 1.36 | 285 | 1.27 | 2.88 | 398 | 4.19E−03 |
| RS10498212 | 249 | IRS1 | 3667 | 2 | 227476780 | 482 | 1.3 | 18 | 1.1 | 2.87 | 498 | 4.28E−03 |
| RS10507930 | 1212 | | | 13 | 82396265 | 494 | 1.29 | 6 | 1.65 | 2.87 | 498 | 4.28E−03 |
| RS1901439 | 204 | | | 2 | 134271203 | 440 | 1.28 | 58 | 1.4 | 2.87 | 496 | 4.28E−03 |
| RS10520494 | 478 | | | 4 | 182747335 | 426 | 1.28 | 72 | 1.39 | 2.87 | 496 | 4.28E−03 |
| RS696428 | 1139 | | | 12 | 76454956 | 293 | 1.33 | 203 | 1.25 | 2.87 | 494 | 4.28E−03 |
| RS770123 | 1140 | NAV3 | 89795 | 12 | 76926472 | 310 | 1.33 | 186 | 1.25 | 2.87 | 494 | 4.28E−03 |
| RS1924794 | 1191 | | | 13 | 47173413 | 283 | 1.27 | 213 | 1.34 | 2.87 | 494 | 4.28E−03 |
| RS4617036 | 729 | FLJ10099 | 55069 | 7 | 65859759 | 345 | 1.32 | 149 | 1.24 | 2.87 | 492 | 4.28E−03 |
| RS943345 | 1007 | SORBS1 | 10580 | 10 | 97240620 | 83 | 1.38 | 411 | 1.28 | 2.87 | 492 | 4.28E−03 |
| RS7874576 | 896 | | | 9 | 12600410 | 487 | 1.29 | 5 | 1.68 | 2.87 | 490 | 4.28E−03 |
| RS917022 | 393 | COL25A1 | 84570 | 4 | 110557948 | 429 | 1.31 | 61 | 1.2 | 2.87 | 488 | 4.28E−03 |
| RS2913260 | 529 | SV2C | 22987 | 5 | 75644923 | 424 | 1.31 | 66 | 1.2 | 2.87 | 488 | 4.28E−03 |
| RS10491705 | 887 | | | 9 | 2259108 | 423 | 1.31 | 67 | 1.2 | 2.87 | 488 | 4.28E−03 |
| RS6740690 | 205 | | | 2 | 134611767 | 243 | 1.26 | 243 | 1.34 | 2.87 | 484 | 4.28E−03 |
| RS297574 | 865 | | | 8 | 96256279 | 120 | 1.37 | 342 | 1.28 | 2.87 | 460 | 4.29E−03 |
| RS9317744 | 1176 | | | 13 | 22963766 | 451 | 1.3 | 11 | 1.56 | 2.87 | 460 | 4.29E−03 |
| RS1959736 | 1245 | | | 14 | 82487626 | 51 | 1.41 | 407 | 1.28 | 2.87 | 456 | 4.30E−03 |
| RS10516114 | 600 | | | 5 | 173638652 | 463 | 1.31 | 37 | 1.16 | 2.86 | 498 | 4.41E−03 |
| RS7465623 | 877 | | | 8 | 119372441 | 116 | 1.37 | 384 | 1.28 | 2.86 | 498 | 4.41E−03 |
| RS10498292 | 1229 | | | 14 | 27284462 | 413 | 1.28 | 87 | 1.38 | 2.86 | 498 | 4.41E−03 |
| RS2964299 | 588 | | | 5 | 165876275 | 478 | 1.31 | 20 | 1.11 | 2.86 | 496 | 4.42E−03 |
| RS10506524 | 1124 | MSRB3 | 253827 | 12 | 64070379 | 480 | 1.29 | 18 | 1.5 | 2.86 | 496 | 4.42E−03 |
| RS1216987 | 1189 | | | 13 | 46240774 | 461 | 1.29 | 37 | 1.43 | 2.86 | 496 | 4.42E−03 |
| RS10511294 | 289 | | | 3 | 112534708 | 394 | 1.32 | 102 | 1.22 | 2.86 | 494 | 4.42E−03 |
| RS10503273 | 764 | CSMD1 | 64478 | 8 | 4592896 | 330 | 1.27 | 166 | 1.35 | 2.86 | 494 | 4.42E−03 |
| RS438548 | 1210 | | | 13 | 81272799 | 447 | 1.31 | 49 | 1.18 | 2.86 | 494 | 4.42E−03 |
| RS2111826 | 309 | CLDN11 | 5010 | 3 | 171623548 | 455 | 1.31 | 39 | 1.16 | 2.86 | 492 | 4.42E−03 |
| RS4521277 | 266 | | | 3 | 30879801 | 171 | 1.35 | 321 | 1.27 | 2.86 | 490 | 4.42E−03 |
| RS10514263 | 545 | EDIL3 | 10085 | 5 | 83426017 | 474 | 1.29 | 18 | 1.49 | 2.86 | 490 | 4.42E−03 |
| RS10489920 | 66 | PAP2D | 163404 | 1 | 99146514 | 436 | 1.28 | 52 | 1.41 | 2.86 | 486 | 4.42E−03 |
| RS9327178 | 568 | | | 5 | 120387547 | 202 | 1.34 | 286 | 1.26 | 2.86 | 486 | 4.42E−03 |
| RS883025 | 935 | RGS3 | 5998 | 9 | 113405053 | 386 | 1.31 | 100 | 1.21 | 2.86 | 484 | 4.42E−03 |
| RS4767227 | 1157 | | | 12 | 113050998 | 194 | 1.25 | 282 | 1.34 | 2.86 | 474 | 4.42E−03 |
| RS9318391 | 1202 | | | 13 | 75517111 | 381 | 1.28 | 67 | 1.39 | 2.86 | 446 | 4.44E−03 |
| RS1916411 | 997 | | | 10 | 65340839 | 222 | 1.25 | 222 | 1.33 | 2.86 | 442 | 4.44E−03 |
| RS7023082 | 910 | | | 9 | 83559554 | 250 | 1.33 | 176 | 1.25 | 2.86 | 424 | 4.45E−03 |
| RS1996970 | 173 | SALF | 286749 | 2 | 48710134 | 316 | 1.28 | 88 | 1.38 | 2.86 | 402 | 4.46E−03 |

TABLE 3
SNP markers with P-value less than 0.005 for HDL from F-test (one-way ANOVA).
Alleles are codes as 1 = A, 2 = C, 3 = G, and 4 = T. For each group number of
observations ‘n’ and average value ‘avg’ are presented.

| RS_id | SEQ_ID_no | Gene | Gene_id | Chr | Position | g1 | n1 |
|---|---|---|---|---|---|---|---|
| RS1410126 | 687 | C6orf103 | 79747 | 6 | 147125370 | 1/1 | 5 |
| RS10498321 | 1231 | NPAS3 | 64067 | 14 | 33263982 | 1/1 | 187 |
| RS744832 | 191 | | | 2 | 117564871 | 1/1 | 1 |
| RS10485909 | 738 | MAGI2 | 9863 | 7 | 77884399 | 2/2 | 227 |
| RS2195989 | 872 | ANGPT1 | 284 | 8 | 108555088 | 2/2 | 9 |
| RS1399503 | 1159 | | | 12 | 127163861 | 3/3 | 204 |
| RS4102401 | 866 | RRM2B | 50484 | 8 | 103288416 | 2/2 | 1 |
| RS10518533 | 420 | | | 4 | 129815863 | 1/1 | 5 |
| RS10504433 | 824 | | | 8 | 70021434 | 2/2 | 196 |
| RS10516418 | 384 | | | 4 | 98106087 | 1/1 | 213 |
| RS2230133 | 580 | TAF7 | 6879 | 5 | 140679241 | 2/2 | 205 |
| RS10498260 | 253 | MGC42174 | 129563 | 2 | 232882126 | 1/1 | 1 |
| RS10486467 | 712 | | | 7 | 26427754 | 1/1 | 1 |
| RS2120650 | 1292 | | | 15 | 84419700 | 1/1 | 223 |
| RS1467596 | 605 | FLJ33708 | 285780 | 6 | 6360933 | 1/1 | 1 |
| RS2170398 | 773 | | | 8 | 13592708 | 2/2 | 1 |
| RS352766 | 775 | | | 8 | 15688930 | 2/2 | 1 |
| RS10488888 | 395 | | | 4 | 111641312 | 2/2 | 1 |
| RS4960221 | 606 | LY86 | 9450 | 6 | 6547432 | 2/2 | 186 |
| RS10494066 | 81 | NTNG1 | 22854 | 1 | 107501550 | 2/2 | 6 |
| RS10517155 | 330 | | | 4 | 28879822 | 2/2 | 1 |
| RS9303878 | 1360 | | | 18 | 1564764 | 1/1 | 202 |
| RS10510995 | 281 | | | 3 | 70525819 | 1/1 | 1 |
| RS718759 | 575 | | | 5 | 125467810 | 2/2 | 1 |
| RS1480145 | 796 | | | 8 | 16181494 | 2/2 | 238 |
| RS10485110 | 682 | | | 6 | 116309557 | 3/3 | 6 |
| RS1902049 | 154 | LTBP1 | 4052 | 2 | 33281233 | 3/3 | 136 |
| RS2235216 | 683 | | | 6 | 116310188 | 1/1 | 6 |
| RS470559 | 1399 | MBP | 4155 | 18 | 72887390 | 2/2 | 3 |
| RS10503573 | 780 | MSR1 | 4481 | 8 | 16047855 | 1/1 | 236 |
| RS2854946 | 1259 | SERPINAS | 5104 | 14 | 94118132 | 2/2 | 13 |
| RS4631686 | 3 | | | 1 | 14322780 | 1/3 | 8 |
| RS6954679 | 728 | | | 7 | 62940814 | 1/1 | 207 |
| RS866651 | 262 | THRB | 7068 | 3 | 24237159 | 2/2 | 167 |
| RS2255565 | 194 | | | 2 | 118114732 | 1/1 | 191 |
| RS10483988 | 1250 | GALC | 2581 | 14 | 87504310 | 2/2 | 178 |
| RS387661 | 587 | GABRG2 | 2566 | 5 | 161476289 | 2/2 | 2 |
| RS2029259 | 484 | | | 4 | 188422292 | 1/1 | 194 |
| RS2113515 | 195 | | | 2 | 118115019 | 2/2 | 187 |
| RS10503586 | 803 | | | 8 | 16792998 | 1/1 | 232 |
| RS285406 | 855 | | | 8 | 87094129 | 2/2 | 228 |
| RS6811055 | 336 | | | 4 | 46732838 | 2/2 | 75 |
| RS397618 | 1063 | TMEM16C | 63982 | 11 | 26546754 | 2/2 | 236 |
| RS896990 | 251 | MGC42174 | 129563 | 2 | 232714361 | 1/1 | 3 |
| RS250387 | 518 | ARL15 | 54622 | 5 | 53620394 | 1/1 | 172 |
| RS9299445 | 993 | LOC644954 | 644954 | 10 | 63244794 | 2/2 | 206 |
| RS10516673 | 370 | HNRPD | 3184 | 4 | 83635972 | 1/1 | 237 |
| RS10520350 | 472 | | | 4 | 177769342 | 2/2 | 1 |
| RS9299446 | 994 | LOC644954 | 644954 | 10 | 63244886 | 1/1 | 3 |
| RS2170116 | 791 | | | 8 | 16157007 | 2/2 | 231 |
| RS4888946 | 1324 | | | 16 | 77874931 | 1/1 | 177 |
| RS10495783 | 155 | LTBP1 | 4052 | 2 | 33313283 | 2/2 | 50 |
| RS10516834 | 378 | | | 4 | 90629156 | 1/1 | 3 |
| RS2207139 | 645 | | | 6 | 50953449 | 2/2 | 8 |
| RS8054578 | 1323 | | | 16 | 77874316 | 1/1 | 3 |
| RS7897958 | 987 | BICC1 | 80114 | 10 | 60121804 | 1/1 | 6 |
| RS1121640 | 1099 | | | 11 | 96694636 | 2/2 | 218 |
| RS10493425 | 9 | | | 1 | 67722148 | 1/1 | 216 |
| RS965158 | 868 | | | 8 | 107564094 | 2/4 | 11 |
| RS10482871 | 1439 | | | 21 | 15700259 | 2/3 | 5 |
| RS10503572 | 789 | | | 8 | 16131101 | 1/3 | 5 |
| RS9316436 | 1168 | EFHA1 | 221154 | 13 | 21000655 | 2/2 | 183 |
| RS812800 | 1338 | | | 17 | 30646887 | 1/1 | 9 |
| RS10508377 | 962 | | | 10 | 10107183 | 1/1 | 218 |
| RS10500985 | 1052 | LUZP2 | 338645 | 11 | 24590527 | 1/1 | 6 |
| RS3116231 | 252 | MGC42174 | 129563 | 2 | 232717788 | 2/2 | 2 |
| RS1490125 | 651 | | | 6 | 66451580 | 3/3 | 181 |
| RS8049647 | 1310 | | | 16 | 50695297 | 1/1 | 166 |
| RS10517562 | 436 | | | 4 | 153393348 | 1/1 | 24 |
| RS2222654 | 430 | | | 4 | 137363666 | 1/1 | 77 |
| RS2357344 | 1242 | SYNE2 | 23224 | 14 | 63578006 | 2/2 | 5 |
| RS4869279 | 547 | | | 5 | 95654674 | 2/2 | 168 |
| RS11232919 | 1087 | LOC646176 | 646176 | 11 | 81284714 | 2/2 | 2 |
| RS9315157 | 1181 | 13CDNA73 | 10129 | 13 | 31642352 | 1/1 | 6 |
| RS1675904 | 695 | | | 6 | 154399717 | 2/2 | 204 |
| RS1502887 | 358 | FRAS1 | 80144 | 4 | 79340562 | 2/2 | 207 |
| RS1380835 | 1390 | | | 18 | 39747045 | 3/3 | 182 |
| RS10509764 | 1013 | CNNM2 | 54805 | 10 | 104766465 | 2/2 | 126 |
| RS10514450 | 1322 | | | 16 | 77874213 | 2/2 | 3 |
| RS2101435 | 254 | MGC42174 | 129563 | 2 | 232940663 | 2/2 | 191 |
| RS9308931 | 156 | LTBP1 | 4052 | 2 | 33333348 | 1/1 | 65 |
| RS2290547 | 272 | HYPB | 29072 | 3 | 47036187 | 1/1 | 13 |
| RS10504709 | 848 | | | 8 | 80781919 | 1/1 | 16 |
| RS10521979 | 1461 | DMD | 1756 | X | 31547276 | 3/3 | 41 |
| RS10496647 | 202 | | | 2 | 126205166 | 1/1 | 1 |
| RS8076457 | 1328 | NTN1 | 9423 | 17 | 8884654 | 2/2 | 169 |
| RS297733 | 1417 | | | 20 | 4357955 | 3/3 | 160 |
| RS10513319 | 952 | | | 9 | 117781003 | 1/1 | 233 |
| RS10496649 | 203 | | | 2 | 126293622 | 2/2 | 207 |
| RS9325158 | 1123 | FLJ32942 | 121355 | 12 | 53146448 | 2/2 | 1 |
| RS1817186 | 319 | | | 4 | 14843817 | 2/2 | 197 |
| RS346421 | 527 | | | 5 | 62565739 | 3/3 | 201 |
| RS3770899 | 163 | CRIM1 | 51232 | 2 | 36566423 | 1/1 | 1 |
| RS10506790 | 1141 | | | 12 | 77762475 | 1/1 | 2 |
| RS1749541 | 57 | ABCD3 | 5825 | 1 | 94670399 | 2/2 | 116 |
| RS1935229 | 1 | CAMTA1 | 23261 | 1 | 7695267 | 2/4 | 33 |
| RS250388 | 519 | ARL15 | 54622 | 5 | 53621019 | 1/1 | 6 |
| RS4073625 | 292 | | | 3 | 118628997 | 2/2 | 3 |
| RS4770403 | 1173 | SGCG | 6445 | 13 | 22653127 | 1/1 | 2 |
| RS2157768 | 1340 | | | 17 | 49582892 | 1/1 | 233 |
| RS10514844 | 1182 | | | 13 | 37808480 | 2/3 | 7 |
| RS10484205 | 1237 | | | 14 | 48644630 | 1/1 | 15 |
| RS7897663 | 1015 | NT5C2 | 22978 | 10 | 104895594 | 1/1 | 123 |
| RS2278016 | 1308 | | | 16 | 50674510 | 2/2 | 6 |
| RS9303347 | 1342 | | | 17 | 49585177 | 1/1 | 1 |
| RS1011439 | 1339 | | | 17 | 49582464 | 1/1 | 1 |
| RS10494663 | 94 | | | 1 | 188507735 | 1/1 | 5 |
| RS3218491 | 753 | XRCC2 | 7516 | 7 | 151791396 | 2/4 | 6 |
| RS1527420 | 196 | | | 2 | 119267521 | 1/1 | 183 |
| RS10509929 | 1021 | | | 10 | 112482278 | 2/2 | 3 |
| RS10508378 | 961 | | | 10 | 10085297 | 2/2 | 1 |
| RS2413809 | 1270 | | | 15 | 44497210 | 2/2 | 102 |
| RS10508940 | 986 | PRKG1 | 5592 | 10 | 52888932 | 2/2 | 160 |
| RS9284617 | 361 | FRAS1 | 80144 | 4 | 79349747 | 1/3 | 41 |
| RS788005 | 232 | | | 2 | 198059567 | 2/2 | 144 |
| RS2056247 | 1092 | | | 11 | 93315872 | 3/3 | 8 |
| RS10495774 | 150 | | | 2 | 30418327 | 2/2 | 11 |
| RS10484317 | 604 | FARS2 | 10667 | 6 | 5614669 | 2/2 | 225 |
| RS10517496 | 353 | | | 4 | 61091463 | 2/2 | 2 |
| RS10485111 | 681 | | | 6 | 116299831 | 2/2 | 186 |
| RS2748324 | 658 | | | 6 | 71789087 | 2/2 | 209 |
| RS4405428 | 1213 | GPC5 | 2262 | 13 | 91916519 | 2/2 | 190 |
| RS1044045 | 1167 | EFHA1 | 221154 | 13 | 20965072 | 1/1 | 165 |
| RS176435 | 680 | | | 6 | 103636845 | 2/2 | 179 |
| RS10513351 | 953 | | | 9 | 119606328 | 3/3 | 67 |
| RS3766988 | 91 | CACNA1E | 777 | 1 | 178398940 | 2/2 | 8 |
| RS8068574 | 1343 | | | 17 | 50327246 | 1/1 | 151 |
| RS10483457 | 1232 | GARNL1 | 253959 | 14 | 35211683 | 1/1 | 217 |
| RS879780 | 1109 | APLP2 | 334 | 11 | 129513314 | 2/2 | 1 |
| RS10513301 | 943 | ASTN2 | 23245 | 9 | 117241331 | 2/2 | 173 |
| RS4121693 | 1108 | | | 11 | 121613919 | 2/2 | 24 |
| RS10508059 | 1223 | VGCNL1 | 259232 | 13 | 100730832 | 2/2 | 171 |
| RS1878144 | 193 | | | 2 | 117895419 | 3/3 | 187 |
| RS10517504 | 354 | | | 4 | 61173582 | 1/1 | 2 |
| RS1356216 | 192 | | | 2 | 117878791 | 3/3 | 195 |
| RS2127258 | 566 | | | 5 | 117512842 | 2/2 | 3 |
| RS1226489 | 634 | GPR110 | 266977 | 6 | 47100017 | 2/2 | 1 |
| RS877984 | 1088 | DLG2 | 1740 | 11 | 83257468 | 1/1 | 48 |
| RS6735220 | 229 | HECW2 | 57520 | 2 | 197080992 | 2/2 | 207 |
| RS3745718 | 1414 | CARD8 | 22900 | 19 | 53406965 | 1/1 | 77 |
| RS9303346 | 1341 | | | 17 | 49583384 | 1/1 | 1 |
| RS877345 | 958 | TAF3 | 83860 | 10 | 8093782 | 1/1 | 186 |
| RS973916 | 892 | | | 9 | 9842432 | 2/2 | 170 |
| RS1371077 | 1144 | | | 12 | 89049534 | 2/2 | 180 |
| RS1013401 | 643 | | | 6 | 48502893 | 1/1 | 85 |
| RS33375 | 598 | | | 5 | 170998718 | 1/1 | 6 |
| RS10490353 | 172 | UNQ6975 | 400952 | 2 | 45328245 | 2/2 | 203 |
| RS10517616 | 441 | | | 4 | 156733549 | 2/2 | 110 |
| RS716224 | 1179 | | | 13 | 27160986 | 1/1 | 5 |
| RS10497783 | 230 | HECW2 | 57520 | 2 | 197081030 | 1/1 | 2 |
| RS9304301 | 1391 | | | 18 | 40017390 | 2/2 | 226 |
| RS4646981 | 1106 | NCAM1 | 4684 | 11 | 112596015 | 1/3 | 10 |
| RS1535053 | 602 | | | 6 | 126089 | 1/1 | 130 |
| RS4146127 | 350 | | | 4 | 59260295 | 1/1 | 168 |
| RS10519371 | 426 | | | 4 | 137332410 | 3/3 | 69 |
| RS10509698 | 1008 | C10orf130 | 387707 | 10 | 97782342 | 1/1 | 1 |
| RS3218177 | 4 | E2F2 | 1870 | 1 | 23590594 | 1/1 | 1 |
| RS7577835 | 233 | | | 2 | 198203562 | 2/2 | 10 |
| RS9312013 | 260 | | | 3 | 8299589 | 3/3 | 223 |
| RS10500668 | 1039 | | | 11 | 6285001 | 2/2 | 1 |
| RS2670607 | 201 | | | 2 | 124113865 | 1/1 | 3 |
| RS1550740 | 1122 | | | 12 | 44813534 | 2/2 | 1 |
| RS6990997 | 867 | ZEPM2 | 23414 | 8 | 106881703 | 2/2 | 6 |
| RS10506265 | 1121 | | | 12 | 44802761 | 2/2 | 1 |
| RS10487745 | 746 | TAS2R16 | 50833 | 7 | 122228360 | 1/1 | 2 |
| RS10494664 | 95 | | | 1 | 188509177 | 1/1 | 184 |
| RS10509702 | 1009 | | | 10 | 97914086 | 1/1 | 237 |
| RS4793547 | 1358 | | | 17 | 66843227 | 2/2 | 36 |
| RS4131522 | 1153 | | | 12 | 102453596 | 2/2 | 220 |
| RS7536312 | 93 | | | 1 | 188459017 | 1/1 | 5 |
| RS1428250 | 508 | | | 5 | 38252169 | 1/1 | 218 |
| RS7718000 | 551 | | | 5 | 112998258 | 1/3 | 5 |
| RS2371319 | 268 | | | 3 | 40701948 | 2/2 | 2 |
| RS10516142 | 601 | FLT4 | 2324 | 5 | 180003769 | 2/2 | 243 |
| RS7597348 | 250 | | | 2 | 232194410 | 2/2 | 29 |
| RS10509161 | 996 | LOC644954 | 644954 | 10 | 63245347 | 1/1 | 2 |
| RS2827082 | 1444 | | | 21 | 22119060 | 2/2 | 5 |
| RS10491068 | 1011 | BLNK | 29760 | 10 | 97946892 | 2/2 | 236 |
| RS190788 | 1221 | | | 13 | 97587028 | 2/2 | 233 |
| RS1023431 | 1464 | GLA | 2717 | X | 100463954 | 3/3 | 214 |
| RS113318 | 548 | PCSK1 | 5122 | 5 | 95767236 | 2/2 | 3 |
| RS4131441 | 283 | CNTN3 | 5067 | 3 | 74561538 | 1/1 | 16 |
| RS1275637 | 1135 | | | 12 | 74445115 | 2/2 | 178 |
| RS953681 | 1442 | | | 21 | 20509158 | 1/1 | 191 |
| RS381765 | 363 | FRAS1 | 80144 | 4 | 79616778 | 2/2 | 232 |
| RS1226491 | 633 | GPR110 | 266977 | 6 | 47097807 | 1/1 | 1 |
| RS10509119 | 988 | ANK3 | 288 | 10 | 61482646 | 1/1 | 200 |
| RS6988377 | 885 | | | 8 | 137390100 | 1/1 | 4 |
| RS7665090 | 385 | | | 4 | 103908806 | 2/2 | 57 |
| RS10509160 | 995 | LOC644954 | 644954 | 10 | 63245218 | 2/2 | 2 |
| RS1332925 | 969 | C10orf112 | 340895 | 10 | 19836964 | 1/1 | 214 |
| RS10516867 | 380 | MGC48628 | 401145 | 4 | 91569270 | 1/1 | 1 |
| RS9285122 | 1183 | | | 13 | 37809901 | 2/4 | 8 |
| RS2150468 | 1184 | UFM1 | 51569 | 13 | 37825888 | 3/3 | 241 |
| RS9284616 | 360 | FRAS1 | 80144 | 4 | 79349710 | 2/2 | 208 |
| RS2969001 | 423 | | | 4 | 131140397 | 2/2 | 93 |
| RS7794555 | 754 | LOC653748 | 653748 | 7 | 153481879 | 2/2 | 214 |
| RS10495785 | 158 | LTBP1 | 4052 | 2 | 33472748 | 1/1 | 193 |
| RS10492506 | 1192 | CDADC1 | 81602 | 13 | 48722637 | 2/2 | 1 |
| RS723043 | 157 | LTBP1 | 4052 | 2 | 33453438 | 3/3 | 69 |
| RS2323936 | 1186 | | | 13 | 38001773 | 2/4 | 8 |
| RS2323937 | 1187 | | | 13 | 38001927 | 1/1 | 242 |
| RS1851006 | 1278 | UNC13C | 440279 | 15 | 52691684 | 2/2 | 45 |
| RS10509705 | 1010 | | | 10 | 97914784 | 2/2 | 1 |
| RS10517294 | 333 | | | 4 | 33603028 | 1/1 | 215 |
| RS6727480 | 231 | | | 2 | 197969377 | 2/2 | 18 |
| RS2169942 | 565 | | | 5 | 117512644 | 1/1 | 206 |
| RS10499227 | 686 | LOC645492 | 645492 | 6 | 142224151 | 1/1 | 9 |
| RS1577879 | 34 | | | 1 | 80924966 | 2/2 | 163 |
| RS1029233 | 1452 | | | 21 | 32327571 | 1/1 | 23 |
| RS7571668 | 178 | FLJ16124 | 440867 | 2 | 66198829 | 2/2 | 106 |
| RS10514845 | 1185 | | | 13 | 37910815 | 3/4 | 8 |
| RS4775359 | 1287 | RORA | 6095 | 15 | 59175796 | 2/2 | 195 |
| RS1889226 | 900 | KIAA1797 | 54914 | 9 | 20670198 | 2/2 | 219 |
| RS10517865 | 447 | | | 4 | 167008621 | 2/2 | 184 |
| RS10516865 | 379 | | | 4 | 91462479 | 3/3 | 245 |
| RS1004246 | 1405 | | | 19 | 16317260 | 1/1 | 93 |
| RS10510714 | 270 | | | 3 | 40736947 | 1/1 | 233 |
| RS4143188 | 1143 | C12orf26 | 84190 | 12 | 81305253 | 1/1 | 1 |
| RS340686 | 500 | | | 5 | 8440200 | 2/2 | 154 |
| RS10486650 | 713 | AAA1 | 404744 | 7 | 34413725 | 1/1 | 1 |
| RS10499017 | 676 | | | 6 | 98831359 | 2/2 | 92 |
| RS10517183 | 337 | GABRB1 | 2560 | 4 | 47209761 | 2/2 | 8 |
| RS10494826 | 98 | LOC647219 | 647219 | 1 | 197817653 | 2/2 | 11 |
| RS2254162 | 774 | | | 8 | 14632707 | 1/1 | 12 |
| RS1887889 | 905 | PGM5 | 5239 | 9 | 68275512 | 2/2 | 90 |
| RS1013459 | 1362 | GNAL | 2774 | 18 | 11690534 | 1/1 | 211 |
| RS4128923 | 461 | ADAM29 | 11086 | 4 | 176264705 | 2/2 | 3 |
| RS2072506 | 722 | AMPH | 273 | 7 | 38275635 | 1/1 | 214 |
| RS2034022 | 1396 | DOK6 | 220164 | 18 | 65341066 | 2/2 | 97 |
| RS924993 | 880 | | | 8 | 128373681 | 1/1 | 1 |
| RS10506869 | 1142 | | | 12 | 81247449 | 2/2 | 1 |
| RS10505540 | 882 | CCDC26 | 137196 | 8 | 130592619 | 2/2 | 1 |
| RS265056 | 383 | UNC5C | 8633 | 4 | 96755318 | 2/2 | 211 |
| RS10495520 | 124 | | | 2 | 5537475 | 2/2 | 4 |
| RS6856098 | 331 | PCDH7 | 5099 | 4 | 30797074 | 2/2 | 11 |
| RS10503419 | 771 | | | 8 | 11164210 | 1/1 | 157 |
| RS439269 | 897 | | | 9 | 14481347 | 2/2 | 196 |
| RS720327 | 382 | | | 4 | 92066267 | 1/1 | 213 |
| RS10513299 | 941 | ASTN2 | 23245 | 9 | 117184531 | 1/1 | 185 |
| RS1917952 | 429 | | | 4 | 137355240 | 2/2 | 75 |
| RS10483252 | 1226 | | | 14 | 21052193 | 2/2 | 1 |
| RS6733858 | 225 | | | 2 | 190290242 | 1/1 | 81 |
| RS1429130 | 432 | | | 4 | 148581368 | 1/1 | 209 |
| RS3118650 | 1194 | | | 13 | 49927325 | 1/1 | 234 |
| RS1959736 | 1245 | | | 14 | 82487626 | 3/3 | 8 |
| RS10511852 | 903 | | | 9 | 30227951 | 2/2 | 231 |
| RS10508692 | 970 | GPR158 | 57512 | 10 | 25677276 | 2/3 | 5 |
| RS2147085 | 37 | | | 1 | 80971158 | 1/1 | 51 |
| RS4611835 | 313 | NAALADL2 | 254827 | 3 | 176610777 | 1/1 | 2 |
| RS715428 | 1365 | KIAA1772 | 80000 | 18 | 17131449 | 1/2 | 12 |
| RS10516872 | 381 | MGC48628 | 401145 | 4 | 91681918 | 1/1 | 218 |
| RS9323955 | 1265 | VRK1 | 7443 | 14 | 96364688 | 1/1 | 56 |
| RS1844510 | 1241 | KCNH5 | 27133 | 14 | 62287241 | 1/1 | 64 |
| RS1204014 | 747 | TAS2R16 | 50833 | 7 | 122228794 | 2/2 | 228 |
| RS2785949 | 1195 | | | 13 | 53806676 | 1/1 | 124 |
| RS2721304 | 559 | | | 5 | 113462331 | 1/1 | 186 |
| RS10508096 | 1224 | BIVM | 54841 | 13 | 102258865 | 2/2 | 6 |
| RS10510100 | 1029 | | | 10 | 123456734 | 2/2 | 233 |
| RS9287120 | 96 | | | 1 | 188558913 | 2/2 | 5 |
| RS1818711 | 1279 | UNC13C | 440279 | 15 | 52692074 | 1/1 | 80 |
| RS10497689 | 224 | | | 2 | 188708247 | 1/3 | 5 |
| RS10492139 | 1115 | GRIN2B | 2904 | 12 | 13877862 | 2/2 | 14 |
| RS3886738 | 992 | LOC644954 | 644954 | 10 | 63234254 | 1/1 | 2 |
| RS10494214 | 85 | | | 1 | 119041077 | 2/2 | 205 |
| RS6798797 | 271 | | | 3 | 40741966 | 1/1 | 229 |
| RS935313 | 1266 | | | 15 | 27377574 | 1/1 | 52 |
| RS1464452 | 443 | | | 4 | 157951744 | 1/1 | 7 |
| RS2277456 | 1238 | PYGL | 5836 | 14 | 50447627 | 2/2 | 181 |
| RS1367719 | 1447 | | | 21 | 27974532 | 1/1 | 124 |
| RS7752110 | 620 | | | 6 | 23138143 | 2/2 | 9 |
| RS4918842 | 1023 | HABP2 | 3026 | 10 | 115306802 | 2/2 | 9 |
| RS1025748 | 438 | | | 4 | 154921502 | 1/1 | 154 |
| RS10514770 | 151 | | | 2 | 30524090 | 1/1 | 104 |
| RS1442799 | 1298 | | | 15 | 96589992 | 2/2 | 215 |
| RS2405708 | 428 | | | 4 | 137339327 | 1/1 | 51 |
| RS7413158 | 53 | GBP2 | 2634 | 1 | 89292717 | 1/1 | 6 |
| RS1407466 | 889 | | | 9 | 7958548 | 1/1 | 165 |
| RS2254764 | 1450 | GRIK1 | 2897 | 21 | 30131325 | 3/3 | 3 |
| RS539254 | 836 | | | 8 | 80079448 | 1/1 | 139 |
| RS2333821 | 170 | LOC646790 | 646790 | 2 | 44288177 | 2/2 | 112 |
| RS1016579 | 881 | | | 8 | 129269143 | 1/1 | 2 |
| RS10484518 | 698 | | | 6 | 164232489 | 3/3 | 25 |
| RS6989370 | 770 | | | 8 | 10653990 | 2/2 | 58 |
| RS10505777 | 1113 | | | 12 | 13378527 | 2/2 | 175 |
| RS717806 | 160 | | | 2 | 36487186 | 1/1 | 11 |
| RS1577302 | 968 | C10orf112 | 340895 | 10 | 19690726 | 2/2 | 6 |
| RS1869733 | 822 | FAM77D | 286183 | 8 | 63970954 | 2/2 | 161 |
| RS1217656 | 759 | CSMD1 | 64478 | 8 | 4293171 | 1/1 | 1 |
| RS2838000 | 1455 | BACE2 | 25825 | 21 | 41568388 | 1/1 | 20 |
| RS6741304 | 175 | LOC400954 | 400954 | 2 | 55099846 | 2/2 | 25 |
| RS2223163 | 1438 | | | 21 | 15149849 | 2/2 | 14 |
| RS4952999 | 169 | PLEKHH2 | 130271 | 2 | 43841904 | 1/1 | 39 |
| RS2973237 | 334 | | | 4 | 37294256 | 2/2 | 28 |
| RS3124806 | 2 | CAMTA1 | 23261 | 1 | 7703744 | 1/3 | 26 |
| RS1485870 | 453 | LOC442117 | 442117 | 4 | 173125585 | 2/2 | 157 |
| RS10518405 | 413 | | | 4 | 123937959 | 1/1 | 2 |
| RS10497650 | 223 | | | 2 | 185048541 | 2/4 | 5 |
| RS10488693 | 1083 | C11orf11 | 747 | 11 | 61241732 | 1/3 | 23 |
| RS10507082 | 1148 | | | 12 | 96255332 | 1/1 | 14 |
| RS10510715 | 269 | | | 3 | 40714716 | 2/2 | 2 |
| RS4684321 | 256 | | | 3 | 1842614 | 1/1 | 227 |
| RS829612 | 152 | | | 2 | 30542738 | 2/2 | 33 |
| RS10489925 | 67 | PAP2D | 163404 | 1 | 99173344 | 2/2 | 10 |
| RS735403 | 386 | MANBA | 4126 | 4 | 103910746 | 1/1 | 59 |
| RS2217486 | 489 | | | 4 | 188707345 | 1/1 | 9 |
| RS10485090 | 673 | | | 6 | 82882374 | 1/1 | 3 |
| RS7181421 | 1291 | | | 15 | 77626966 | 2/2 | 97 |
| RS3964705 | 1267 | LOC440261 | 440261 | 15 | 28822861 | 1/1 | 158 |
| RS1935720 | 68 | | | 1 | 99638912 | 3/3 | 191 |
| RS1433450 | 1293 | | | 15 | 85342066 | 1/1 | 2 |
| RS1452528 | 670 | | | 6 | 79155079 | 1/1 | 63 |
| RS10486694 | 724 | | | 7 | 40687431 | 1/1 | 1 |
| RS2089907 | 122 | | | 2 | 2454467 | 1/1 | 133 |
| RS10484693 | 603 | GMDS | 2762 | 6 | 1855107 | 1/3 | 12 |
| RS1922036 | 207 | | | 2 | 146473771 | 1/1 | 130 |
| RS868691 | 971 | | | 10 | 25945882 | 2/2 | 211 |
| RS745708 | 976 | FRMPD2 | 143162 | 10 | 49115681 | 1/1 | 150 |
| RS10502055 | 1104 | | | 11 | 104747888 | 2/2 | 72 |
| RS1369763 | 1397 | | | 18 | 71200032 | 2/2 | 210 |
| RS2825652 | 1441 | | | 21 | 19865219 | 2/2 | 239 |
| RS10521241 | 1303 | | | 16 | 49914787 | 1/1 | 4 |
| RS10486012 | 742 | | | 7 | 105824729 | 2/4 | 9 |
| RS1852561 | 731 | | | 7 | 69770184 | 2/4 | 15 |
| RS296032 | 200 | | | 2 | 123318299 | 2/2 | 8 |
| RS10495667 | 141 | VSNL1 | 7447 | 2 | 17745120 | 1/3 | 5 |
| RS950796 | 198 | | | 2 | 123281107 | 1/1 | 159 |
| RS10510155 | 1037 | | | 10 | 128313990 | 2/2 | 188 |
| RS10511356 | 291 | LSAMP | 4045 | 3 | 117596428 | 2/2 | 219 |
| RS1025927 | 831 | TRPA1 | 8989 | 8 | 73125689 | 1/1 | 221 |
| RS285098 | 1222 | FARP1 | 10160 | 13 | 97625036 | 2/2 | 42 |
| RS9315749 | 1188 | | | 13 | 39530110 | 2/2 | 27 |
| RS3850079 | 1196 | | | 13 | 56263149 | 1/1 | 21 |
| RS340680 | 499 | | | 5 | 8438180 | 1/1 | 12 |
| RS10520754 | 1294 | MCTP2 | 55784 | 15 | 92760413 | 2/2 | 66 |
| RS950016 | 1244 | | | 14 | 81514393 | 2/2 | 220 |
| RS1936671 | 97 | | | 1 | 189055410 | 2/2 | 139 |
| RS1371076 | 1146 | | | 12 | 89063918 | 1/1 | 21 |
| RS719025 | 1448 | | | 21 | 28674882 | 3/3 | 9 |
| RS717694 | 442 | | | 4 | 156772800 | 2/2 | 8 |
| RS2893569 | 723 | POU6F2 | 11281 | 7 | 38839984 | 1/1 | 227 |
| RS641445 | 1398 | | | 18 | 72182254 | 3/3 | 2 |
| RS4697055 | 322 | | | 4 | 23943605 | 1/1 | 142 |
| RS9318541 | 1206 | | | 13 | 77856249 | 2/2 | 24 |
| RS720613 | 749 | | | 7 | 124046621 | 2/2 | 146 |
| RS1465135 | 697 | | | 6 | 164166038 | 1/1 | 123 |
| RS295577 | 524 | mimitin | 91942 | 5 | 60457592 | 2/2 | 2 |
| RS7574093 | 176 | | | 2 | 56818904 | 1/3 | 10 |
| RS10517442 | 351 | | | 4 | 59273158 | 2/2 | 5 |
| RS7768538 | 630 | HLA-DQB2 | 3120 | 6 | 32837799 | 1/1 | 85 |
| RS1566728 | 1075 | PTPRJ | 5795 | 11 | 48121423 | 1/1 | 139 |
| RS3805134 | 295 | SLC15A2 | 6565 | 3 | 123100746 | 2/2 | 43 |
| RS7075669 | 1018 | | | 10 | 107583390 | 1/1 | 59 |
| RS10502056 | 1103 | | | 11 | 104677259 | 2/2 | 55 |
| RS1385080 | 1271 | | | 15 | 44513999 | 3/3 | 225 |
| RS10507553 | 1190 | | | 13 | 46687101 | 2/4 | 5 |
| RS1411315 | 1207 | | | 13 | 78638797 | 1/1 | 21 |
| RS7139422 | 1152 | FLJ25323 | 374470 | 12 | 102339523 | 1/1 | 1 |
| RS1235012 | 760 | CSMD1 | 64478 | 8 | 4294524 | 2/2 | 210 |
| RS7174236 | 1284 | RORA | 6095 | 15 | 58970994 | 2/2 | 8 |
| RS1993745 | 1055 | TMEM16C | 63982 | 11 | 26376767 | 2/2 | 37 |
| RS1341002 | 1264 | | | 14 | 94432505 | 2/2 | 102 |
| RS10498032 | 239 | ABCA12 | 26154 | 2 | 215779991 | 1/1 | 31 |
| RS989177 | 748 | | | 7 | 124006837 | 2/2 | 15 |
| RS1217654 | 761 | CSMD1 | 64478 | 8 | 4294769 | 2/2 | 203 |
| RS7453920 | 631 | HLA-DQB2 | 3120 | 6 | 32837990 | 1/1 | 32 |
| RS1604613 | 1197 | | | 13 | 69021604 | 2/2 | 47 |
| RS548533 | 1462 | LOC139060 | 139060 | X | 45600270 | 2/2 | 187 |
| RS479554 | 1463 | | | X | 45608877 | 1/1 | 59 |
| RS4686384 | 255 | | | 3 | 1036761 | 1/1 | 104 |
| RS10506626 | 1128 | TSPAN8 | 7103 | 12 | 69815758 | 1/1 | 18 |
| RS198278 | 711 | | | 7 | 24021433 | 1/1 | 55 |
| RS295525 | 525 | | | 5 | 60508623 | 2/2 | 2 |
| RS1886452 | 1200 | | | 13 | 72884855 | 1/1 | 158 |
| RS2009373 | 1415 | CARD8 | 22900 | 19 | 53411226 | 2/2 | 44 |
| RS6086149 | 1420 | | | 20 | 7586703 | 1/1 | 142 |
| RS10500986 | 1053 | LUZP2 | 338645 | 11 | 24592114 | 2/2 | 145 |
| RS10497776 | 228 | | | 2 | 196795652 | 1/1 | 244 |
| RS2832726 | 1451 | | | 21 | 30552005 | 1/1 | 78 |
| RS10495519 | 123 | | | 2 | 5524537 | 1/1 | 6 |
| RS10492135 | 1114 | GRIN2B | 2904 | 12 | 13777535 | 2/2 | 13 |
| RS10508840 | 973 | | | 10 | 36638433 | 1/1 | 6 |
| RS167164 | 199 | | | 2 | 123281157 | 2/2 | 157 |
| RS967220 | 264 | | | 3 | 29275054 | 3/3 | 5 |
| RS10493306 | 6 | NFIA | 4774 | 1 | 61389723 | 2/2 | 5 |
| RS2111826 | 309 | CLDN11 | 5010 | 3 | 171623548 | 1/3 | 39 |
| RS722508 | 1084 | | | 11 | 62792962 | 1/1 | 46 |
| RS7220359 | 1354 | RGS9 | 8787 | 17 | 60614155 | 1/3 | 14 |
| RS10490838 | 263 | | | 3 | 24574382 | 1/1 | 234 |
| RS2069837 | 710 | IL6 | 3569 | 7 | 22541267 | 1/1 | 217 |
| RS1485871 | 454 | LOC442117 | 442117 | 4 | 173125753 | 1/1 | 14 |
| RS2113487 | 153 | GALNT14 | 79623 | 2 | 31164203 | 1/1 | 87 |
| RS10519370 | 425 | | | 4 | 137332061 | 2/2 | 78 |
| RS1905155 | 556 | | | 5 | 113301097 | 3/3 | 116 |
| RS3827702 | 1423 | TASP1 | 55617 | 20 | 13446812 | 2/2 | 126 |
| RS10500783 | 1049 | | | 11 | 13530190 | 2/2 | 199 |
| RS724334 | 273 | CAST1 | 26059 | 3 | 55933122 | 2/2 | 174 |
| RS6852538 | 427 | | | 4 | 137338578 | 1/1 | 79 |
| RS6954168 | 755 | LOC653748 | 653748 | 7 | 153533800 | 2/2 | 241 |
| RS10512680 | 509 | FLJ39155 | 133584 | 5 | 38307189 | 1/1 | 62 |
| RS10518649 | 424 | | | 4 | 135764876 | 2/2 | 182 |
| RS2244327 | 899 | | | 9 | 18249760 | 2/2 | 89 |
| RS1153350 | 1437 | | | 21 | 15008235 | 1/1 | 3 |
| RS4510935 | 945 | | | 9 | 117364905 | 2/2 | 6 |
| RS1942578 | 1402 | | | 18 | 73322001 | 2/2 | 51 |
| RS953177 | 823 | DNAJC5B | 85479 | 8 | 67096174 | 2/2 | 222 |
| RS7701903 | 549 | | | 5 | 97968838 | 2/2 | 3 |
| RS309274 | 137 | | | 2 | 7151446 | 1/1 | 27 |
| RS1556082 | 1215 | GPC6 | 10082 | 13 | 93502465 | 1/1 | 7 |
| RS2616170 | 809 | | | 8 | 20610425 | 1/1 | 32 |
| RS10503626 | 808 | PSD3 | 23362 | 8 | 18591364 | 1/1 | 2 |
| RS2831853 | 1449 | | | 21 | 28781867 | 2/2 | 236 |
| RS2969004 | 422 | | | 4 | 131139275 | 3/3 | 39 |
| RS10501160 | 1070 | LOC119710 | 119710 | 11 | 36611046 | 1/1 | 1 |
| RS752355 | 213 | | | 2 | 176545532 | 1/1 | 15 |
| RS1372338 | 936 | ASTN2 | 23245 | 9 | 116620415 | 2/2 | 65 |
| RS1931610 | 1019 | LOC645254 | 645254 | 10 | 109813852 | 1/1 | 1 |
| RS1641354 | 1311 | | | 16 | 50778711 | 1/1 | 36 |
| RS6919514 | 688 | UST | 10090 | 6 | 149151922 | 3/4 | 43 |
| RS10498836 | 654 | | | 6 | 66585591 | 2/2 | 186 |
| RS1381181 | 1054 | TMEM16C | 63982 | 11 | 26340397 | 1/1 | 37 |
| RS1881014 | 732 | | | 7 | 69770606 | 2/2 | 212 |
| RS10501161 | 1072 | LOC119710 | 119710 | 11 | 36628245 | 2/2 | 215 |
| RS1351650 | 1040 | | | 11 | 7703386 | 2/2 | 63 |
| RS10489249 | 88 | | | 1 | 168446178 | 2/2 | 182 |
| RS10518089 | 351 | SLC4A4 | 8671 | 4 | 72794260 | 2/2 | 136 |
| RS4816018 | 1421 | | | 20 | 7604614 | 1/1 | 147 |
| RS950085 | 1006 | | | 10 | 95592637 | 1/1 | 24 |
| RS1823913 | 226 | MYO1B | 4430 | 2 | 191943553 | 2/2 | 147 |
| RS2383024 | 898 | C9orf39 | 54875 | 9 | 17477945 | 1/1 | 105 |
| RS9306295 | 1458 | | | 22 | 33775788 | 1/1 | 206 |
| RS10508842 | 974 | | | 10 | 36646002 | 2/2 | 7 |
| RS725500 | 364 | | | 4 | 80236951 | 2/2 | 10 |
| RS9326506 | 975 | | | 10 | 43388564 | 3/3 | 90 |
| RS9312592 | 462 | SPATA4 | 132851 | 4 | 177493018 | 2/2 | 7 |
| RS10512935 | 304 | EPHB1 | 2047 | 3 | 136107953 | 2/2 | 156 |
| RS722569 | 963 | | | 10 | 10538667 | 1/1 | 42 |
| RS1503453 | 833 | STAU2 | 27067 | 8 | 74666062 | 3/3 | 173 |
| RS719484 | 1228 | | | 14 | 26547587 | 1/1 | 172 |
| RS2477035 | 965 | | | 10 | 10571483 | 1/1 | 41 |
| RS10495033 | 112 | ESRRG | 2104 | 1 | 213264616 | 2/2 | 2 |
| RS718993 | 1336 | | | 17 | 14433113 | 1/1 | 11 |
| RS1607664 | 1410 | ZNF429 | 353088 | 19 | 21497820 | 2/2 | 9 |
| RS1073913 | 768 | | | 8 | 10649118 | 3/3 | 66 |
| RS10510104 | 1030 | ATE1 | 11101 | 10 | 123521812 | 2/2 | 5 |
| RS1927404 | 1205 | | | 13 | 76845208 | 1/1 | 95 |
| RS196584 | 715 | KIAA1706 | 80820 | 7 | 36090936 | 2/2 | 208 |
| RS9298895 | 902 | LRRN6C | 158038 | 9 | 28417657 | 1/3 | 34 |
| RS2991463 | 1209 | | | 13 | 78682643 | 2/2 | 18 |
| RS10501159 | 1069 | LOC119710 | 119710 | 11 | 36583337 | 1/1 | 1 |
| RS1541503 | 703 | LOC285929 | 285929 | 7 | 7173834 | 2/2 | 201 |
| RS4757441 | 1051 | PLEKHA7 | 144100 | 11 | 16859250 | 2/2 | 193 |
| RS9302933 | 1357 | | | 17 | 66750402 | 2/2 | 177 |
| RS309275 | 136 | | | 2 | 7150593 | 3/3 | 32 |
| RS10491279 | 578 | GDF9 | 2661 | 5 | 132225999 | 1/1 | 3 |
| RS10509153 | 989 | | | 10 | 63041318 | 2/2 | 2 |
| RS10485864 | 702 | SDK1 | 221935 | 7 | 3696897 | 2/2 | 187 |
| RS10510156 | 1036 | | | 10 | 128294406 | 1/1 | 5 |
| RS10520762 | 1295 | | | 15 | 93063543 | 2/2 | 169 |
| RS7530592 | 82 | VAV3 | 10451 | 1 | 107947761 | 3/3 | 96 |
| RS1867584 | 985 | | | 10 | 49321355 | 2/2 | 75 |
| RS10509772 | 1017 | C10orf80 | 159686 | 10 | 106149295 | 2/2 | 58 |
| RS6911443 | 675 | | | 6 | 98829624 | 1/1 | 98 |
| RS9297984 | 821 | | | 8 | 59098733 | 3/3 | 5 |
| RS10493822 | 52 | GBP1 | 2633 | 1 | 89241701 | 2/2 | 5 |
| RS165044 | 1272 | | | 15 | 44523246 | 1/1 | 101 |
| RS1928274 | 632 | GPR116 | 221395 | 6 | 46945349 | 1/1 | 11 |
| RS10508554 | 967 | SLC39A12 | 221074 | 10 | 18282641 | 2/2 | 27 |
| RS958938 | 8 | | | 1 | 66985931 | 1/1 | 2 |
| RS2964299 | 588 | | | 5 | 165876275 | 2/2 | 229 |
| RS1861373 | 1150 | | | 12 | 96275336 | 1/1 | 46 |
| RS1395817 | 431 | | | 4 | 148476328 | 1/3 | 27 |
| RS497197 | 772 | | | 8 | 12981181 | 2/2 | 2 |
| RS10498212 | 249 | IRS1 | 3667 | 2 | 227476780 | 1/1 | 232 |
| RS823986 | 579 | FSTL4 | 23105 | 5 | 132894139 | 1/1 | 22 |
| RS1544589 | 733 | PTPN12 | 5782 | 7 | 76874004 | 1/1 | 55 |
| RS7004663 | 834 | STAU2 | 27067 | 8 | 74692946 | 1/1 | 166 |
| RS4774367 | 1283 | RORA | 6095 | 15 | 58619313 | 2/2 | 131 |
| RS6863889 | 528 | | | 5 | 73560159 | 1/1 | 225 |
| RS868271 | 214 | ZNF533 | 151126 | 2 | 180255606 | 2/2 | 43 |
| RS1525489 | 745 | | | 7 | 122227424 | 2/2 | 1 |
| RS548987 | 629 | SLC17A3 | 10786 | 6 | 25977350 | 2/2 | 212 |
| RS10501933 | 1100 | CNTN5 | 53942 | 11 | 99284564 | 2/2 | 9 |
| RS1477523 | 750 | | | 7 | 136262393 | 2/2 | 222 |
| RS2163120 | 792 | | | 8 | 16165538 | 2/4 | 12 |
| RS10506524 | 1124 | MSRB3 | 253827 | 12 | 64070379 | 1/3 | 18 |
| RS4131874 | 819 | | | 8 | 42051123 | 2/2 | 136 |
| RS2981069 | 826 | | | 8 | 70100501 | 2/2 | 184 |
| RS1460597 | 1383 | DSG4 | 147409 | 18 | 27220516 | 2/2 | 14 |
| RS10511729 | 901 | | | 9 | 23547227 | 1/1 | 90 |
| RS1983726 | 86 | LOC441900 | 441900 | 1 | 119800976 | 2/2 | 12 |
| RS10511482 | 888 | | | 9 | 7512604 | 2/2 | 1 |
| RS2711452 | 185 | | | 2 | 82108603 | 1/1 | 120 |
| RS10497723 | 227 | TMEFF2 | 23671 | 2 | 192817829 | 1/1 | 216 |
| RS10500130 | 752 | | | 7 | 138273168 | 3/3 | 189 |
| RS1841914 | 261 | ZNF659 | 79750 | 3 | 21598635 | 1/1 | 47 |
| RS1938105 | 652 | | | 6 | 66567647 | 1/1 | 6 |
| RS1341001 | 1263 | | | 14 | 94432449 | 1/1 | 18 |
| RS10488204 | 706 | | | 7 | 11930380 | 2/2 | 230 |
| RS7670292 | 433 | DCAMKL2 | 166614 | 4 | 151461475 | 2/2 | 159 |
| RS7844425 | 818 | | | 8 | 32495159 | 1/1 | 101 |
| RS10510344 | 259 | GRM7 | 2917 | 3 | 7064629 | 1/3 | 34 |
| RS10516032 | 590 | LOC134541 | 134541 | 5 | 166726668 | 2/2 | 3 |
| RS6852312 | 389 | | | 4 | 105871774 | 3/3 | 225 |
| RS9317744 | 1176 | | | 13 | 22963766 | 2/4 | 11 |
| RS4368350 | 211 | | | 2 | 167367090 | 2/2 | 4 |
| RS7577293 | 186 | | | 2 | 85846940 | 1/1 | 13 |
| RS10514263 | 545 | EDIL3 | 10085 | 5 | 83426017 | 1/1 | 299 |
| RS10514632 | 212 | RAPGEF4 | 11069 | 2 | 173530419 | 1/1 | 6 |
| RS1376499 | 674 | | | 6 | 98759316 | 3/3 | 70 |
| RS7148756 | 1240 | | | 14 | 58616282 | 1/1 | 4 |
| RS2165347 | 1317 | | | 16 | 58809040 | 2/2 | 24 |
| RS1199292 | 741 | ACN9 | 57001 | 7 | 96432248 | 2/2 | 33 |
| RS1441541 | 490 | | | 5 | 3303352 | 1/1 | 5 |
| RS171785 | 596 | | | 5 | 170994432 | 1/1 | 30 |
| RS10484331 | 608 | | | 6 | 8940232 | 2/2 | 87 |
| RS2572276 | 1389 | | | 18 | 39324404 | 1/1 | 23 |
| RS10483323 | 1227 | | | 14 | 26354562 | 1/1 | 11 |
| RS3859362 | 1382 | DSG4 | 147409 | 18 | 27218666 | 2/2 | 14 |
| RS10513765 | 316 | PEX5L | 51555 | 3 | 181027572 | 2/2 | 32 |
| RS964059 | 879 | | | 8 | 124904538 | 3/3 | 159 |
| RS2825583 | 1440 | | | 21 | 19734374 | 2/2 | 85 |
| RS8137239 | 1456 | LOC644802 | 644802 | 22 | 15504963 | 1/1 | 1 |
| RS6699989 | 44 | | | 1 | 87784403 | 1/1 | 9 |
| RS2396821 | 635 | | | 6 | 47183758 | 1/1 | 166 |
| RS10507930 | 1212 | | | 13 | 82396265 | 1/3 | 6 |
| RS7324065 | 1220 | ABCC4 | 10257 | 13 | 94503308 | 1/3 | 13 |
| RS1329983 | 29 | | | 1 | 73801814 | 1/1 | 3 |
| RS7874576 | 896 | | | 9 | 12600410 | 1/3 | 5 |
| RS10513275 | 308 | | | 3 | 147165932 | 2/2 | 192 |
| RS1324088 | 628 | | | 6 | 25949101 | 2/2 | 212 |
| RS10495466 | 118 | FMN2 | 56776 | 1 | 236834608 | 2/2 | 227 |
| RS718911 | 1286 | RORA | 6095 | 15 | 59169716 | 1/1 | 194 |
| RS4952676 | 168 | PLEKHH2 | 130271 | 2 | 43841634 | 1/1 | 100 |
| RS10518774 | 1282 | UNC13C | 440279 | 15 | 52700466 | 1/1 | 61 |
| RS488673 | 694 | SYNE1 | 23345 | 6 | 152811716 | 2/2 | 88 |
| RS3815035 | 1355 | MAP2K6 | 5608 | 17 | 65043983 | 2/2 | 5 |
| RS3890717 | 114 | | | 1 | 217860308 | 3/3 | 45 |
| RS188014 | 1193 | LOC647154 | 647154 | 13 | 49867924 | 1/3 | 15 |
| RS10508318 | 957 | | | 10 | 7090365 | 1/1 | 8 |
| RS472154 | 43 | COL24A1 | 255631 | 1 | 86167436 | 2/2 | 19 |
| RS9300024 | 1073 | | | 11 | 37240693 | 1/1 | 65 |
| RS10512405 | 927 | SVEP1 | 79987 | 9 | 110316352 | 2/2 | 19 |
| RS7009058 | 820 | SNTG1 | 54212 | 8 | 51656260 | 3/3 | 181 |
| RS341148 | 696 | TULP4 | 56995 | 6 | 158810407 | 2/2 | 101 |
| RS9302288 | 1290 | | | 15 | 77462356 | 1/1 | 155 |
| Rs10498604 | 1249 | | | 14 | 86238836 | 2/2 | 17 |
| RS6812018 | 355 | | | 4 | 66863258 | 3/3 | 197 |
| RS10514569 | 1325 | CDH13 | 1012 | 16 | 82033605 | 3/4 | 13 |
| RS3751462 | 1239 | LOC283553 | 283553 | 14 | 50897971 | 1/1 | 150 |
| RS987479 | 714 | | | 7 | 34729664 | 1/1 | 149 |
| RS1233853 | 1334 | FLJ34690 | 284034 | 17 | 12474944 | 1/1 | 2 |
| RS979323 | 1248 | | | 14 | 84118710 | 1/1 | 40 |
| RS962592 | 476 | | | 4 | 181367262 | 2/2 | 243 |
| RS5962018 | 1459 | | | X | 6207596 | 2/2 | 210 |
| RS362988 | 1422 | SNAP25 | 6616 | 20 | 10229370 | 1/1 | 77 |
| RS945849 | 933 | C9orf84 | 158401 | 9 | 111585474 | 1/1 | 166 |
| RS7078806 | 1026 | | | 10 | 11845599 | 1/1 | 224 |
| RS8089476 | 1392 | | | 18 | 46107418 | 2/2 | 35 |
| RS9293488 | 546 | | | 5 | 87103437 | 2/2 | 218 |
| RS1340169 | 1198 | | | 13 | 71876062 | 3/4 | 9 |
| RS6109935 | 1424 | TASP1 | 55617 | 20 | 13467837 | 2/2 | 19 |
| RS10502776 | 1385 | PIK3C3 | 5289 | 18 | 37832275 | 1/1 | 237 |
| RS1403635 | 257 | CNTN4 | 152330 | 3 | 2264798 | 2/2 | 8 |
| RS3800274 | 629 | C6orf96 | 55005 | 6 | 151851306 | 3/3 | 13 |
| RS1459914 | 280 | | | 3 | 67849940 | 3/3 | 203 |
| RS1233775 | 1425 | C20orf133 | 140733 | 20 | 15451543 | 2/3 | 137 |
| RS9294240 | 672 | | | 6 | 82703881 | 1/1 | 19 |
| RS1407390 | 931 | C9orf84 | 158401 | 9 | 111544042 | 2/2 | 170 |
| RS10495804 | 159 | | | 2 | 34169804 | 2/2 | 26 |
| RS6477845 | 930 | C9orf84 | 158401 | 9 | 111541877 | 1/1 | 3 |
| RS10501158 | 1068 | LOC119710 | 119710 | 11 | 36582180 | 1/1 | 217 |
| RS1945654 | 1086 | LOC646169 | 646169 | 11 | 80901934 | 2/2 | 186 |
| RS3814860 | 1235 | FBXO33 | 254170 | 14 | 38938397 | 3/3 | 160 |
| RS2881836 | 234 | AOX2 | 344454 | 2 | 201469088 | 3/3 | 110 |
| RS1805781 | 1111 | PHC1 | 1911 | 12 | 8957155 | 1/3 | 8 |
| RS294665 | 197 | | | 2 | 123276926 | 2/2 | 159 |
| RS3897748 | 113 | | | 1 | 214677604 | 2/2 | 170 |
| RS10517222 | 332 | | | 4 | 30831187 | 1/1 | 151 |
| RS10507083 | 1149 | | | 12 | 96255384 | 1/1 | 16 |
| RS796892 | 296 | | | 3 | 123868092 | 2/2 | 19 |
| RS7002137 | 769 | | | 8 | 10653610 | 2/2 | 180 |
| RS7719212 | 501 | | | 5 | 23658681 | 2/2 | 231 |
| RS713265 | 70 | | | 1 | 99649058 | 2/2 | 1 |
| RS6848015 | 448 | PALLD | 23022 | 4 | 169822829 | 2/2 | 176 |
| RS10495202 | 115 | LOC644053 | 644053 | 1 | 219385670 | 1/1 | 146 |
| RS4129693 | 142 | | | 2 | 18864906 | 2/2 | 148 |
| RS4073474 | 294 | IQCB1 | 9657 | 3 | 122990105 | 1/1 | 103 |
| RS150344 | 1302 | | | 16 | 22728560 | 3/3 | 176 |
| RS1915552 | 844 | | | 8 | 80237554 | 1/1 | 34 |
| RS1440063 | 248 | | | 2 | 223677858 | 1/1 | 85 |
| RS494739 | 685 | | | 6 | 137969137 | 2/2 | 209 |
| RS2391476 | 421 | | | 4 | 130916643 | 3/3 | 4 |
| RS2054915 | 1089 | FLJ22104 | 65084 | 11 | 86668250 | 1/1 | 66 |
| RS989766 | 1431 | PTPRT | 11122 | 20 | 40443639 | 2/2 | 129 |
| RS7675645 | 352 | | | 4 | 59309086 | 1/1 | 167 |
| RS1571502 | 65 | PAP2D | 163404 | 1 | 99137091 | 2/2 | 1 |
| RS1661405 | 699 | | | 6 | 165561151 | 1/3 | 10 |
| RS2675200 | 258 | | | 3 | 4118873 | 2/4 | 32 |
| RS3905336 | 906 | | | 9 | 72699914 | 2/4 | 7 |
| RS652238 | 1395 | | | 18 | 64109605 | 2/2 | 181 |
| RS3852745 | 1301 | GRIN2A | 2903 | 16 | 10156361 | 1/1 | 12 |
| RS10491948 | 895 | | | 9 | 11522517 | 2/2 | 228 |
| RS10490414 | 177 | | | 2 | 56927557 | 2/4 | 8 |
| RS3751337 | 1180 | KIAA0774 | 23281 | 13 | 28796573 | 1/1 | 21 |
| RS1041802 | 1446 | | | 21 | 27965603 | 3/3 | 124 |
| RS10505725 | 1110 | DCP1B | 196513 | 12 | 1926624 | 2/2 | 131 |
| RS1207613 | 1460 | | | X | 24327376 | 2/2 | 194 |
| RS4981115 | 1230 | NUBPL | 80224 | 14 | 31240826 | 1/1 | 4 |
| RS1150030 | 959 | | | 10 | 9405319 | 1/1 | 1 |
| RS2217610 | 700 | SDK1 | 221935 | 7 | 3503877 | 1/1 | 142 |
| RS596255 | 837 | | | 8 | 80164544 | 2/2 | 22 |
| RS7906587 | 1025 | PNLIPRP3 | 119548 | 10 | 118221656 | 2/2 | 227 |
| RS10489251 | 89 | | | 1 | 168449191 | 3/3 | 5 |
| RS10509766 | 1016 | NT5C2 | 22978 | 10 | 104916324 | 2/2 | 29 |
| RS4890429 | 1387 | RIT2 | 6014 | 18 | 38951479 | 1/1 | 129 |
| RS7694034 | 377 | MAPK10 | 5602 | 4 | 87647635 | 1/1 | 8 |
| RS7963889 | 1134 | | | 12 | 73136950 | 1/1 | 31 |
| RS286300 | 1404 | LOC401898 | 401898 | 19 | 11658308 | 2/2 | 218 |
| RS10493985 | 78 | COL11A1 | 1301 | 1 | 103282453 | 1/3 | 7 |
| RS6823379 | 335 | | | 4 | 41552790 | 1/1 | 237 |
| RS499891 | 1076 | GLYAT | 10249 | 11 | 58246797 | 1/1 | 241 |
| RS539085 | 1077 | GLYAT | 10249 | 11 | 58255401 | 2/2 | 241 |
| RS2156348 | 1078 | GLYATL2 | 219970 | 11 | 58360325 | 2/2 | 241 |
| RS477892 | 1079 | | | 11 | 58375953 | 1/1 | 241 |
| RS480413 | 1080 | | | 11 | 58376174 | 1/3 | 9 |
| RS2856259 | 1081 | | | 11 | 58376529 | 1/3 | 9 |
| RS4939237 | 1082 | | | 11 | 58419301 | 1/3 | 9 |
| RS10502807 | 1386 | RIT2 | 6014 | 18 | 38802002 | 2/2 | 185 |
| RS10512282 | 923 | GRIN3A | 116443 | 9 | 101413888 | 1/1 | 205 |
| RS10500585 | 1319 | | | 16 | 72882363 | 2/2 | 118 |
| RS823638 | 934 | C9orf84 | 158401 | 9 | 111622316 | 1/1 | 3 |
| RS697902 | 318 | | | 3 | 192932251 | 2/2 | 91 |
| RS10493987 | 77 | COL11A1 | 1301 | 1 | 103278613 | 1/3 | 5 |
| RS4733224 | 817 | WRN | 7486 | 8 | 31086911 | 1/1 | 90 |
| RS2731239 | 1145 | | | 12 | 89061969 | 2/2 | 22 |
| RS7459039 | 705 | | | 7 | 9668794 | 1/1 | 13 |
| RS1156968 | 1233 | | | 14 | 38232528 | 2/2 | 194 |
| RS7470491 | 932 | C9orf84 | 158401 | 9 | 111569863 | 2/2 | 166 |
| RS10485148 | 619 | | | 6 | 23080312 | 2/2 | 26 |
| RS564896 | 143 | FLJ21820 | 60526 | 2 | 20899161 | 2/2 | 23 |
| RS666941 | 240 | | | 2 | 221567963 | 1/1 | 47 |
| RS10506603 | 1126 | PTPRR | 5801 | 12 | 69367960 | 2/3 | 7 |
| RS2669892 | 290 | ZBTB20 | 26137 | 3 | 115817713 | 1/1 | 165 |
| RS6477360 | 891 | PTPRD | 5789 | 9 | 8887230 | 1/1 | 69 |
| RS10506602 | 1125 | PTPRR | 5801 | 12 | 69348510 | 2/2 | 227 |
| RS10518821 | 1289 | | | 15 | 67669717 | 1/1 | 161 |
| RS10508269 | 956 | | | 10 | 3903483 | 1/1 | 4 |
| RS10512501 | 1353 | TEX2 | 55852 | 17 | 59677087 | 1/1 | 1 |
| RS757668 | 744 | | | 7 | 122175535 | 1/1 | 35 |
| RS10518404 | 412 | | | 4 | 123936746 | 2/2 | 216 |
| RS1813404 | 1246 | | | 14 | 84116590 | 2/2 | 114 |
| RS10510020 | 1024 | PNLIPRP3 | 119548 | 10 | 118221295 | 1/1 | 227 |
| RS10510022 | 1027 | | | 10 | 118275623 | 1/1 | 227 |
| RS10510023 | 1028 | | | 10 | 118275843 | 1/1 | 1 |
| RS723855 | 1436 | | | 21 | 14771745 | 1/1 | 12 |
| RS10491340 | 581 | | | 5 | 147370294 | 2/2 | 7 |
| RS713266 | 69 | | | 1 | 99648777 | 2/2 | 217 |
| RS2011688 | 83 | SLC6A17 | 388662 | 1 | 110441446 | 2/2 | 211 |
| RS2826847 | 1443 | NCAM2 | 4685 | 21 | 21749297 | 2/2 | 195 |
| RS869206 | 1071 | LOC119710 | 119710 | 11 | 36615190 | 2/2 | 218 |
| RS1355296 | 1268 | | | 15 | 34027338 | 1/1 | 3 |
| RS9298249 | 835 | | | 8 | 7620083 | 1/1 | 1 |
| RS580758 | 390 | | | 4 | 105928555 | 1/1 | 4 |
| RS10517618 | 439 | | | 4 | 156628360 | 3/3 | 122 |
| RS979322 | 1247 | | | 14 | 84118568 | 2/2 | 82 |
| RS9298675 | 886 | | | 9 | 1242572 | 2/2 | 1 |
| RS2863171 | 1074 | | | 11 | 45207308 | 3/3 | 5 |
| RS1459506 | 446 | | | 4 | 166905208 | 1/1 | 29 |
| RS1822539 | 924 | | | 9 | 103821410 | 2/2 | 177 |
| RS10488521 | 740 | CDK6 | 1021 | 7 | 91987519 | 2/2 | 7 |
| RS10504894 | 864 | | | 8 | 91585612 | 1/1 | 24 |
| RS2255751 | 964 | | | 10 | 10568976 | 2/2 | 45 |
| RS7827548 | 765 | CSMD1 | 64478 | 8 | 4593724 | 2/2 | 111 |
| RS1451336 | 677 | | | 6 | 98849320 | 1/1 | 74 |
| RS10493355 | 7 | ROR1 | 4919 | 1 | 64255969 | 1/1 | 1 |
| RS1926029 | 1014 | NT5C2 | 22978 | 10 | 104845660 | 2/2 | 129 |
| RS2572269 | 1388 | | | 18 | 39310973 | 1/1 | 22 |
| RS10507118 | 1151 | TMEM16D | 121601 | 12 | 99722902 | 2/2 | 4 |

| RS_id | avg1 | g2 | n2 | avg2 | g3 | n3 | avg3 | F | dft | dfe | P-value |
|---|---|---|---|---|---|---|---|---|---|---|---|
| RS1410126 | 1.97 | 1/2 | 60 | 1.23 | 2/2 | 181 | 1.3 | 15.35 | 2 | 243 | 5.28E−07 |
| RS10498321 | 1.27 | 1/2 | 59 | 1.36 | 2/2 | 1 | 2.77 | 15.31 | 2 | 244 | 5.45E−07 |
| RS744832 | 2.77 | 1/3 | 53 | 1.36 | 3/3 | 192 | 1.27 | 14.8 | 2 | 243 | 8.61E−07 |
| RS10485909 | 1.3 | 2/4 | 20 | 1.21 | 4/4 | 1 | 2.77 | 13.9 | 2 | 245 | 1.91E−06 |
| RS2195989 | 1.76 | 2/4 | 53 | 1.35 | 4/4 | 178 | 1.26 | 13.72 | 2 | 237 | 2.30E−06 |
| RS1399503 | 1.29 | 3/4 | 41 | 1.24 | 4/4 | 2 | 2.33 | 13.64 | 2 | 244 | 2.42E−06 |
| RS4102401 | 2.77 | 2/4 | 19 | 1.36 | 4/4 | 229 | 1.29 | 13.6 | 2 | 246 | 2.50E−06 |
| RS10518533 | 1.96 | 1/2 | 104 | 1.3 | 2/2 | 126 | 1.26 | 13.6 | 2 | 232 | 2.60E−06 |
| RS10504433 | 1.28 | 2/4 | 53 | 1.33 | 4/4 | 1 | 2.77 | 13.51 | 2 | 247 | 2.70E−06 |
| RS10516418 | 1.29 | 1/3 | 27 | 1.36 | 3/3 | 1 | 2.77 | 13.43 | 2 | 238 | 2.98E−06 |
| RS2230133 | 1.3 | 2/3 | 43 | 1.25 | 3/3 | 1 | 2.77 | 13.35 | 2 | 246 | 3.13E−06 |
| RS10498260 | 2.77 | 1/3 | 38 | 1.3 | 3/3 | 192 | 1.28 | 13.31 | 2 | 228 | 3.41E−06 |
| RS10486467 | 2.77 | 1/3 | 9 | 1.21 | 3/3 | 237 | 1.29 | 13.22 | 2 | 244 | 3.54E−06 |
| RS2120650 | 1.29 | 1/3 | 26 | 1.32 | 3/3 | 1 | 2.77 | 13.07 | 2 | 247 | 4.02E−06 |
| RS1467596 | 2.77 | 1/3 | 12 | 1.26 | 3/3 | 235 | 1.29 | 13.06 | 2 | 245 | 4.08E−06 |
| RS2170398 | 2.77 | 2/4 | 41 | 1.31 | 4/4 | 206 | 1.29 | 13.02 | 2 | 245 | 4.23E−06 |
| RS352766 | 2.77 | 2/4 | 30 | 1.27 | 4/4 | 218 | 1.29 | 12.97 | 2 | 246 | 4.41E−06 |
| RS10488888 | 2.77 | 2/4 | 6 | 1.33 | 4/4 | 242 | 1.29 | 12.95 | 2 | 246 | 4.49E−06 |
| RS4960221 | 1.29 | 2/4 | 61 | 1.31 | 4/4 | 1 | 2.77 | 12.94 | 2 | 245 | 4.55E−06 |
| RS10494066 | 1.89 | 2/4 | 86 | 1.29 | 4/4 | 155 | 1.28 | 12.94 | 2 | 244 | 4.56E−06 |
| RS10517155 | 2.77 | 2/4 | 51 | 1.29 | 4/4 | 198 | 1.29 | 12.92 | 2 | 247 | 4.61E−06 |
| RS9303878 | 1.3 | 1/4 | 34 | 1.24 | 4/4 | 2 | 2.32 | 12.82 | 2 | 235 | 5.20E−06 |
| RS10510995 | 2.77 | 1/3 | 12 | 1.27 | 3/3 | 232 | 1.3 | 12.77 | 2 | 242 | 5.34E−06 |
| RS718759 | 2.77 | 2/3 | 26 | 1.27 | 3/3 | 205 | 1.3 | 12.54 | 2 | 229 | 6.79E−06 |
| RS1480145 | 1.28 | 2/3 | 12 | 1.67 | 0/0 | 0 | 0 | 20.78 | 1 | 248 | 8.10E−06 |
| RS10485110 | 1.87 | 3/4 | 70 | 1.29 | 4/4 | 171 | 1.27 | 12.1 | 2 | 244 | 9.76E−06 |
| RS1902049 | 1.27 | 3/4 | 92 | 1.25 | 4/4 | 18 | 1.61 | 12.06 | 2 | 243 | 1.01E−05 |
| RS2235216 | 1.87 | 1/3 | 72 | 1.3 | 3/3 | 150 | 1.28 | 11.62 | 2 | 225 | 1.58E−05 |
| RS470559 | 1.94 | 2/3 | 53 | 1.19 | 3/3 | 194 | 1.32 | 11.55 | 2 | 247 | 1.60E−05 |
| RS10503573 | 1.28 | 1/2 | 14 | 1.63 | 0/0 | 0 | 0 | 19.29 | 1 | 248 | 1.66E−05 |
| RS2854946 | 1.66 | 2/3 | 66 | 1.3 | 3/3 | 134 | 1.25 | 11.44 | 2 | 210 | 1.92E−05 |
| RS4631686 | 1.74 | 3/3 | 237 | 1.29 | 0/0 | 0 | 0 | 18.76 | 1 | 243 | 2.17E−05 |
| RS6954679 | 1.27 | 1/3 | 38 | 1.35 | 3/3 | 3 | 2.05 | 11.12 | 2 | 245 | 2.38E−05 |
| RS866651 | 1.29 | 2/3 | 72 | 1.25 | 3/3 | 10 | 1.71 | 11.01 | 2 | 246 | 2.63E−05 |
| RS2255565 | 1.29 | 1/2 | 55 | 1.25 | 2/2 | 4 | 1.96 | 10.93 | 2 | 247 | 2.83E−05 |
| RS10483988 | 1.29 | 2/4 | 66 | 1.26 | 4/4 | 4 | 1.96 | 10.81 | 2 | 245 | 3.17E−05 |
| RS387661 | 2.25 | 2/4 | 39 | 1.27 | 4/4 | 208 | 1.29 | 10.66 | 2 | 246 | 3.63E−05 |
| RS2029259 | 1.26 | 1/2 | 49 | 1.41 | 2/2 | 4 | 1.77 | 10.63 | 2 | 244 | 3.75E−05 |
| RS2113515 | 1.29 | 2/4 | 57 | 1.27 | 4/4 | 4 | 1.96 | 10.6 | 2 | 245 | 3.84E−05 |
| RS10503586 | 1.28 | 1/3 | 12 | 1.65 | 0/0 | 0 | 0 | 17.39 | 1 | 242 | 4.24E−05 |
| RS285406 | 1.27 | 2/4 | 22 | 1.55 | 0/0 | 0 | 0 | 17.25 | 1 | 248 | 4.51E−05 |
| RS6811055 | 1.27 | 2/4 | 102 | 1.4 | 4/4 | 54 | 1.18 | 10.43 | 2 | 228 | 4.63E−05 |
| RS397618 | 1.28 | 2/4 | 14 | 1.61 | 0/0 | 0 | 0 | 17.06 | 1 | 248 | 4.95E−05 |
| RS896990 | 2.03 | 1/2 | 41 | 1.35 | 2/2 | 198 | 1.28 | 10.31 | 2 | 239 | 5.07E−05 |
| RS250387 | 1.24 | 1/3 | 67 | 1.43 | 3/3 | 8 | 1.4 | 10.24 | 2 | 244 | 5.37E−05 |
| RS9299445 | 1.27 | 2/4 | 34 | 1.38 | 4/4 | 3 | 1.97 | 10.01 | 2 | 240 | 6.68E−05 |
| RS10516673 | 1.28 | 1/3 | 13 | 1.62 | 0/0 | 0 | 0 | 16.13 | 1 | 248 | 7.84E−05 |
| RS10520350 | 2.38 | 2/3 | 18 | 1.45 | 3/3 | 230 | 1.28 | 9.8 | 2 | 246 | 8.03E−05 |
| RS9299446 | 1.97 | 1/2 | 37 | 1.37 | 2/2 | 210 | 1.27 | 9.7 | 2 | 247 | 8.80E−05 |
| RS2170116 | 1.27 | 2/4 | 19 | 1.56 | 0/0 | 0 | 0 | 15.88 | 1 | 248 | 8.88E−05 |
| RS4888946 | 1.27 | 1/4 | 62 | 1.32 | 4/4 | 3 | 2.01 | 9.67 | 2 | 239 | 9.15E−05 |
| RS10495783 | 1.44 | 2/4 | 130 | 1.23 | 4/4 | 67 | 1.32 | 9.65 | 2 | 244 | 9.26E−05 |
| RS10516834 | 1.95 | 1/3 | 25 | 1.18 | 3/3 | 217 | 1.3 | 9.34 | 2 | 242 | 1.24E−04 |
| RS2207139 | 1.74 | 2/4 | 76 | 1.26 | 4/4 | 164 | 1.29 | 9.33 | 2 | 245 | 1.24E−04 |
| RS8054578 | 2.01 | 1/3 | 65 | 1.32 | 3/3 | 176 | 1.27 | 9.33 | 2 | 241 | 1.25E−04 |
| RS7897958 | 1.77 | 1/4 | 63 | 1.24 | 4/4 | 179 | 1.3 | 9.31 | 2 | 245 | 1.27E−04 |
| RS1121640 | 1.28 | 2/4 | 28 | 1.36 | 4/4 | 2 | 2.15 | 9.26 | 2 | 245 | 1.33E−04 |
| RS10493425 | 1.28 | 1/2 | 13 | 1.23 | 2/2 | 2 | 2.12 | 9.28 | 2 | 228 | 1.33E−04 |
| RS965158 | 1.64 | 4/4 | 239 | 1.28 | 0/0 | 0 | 0 | 15 | 1 | 248 | 1.38E−04 |
| RS10482871 | 1.8 | 3/3 | 245 | 1.29 | 0/0 | 0 | 0 | 15 | 1 | 248 | 1.38E−04 |
| RS10503572 | 1.81 | 3/3 | 243 | 1.29 | 0/0 | 0 | 0 | 14.97 | 1 | 246 | 1.40E−04 |
| RS9316436 | 1.34 | 2/3 | 58 | 1.16 | 3/3 | 5 | 1.31 | 9.15 | 2 | 243 | 1.47E−04 |
| RS812800 | 1.71 | 1/3 | 73 | 1.28 | 3/3 | 163 | 1.28 | 9.11 | 2 | 242 | 1.53E−04 |
| RS10508377 | 1.31 | 1/3 | 29 | 1.18 | 3/3 | 1 | 2.38 | 9.05 | 2 | 245 | 1.61E−04 |
| RS10500985 | 1.73 | 1/2 | 75 | 1.31 | 2/2 | 146 | 1.26 | 8.98 | 2 | 224 | 1.77E−04 |
| RS3116231 | 2.12 | 2/3 | 49 | 1.34 | 3/3 | 196 | 1.27 | 8.95 | 2 | 244 | 1.77E−04 |
| RS1490125 | 1.34 | 3/4 | 66 | 1.18 | 0/0 | 0 | 0 | 14.45 | 1 | 245 | 1.82E−04 |
| RS8049647 | 1.26 | 1/3 | 76 | 1.34 | 3/3 | 7 | 1.71 | 8.91 | 2 | 246 | 1.84E−04 |
| RS10517562 | 1.19 | 1/3 | 116 | 1.23 | 3/3 | 106 | 1.38 | 8.83 | 2 | 243 | 1.99E−04 |
| RS2222654 | 1.19 | 1/3 | 88 | 1.37 | 3/3 | 54 | 1.34 | 8.83 | 2 | 216 | 2.06E−04 |
| RS2357344 | 1.83 | 2/4 | 26 | 1.3 | 4/4 | 189 | 1.27 | 8.8 | 2 | 217 | 2.11E−04 |
| RS4869279 | 1.31 | 2/4 | 75 | 1.23 | 4/4 | 4 | 1.83 | 8.75 | 2 | 244 | 2.14E−04 |
| RS11232919 | 2.15 | 2/4 | 64 | 1.27 | 4/4 | 183 | 1.3 | 8.74 | 2 | 246 | 2.15E−04 |
| RS9315157 | 1.38 | 1/3 | 49 | 1.45 | 3/3 | 193 | 1.25 | 8.71 | 2 | 245 | 2.22E−04 |
| RS1675904 | 1.27 | 2/4 | 30 | 1.35 | 4/4 | 1 | 2.38 | 8.69 | 2 | 232 | 2.29E−04 |
| RS1502887 | 1.27 | 2/4 | 41 | 1.46 | 0/0 | 0 | 0 | 13.98 | 1 | 246 | 2.30E−04 |
| RS1380835 | 1.31 | 3/4 | 65 | 1.22 | 4/4 | 3 | 1.88 | 8.61 | 2 | 247 | 2.43E−04 |
| RS10509764 | 1.37 | 2/4 | 96 | 1.21 | 4/4 | 27 | 1.25 | 8.59 | 2 | 246 | 2.48E−04 |
| RS10514450 | 2.01 | 2/4 | 56 | 1.3 | 4/4 | 174 | 1.29 | 8.59 | 2 | 230 | 2.52E−04 |
| RS2101435 | 1.28 | 2/3 | 33 | 1.3 | 3/3 | 2 | 2.13 | 8.58 | 2 | 223 | 2.57E−04 |
| RS9308931 | 1.33 | 1/2 | 107 | 1.23 | 2/2 | 50 | 1.44 | 8.54 | 2 | 219 | 2.68E−04 |
| RS2290547 | 1.1 | 1/3 | 91 | 1.39 | 3/3 | 143 | 1.26 | 8.5 | 2 | 244 | 2.70E−04 |
| RS10504709 | 1.58 | 1/3 | 86 | 1.31 | 3/3 | 143 | 1.26 | 8.47 | 2 | 242 | 2.78E−04 |
| RS10521979 | 1.45 | 4/4 | 207 | 1.27 | 0/0 | 0 | 0 | 13.51 | 1 | 246 | 2.91E−04 |
| RS10496647 | 2.38 | 1/3 | 33 | 1.21 | 3/3 | 216 | 1.31 | 8.37 | 2 | 247 | 3.04E−04 |
| RS8076457 | 1.27 | 2/4 | 71 | 1.3 | 4/4 | 8 | 1.7 | 8.28 | 2 | 245 | 3.31E−04 |
| RS297733 | 1.27 | 3/4 | 81 | 1.31 | 4/4 | 6 | 1.76 | 8.25 | 2 | 244 | 3.41E−04 |
| RS10513319 | 1.28 | 1/3 | 16 | 1.56 | 0/0 | 0 | 0 | 13.18 | 1 | 247 | 3.44E−04 |
| RS10496649 | 1.31 | 2/4 | 40 | 1.22 | 4/4 | 1 | 2.38 | 8.22 | 2 | 245 | 3.51E−04 |
| RS9325158 | 2.38 | 2/4 | 24 | 1.39 | 4/4 | 225 | 1.28 | 8.17 | 2 | 247 | 3.67E−04 |
| RS1817186 | 1.27 | 2/4 | 51 | 1.35 | 4/4 | 2 | 2.06 | 8.16 | 2 | 247 | 3.70E−04 |
| RS346421 | 1.26 | 3/4 | 45 | 1.45 | 4/4 | 1 | 1.57 | 8.15 | 2 | 244 | 3.75E−04 |
| RS3770899 | 1.43 | 1/3 | 34 | 1.49 | 3/3 | 210 | 1.27 | 8.13 | 2 | 242 | 3.83E−04 |
| RS10506790 | 1.4 | 1/3 | 40 | 1.47 | 3/3 | 208 | 1.26 | 8.1 | 2 | 247 | 3.92E−04 |
| RS1749541 | 1.26 | 2/4 | 106 | 1.29 | 4/4 | 21 | 1.54 | 8.06 | 2 | 240 | 4.09E−04 |
| RS1935229 | 1.47 | 4/4 | 215 | 1.27 | 0/0 | 0 | 0 | 12.76 | 1 | 246 | 4.26E−04 |
| RS250388 | 1.38 | 1/2 | 53 | 1.43 | 2/2 | 185 | 1.25 | 7.99 | 2 | 241 | 4.37E−04 |
| RS4073625 | 1.98 | 2/4 | 68 | 1.29 | 4/4 | 177 | 1.29 | 7.98 | 2 | 245 | 4.39E−04 |
| RS4770403 | 1.61 | 1/3 | 48 | 1.44 | 3/3 | 200 | 1.26 | 7.96 | 2 | 247 | 4.47E−04 |
| RS2157768 | 1.28 | 1/3 | 15 | 1.56 | 3/3 | 1 | 0.76 | 7.96 | 2 | 246 | 4.47E−04 |
| RS10514844 | 1.69 | 3/3 | 241 | 1.29 | 0/0 | 0 | 0 | 12.65 | 1 | 246 | 4.50E−04 |
| RS10484205 | 1.18 | 1/3 | 90 | 1.39 | 3/3 | 145 | 1.25 | 7.92 | 2 | 247 | 4.64E−04 |
| RS7897663 | 1.37 | 1/3 | 97 | 1.21 | 3/3 | 28 | 1.26 | 7.87 | 2 | 245 | 4.87E−04 |
| RS2278016 | 1.72 | 2/4 | 74 | 1.34 | 4/4 | 164 | 1.26 | 7.87 | 2 | 241 | 4.89E−04 |
| RS9303347 | 0.76 | 1/3 | 15 | 1.55 | 3/3 | 228 | 1.28 | 7.87 | 2 | 241 | 4.89E−04 |
| RS1011439 | 0.76 | 1/3 | 15 | 1.56 | 3/3 | 234 | 1.28 | 7.86 | 2 | 247 | 4.91E−04 |
| RS10494663 | 1.75 | 1/3 | 51 | 1.22 | 3/3 | 188 | 1.31 | 7.85 | 2 | 241 | 4.98E−04 |
| RS3218491 | 1.72 | 4/4 | 238 | 1.29 | 0/0 | 0 | 0 | 12.42 | 1 | 242 | 5.08E−04 |
| RS1527420 | 1.28 | 1/2 | 45 | 1.3 | 2/2 | 5 | 1.82 | 7.8 | 2 | 230 | 5.28E−04 |
| RS10509929 | 1.94 | 2/3 | 49 | 1.34 | 3/3 | 187 | 1.28 | 7.79 | 2 | 236 | 5.29E−04 |
| RS10508378 | 2.38 | 2/4 | 16 | 1.19 | 4/4 | 233 | 1.3 | 7.74 | 2 | 247 | 5.49E−04 |
| RS2413809 | 1.26 | 2/3 | 118 | 1.28 | 3/3 | 25 | 1.51 | 7.63 | 2 | 242 | 6.12E−04 |
| RS10508940 | 1.28 | 2/4 | 78 | 1.28 | 4/4 | 11 | 1.64 | 7.62 | 2 | 246 | 6.15E−04 |
| RS9284617 | 1.45 | 3/3 | 208 | 1.27 | 0/0 | 0 | 0 | 12.03 | 1 | 247 | 6.17E−04 |
| RS788005 | 1.31 | 2/4 | 64 | 1.24 | 4/4 | 12 | 1.61 | 7.62 | 2 | 217 | 6.33E−04 |
| RS2056247 | 1.61 | 3/4 | 78 | 1.35 | 4/4 | 164 | 1.25 | 7.58 | 2 | 247 | 6.38E−04 |
| RS10495774 | 1.63 | 2/4 | 76 | 1.26 | 4/4 | 161 | 1.29 | 7.58 | 2 | 245 | 6.40E−04 |
| RS10484317 | 1.28 | 2/3 | 19 | 1.34 | 3/3 | 2 | 2.08 | 7.57 | 2 | 243 | 6.47E−04 |
| RS10517496 | 2.04 | 2/4 | 38 | 1.22 | 4/4 | 204 | 1.3 | 7.55 | 2 | 241 | 6.60E−04 |
| RS10485111 | 1.26 | 2/4 | 56 | 1.39 | 4/4 | 4 | 1.68 | 7.54 | 2 | 243 | 6.65E−04 |
| RS2748324 | 1.3 | 2/4 | 33 | 1.22 | 4/4 | 4 | 1.82 | 7.53 | 2 | 243 | 6.72E−04 |
| RS4405428 | 1.29 | 2/4 | 54 | 1.27 | 4/4 | 2 | 2.08 | 7.52 | 2 | 243 | 6.78E−04 |
| RS1044045 | 1.35 | 1/3 | 68 | 1.19 | 3/3 | 9 | 1.21 | 7.5 | 2 | 239 | 6.93E−04 |
| RS176435 | 1.29 | 2/4 | 51 | 1.26 | 4/4 | 8 | 1.69 | 7.5 | 2 | 235 | 6.96E−04 |
| RS10513351 | 1.22 | 3/4 | 117 | 1.38 | 4/4 | 43 | 1.25 | 7.5 | 2 | 224 | 7.03E−04 |
| RS3766988 | 1.69 | 2/4 | 51 | 1.25 | 4/4 | 179 | 1.29 | 7.48 | 2 | 235 | 7.09E−04 |
| RS8068574 | 1.28 | 1/3 | 80 | 1.27 | 3/3 | 15 | 1.59 | 7.47 | 2 | 243 | 7.11E−04 |
| RS10483457 | 1.28 | 1/3 | 30 | 1.37 | 3/3 | 2 | 2.04 | 7.46 | 2 | 246 | 7.16E−04 |
| RS879780 | 2.38 | 2/4 | 27 | 1.22 | 4/4 | 218 | 1.3 | 7.45 | 2 | 243 | 7.24E−04 |
| RS10513301 | 1.25 | 2/4 | 68 | 1.41 | 4/4 | 7 | 1.33 | 7.41 | 2 | 245 | 7.51E−04 |
| RS4121693 | 1.25 | 2/4 | 100 | 1.38 | 4/4 | 113 | 1.23 | 7.42 | 2 | 234 | 7.51E−04 |
| RS10508059 | 1.25 | 2/4 | 65 | 1.42 | 4/4 | 8 | 1.25 | 7.41 | 2 | 241 | 7.53E−04 |
| RS1878144 | 1.3 | 3/4 | 48 | 1.24 | 4/4 | 5 | 1.77 | 7.41 | 2 | 237 | 7.56E−04 |
| RS10517504 | 2.04 | 1/3 | 28 | 1.21 | 3/3 | 204 | 1.3 | 7.41 | 2 | 231 | 7.60E−04 |
| RS1356216 | 1.29 | 3/4 | 48 | 1.24 | 4/4 | 5 | 1.77 | 7.39 | 2 | 245 | 7.65E−04 |
| RS2127258 | 1.85 | 2/4 | 39 | 1.38 | 4/4 | 206 | 1.27 | 7.37 | 2 | 245 | 7.80E−04 |
| RS1226489 | 2.26 | 2/4 | 8 | 1.5 | 4/4 | 235 | 1.28 | 7.37 | 2 | 241 | 7.82E−04 |
| RS877984 | 1.21 | 1/4 | 94 | 1.37 | 4/4 | 36 | 1.18 | 7.45 | 2 | 175 | 7.85E−04 |
| RS6735220 | 1.29 | 2/4 | 41 | 1.27 | 4/4 | 2 | 2.09 | 7.36 | 2 | 247 | 7.86E−04 |
| RS3745718 | 1.37 | 1/3 | 125 | 1.23 | 3/3 | 48 | 1.36 | 7.36 | 2 | 247 | 7.86E−04 |
| RS9303346 | 0.76 | 1/4 | 14 | 1.56 | 4/4 | 233 | 1.28 | 7.36 | 2 | 245 | 7.87E−04 |
| RS877345 | 1.3 | 1/2 | 42 | 1.28 | 2/2 | 3 | 1.95 | 7.35 | 2 | 228 | 8.07E−04 |
| RS973916 | 1.3 | 2/4 | 66 | 1.25 | 4/4 | 6 | 1.73 | 7.31 | 2 | 239 | 8.29E−04 |
| RS1371077 | 1.33 | 2/4 | 63 | 1.21 | 4/4 | 1 | 2.08 | 7.29 | 2 | 241 | 8.43E−04 |
| RS1013401 | 1.38 | 1/3 | 114 | 1.22 | 3/3 | 36 | 1.27 | 7.29 | 2 | 232 | 8.50E−04 |
| RS33375 | 1.58 | 1/3 | 49 | 1.41 | 3/3 | 179 | 1.26 | 7.29 | 2 | 231 | 8.51E−04 |
| RS10490353 | 1.3 | 2/3 | 45 | 1.25 | 3/3 | 2 | 2.07 | 7.27 | 2 | 247 | 8.55E−04 |
| RS10517616 | 1.38 | 2/4 | 115 | 1.23 | 4/4 | 24 | 1.24 | 7.26 | 2 | 246 | 8.64E−04 |
| RS716224 | 1.57 | 1/3 | 69 | 1.19 | 3/3 | 173 | 1.33 | 7.26 | 2 | 244 | 8.66E−04 |
| RS10497783 | 2.09 | 1/4 | 35 | 1.28 | 4/4 | 209 | 1.29 | 7.26 | 2 | 243 | 8.66E−04 |
| RS9304301 | 1.3 | 2/4 | 19 | 1.14 | 4/4 | 1 | 2.2 | 7.26 | 2 | 243 | 8.66E−04 |
| RS4646981 | 1.61 | 3/3 | 239 | 1.28 | 0/0 | 0 | 0 | 11.36 | 1 | 247 | 8.71E−04 |
| RS1535053 | 1.28 | 1/3 | 95 | 1.28 | 3/3 | 16 | 1.57 | 7.25 | 2 | 238 | 8.78E−04 |
| RS4146127 | 1.26 | 1/3 | 72 | 1.37 | 3/3 | 4 | 1.7 | 7.24 | 2 | 241 | 8.84E−04 |
| RS10519371 | 1.18 | 3/4 | 118 | 1.34 | 4/4 | 52 | 1.34 | 7.24 | 2 | 236 | 8.88E−04 |
| RS10509698 | 2.38 | 1/3 | 12 | 1.38 | 3/3 | 236 | 1.29 | 7.23 | 2 | 246 | 8.89E−04 |
| RS3218177 | 2.26 | 1/3 | 46 | 1.22 | 3/3 | 200 | 1.31 | 7.23 | 2 | 244 | 8.90E−04 |
| RS7577835 | 1.62 | 2/4 | 69 | 1.24 | 4/4 | 171 | 1.3 | 7.22 | 2 | 247 | 8.97E−04 |
| RS9312013 | 1.29 | 3/4 | 11 | 1.6 | 0/0 | 0 | 0 | 11.29 | 1 | 232 | 9.11E−04 |
| RS10500668 | 2.2 | 2/4 | 29 | 1.18 | 4/4 | 220 | 1.31 | 7.2 | 2 | 247 | 9.14E−04 |
| RS2670607 | 1.93 | 1/4 | 44 | 1.32 | 4/4 | 199 | 1.28 | 7.2 | 2 | 243 | 9.17E−04 |
| RS1550740 | 2.38 | 2/4 | 20 | 1.36 | 4/4 | 223 | 1.29 | 7.2 | 2 | 241 | 9.18E−04 |
| RS6990997 | 1.47 | 2/4 | 39 | 1.45 | 4/4 | 202 | 1.26 | 7.15 | 2 | 244 | 9.60E−04 |
| RS10506265 | 2.38 | 2/4 | 26 | 1.34 | 4/4 | 223 | 1.29 | 7.13 | 2 | 247 | 9.76E−04 |
| RS10487745 | 2.02 | 1/2 | 12 | 1.16 | 2/2 | 203 | 1.29 | 7.16 | 2 | 214 | 9.77E−04 |
| RS10494664 | 1.3 | 1/2 | 59 | 1.24 | 2/2 | 5 | 1.75 | 7.11 | 2 | 245 | 9.96E−04 |
| RS10509702 | 1.29 | 1/3 | 10 | 1.38 | 3/3 | 1 | 2.38 | 7.11 | 2 | 245 | 9.96E−04 |
| RS4793547 | 1.18 | 2/4 | 95 | 1.34 | 4/4 | 66 | 1.2 | 7.14 | 2 | 194 | 1.02E−03 |
| RS4131522 | 1.27 | 2/4 | 17 | 1.55 | 4/4 | 2 | 1.1 | 7.09 | 2 | 236 | 1.02E−03 |
| RS7536312 | 1.75 | 1/3 | 67 | 1.24 | 3/3 | 178 | 1.3 | 7.08 | 2 | 247 | 1.02E−03 |
| RS1428250 | 1.28 | 1/2 | 14 | 1.55 | 0/0 | 0 | 0 | 11.03 | 1 | 230 | 1.04E−03 |
| RS7718000 | 1.74 | 3/3 | 241 | 1.29 | 0/0 | 0 | 0 | 11 | 1 | 244 | 1.05E−03 |
| RS2371319 | 2.02 | 2/4 | 18 | 1.19 | 4/4 | 230 | 1.3 | 7.05 | 2 | 247 | 1.05E−03 |
| RS10516142 | 1.29 | 2/4 | 6 | 1.4 | 4/4 | 1 | 2.38 | 7.05 | 2 | 247 | 1.05E−03 |
| RS7597348 | 1.49 | 2/4 | 107 | 1.26 | 4/4 | 113 | 1.29 | 7.05 | 2 | 246 | 1.05E−03 |
| RS10509161 | 1.92 | 1/3 | 34 | 1.4 | 3/3 | 210 | 1.27 | 7.05 | 2 | 243 | 1.06E−03 |
| RS2827082 | 1.78 | 2/4 | 82 | 1.28 | 4/4 | 154 | 1.28 | 7.05 | 2 | 238 | 1.06E−03 |
| RS10491068 | 1.29 | 2/3 | 13 | 1.36 | 3/3 | 1 | 2.38 | 7.04 | 2 | 247 | 1.06E−03 |
| RS190788 | 1.29 | 2/3 | 16 | 1.35 | 3/3 | 1 | 2.38 | 7.04 | 2 | 247 | 1.06E−03 |
| RS1023431 | 1.27 | 4/4 | 32 | 1.46 | 0/0 | 0 | 0 | 10.96 | 1 | 244 | 1.07E−03 |
| RS113318 | 1.89 | 2/4 | 93 | 1.26 | 4/4 | 147 | 1.32 | 7.02 | 2 | 240 | 1.09E−03 |
| RS4131441 | 1.57 | 1/3 | 113 | 1.29 | 3/3 | 116 | 1.27 | 7.01 | 2 | 242 | 1.10E−03 |
| RS1275637 | 1.26 | 2/4 | 67 | 1.4 | 0/0 | 0 | 0 | 10.9 | 1 | 243 | 1.11E−03 |
| RS953681 | 1.3 | 1/3 | 54 | 1.23 | 3/3 | 5 | 1.74 | 6.99 | 2 | 247 | 1.11E−03 |
| RS381765 | 1.29 | 2/4 | 16 | 1.25 | 4/4 | 1 | 2.38 | 6.99 | 2 | 246 | 1.12E−03 |
| RS1226491 | 2.26 | 1/3 | 12 | 1.45 | 3/3 | 237 | 1.28 | 6.97 | 2 | 247 | 1.14E−03 |
| RS10509119 | 1.3 | 1/3 | 48 | 1.26 | 3/3 | 2 | 2.06 | 6.96 | 2 | 247 | 1.15E−03 |
| RS6988377 | 1.82 | 1/4 | 56 | 1.33 | 4/4 | 188 | 1.28 | 6.95 | 2 | 245 | 1.16E−03 |
| RS7665090 | 1.25 | 2/4 | 109 | 1.37 | 4/4 | 67 | 1.21 | 6.96 | 2 | 230 | 1.16E−03 |
| RS10509160 | 1.92 | 2/3 | 39 | 1.39 | 3/3 | 209 | 1.27 | 6.92 | 2 | 247 | 1.19E−03 |
| RS1332925 | 1.29 | 1/3 | 32 | 1.32 | 3/3 | 3 | 1.92 | 6.9 | 2 | 246 | 1.21E−03 |
| RS10516867 | 2.38 | 1/3 | 48 | 1.26 | 3/3 | 192 | 1.3 | 6.9 | 2 | 238 | 1.22E−03 |
| RS9285122 | 1.64 | 4/4 | 241 | 1.28 | 0/0 | 0 | 0 | 10.7 | 1 | 247 | 1.22E−03 |
| RS2150468 | 1.28 | 3/4 | 8 | 1.64 | 0/0 | 0 | 0 | 10.7 | 1 | 247 | 1.22E−03 |
| RS9284616 | 1.27 | 2/4 | 32 | 1.45 | 0/0 | 0 | 0 | 10.7 | 1 | 238 | 1.23E−03 |
| RS2969001 | 1.25 | 2/3 | 119 | 1.28 | 3/3 | 38 | 1.46 | 6.88 | 2 | 247 | 1.24E−03 |
| RS7794555 | 1.27 | 2/4 | 34 | 1.45 | 0/0 | 0 | 0 | 10.68 | 1 | 246 | 1.24E−03 |
| RS10495785 | 1.28 | 1/3 | 41 | 1.28 | 3/3 | 4 | 1.82 | 6.88 | 2 | 235 | 1.25E−03 |
| RS10492506 | 2.26 | 2/4 | 5 | 1.06 | 4/4 | 244 | 1.3 | 6.87 | 2 | 247 | 1.25E−03 |
| RS723043 | 1.21 | 3/4 | 117 | 1.3 | 4/4 | 36 | 1.42 | 6.89 | 2 | 219 | 1.25E−03 |
| RS2323936 | 1.64 | 4/4 | 242 | 1.29 | 0/0 | 0 | 0 | 10.62 | 1 | 248 | 1.28E−03 |
| RS2323937 | 1.29 | 1/4 | 8 | 1.64 | 0/0 | 0 | 0 | 10.62 | 1 | 248 | 1.28E−03 |
| RS1851006 | 1.19 | 2/4 | 66 | 1.26 | 4/4 | 73 | 1.38 | 6.91 | 2 | 181 | 1.28E−03 |
| RS10509705 | 2.38 | 2/4 | 12 | 1.34 | 4/4 | 237 | 1.29 | 6.84 | 2 | 247 | 1.28E−03 |
| RS10517294 | 1.29 | 1/3 | 33 | 1.32 | 3/3 | 1 | 2.38 | 6.84 | 2 | 246 | 1.29E−03 |
| RS6727480 | 1.53 | 2/4 | 82 | 1.25 | 4/4 | 146 | 1.3 | 6.83 | 2 | 243 | 1.30E−03 |
| RS2169942 | 1.27 | 1/3 | 37 | 1.37 | 3/3 | 3 | 1.85 | 6.83 | 2 | 243 | 1.30E−03 |
| RS10499227 | 1.1 | 1/2 | 28 | 1.46 | 2/2 | 172 | 1.28 | 6.86 | 2 | 206 | 1.31E−03 |
| RS1577879 | 1.27 | 2/4 | 59 | 1.33 | 4/4 | 15 | 1.55 | 6.82 | 2 | 234 | 1.32E−03 |
| RS1029233 | 1.15 | 1/2 | 85 | 1.39 | 2/2 | 139 | 1.27 | 6.81 | 2 | 244 | 1.32E−03 |
| RS7571668 | 1.32 | 2/4 | 112 | 1.24 | 4/4 | 22 | 1.49 | 6.8 | 2 | 237 | 1.34E−03 |
| RS10514845 | 1.64 | 4/4 | 240 | 1.29 | 0/0 | 0 | 0 | 10.52 | 1 | 246 | 1.34E−03 |
| RS4775359 | 1.3 | 2/4 | 53 | 1.27 | 4/4 | 1 | 2.38 | 6.79 | 2 | 246 | 1.35E−03 |
| RS1889226 | 1.29 | 2/4 | 23 | 1.29 | 4/4 | 1 | 2.38 | 6.79 | 2 | 240 | 1.35E−03 |
| RS10517865 | 1.28 | 2/4 | 47 | 1.29 | 4/4 | 10 | 1.64 | 6.77 | 2 | 238 | 1.38E−03 |
| RS10516865 | 1.29 | 3/4 | 5 | 1.72 | 0/0 | 0 | 0 | 10.46 | 1 | 248 | 1.39E−03 |
| RS1004246 | 1.31 | 1/3 | 106 | 1.35 | 3/3 | 37 | 1.14 | 6.77 | 2 | 233 | 1.39E−03 |
| RS10510714 | 1.3 | 1/3 | 15 | 1.2 | 3/3 | 2 | 2.02 | 6.75 | 2 | 247 | 1.40E−03 |
| RS4143188 | 2.38 | 1/2 | 34 | 1.31 | 2/2 | 215 | 1.29 | 6.75 | 2 | 247 | 1.40E−03 |
| RS340686 | 1.35 | 2/4 | 83 | 1.21 | 4/4 | 12 | 1.18 | 6.74 | 2 | 246 | 1.41E−03 |
| RS10486650 | 2.38 | 1/3 | 8 | 1.25 | 3/3 | 240 | 1.29 | 6.74 | 2 | 246 | 1.41E−03 |
| RS10499017 | 1.27 | 2/3 | 124 | 1.27 | 3/3 | 30 | 1.48 | 6.74 | 2 | 243 | 1.42E−03 |
| RS10517183 | 1.67 | 2/4 | 77 | 1.27 | 4/4 | 164 | 1.29 | 6.73 | 2 | 246 | 1.43E−03 |
| RS10494826 | 1.19 | 2/4 | 73 | 1.4 | 4/4 | 163 | 1.25 | 6.73 | 2 | 244 | 1.43E−03 |
| RS2254162 | 1.38 | 1/4 | 52 | 1.4 | 4/4 | 113 | 1.24 | 6.8 | 2 | 174 | 1.43E−03 |
| RS1887889 | 1.29 | 2/3 | 123 | 1.25 | 3/3 | 37 | 1.46 | 6.72 | 2 | 247 | 1.44E−03 |
| RS1013459 | 1.3 | 1/3 | 35 | 1.27 | 3/3 | 1 | 2.38 | 6.72 | 2 | 244 | 1.44E−03 |
| RS4128923 | 1.89 | 2/4 | 75 | 1.32 | 4/4 | 168 | 1.27 | 6.71 | 2 | 243 | 1.46E−03 |
| RS2072506 | 1.27 | 1/4 | 31 | 1.46 | 0/0 | 0 | 0 | 10.34 | 1 | 243 | 1.48E−03 |
| RS2034022 | 1.38 | 2/4 | 113 | 1.25 | 4/4 | 32 | 1.21 | 6.69 | 2 | 239 | 1.49E−03 |
| RS924993 | 2.38 | 1/3 | 32 | 1.28 | 3/3 | 217 | 1.29 | 6.68 | 2 | 247 | 1.50E−03 |
| RS10506869 | 2.38 | 2/3 | 32 | 1.3 | 3/3 | 217 | 1.29 | 6.68 | 2 | 247 | 1.50E−03 |
| RS10505540 | 2.15 | 2/4 | 19 | 1.44 | 4/4 | 230 | 1.28 | 6.67 | 2 | 247 | 1.51E−03 |
| RS265056 | 1.29 | 2/3 | 36 | 1.3 | 3/3 | 1 | 2.38 | 6.67 | 2 | 245 | 1.51E−03 |
| RS10495520 | 1.77 | 2/4 | 56 | 1.23 | 4/4 | 188 | 1.3 | 6.66 | 2 | 245 | 1.53E−03 |
| RS6856098 | 1.58 | 2/4 | 75 | 1.33 | 4/4 | 159 | 1.26 | 6.66 | 2 | 242 | 1.53E−03 |
| RS10503419 | 1.28 | 1/2 | 70 | 1.3 | 2/2 | 4 | 1.84 | 6.67 | 2 | 228 | 1.53E−03 |
| RS439269 | 1.26 | 2/4 | 48 | 1.39 | 4/4 | 5 | 1.64 | 6.65 | 2 | 246 | 1.54E−03 |
| RS720327 | 1.29 | 1/2 | 31 | 1.3 | 2/2 | 1 | 2.38 | 6.65 | 2 | 242 | 1.54E−03 |
| RS10513299 | 1.26 | 1/4 | 58 | 1.38 | 4/4 | 3 | 1.76 | 6.64 | 2 | 243 | 1.56E−03 |
| RS1917952 | 1.19 | 2/4 | 101 | 1.35 | 4/4 | 52 | 1.34 | 6.65 | 2 | 225 | 1.56E−03 |
| RS10483252 | 2.38 | 2/4 | 42 | 1.29 | 4/4 | 205 | 1.29 | 6.63 | 2 | 245 | 1.57E−03 |
| RS6733858 | 1.23 | 1/3 | 126 | 1.37 | 3/3 | 40 | 1.23 | 6.63 | 2 | 244 | 1.57E−03 |
| RS1429130 | 1.28 | 1/3 | 25 | 1.48 | 0/0 | 0 | 0 | 10.21 | 1 | 232 | 1.59E−03 |
| RS3118650 | 1.28 | 1/3 | 14 | 1.54 | 0/0 | 0 | 0 | 10.19 | 1 | 246 | 1.60E−03 |
| RS1959736 | 1.66 | 3/4 | 35 | 1.29 | 4/4 | 186 | 1.28 | 6.62 | 2 | 226 | 1.61E−03 |
| RS10511852 | 1.28 | 2/4 | 15 | 1.54 | 0/0 | 0 | 0 | 10.17 | 1 | 244 | 1.61E−03 |
| RS10508692 | 1.72 | 3/3 | 241 | 1.29 | 0/0 | 0 | 0 | 10.12 | 1 | 244 | 1.66E−03 |
| RS2147085 | 1.43 | 1/3 | 130 | 1.26 | 3/3 | 64 | 1.25 | 6.57 | 2 | 242 | 1.67E−03 |
| RS4611835 | 1.92 | 1/2 | 19 | 1.44 | 2/2 | 220 | 1.28 | 6.57 | 2 | 238 | 1.67E−03 |
| RS715428 | 1.56 | 2/2 | 237 | 1.28 | 0/0 | 0 | 0 | 10.09 | 1 | 247 | 1.68E−03 |
| RS10516872 | 1.3 | 1/3 | 24 | 1.31 | 3/3 | 1 | 2.38 | 6.56 | 2 | 240 | 1.68E−03 |
| RS9323955 | 1.17 | 1/3 | 123 | 1.34 | 3/3 | 64 | 1.33 | 6.56 | 2 | 240 | 1.68E−03 |
| RS1844510 | 1.32 | 1/3 | 121 | 1.24 | 3/3 | 56 | 1.41 | 6.55 | 2 | 238 | 1.70E−03 |
| RS1204014 | 1.29 | 2/4 | 19 | 1.22 | 4/4 | 2 | 2.02 | 6.53 | 2 | 246 | 1.72E−03 |
| RS2785949 | 1.26 | 1/3 | 79 | 1.29 | 3/3 | 31 | 1.47 | 6.54 | 2 | 231 | 1.73E−03 |
| RS2721304 | 1.27 | 1/3 | 56 | 1.41 | 0/0 | 0 | 0 | 10.03 | 1 | 240 | 1.74E−03 |
| RS10508096 | 1.05 | 2/4 | 68 | 1.39 | 4/4 | 173 | 1.27 | 6.52 | 2 | 244 | 1.74E−03 |
| RS10510100 | 1.3 | 2/4 | 12 | 1.15 | 4/4 | 3 | 1.84 | 6.51 | 2 | 245 | 1.76E−03 |
| RS9287120 | 1.75 | 2/3 | 32 | 1.23 | 3/3 | 181 | 1.3 | 6.53 | 2 | 215 | 1.77E−03 |
| RS1818711 | 1.25 | 1/3 | 128 | 1.28 | 3/3 | 41 | 1.45 | 6.49 | 2 | 246 | 1.79E−03 |
| RS10497689 | 1.71 | 3/3 | 245 | 1.29 | 0/0 | 0 | 0 | 9.95 | 1 | 248 | 1.81E−03 |
| RS10492139 | 1.32 | 2/4 | 85 | 1.39 | 4/4 | 149 | 1.24 | 6.48 | 2 | 245 | 1.81E−03 |
| RS3886738 | 1.92 | 1/3 | 35 | 1.38 | 3/3 | 208 | 1.27 | 6.48 | 2 | 242 | 1.81E−03 |
| RS10494214 | 1.3 | 2/4 | 34 | 1.28 | 4/4 | 1 | 2.38 | 6.48 | 2 | 237 | 1.82E−03 |
| RS6798797 | 1.3 | 1/3 | 14 | 1.21 | 3/3 | 2 | 2.02 | 6.46 | 2 | 242 | 1.85E−03 |
| RS935313 | 1.28 | 1/2 | 91 | 1.38 | 2/2 | 84 | 1.22 | 6.47 | 2 | 224 | 1.85E−03 |
| RS1464452 | 1.25 | 1/3 | 44 | 1.44 | 3/3 | 190 | 1.26 | 6.45 | 2 | 238 | 1.87E−03 |
| RS2277456 | 1.26 | 2/4 | 28 | 1.46 | 4/4 | 3 | 1.58 | 6.47 | 2 | 209 | 1.88E−03 |
| RS1367719 | 1.25 | 1/2 | 103 | 1.37 | 2/2 | 22 | 1.19 | 6.44 | 2 | 246 | 1.88E−03 |
| RS7752110 | 1.65 | 2/4 | 66 | 1.3 | 4/4 | 173 | 1.28 | 6.43 | 2 | 245 | 1.90E−03 |
| RS4918842 | 1.63 | 2/4 | 72 | 1.25 | 4/4 | 167 | 1.3 | 6.43 | 2 | 245 | 1.90E−03 |
| RS1025748 | 1.31 | 1/2 | 72 | 1.23 | 2/2 | 16 | 1.52 | 6.43 | 2 | 239 | 1.91E−03 |
| RS10514770 | 1.36 | 1/3 | 111 | 1.22 | 3/3 | 33 | 1.32 | 6.42 | 2 | 245 | 1.92E−03 |
| RS1442799 | 1.29 | 2/4 | 33 | 1.32 | 4/4 | 2 | 2.04 | 6.41 | 2 | 247 | 1.93E−03 |
| RS2405708 | 1.35 | 1/3 | 67 | 1.37 | 3/3 | 82 | 1.2 | 6.45 | 2 | 197 | 1.94E−03 |
| RS7413158 | 1.71 | 1/2 | 64 | 1.26 | 2/2 | 180 | 1.3 | 6.4 | 2 | 247 | 1.95E−03 |
| RS1407466 | 1.25 | 1/3 | 73 | 1.38 | 3/3 | 10 | 1.46 | 6.4 | 2 | 245 | 1.95E−03 |
| RS2254764 | 1.9 | 3/4 | 37 | 1.31 | 4/4 | 210 | 1.29 | 6.39 | 2 | 247 | 1.97E−03 |
| RS539254 | 1.25 | 1/3 | 64 | 1.38 | 3/3 | 13 | 1.5 | 6.41 | 2 | 213 | 1.98E−03 |
| RS2333821 | 1.23 | 2/4 | 106 | 1.33 | 4/4 | 20 | 1.47 | 6.39 | 2 | 235 | 1.98E−03 |
| RS1016579 | 1.3 | 1/3 | 24 | 1.49 | 3/3 | 194 | 1.27 | 6.39 | 2 | 217 | 2.01E−03 |
| RS10484518 | 1.26 | 3/4 | 104 | 1.38 | 4/4 | 114 | 1.23 | 6.37 | 2 | 240 | 2.02E−03 |
| RS6989370 | 1.32 | 2/4 | 126 | 1.34 | 4/4 | 65 | 1.19 | 6.36 | 2 | 246 | 2.03E−03 |
| RS10505777 | 1.29 | 2/3 | 4 | 1.77 | 0/0 | 0 | 0 | 9.81 | 1 | 117 | 2.03E−03 |
| RS717806 | 1.48 | 1/4 | 100 | 1.35 | 4/4 | 129 | 1.24 | 6.36 | 2 | 237 | 2.04E−03 |
| RS1577302 | 1.66 | 2/4 | 35 | 1.2 | 4/4 | 198 | 1.3 | 6.36 | 2 | 236 | 2.04E−03 |
| RS1869733 | 1.27 | 2/4 | 76 | 1.39 | 4/4 | 11 | 1.11 | 6.35 | 2 | 245 | 2.05E−03 |
| RS1217656 | 2.2 | 1/2 | 38 | 1.21 | 2/2 | 209 | 1.31 | 6.34 | 2 | 245 | 2.07E−03 |
| RS2838000 | 1.49 | 1/3 | 98 | 1.24 | 3/3 | 129 | 1.31 | 6.34 | 2 | 244 | 2.07E−03 |
| RS6741304 | 1.47 | 2/4 | 102 | 1.31 | 4/4 | 117 | 1.24 | 6.34 | 2 | 241 | 2.07E−03 |
| RS2223163 | 1.2 | 2/4 | 116 | 1.36 | 4/4 | 106 | 1.23 | 6.34 | 2 | 233 | 2.08E−03 |
| RS4952999 | 1.3 | 1/4 | 109 | 1.36 | 4/4 | 100 | 1.22 | 6.33 | 2 | 245 | 2.09E−03 |
| RS2973237 | 1.48 | 2/4 | 90 | 1.26 | 4/4 | 116 | 1.28 | 6.33 | 2 | 231 | 2.11E−03 |
| RS3124806 | 1.45 | 3/3 | 215 | 1.27 | 0/0 | 0 | 0 | 9.66 | 1 | 239 | 2.11E−03 |
| RS1485870 | 1.35 | 2/4 | 72 | 1.2 | 4/4 | 14 | 1.28 | 6.32 | 2 | 240 | 2.11E−03 |
| RS10518405 | 1.97 | 1/2 | 30 | 1.21 | 2/2 | 210 | 1.3 | 6.32 | 2 | 239 | 2.12E−03 |
| RS10497650 | 1.71 | 4/4 | 232 | 1.29 | 0/0 | 0 | 0 | 9.65 | 1 | 235 | 2.13E−03 |
| RS10488693 | 1.48 | 3/3 | 222 | 1.28 | 0/0 | 0 | 0 | 9.64 | 1 | 243 | 2.13E−03 |
| RS10507082 | 1.04 | 1/3 | 78 | 1.27 | 3/3 | 146 | 1.33 | 6.31 | 2 | 235 | 2.14E−03 |
| RS10510715 | 2.02 | 2/4 | 8 | 1.2 | 4/4 | 239 | 1.29 | 6.3 | 2 | 246 | 2.15E−03 |
| RS4684321 | 1.28 | 1/3 | 23 | 1.48 | 0/0 | 0 | 0 | 9.61 | 1 | 248 | 2.16E−03 |
| RS829612 | 1.32 | 2/4 | 111 | 1.22 | 4/4 | 105 | 1.36 | 6.29 | 2 | 246 | 2.17E−03 |
| RS10489925 | 1.33 | 2/4 | 87 | 1.38 | 4/4 | 137 | 1.24 | 6.3 | 2 | 231 | 2.17E−03 |
| RS735403 | 1.25 | 1/3 | 115 | 1.37 | 3/3 | 69 | 1.22 | 6.29 | 2 | 240 | 2.18E−03 |
| RS2217486 | 1.37 | 1/3 | 85 | 1.38 | 3/3 | 154 | 1.24 | 6.28 | 2 | 245 | 2.19E−03 |
| RS10485090 | 1.9 | 1/2 | 50 | 1.3 | 2/2 | 195 | 1.29 | 6.28 | 2 | 245 | 2.19E−03 |
| RS7181421 | 1.24 | 2/4 | 118 | 1.37 | 4/4 | 31 | 1.22 | 6.28 | 2 | 243 | 2.19E−03 |
| RS3964705 | 1.27 | 1/3 | 78 | 1.3 | 3/3 | 9 | 1.63 | 6.28 | 2 | 242 | 2.19E−03 |
| RS1935720 | 1.29 | 3/4 | 56 | 1.3 | 4/4 | 2 | 2.04 | 6.27 | 2 | 246 | 2.21E−03 |
| RS1433450 | 0.91 | 1/3 | 42 | 1.17 | 3/3 | 204 | 1.33 | 6.27 | 2 | 245 | 2.21E−03 |
| RS1452528 | 1.38 | 1/3 | 121 | 1.31 | 3/3 | 60 | 1.19 | 6.27 | 2 | 241 | 2.22E−03 |
| RS10486694 | 2.26 | 1/3 | 10 | 1.17 | 3/3 | 237 | 1.29 | 6.26 | 2 | 245 | 2.23E−03 |
| RS2089907 | 1.24 | 1/4 | 76 | 1.39 | 4/4 | 18 | 1.35 | 6.27 | 2 | 224 | 2.24E−03 |
| RS10484693 | 1.56 | 3/3 | 235 | 1.29 | 0/0 | 0 | 0 | 9.54 | 1 | 245 | 2.24E−03 |
| RS1922036 | 1.26 | 1/2 | 89 | 1.39 | 2/2 | 29 | 1.22 | 6.25 | 2 | 245 | 2.25E−03 |
| RS868691 | 1.28 | 2/4 | 26 | 1.39 | 4/4 | 3 | 1.82 | 6.24 | 2 | 237 | 2.29E−03 |
| RS745708 | 1.24 | 1/3 | 63 | 1.38 | 3/3 | 8 | 1.42 | 6.25 | 2 | 218 | 2.29E−03 |
| RS10502055 | 1.26 | 2/4 | 116 | 1.37 | 4/4 | 58 | 1.21 | 6.23 | 2 | 243 | 2.30E−03 |
| RS1369763 | 1.29 | 2/4 | 7 | 1.69 | 4/4 | 1 | 1.08 | 6.25 | 2 | 215 | 2.30E−03 |
| RS2825652 | 1.28 | 2/3 | 10 | 1.58 | 0/0 | 0 | 0 | 9.49 | 1 | 247 | 2.30E−03 |
| RS10521241 | 1.63 | 1/3 | 74 | 1.21 | 3/3 | 167 | 1.32 | 6.23 | 2 | 242 | 2.30E−03 |
| RS10486012 | 1.6 | 4/4 | 241 | 1.29 | 0/0 | 0 | 0 | 9.46 | 1 | 248 | 2.34E−03 |
| RS1852561 | 1.52 | 4/4 | 234 | 1.28 | 0/0 | 0 | 0 | 9.46 | 1 | 247 | 2.34E−03 |
| RS296032 | 1.11 | 2/3 | 83 | 1.38 | 3/3 | 152 | 1.26 | 6.21 | 2 | 240 | 2.35E−03 |
| RS10495667 | 1.7 | 3/3 | 244 | 1.29 | 0/0 | 0 | 0 | 9.45 | 1 | 247 | 2.35E−03 |
| RS950796 | 1.26 | 1/3 | 61 | 1.4 | 3/3 | 7 | 1.18 | 6.22 | 2 | 224 | 2.35E−03 |
| RS10510155 | 1.26 | 2/4 | 48 | 1.43 | 4/4 | 1 | 1.23 | 6.21 | 2 | 234 | 2.36E−03 |
| RS10511356 | 1.29 | 2/4 | 29 | 1.3 | 4/4 | 2 | 2.04 | 6.2 | 2 | 247 | 2.36E−03 |
| RS1025927 | 1.27 | 1/3 | 27 | 1.49 | 3/3 | 1 | 1.4 | 6.2 | 2 | 246 | 2.36E−03 |
| RS285098 | 1.44 | 2/3 | 107 | 1.25 | 3/3 | 94 | 1.28 | 6.2 | 2 | 240 | 2.37E−03 |
| RS9315749 | 1.17 | 2/3 | 65 | 1.38 | 3/3 | 124 | 1.26 | 6.22 | 2 | 213 | 2.37E−03 |
| RS3850079 | 1.18 | 1/2 | 111 | 1.36 | 2/2 | 109 | 1.24 | 6.2 | 2 | 238 | 2.37E−03 |
| RS340680 | 1.17 | 1/3 | 76 | 1.22 | 3/3 | 154 | 1.35 | 6.19 | 2 | 239 | 2.39E−03 |
| RS10520754 | 1.24 | 2/4 | 125 | 1.35 | 4/4 | 48 | 1.19 | 6.19 | 2 | 236 | 2.40E−03 |
| RS950016 | 1.27 | 2/4 | 27 | 1.46 | 0/0 | 0 | 0 | 9.39 | 1 | 245 | 2.43E−03 |
| RS1936671 | 1.24 | 2/4 | 95 | 1.37 | 4/4 | 13 | 1.38 | 6.17 | 2 | 244 | 2.43E−03 |
| RS1371076 | 1.29 | 1/3 | 101 | 1.22 | 3/3 | 124 | 1.36 | 6.17 | 2 | 243 | 2.43E−03 |
| RS719025 | 1.29 | 3/4 | 65 | 1.41 | 4/4 | 172 | 1.25 | 6.17 | 2 | 243 | 2.43E−03 |
| RS717694 | 1.16 | 2/4 | 88 | 1.22 | 4/4 | 146 | 1.35 | 6.17 | 2 | 239 | 2.44E−03 |
| RS2893569 | 1.28 | 1/3 | 7 | 1.64 | 0/0 | 0 | 0 | 9.39 | 1 | 232 | 2.44E−03 |
| RS641445 | 2.04 | 3/4 | 22 | 1.26 | 4/4 | 197 | 1.3 | 6.18 | 2 | 218 | 2.45E−03 |
| RS4697055 | 1.33 | 1/3 | 76 | 1.2 | 3/3 | 16 | 1.4 | 6.17 | 2 | 231 | 2.45E−03 |
| RS9318541 | 1.15 | 2/4 | 100 | 1.36 | 4/4 | 125 | 1.27 | 6.16 | 2 | 246 | 2.45E−03 |
| RS720613 | 1.27 | 2/4 | 74 | 1.31 | 4/4 | 12 | 1.58 | 6.17 | 2 | 229 | 2.46E−03 |
| RS1465135 | 1.24 | 1/3 | 79 | 1.38 | 3/3 | 21 | 1.28 | 6.17 | 2 | 220 | 2.47E−03 |
| RS295577 | 1.99 | 2/3 | 10 | 1.41 | 3/3 | 231 | 1.28 | 6.15 | 2 | 240 | 2.48E−03 |
| RS7574093 | 1.58 | 3/3 | 240 | 1.28 | 0/0 | 0 | 0 | 9.34 | 1 | 248 | 2.49E−03 |
| RS10517442 | 1.63 | 2/4 | 78 | 1.36 | 4/4 | 167 | 1.26 | 6.14 | 2 | 247 | 2.50E−03 |
| RS7768538 | 1.23 | 1/2 | 129 | 1.3 | 2/2 | 32 | 1.45 | 6.14 | 2 | 243 | 2.50E−03 |
| RS1566728 | 1.25 | 1/3 | 95 | 1.38 | 3/3 | 15 | 1.26 | 6.13 | 2 | 246 | 2.52E−03 |
| RS3805134 | 1.44 | 2/4 | 103 | 1.28 | 4/4 | 87 | 1.25 | 6.14 | 2 | 230 | 2.52E−03 |
| RS7075669 | 1.31 | 1/3 | 116 | 1.36 | 3/3 | 69 | 1.2 | 6.13 | 2 | 241 | 2.53E−03 |
| RS10502056 | 1.2 | 2/4 | 114 | 1.36 | 4/4 | 70 | 1.25 | 6.13 | 2 | 236 | 2.54E−03 |
| RS1385080 | 1.28 | 3/4 | 20 | 1.5 | 0/0 | 0 | 0 | 9.3 | 1 | 243 | 2.55E−03 |
| RS10507553 | 1.7 | 4/4 | 245 | 1.29 | 0/0 | 0 | 0 | 9.27 | 1 | 248 | 2.58E−03 |
| RS1411315 | 1.3 | 1/3 | 92 | 1.38 | 3/3 | 130 | 1.23 | 6.11 | 2 | 240 | 2.58E−03 |
| RS7139422 | 2.26 | 1/4 | 36 | 1.35 | 4/4 | 212 | 1.28 | 6.1 | 2 | 246 | 2.60E−03 |
| RS1235012 | 1.31 | 2/4 | 34 | 1.21 | 4/4 | 1 | 2.2 | 6.1 | 2 | 242 | 2.60E−03 |
| RS7174236 | 1.66 | 2/3 | 66 | 1.3 | 3/3 | 176 | 1.28 | 6.09 | 2 | 247 | 2.62E−03 |
| RS1993745 | 1.15 | 2/4 | 107 | 1.35 | 4/4 | 105 | 1.3 | 6.09 | 2 | 246 | 2.62E−03 |
| RS1341002 | 1.31 | 2/3 | 124 | 1.25 | 3/3 | 18 | 1.5 | 6.09 | 2 | 241 | 2.63E−03 |
| RS10498032 | 1.14 | 1/4 | 100 | 1.35 | 4/4 | 84 | 1.28 | 6.11 | 2 | 212 | 2.63E−03 |
| RS989177 | 1.56 | 2/4 | 40 | 1.29 | 4/4 | 159 | 1.27 | 6.11 | 2 | 211 | 2.63E−03 |
| RS1217654 | 1.31 | 2/4 | 36 | 1.22 | 4/4 | 1 | 2.2 | 6.08 | 2 | 237 | 2.66E−03 |
| RS7453920 | 1.45 | 1/3 | 123 | 1.3 | 3/3 | 84 | 1.24 | 6.08 | 2 | 236 | 2.66E−03 |
| RS1604613 | 1.36 | 2/4 | 123 | 1.31 | 4/4 | 66 | 1.19 | 6.08 | 2 | 233 | 2.67E−03 |
| RS548533 | 1.26 | 4/4 | 59 | 1.4 | 0/0 | 0 | 0 | 9.21 | 1 | 244 | 2.67E−03 |
| RS479554 | 1.4 | 3/3 | 187 | 1.26 | 0/0 | 0 | 0 | 9.21 | 1 | 244 | 2.67E−03 |
| RS4686384 | 1.35 | 1/3 | 120 | 1.23 | 3/3 | 23 | 1.41 | 6.07 | 2 | 244 | 2.67E−03 |
| RS10506626 | 1.49 | 1/3 | 117 | 1.32 | 3/3 | 112 | 1.24 | 6.07 | 2 | 244 | 2.67E−03 |
| RS198278 | 1.18 | 1/3 | 123 | 1.29 | 3/3 | 60 | 1.36 | 6.07 | 2 | 235 | 2.69E−03 |
| RS295525 | 1.99 | 2/4 | 9 | 1.41 | 4/4 | 239 | 1.29 | 6.06 | 2 | 247 | 2.70E−03 |
| RS1886452 | 1.25 | 1/4 | 79 | 1.39 | 4/4 | 8 | 1.38 | 6.06 | 2 | 242 | 2.70E−03 |
| RS2009373 | 1.35 | 2/4 | 125 | 1.24 | 4/4 | 74 | 1.38 | 6.06 | 2 | 240 | 2.71E−03 |
| RS6086149 | 1.26 | 1/3 | 95 | 1.32 | 3/3 | 9 | 1.6 | 6.05 | 2 | 243 | 2.73E−03 |
| RS10500986 | 1.26 | 2/3 | 72 | 1.34 | 3/3 | 11 | 1.54 | 6.06 | 2 | 225 | 2.73E−03 |
| RS10497776 | 1.29 | 1/3 | 5 | 1.7 | 0/0 | 0 | 0 | 9.16 | 1 | 247 | 2.74E−03 |
| RS2832726 | 1.3 | 1/3 | 134 | 1.25 | 3/3 | 38 | 1.44 | 6.04 | 2 | 247 | 2.75E−03 |
| RS10495519 | 1.63 | 1/3 | 62 | 1.22 | 3/3 | 176 | 1.31 | 6.04 | 2 | 241 | 2.76E−03 |
| RS10492135 | 1.35 | 2/4 | 84 | 1.38 | 4/4 | 152 | 1.24 | 6.03 | 2 | 246 | 2.78E−03 |
| RS10508840 | 1.12 | 1/2 | 55 | 1.41 | 2/2 | 187 | 1.27 | 6.03 | 2 | 245 | 2.78E−03 |
| RS167164 | 1.26 | 2/4 | 82 | 1.39 | 4/4 | 8 | 1.17 | 6.03 | 2 | 244 | 2.78E−03 |
| RS967220 | 1.02 | 3/4 | 62 | 1.22 | 4/4 | 143 | 1.35 | 6.05 | 2 | 207 | 2.80E−03 |
| RS10493306 | 1.73 | 2/4 | 73 | 1.26 | 4/4 | 171 | 1.3 | 6.02 | 2 | 246 | 2.80E−03 |
| RS2111826 | 1.16 | 3/3 | 208 | 1.32 | 0/0 | 0 | 0 | 9.11 | 1 | 245 | 2.81E−03 |
| RS722508 | 1.35 | 1/3 | 121 | 1.23 | 3/3 | 83 | 1.37 | 6.01 | 2 | 247 | 2.83E−03 |
| RS7220359 | 1.53 | 3/3 | 229 | 1.28 | 0/0 | 0 | 0 | 9.1 | 1 | 241 | 2.83E−03 |
| RS10490838 | 1.28 | 1/3 | 15 | 1.52 | 0/0 | 0 | 0 | 9.09 | 1 | 247 | 2.84E−03 |
| RS2069837 | 1.27 | 1/3 | 24 | 1.47 | 0/0 | 0 | 0 | 9.09 | 1 | 239 | 2.85E−03 |
| RS1485871 | 1.28 | 1/3 | 63 | 1.19 | 3/3 | 157 | 1.35 | 6 | 2 | 231 | 2.88E−03 |
| RS2113487 | 1.37 | 1/3 | 106 | 1.23 | 3/3 | 26 | 1.31 | 6.01 | 2 | 216 | 2.88E−03 |
| RS10519370 | 1.2 | 2/4 | 119 | 1.34 | 4/4 | 51 | 1.34 | 5.99 | 2 | 245 | 2.89E−03 |
| RS1905155 | 1.33 | 3/4 | 81 | 1.28 | 4/4 | 30 | 1.13 | 6 | 2 | 224 | 2.89E−03 |
| RS3827702 | 1.27 | 2/4 | 63 | 1.26 | 4/4 | 19 | 1.52 | 6.01 | 2 | 205 | 2.91E−03 |
| RS10500783 | 1.27 | 2/4 | 46 | 1.43 | 4/4 | 3 | 1.49 | 5.98 | 2 | 245 | 2.91E−03 |
| RS724334 | 1.27 | 2/4 | 63 | 1.34 | 4/4 | 8 | 1.62 | 5.98 | 2 | 242 | 2.92E−03 |
| RS6852538 | 1.2 | 1/3 | 117 | 1.33 | 3/3 | 52 | 1.35 | 5.97 | 2 | 245 | 2.94E−03 |
| RS6954168 | 1.29 | 2/4 | 7 | 1.63 | 0/0 | 0 | 0 | 9.02 | 1 | 246 | 2.95E−03 |
| RS10512680 | 1.42 | 1/3 | 108 | 1.26 | 3/3 | 59 | 1.26 | 5.98 | 2 | 226 | 2.95E−03 |
| RS10518649 | 1.29 | 2/3 | 66 | 1.28 | 3/3 | 2 | 2.02 | 5.96 | 2 | 247 | 2.97E−03 |
| RS2244327 | 1.25 | 2/4 | 136 | 1.35 | 4/4 | 25 | 1.16 | 5.96 | 2 | 247 | 2.97E−03 |
| RS1153350 | 1.85 | 1/2 | 66 | 1.25 | 2/2 | 181 | 1.3 | 5.96 | 2 | 247 | 2.97E−03 |
| RS4510935 | 1.59 | 2/4 | 62 | 1.37 | 4/4 | 181 | 1.26 | 5.96 | 2 | 246 | 2.97E−03 |
| RS1942578 | 1.2 | 2/4 | 109 | 1.27 | 4/4 | 85 | 1.37 | 5.96 | 2 | 242 | 2.97E−03 |
| RS953177 | 1.28 | 2/4 | 26 | 1.47 | 0/0 | 0 | 0 | 8.98 | 1 | 246 | 3.01E−03 |
| RS7701903 | 1.08 | 2/3 | 31 | 1.47 | 3/3 | 192 | 1.28 | 5.96 | 2 | 223 | 3.01E−03 |
| RS309274 | 1.26 | 1/3 | 104 | 1.37 | 3/3 | 107 | 1.23 | 5.95 | 2 | 235 | 3.01E−03 |
| RS1556082 | 1.38 | 1/3 | 81 | 1.38 | 3/3 | 160 | 1.25 | 5.94 | 2 | 245 | 3.03E−03 |
| RS2616170 | 1.33 | 1/4 | 105 | 1.22 | 4/4 | 107 | 1.36 | 5.94 | 2 | 241 | 3.03E−03 |
| RS10503626 | 1.96 | 1/3 | 19 | 1.39 | 3/3 | 228 | 1.28 | 5.93 | 2 | 246 | 3.05E−03 |
| RS2831853 | 1.31 | 2/3 | 10 | 1.02 | 0/0 | 0 | 0 | 8.95 | 1 | 244 | 3.06E−03 |
| RS2969004 | 1.45 | 3/4 | 104 | 1.29 | 4/4 | 86 | 1.25 | 5.94 | 2 | 226 | 3.06E−03 |
| RS10501160 | 2.26 | 1/3 | 24 | 1.37 | 3/3 | 217 | 1.29 | 5.93 | 2 | 239 | 3.07E−03 |
| RS752355 | 1.56 | 1/2 | 104 | 1.3 | 2/2 | 114 | 1.28 | 5.93 | 2 | 230 | 3.08E−03 |
| RS1372338 | 1.28 | 2/4 | 114 | 1.24 | 4/4 | 65 | 1.4 | 5.92 | 2 | 241 | 3.09E−03 |
| RS1931610 | 2.26 | 1/3 | 15 | 1.38 | 3/3 | 232 | 1.29 | 5.91 | 2 | 245 | 3.11E−03 |
| RS1641354 | 1.45 | 1/3 | 114 | 1.26 | 3/3 | 98 | 1.27 | 5.91 | 2 | 245 | 3.11E−03 |
| RS6919514 | 1.42 | 4/4 | 206 | 1.27 | 0/0 | 0 | 0 | 8.91 | 1 | 247 | 3.12E−03 |
| RS10498836 | 1.33 | 2/4 | 49 | 1.17 | 4/4 | 7 | 1.24 | 5.91 | 2 | 239 | 3.12E−03 |
| RS1381181 | 1.15 | 1/3 | 107 | 1.34 | 3/3 | 105 | 1.3 | 5.9 | 2 | 246 | 3.14E−03 |
| RS1881014 | 1.29 | 2/4 | 17 | 1.52 | 0/0 | 0 | 0 | 8.91 | 1 | 227 | 3.15E−03 |
| RS10501161 | 1.28 | 2/4 | 30 | 1.35 | 4/4 | 1 | 2.26 | 5.9 | 2 | 243 | 3.15E−03 |
| RS1351650 | 1.25 | 2/4 | 97 | 1.38 | 4/4 | 80 | 1.24 | 5.9 | 2 | 237 | 3.16E−03 |
| RS10489249 | 1.3 | 2/3 | 62 | 1.24 | 3/3 | 5 | 1.71 | 5.89 | 2 | 246 | 3.17E−03 |
| RS10518089 | 1.25 | 2/3 | 95 | 1.32 | 3/3 | 18 | 1.5 | 5.89 | 2 | 246 | 3.17E−03 |
| RS4816018 | 1.26 | 1/2 | 93 | 1.31 | 2/2 | 9 | 1.6 | 5.89 | 2 | 246 | 3.17E−03 |
| RS950085 | 1.47 | 1/4 | 95 | 1.27 | 4/4 | 56 | 1.24 | 5.94 | 2 | 172 | 3.20E−03 |
| RS1823913 | 1.3 | 2/4 | 81 | 1.25 | 4/4 | 19 | 1.51 | 5.88 | 2 | 244 | 3.21E−03 |
| RS2383024 | 1.24 | 1/2 | 104 | 1.38 | 2/2 | 35 | 1.25 | 5.88 | 2 | 241 | 3.21E−03 |
| RS9306295 | 1.3 | 1/4 | 29 | 1.16 | 4/4 | 6 | 1.58 | 5.88 | 2 | 238 | 3.22E−03 |
| RS10508842 | 1.15 | 2/3 | 55 | 1.41 | 3/3 | 185 | 1.27 | 5.87 | 2 | 244 | 3.24E−03 |
| RS725500 | 1.08 | 2/3 | 86 | 1.24 | 3/3 | 149 | 1.33 | 5.87 | 2 | 242 | 3.24E−03 |
| RS9326506 | 1.24 | 3/4 | 110 | 1.37 | 4/4 | 36 | 1.23 | 5.87 | 2 | 233 | 3.26E−03 |
| RS9312592 | 1.52 | 2/4 | 46 | 1.4 | 4/4 | 181 | 1.26 | 5.87 | 2 | 231 | 3.26E−03 |
| RS10512935 | 1.27 | 2/4 | 69 | 1.3 | 4/4 | 20 | 1.51 | 5.86 | 2 | 242 | 3.27E−03 |
| RS722569 | 1.17 | 1/3 | 117 | 1.3 | 3/3 | 86 | 1.36 | 5.86 | 2 | 242 | 3.27E−03 |
| RS1503453 | 1.31 | 3/4 | 62 | 1.24 | 4/4 | 6 | 1.67 | 5.86 | 2 | 238 | 3.28E−03 |
| RS719484 | 1.26 | 1/2 | 64 | 1.4 | 2/2 | 5 | 1.5 | 5.86 | 2 | 238 | 3.28E−03 |
| RS2477035 | 1.16 | 1/3 | 111 | 1.31 | 3/3 | 88 | 1.35 | 5.86 | 2 | 237 | 3.28E−03 |
| RS10495033 | 1.94 | 2/4 | 65 | 1.34 | 4/4 | 182 | 1.28 | 5.85 | 2 | 246 | 3.30E−03 |
| RS718993 | 1.49 | 1/3 | 72 | 1.21 | 3/3 | 163 | 1.32 | 5.85 | 2 | 243 | 3.30E−03 |
| RS1607664 | 1.46 | 2/4 | 72 | 1.38 | 4/4 | 162 | 1.25 | 5.85 | 2 | 240 | 3.31E−03 |
| RS1073913 | 1.33 | 3/4 | 116 | 1.34 | 4/4 | 57 | 1.18 | 5.85 | 2 | 236 | 3.31E−03 |
| RS10510104 | 1.72 | 2/4 | 81 | 1.32 | 4/4 | 164 | 1.27 | 5.84 | 2 | 247 | 3.33E−03 |
| RS1927404 | 1.23 | 1/3 | 88 | 1.38 | 3/3 | 43 | 1.28 | 5.85 | 2 | 223 | 3.34E−03 |
| RS196584 | 1.28 | 2/4 | 26 | 1.4 | 4/4 | 1 | 2.15 | 5.84 | 2 | 232 | 3.35E−03 |
| RS9298895 | 1.44 | 3/3 | 215 | 1.27 | 0/0 | 0 | 0 | 8.77 | 1 | 247 | 3.36E−03 |
| RS2991463 | 1.33 | 2/4 | 98 | 1.36 | 4/4 | 132 | 1.24 | 5.83 | 2 | 245 | 3.36E−03 |
| RS10501159 | 2.26 | 1/4 | 21 | 1.36 | 4/4 | 233 | 1.29 | 5.83 | 2 | 242 | 3.37E−03 |
| RS1541503 | 1.27 | 2/4 | 38 | 1.41 | 4/4 | 2 | 1.73 | 5.83 | 2 | 238 | 3.37E−03 |
| RS4757441 | 1.33 | 2/4 | 54 | 1.18 | 4/4 | 3 | 1.25 | 5.82 | 2 | 247 | 3.39E−03 |
| RS9302933 | 1.26 | 2/4 | 69 | 1.4 | 4/4 | 3 | 1.18 | 5.82 | 2 | 246 | 3.39E−03 |
| RS309275 | 1.26 | 3/4 | 109 | 1.37 | 4/4 | 106 | 1.23 | 5.82 | 2 | 244 | 3.40E−03 |
| RS10491279 | 1.78 | 1/3 | 70 | 1.23 | 3/3 | 174 | 1.31 | 5.82 | 2 | 244 | 3.40E−03 |
| RS10509153 | 1.75 | 2/4 | 37 | 1.4 | 4/4 | 189 | 1.27 | 5.83 | 2 | 225 | 3.40E−03 |
| RS10485864 | 1.27 | 2/3 | 52 | 1.34 | 3/3 | 4 | 1.75 | 5.82 | 2 | 240 | 3.40E−03 |
| RS10510156 | 1.26 | 1/4 | 50 | 1.42 | 4/4 | 180 | 1.26 | 5.82 | 2 | 232 | 3.42E−03 |
| RS10520762 | 1.3 | 2/4 | 62 | 1.35 | 4/4 | 13 | 1.04 | 5.81 | 2 | 241 | 3.43E−03 |
| RS7530592 | 1.24 | 3/4 | 120 | 1.36 | 4/4 | 27 | 1.21 | 5.81 | 2 | 240 | 3.44E−03 |
| RS1867584 | 1.21 | 2/4 | 104 | 1.33 | 4/4 | 46 | 1.38 | 5.82 | 2 | 222 | 3.44E−03 |
| RS10509772 | 1.2 | 2/4 | 112 | 1.36 | 4/4 | 68 | 1.28 | 5.81 | 2 | 235 | 3.44E−03 |
| RS6911443 | 1.28 | 1/3 | 107 | 1.27 | 3/3 | 29 | 1.48 | 5.81 | 2 | 231 | 3.45E−03 |
| RS9297984 | 1.75 | 3/4 | 90 | 1.29 | 4/4 | 139 | 1.29 | 5.81 | 2 | 231 | 3.45E−03 |
| RS10493822 | 1.72 | 2/4 | 57 | 1.25 | 4/4 | 188 | 1.3 | 5.79 | 2 | 247 | 3.49E−03 |
| RS165044 | 1.26 | 1/3 | 121 | 1.29 | 3/3 | 25 | 1.49 | 5.79 | 2 | 244 | 3.49E−03 |
| RS1928274 | 1.6 | 1/3 | 66 | 1.27 | 3/3 | 169 | 1.29 | 5.79 | 2 | 243 | 3.50E−03 |
| RS10508554 | 1.27 | 2/4 | 117 | 1.24 | 4/4 | 101 | 1.37 | 5.79 | 2 | 242 | 3.50E−03 |
| RS958938 | 1.96 | 1/3 | 32 | 1.35 | 3/3 | 215 | 1.28 | 5.78 | 2 | 246 | 3.52E−03 |
| RS2964299 | 1.31 | 2/4 | 20 | 1.11 | 0/0 | 0 | 0 | 8.68 | 1 | 247 | 3.52E−03 |
| RS1861373 | 1.23 | 1/2 | 120 | 1.26 | 2/2 | 81 | 1.39 | 5.78 | 2 | 244 | 3.53E−03 |
| RS1395817 | 1.47 | 3/3 | 170 | 1.28 | 0/0 | 0 | 0 | 8.71 | 1 | 195 | 3.55E−03 |
| RS497197 | 2.02 | 2/4 | 29 | 1.32 | 4/4 | 203 | 1.29 | 5.78 | 2 | 231 | 3.55E−03 |
| RS10498212 | 1.31 | 1/4 | 18 | 1.1 | 0/0 | 0 | 0 | 8.66 | 1 | 248 | 3.56E−03 |
| RS823986 | 1.32 | 1/3 | 133 | 1.24 | 3/3 | 81 | 1.38 | 5.77 | 2 | 233 | 3.58E−03 |
| RS1544589 | 1.37 | 1/3 | 116 | 1.32 | 3/3 | 79 | 1.21 | 5.76 | 2 | 247 | 3.59E−03 |
| RS7004663 | 1.32 | 1/4 | 47 | 1.21 | 4/4 | 3 | 1.72 | 5.78 | 2 | 213 | 3.59E−03 |
| RS4774367 | 1.28 | 2/3 | 103 | 1.35 | 3/3 | 16 | 1.08 | 5.75 | 2 | 247 | 3.62E−03 |
| RS6863889 | 1.31 | 1/3 | 25 | 1.13 | 0/0 | 0 | 0 | 8.62 | 1 | 248 | 3.64E−03 |
| RS868271 | 1.31 | 2/4 | 93 | 1.36 | 4/4 | 80 | 1.21 | 5.76 | 2 | 213 | 3.66E−03 |
| RS1525489 | 2.05 | 2/4 | 29 | 1.18 | 4/4 | 218 | 1.31 | 5.74 | 2 | 245 | 3.66E−03 |
| RS548987 | 1.28 | 2/3 | 34 | 1.36 | 3/3 | 1 | 2.2 | 5.74 | 2 | 244 | 3.66E−03 |
| RS10501933 | 1.58 | 2/4 | 73 | 1.34 | 4/4 | 165 | 1.26 | 5.74 | 2 | 244 | 3.66E−03 |
| RS1477523 | 1.32 | 2/4 | 28 | 1.14 | 0/0 | 0 | 0 | 8.6 | 1 | 248 | 3.68E−03 |
| RS2163120 | 1.54 | 4/4 | 238 | 1.28 | 0/0 | 0 | 0 | 8.6 | 1 | 248 | 3.68E−03 |
| RS10506524 | 1.5 | 3/3 | 231 | 1.28 | 0/0 | 0 | 0 | 8.6 | 1 | 247 | 3.68E−03 |
| RS4131874 | 1.35 | 2/3 | 92 | 1.22 | 3/3 | 16 | 1.23 | 5.73 | 2 | 241 | 3.71E−03 |
| RS2981069 | 1.26 | 2/4 | 57 | 1.41 | 4/4 | 3 | 1.26 | 5.73 | 2 | 241 | 3.71E−03 |
| RS1460597 | 1.52 | 2/3 | 109 | 1.25 | 3/3 | 119 | 1.32 | 5.73 | 2 | 239 | 3.71E−03 |
| RS10511729 | 1.28 | 1/2 | 121 | 1.26 | 2/2 | 38 | 1.45 | 5.72 | 2 | 246 | 3.73E−03 |
| RS1983726 | 1.56 | 2/4 | 101 | 1.26 | 4/4 | 135 | 1.29 | 5.72 | 2 | 245 | 3.73E−03 |
| RS10511482 | 2.26 | 2/4 | 59 | 1.33 | 4/4 | 186 | 1.28 | 5.72 | 2 | 243 | 3.74E−03 |
| RS2711452 | 1.26 | 1/3 | 83 | 1.38 | 3/3 | 25 | 1.21 | 5.73 | 2 | 225 | 3.74E−03 |
| RS10497723 | 1.32 | 1/3 | 32 | 1.15 | 0/0 | 0 | 0 | 8.56 | 1 | 246 | 3.76E−03 |
| RS10500130 | 1.27 | 3/4 | 41 | 1.3 | 4/4 | 4 | 1.75 | 5.72 | 2 | 231 | 3.76E−03 |
| RS1841914 | 1.27 | 1/3 | 131 | 1.25 | 3/3 | 72 | 1.4 | 5.71 | 2 | 247 | 3.77E−03 |
| RS1938105 | 1.26 | 1/3 | 57 | 1.18 | 3/3 | 185 | 1.34 | 5.71 | 2 | 245 | 3.77E−03 |
| RS1341001 | 1.5 | 1/3 | 123 | 1.25 | 3/3 | 106 | 1.31 | 5.71 | 2 | 244 | 3.77E−03 |
| RS10488204 | 1.28 | 2/3 | 18 | 1.5 | 0/0 | 0 | 0 | 8.55 | 1 | 246 | 3.78E−03 |
| RS7670292 | 1.33 | 2/4 | 75 | 1.21 | 4/4 | 6 | 1.53 | 5.71 | 2 | 237 | 3.79E−03 |
| RS7844425 | 1.24 | 1/2 | 119 | 1.32 | 2/2 | 18 | 1.48 | 5.71 | 2 | 235 | 3.79E−03 |
| RS10510344 | 1.16 | 3/3 | 208 | 1.32 | 0/0 | 0 | 0 | 8.54 | 1 | 240 | 3.81E−03 |
| RS10516032 | 1.87 | 2/4 | 42 | 1.27 | 4/4 | 201 | 1.29 | 5.7 | 2 | 243 | 3.81E−03 |
| RS6852312 | 1.28 | 3/4 | 25 | 1.46 | 0/0 | 0 | 0 | 8.53 | 1 | 248 | 3.82E−03 |
| RS9317744 | 1.56 | 4/4 | 220 | 1.29 | 0/0 | 0 | 0 | 8.54 | 1 | 229 | 3.82E−03 |
| RS4368350 | 1.75 | 2/4 | 36 | 1.21 | 4/4 | 180 | 1.31 | 5.71 | 2 | 217 | 3.83E−03 |
| RS7577293 | 1.19 | 1/3 | 101 | 1.37 | 3/3 | 135 | 1.25 | 5.69 | 2 | 246 | 3.84E−03 |
| RS10514263 | 1.28 | 1/2 | 16 | 1.54 | 2/2 | 1 | 1.16 | 5.69 | 2 | 243 | 3.85E−03 |
| RS10514632 | 1.03 | 1/3 | 83 | 1.23 | 3/3 | 154 | 1.34 | 5.69 | 2 | 240 | 3.85E−03 |
| RS1376499 | 1.27 | 3/4 | 112 | 1.25 | 4/4 | 43 | 1.42 | 5.7 | 2 | 222 | 3.85E−03 |
| RS7148756 | 1.48 | 1/4 | 55 | 1.19 | 4/4 | 150 | 1.34 | 5.71 | 2 | 206 | 3.86E−03 |
| RS2165347 | 1.26 | 2/4 | 111 | 1.35 | 4/4 | 85 | 1.22 | 5.7 | 2 | 217 | 3.87E−03 |
| RS1199292 | 1.46 | 2/4 | 111 | 1.27 | 4/4 | 89 | 1.28 | 5.69 | 2 | 230 | 3.87E−03 |
| RS1441541 | 1.51 | 1/3 | 43 | 1.17 | 3/3 | 201 | 1.32 | 5.68 | 2 | 246 | 3.88E−03 |
| RS171785 | 1.4 | 1/3 | 114 | 1.33 | 3/3 | 104 | 1.22 | 5.68 | 2 | 245 | 3.88E−03 |
| RS10484331 | 1.38 | 2/4 | 122 | 1.24 | 4/4 | 39 | 1.31 | 5.68 | 2 | 245 | 3.88E−03 |
| RS2572276 | 1.48 | 1/3 | 96 | 1.25 | 3/3 | 129 | 1.3 | 5.68 | 2 | 245 | 3.88E−03 |
| RS10483323 | 1.15 | 1/3 | 74 | 1.39 | 3/3 | 162 | 1.27 | 5.68 | 2 | 244 | 3.88E−03 |
| RS3859362 | 1.52 | 2/4 | 95 | 1.24 | 4/4 | 119 | 1.32 | 5.69 | 2 | 225 | 3.88E−03 |
| RS10513765 | 1.27 | 2/4 | 115 | 1.36 | 4/4 | 95 | 1.23 | 5.68 | 2 | 239 | 3.89E−03 |
| RS964059 | 1.25 | 3/4 | 74 | 1.39 | 4/4 | 9 | 1.24 | 5.68 | 2 | 239 | 3.89E−03 |
| RS2825583 | 1.22 | 2/4 | 107 | 1.37 | 4/4 | 53 | 1.28 | 5.67 | 2 | 242 | 3.92E−03 |
| RS8137239 | 1.19 | 1/3 | 19 | 1.52 | 3/3 | 221 | 1.28 | 5.67 | 2 | 238 | 3.93E−03 |
| RS6699989 | 1.55 | 1/3 | 62 | 1.36 | 3/3 | 175 | 1.26 | 5.66 | 2 | 243 | 3.96E−03 |
| RS2396821 | 1.25 | 1/3 | 69 | 1.4 | 3/3 | 10 | 1.33 | 5.66 | 2 | 242 | 3.96E−03 |
| RS10507930 | 1.65 | 3/3 | 244 | 1.29 | 0/0 | 0 | 0 | 8.46 | 1 | 248 | 3.96E−03 |
| RS7324065 | 1.53 | 3/3 | 234 | 1.28 | 0/0 | 0 | 0 | 8.45 | 1 | 245 | 3.98E−03 |
| RS1329983 | 0.93 | 1/3 | 52 | 1.39 | 3/3 | 193 | 1.27 | 5.65 | 2 | 245 | 3.99E−03 |
| RS7874576 | 1.68 | 3/3 | 241 | 1.29 | 0/0 | 0 | 0 | 8.44 | 1 | 244 | 4.01E−03 |
| RS10513275 | 1.29 | 2/4 | 50 | 1.28 | 4/4 | 7 | 1.67 | 5.64 | 2 | 246 | 4.03E−03 |
| RS1324088 | 1.28 | 2/4 | 36 | 1.35 | 4/4 | 1 | 2.2 | 5.64 | 2 | 246 | 4.03E−03 |
| RS10495466 | 1.28 | 2/4 | 20 | 1.51 | 4/4 | 1 | 1.39 | 5.64 | 2 | 245 | 4.03E−03 |
| RS718911 | 1.3 | 1/3 | 51 | 1.27 | 3/3 | 2 | 2 | 5.64 | 2 | 244 | 4.03E−03 |
| RS4952676 | 1.22 | 1/3 | 107 | 1.35 | 3/3 | 39 | 1.3 | 5.64 | 2 | 243 | 4.03E−03 |
| RS10518774 | 1.41 | 1/3 | 127 | 1.27 | 3/3 | 55 | 1.25 | 5.64 | 2 | 240 | 4.04E−03 |
| RS488673 | 1.31 | 2/4 | 87 | 1.22 | 4/4 | 32 | 1.43 | 5.66 | 2 | 204 | 4.05E−03 |
| RS3815035 | 1.39 | 2/4 | 48 | 1.42 | 4/4 | 195 | 1.26 | 5.63 | 2 | 245 | 4.07E−03 |
| RS3890717 | 1.4 | 3/4 | 124 | 1.32 | 4/4 | 76 | 1.21 | 5.63 | 2 | 242 | 4.07E−03 |
| RS188014 | 1.51 | 3/3 | 229 | 1.28 | 0/0 | 0 | 0 | 8.41 | 1 | 242 | 4.07E−03 |
| RS10508318 | 1.38 | 1/2 | 81 | 1.2 | 2/2 | 146 | 1.34 | 5.63 | 2 | 232 | 4.10E−03 |
| RS472154 | 1.29 | 2/3 | 78 | 1.2 | 3/3 | 150 | 1.34 | 5.62 | 2 | 244 | 4.11E−03 |
| RS9300024 | 1.24 | 1/3 | 113 | 1.37 | 3/3 | 69 | 1.24 | 5.62 | 2 | 244 | 4.11E−03 |
| RS10512405 | 1.45 | 2/4 | 54 | 1.39 | 4/4 | 127 | 1.26 | 5.65 | 2 | 197 | 4.11E−03 |
| RS7009058 | 1.31 | 3/4 | 56 | 1.2 | 4/4 | 9 | 1.5 | 5.62 | 2 | 243 | 4.11E−03 |
| RS341148 | 1.35 | 2/4 | 49 | 1.24 | 4/4 | 28 | 1.16 | 5.67 | 2 | 175 | 4.11E−03 |
| RS9302288 | 1.34 | 1/3 | 83 | 1.21 | 3/3 | 7 | 1.43 | 5.62 | 2 | 242 | 4.11E−03 |
| Rs10498604 | 1.27 | 2/4 | 80 | 1.21 | 4/4 | 153 | 1.35 | 5.61 | 2 | 247 | 4.14E−03 |
| RS6812018 | 1.28 | 3/4 | 44 | 1.34 | 4/4 | 5 | 1.72 | 5.61 | 2 | 243 | 4.15E−03 |
| RS10514569 | 1.07 | 4/4 | 180 | 1.31 | 0/0 | 0 | 0 | 8.41 | 1 | 191 | 4.17E−03 |
| RS3751462 | 1.34 | 1/4 | 46 | 1.18 | 4/4 | 10 | 1.23 | 5.63 | 2 | 203 | 4.17E−03 |
| RS987479 | 1.28 | 1/3 | 78 | 1.29 | 3/3 | 14 | 1.54 | 5.6 | 2 | 238 | 4.20E−03 |
| RS1233853 | 2 | 1/3 | 26 | 1.31 | 3/3 | 207 | 1.29 | 5.6 | 2 | 232 | 4.22E−03 |
| RS979323 | 1.43 | 1/2 | 104 | 1.31 | 2/2 | 85 | 1.23 | 5.6 | 2 | 226 | 4.23E−03 |
| RS962592 | 1.29 | 2/4 | 6 | 1.65 | 0/0 | 0 | 0 | 8.33 | 1 | 247 | 4.24E−03 |
| RS5962018 | 1.28 | 4/4 | 35 | 1.44 | 0/0 | 0 | 0 | 8.33 | 1 | 243 | 4.25E−03 |
| RS362988 | 1.36 | 1/3 | 87 | 1.3 | 3/3 | 54 | 1.19 | 5.6 | 2 | 215 | 4.26E−03 |
| RS945849 | 1.3 | 1/3 | 80 | 1.28 | 3/3 | 3 | 1.86 | 5.58 | 2 | 246 | 4.27E−03 |
| RS7078806 | 1.29 | 1/3 | 21 | 1.35 | 3/3 | 1 | 2.26 | 5.58 | 2 | 243 | 4.27E−03 |
| RS8089476 | 1.29 | 2/4 | 115 | 1.24 | 4/4 | 95 | 1.37 | 5.58 | 2 | 242 | 4.27E−03 |
| RS9293488 | 1.28 | 2/4 | 13 | 1.51 | 0/0 | 0 | 0 | 8.32 | 1 | 229 | 4.30E−03 |
| RS1340169 | 1.58 | 4/4 | 239 | 1.29 | 0/0 | 0 | 0 | 8.3 | 1 | 246 | 4.31E−03 |
| RS6109935 | 1.52 | 2/4 | 84 | 1.29 | 4/4 | 123 | 1.27 | 5.58 | 2 | 223 | 4.32E−03 |
| RS10502776 | 1.31 | 1/3 | 13 | 1.06 | 0/0 | 0 | 0 | 8.29 | 1 | 248 | 4.33E−03 |
| RS1403635 | 1.64 | 2/4 | 74 | 1.27 | 4/4 | 168 | 1.29 | 5.56 | 2 | 247 | 4.35E−03 |
| RS3800274 | 1.56 | 3/4 | 85 | 1.3 | 4/4 | 133 | 1.27 | 5.57 | 2 | 228 | 4.35E−03 |
| RS1459914 | 1.3 | 3/4 | 44 | 1.24 | 4/4 | 1 | 2.2 | 5.56 | 2 | 245 | 4.35E−03 |
| RS1233775 | 1.34 | 2/4 | 105 | 1.24 | 4/4 | 6 | 1.05 | 5.56 | 2 | 245 | 4.35E−03 |
| RS9294240 | 1.2 | 1/3 | 81 | 1.38 | 3/3 | 143 | 1.26 | 5.56 | 2 | 240 | 4.36E−03 |
| RS1407390 | 1.29 | 2/4 | 76 | 1.28 | 4/4 | 3 | 1.86 | 5.55 | 2 | 246 | 4.39E−03 |
| RS10495804 | 1.43 | 2/4 | 105 | 1.24 | 4/4 | 117 | 1.33 | 5.55 | 2 | 245 | 4.39E−03 |
| RS6477845 | 1.86 | 1/3 | 75 | 1.28 | 3/3 | 170 | 1.29 | 5.55 | 2 | 245 | 4.39E−03 |
| RS10501158 | 1.28 | 1/3 | 30 | 1.32 | 3/3 | 1 | 2.26 | 5.55 | 2 | 245 | 4.39E−03 |
| RS1945654 | 1.3 | 2/3 | 58 | 1.27 | 3/3 | 3 | 1.86 | 5.55 | 2 | 244 | 4.39E−03 |
| RS3814860 | 1.26 | 3/4 | 75 | 1.4 | 4/4 | 11 | 1.22 | 5.55 | 2 | 243 | 4.40E−03 |
| RS2881836 | 1.22 | 3/4 | 106 | 1.35 | 4/4 | 21 | 1.38 | 5.55 | 2 | 234 | 4.42E−03 |
| RS1805781 | 1.6 | 3/3 | 238 | 1.29 | 0/0 | 0 | 0 | 8.25 | 1 | 244 | 4.43E−03 |
| RS294665 | 1.26 | 2/4 | 78 | 1.38 | 4/4 | 8 | 1.17 | 5.54 | 2 | 242 | 4.44E−03 |
| RS3897748 | 1.33 | 2/3 | 67 | 1.2 | 3/3 | 6 | 1.19 | 5.54 | 2 | 240 | 4.45E−03 |
| RS10517222 | 1.25 | 1/3 | 81 | 1.32 | 3/3 | 15 | 1.5 | 5.53 | 2 | 244 | 4.48E−03 |
| RS10507083 | 1.08 | 1/3 | 82 | 1.27 | 3/3 | 145 | 1.33 | 5.53 | 2 | 240 | 4.49E−03 |
| RS796892 | 1.29 | 2/4 | 108 | 1.37 | 4/4 | 122 | 1.24 | 5.52 | 2 | 246 | 4.52E−03 |
| RS7002137 | 1.26 | 2/3 | 65 | 1.38 | 3/3 | 4 | 1.59 | 5.52 | 2 | 246 | 4.52E−03 |
| RS7719212 | 1.29 | 2/3 | 16 | 1.4 | 3/3 | 1 | 2.2 | 5.52 | 2 | 245 | 4.52E−03 |
| RS713265 | 2.26 | 2/4 | 19 | 1.34 | 4/4 | 224 | 1.29 | 5.52 | 2 | 241 | 4.53E−03 |
| RS6848015 | 1.3 | 2/3 | 64 | 1.24 | 3/3 | 4 | 1.74 | 5.52 | 2 | 241 | 4.53E−03 |
| RS10495202 | 1.25 | 1/3 | 90 | 1.36 | 3/3 | 14 | 1.44 | 5.51 | 2 | 247 | 4.56E−03 |
| RS4129693 | 1.29 | 2/4 | 90 | 1.27 | 4/4 | 11 | 1.59 | 5.51 | 2 | 246 | 4.56E−03 |
| RS4073474 | 1.34 | 1/3 | 109 | 1.23 | 3/3 | 37 | 1.37 | 5.51 | 2 | 246 | 4.56E−03 |
| RS150344 | 1.27 | 3/4 | 62 | 1.34 | 4/4 | 7 | 1.61 | 5.51 | 2 | 242 | 4.57E−03 |
| RS1915552 | 1.33 | 1/4 | 133 | 1.34 | 4/4 | 75 | 1.2 | 5.51 | 2 | 239 | 4.58E−03 |
| RS1440063 | 1.37 | 1/3 | 98 | 1.23 | 3/3 | 39 | 1.25 | 5.52 | 2 | 219 | 4.58E−03 |
| RS494739 | 1.27 | 2/4 | 36 | 1.45 | 4/4 | 3 | 1.19 | 5.5 | 2 | 245 | 4.61E−03 |
| RS2391476 | 1.18 | 3/4 | 43 | 1.43 | 4/4 | 198 | 1.27 | 5.5 | 2 | 242 | 4.61E−03 |
| RS2054915 | 1.26 | 1/2 | 128 | 1.35 | 2/2 | 46 | 1.19 | 5.5 | 2 | 237 | 4.63E−03 |
| RS989766 | 1.27 | 2/3 | 62 | 1.28 | 3/3 | 27 | 1.48 | 5.51 | 2 | 215 | 4.64E−03 |
| RS7675645 | 1.26 | 1/3 | 76 | 1.35 | 3/3 | 5 | 1.63 | 5.49 | 2 | 245 | 4.65E−03 |
| RS1571502 | 1.03 | 2/4 | 46 | 1.43 | 4/4 | 199 | 1.27 | 5.49 | 2 | 243 | 4.66E−03 |
| RS1661405 | 1.56 | 3/3 | 240 | 1.29 | 0/0 | 0 | 0 | 8.15 | 1 | 248 | 4.67E−03 |
| RS2675200 | 1.16 | 4/4 | 218 | 1.32 | 0/0 | 0 | 0 | 8.14 | 1 | 248 | 4.70E−03 |
| RS3905336 | 1.61 | 4/4 | 242 | 1.28 | 0/0 | 0 | 0 | 8.14 | 1 | 247 | 4.70E−03 |
| RS652238 | 1.33 | 2/4 | 55 | 1.19 | 4/4 | 11 | 1.22 | 5.48 | 2 | 244 | 4.70E−03 |
| RS3852745 | 1.02 | 1/2 | 106 | 1.3 | 2/2 | 127 | 1.32 | 5.48 | 2 | 242 | 4.70E−03 |
| RS10491948 | 1.28 | 2/3 | 11 | 1.55 | 0/0 | 0 | 0 | 8.14 | 1 | 237 | 4.71E−03 |
| RS10490414 | 1.59 | 4/4 | 239 | 1.29 | 0/0 | 0 | 0 | 8.13 | 1 | 245 | 4.73E−03 |
| RS3751337 | 1.11 | 1/3 | 107 | 1.34 | 3/3 | 121 | 1.29 | 5.47 | 2 | 246 | 4.74E−03 |
| RS1041802 | 1.26 | 3/4 | 102 | 1.37 | 4/4 | 21 | 1.19 | 5.47 | 2 | 244 | 4.74E−03 |
| RS10505725 | 1.32 | 2/4 | 90 | 1.23 | 4/4 | 20 | 1.46 | 5.47 | 2 | 238 | 4.76E−03 |
| RS1207613 | 1.27 | 3/3 | 44 | 1.41 | 0/0 | 0 | 0 | 8.12 | 1 | 236 | 4.76E−03 |
| RS4981115 | 1.77 | 1/3 | 31 | 1.33 | 3/3 | 199 | 1.28 | 5.47 | 2 | 231 | 4.77E−03 |
| RS1150030 | 1.19 | 1/3 | 29 | 1.47 | 3/3 | 219 | 1.27 | 5.46 | 2 | 246 | 4.78E−03 |
| RS2217610 | 1.25 | 1/3 | 77 | 1.39 | 3/3 | 11 | 1.27 | 5.47 | 2 | 227 | 4.78E−03 |
| RS596255 | 1.44 | 2/3 | 97 | 1.34 | 3/3 | 129 | 1.24 | 5.46 | 2 | 245 | 4.79E−03 |
| RS7906587 | 1.29 | 2/4 | 20 | 1.27 | 4/4 | 1 | 2.26 | 5.46 | 2 | 245 | 4.79E−03 |
| RS10489251 | 1.71 | 3/4 | 58 | 1.25 | 4/4 | 183 | 1.3 | 5.46 | 2 | 243 | 4.79E−03 |
| RS10509766 | 1.25 | 2/4 | 93 | 1.24 | 4/4 | 123 | 1.36 | 5.46 | 2 | 242 | 4.79E−03 |
| RS4890429 | 1.25 | 1/3 | 94 | 1.33 | 3/3 | 22 | 1.46 | 5.46 | 2 | 242 | 4.79E−03 |
| RS7694034 | 1.23 | 1/3 | 68 | 1.2 | 3/3 | 167 | 1.34 | 5.46 | 2 | 240 | 4.80E−03 |
| RS7963889 | 1.36 | 1/3 | 94 | 1.34 | 3/3 | 95 | 1.21 | 5.47 | 2 | 217 | 4.81E−03 |
| RS286300 | 1.27 | 2/4 | 30 | 1.46 | 4/4 | 1 | 1.09 | 5.45 | 2 | 246 | 4.83E−03 |
| RS10493985 | 1.61 | 3/3 | 243 | 1.29 | 0/0 | 0 | 0 | 8.08 | 1 | 248 | 4.85E−03 |
| RS6823379 | 1.28 | 1/3 | 13 | 1.53 | 0/0 | 0 | 0 | 8.08 | 1 | 248 | 4.85E−03 |
| RS499891 | 1.29 | 1/3 | 9 | 1.58 | 0/0 | 0 | 0 | 8.07 | 1 | 248 | 4.87E−03 |
| RS539085 | 1.29 | 2/3 | 9 | 1.58 | 0/0 | 0 | 0 | 8.07 | 1 | 248 | 4.87E−03 |
| RS2156348 | 1.29 | 2/4 | 9 | 1.58 | 0/0 | 0 | 0 | 8.07 | 1 | 248 | 4.87E−03 |
| RS477892 | 1.29 | 1/3 | 9 | 1.58 | 0/0 | 0 | 0 | 8.07 | 1 | 248 | 4.87E−03 |
| RS480413 | 1.58 | 3/3 | 241 | 1.29 | 0/0 | 0 | 0 | 8.07 | 1 | 248 | 4.87E−03 |
| RS2856259 | 1.58 | 3/3 | 241 | 1.29 | 0/0 | 0 | 0 | 8.07 | 1 | 248 | 4.87E−03 |
| RS4939237 | 1.58 | 3/3 | 241 | 1.29 | 0/0 | 0 | 0 | 8.07 | 1 | 248 | 4.87E−03 |
| RS10502807 | 1.28 | 2/4 | 40 | 1.32 | 4/4 | 5 | 1.7 | 5.45 | 2 | 227 | 4.88E−03 |
| RS10512282 | 1.32 | 1/2 | 38 | 1.16 | 2/2 | 3 | 1.53 | 5.44 | 2 | 243 | 4.88E−03 |
| RS10500585 | 1.36 | 2/4 | 111 | 1.24 | 4/4 | 14 | 1.21 | 5.44 | 2 | 240 | 4.89E−03 |
| RS823638 | 1.86 | 1/3 | 59 | 1.28 | 3/3 | 160 | 1.29 | 5.45 | 2 | 219 | 4.90E−03 |
| RS697902 | 1.26 | 2/4 | 82 | 1.24 | 4/4 | 32 | 1.43 | 5.46 | 2 | 202 | 4.90E−03 |
| RS10493987 | 1.67 | 3/3 | 242 | 1.29 | 0/0 | 0 | 0 | 8.06 | 1 | 245 | 4.91E−03 |
| RS4733224 | 1.22 | 1/3 | 100 | 1.3 | 3/3 | 41 | 1.4 | 5.44 | 2 | 228 | 4.92E−03 |
| RS2731239 | 1.29 | 2/4 | 116 | 1.23 | 4/4 | 112 | 1.36 | 5.43 | 2 | 247 | 4.92E−03 |
| RS7459039 | 1.29 | 1/3 | 104 | 1.23 | 3/3 | 131 | 1.35 | 5.43 | 2 | 245 | 4.93E−03 |
| RS1156968 | 1.26 | 2/4 | 51 | 1.41 | 4/4 | 3 | 1.49 | 5.43 | 2 | 245 | 4.93E−03 |
| RS7470491 | 1.3 | 2/4 | 78 | 1.28 | 4/4 | 3 | 1.86 | 5.43 | 2 | 244 | 4.93E−03 |
| RS10485148 | 1.39 | 2/4 | 98 | 1.35 | 4/4 | 121 | 1.24 | 5.43 | 2 | 242 | 4.93E−03 |
| RS564896 | 1.24 | 2/4 | 106 | 1.36 | 4/4 | 113 | 1.24 | 5.43 | 2 | 239 | 4.94E−03 |
| RS666941 | 1.21 | 1/4 | 112 | 1.27 | 4/4 | 75 | 1.38 | 5.43 | 2 | 231 | 4.96E−03 |
| RS10506603 | 1.62 | 3/3 | 237 | 1.29 | 0/0 | 0 | 0 | 8.04 | 1 | 242 | 4.96E−03 |
| RS2669892 | 1.25 | 1/3 | 73 | 1.39 | 3/3 | 9 | 1.32 | 5.42 | 2 | 244 | 4.98E−03 |
| RS6477360 | 1.35 | 1/3 | 103 | 1.22 | 3/3 | 68 | 1.34 | 5.42 | 2 | 237 | 4.99E−03 |
| RS10506602 | 1.28 | 2/3 | 7 | 1.6 | 0/0 | 0 | 0 | 8.03 | 1 | 232 | 5.01E−03 |
| RS10518821 | 1.29 | 1/3 | 82 | 1.28 | 3/3 | 6 | 1.69 | 5.41 | 2 | 246 | 5.02E−03 |
| RS10508269 | 1.19 | 1/2 | 37 | 1.44 | 2/2 | 206 | 1.27 | 5.41 | 2 | 244 | 5.02E−03 |
| RS10512501 | 2.15 | 1/3 | 28 | 1.21 | 3/3 | 218 | 1.3 | 5.41 | 2 | 244 | 5.02E−03 |
| RS757668 | 1.45 | 1/3 | 120 | 1.29 | 3/3 | 90 | 1.26 | 5.41 | 2 | 242 | 5.03E−03 |
| RS10518404 | 1.3 | 2/4 | 21 | 1.24 | 4/4 | 2 | 1.97 | 5.41 | 2 | 236 | 5.04E−03 |
| RS1813404 | 1.23 | 2/4 | 101 | 1.33 | 4/4 | 23 | 1.43 | 5.41 | 2 | 235 | 5.05E−03 |
| RS10510020 | 1.29 | 1/3 | 22 | 1.33 | 3/3 | 1 | 2.26 | 5.4 | 2 | 247 | 5.07E−03 |
| RS10510022 | 1.29 | 1/3 | 22 | 1.33 | 3/3 | 1 | 2.26 | 5.4 | 2 | 247 | 5.07E−03 |
| RS10510023 | 2.26 | 1/3 | 22 | 1.33 | 3/3 | 227 | 1.29 | 5.4 | 2 | 247 | 5.07E−03 |
| RS723855 | 1.53 | 1/3 | 98 | 1.31 | 3/3 | 118 | 1.25 | 5.41 | 2 | 225 | 5.07E−03 |
| RS10491340 | 1.6 | 2/4 | 31 | 1.2 | 4/4 | 202 | 1.3 | 5.4 | 2 | 237 | 5.09E−03 |
| RS713266 | 1.29 | 2/4 | 31 | 1.32 | 4/4 | 1 | 2.26 | 5.39 | 2 | 246 | 5.12E−03 |
| RS2011688 | 1.29 | 2/4 | 37 | 1.28 | 4/4 | 1 | 2.26 | 5.39 | 2 | 246 | 5.12E−03 |
| RS2826847 | 1.29 | 2/4 | 50 | 1.32 | 4/4 | 1 | 2.26 | 5.39 | 2 | 243 | 5.12E−03 |
| RS869206 | 1.29 | 2/4 | 19 | 1.32 | 4/4 | 1 | 2.26 | 5.39 | 2 | 235 | 5.14E−03 |
| RS1355296 | 1.85 | 1/3 | 41 | 1.32 | 3/3 | 193 | 1.29 | 5.39 | 2 | 234 | 5.15E−03 |
| RS9298249 | 2.26 | 1/3 | 13 | 1.25 | 3/3 | 236 | 1.3 | 5.38 | 2 | 247 | 5.16E−03 |
| RS580758 | 1.75 | 1/2 | 42 | 1.34 | 2/2 | 203 | 1.28 | 5.38 | 2 | 246 | 5.17E−03 |
| RS10517618 | 1.24 | 3/4 | 83 | 1.31 | 4/4 | 24 | 1.45 | 5.39 | 2 | 226 | 5.17E−03 |
| RS979322 | 1.24 | 2/4 | 124 | 1.29 | 4/4 | 41 | 1.42 | 5.38 | 2 | 244 | 5.17E−03 |
| RS9298675 | 2.15 | 2/4 | 35 | 1.21 | 4/4 | 208 | 1.3 | 5.38 | 2 | 241 | 5.18E−03 |
| RS2863171 | 1.57 | 3/4 | 41 | 1.41 | 4/4 | 172 | 1.27 | 5.39 | 2 | 215 | 5.20E−03 |
| RS1459506 | 1.22 | 1/3 | 104 | 1.37 | 3/3 | 83 | 1.25 | 5.39 | 2 | 213 | 5.21E−03 |
| RS1822539 | 1.26 | 2/4 | 47 | 1.42 | 4/4 | 7 | 1.22 | 5.38 | 2 | 228 | 5.21E−03 |
| RS10488521 | 1.65 | 2/4 | 58 | 1.25 | 4/4 | 185 | 1.3 | 5.37 | 2 | 247 | 5.21E−03 |
| RS10504894 | 1.49 | 1/3 | 107 | 1.27 | 3/3 | 110 | 1.29 | 5.37 | 2 | 238 | 5.24E−03 |
| RS2255751 | 1.16 | 2/4 | 121 | 1.32 | 4/4 | 64 | 1.33 | 5.37 | 2 | 227 | 5.26E−03 |
| RS7827548 | 1.23 | 2/3 | 109 | 1.35 | 3/3 | 29 | 1.36 | 5.36 | 2 | 246 | 5.27E−03 |
| RS1451336 | 1.29 | 1/3 | 131 | 1.26 | 3/3 | 43 | 1.43 | 5.36 | 2 | 245 | 5.27E−03 |
| RS10493355 | 0.97 | 1/3 | 37 | 1.44 | 3/3 | 210 | 1.27 | 5.35 | 2 | 245 | 5.32E−03 |
| RS1926029 | 1.36 | 2/4 | 56 | 1.21 | 4/4 | 12 | 1.22 | 5.38 | 2 | 194 | 5.32E−03 |
| RS2572269 | 1.47 | 1/3 | 90 | 1.24 | 3/3 | 135 | 1.3 | 5.35 | 2 | 244 | 5.32E−03 |
| RS10507118 | 1.64 | 2/3 | 42 | 1.19 | 3/3 | 140 | 1.32 | 5.38 | 2 | 183 | 5.37E−03 |

TABLE 4
The most significant haplotype regions for HDL based
on HaploRec + HPM analysis.

| RS_id | SEQ_ID_no | Gene | Gene_id | Chr | Position | P-value |
|---|---|---|---|---|---|---|
| RS10518315 | 24 | | | 1 | 69591823 | 9.00E−05 |
| RS2181719 | 36 | | | 1 | 80970894 | 9.00E−05 |
| RS10516673 | 370 | HNRPD | 3184 | 4 | 83635972 | 9.00E−05 |
| RS2069080 | 371 | LOC391672 | 391672 | 4 | 83679770 | 9.00E−05 |
| RS2029259 | 484 | | | 4 | 188422292 | 9.00E−05 |
| RS2195989 | 872 | ANGPT1 | 284 | 8 | 108555088 | 9.00E−05 |
| RS10493677 | 33 | | | 1 | 80890427 | 1.00E−04 |
| RS1577879 | 34 | | | 1 | 80924966 | 1.00E−04 |
| RS10489696 | 35 | | | 1 | 80942453 | 1.00E−04 |
| RS1352276 | 483 | | | 4 | 188415169 | 1.00E−04 |
| RS963204 | 859 | CNGB3 | 54714 | 8 | 87658121 | 1.00E−04 |
| RS9297941 | 860 | CNGB3 | 54714 | 8 | 87727855 | 1.00E−04 |
| RS2147085 | 37 | | | 1 | 80971158 | 2.00E−04 |
| RS2288338 | 369 | HNRPD | 3184 | 4 | 83631813 | 2.00E−04 |
| RS9296540 | 638 | | | 6 | 47418964 | 2.00E−04 |
| RS9296545 | 639 | | | 6 | 47433570 | 2.00E−04 |
| RS10504709 | 848 | | | 8 | 80781919 | 2.00E−04 |
| RS6992703 | 850 | | | 8 | 80831834 | 2.00E−04 |
| RS10507326 | 1171 | | | 13 | 22616744 | 2.00E−04 |
| RS1998846 | 25 | | | 1 | 69613578 | 3.00E−04 |
| RS841665 | 32 | | | 1 | 80889752 | 3.00E−04 |
| RS4847303 | 56 | ABCD3 | 5825 | 1 | 94602933 | 3.00E−04 |
| RS3770874 | 165 | CRIM1 | 51232 | 2 | 36595648 | 3.00E−04 |
| RS7695741 | 372 | LOC391672 | 391672 | 4 | 83680241 | 3.00E−04 |
| RS10516562 | 400 | | | 4 | 111663867 | 3.00E−04 |
| RS2979710 | 849 | | | 8 | 80814997 | 3.00E−04 |
| RS10504826 | 861 | CNGB3 | 54714 | 8 | 87759135 | 3.00E−04 |
| RS1283713 | 870 | ANGPT1 | 284 | 8 | 108535493 | 3.00E−04 |
| RS1283726 | 871 | ANGPT1 | 284 | 8 | 108545032 | 3.00E−04 |
| RS1881740 | 979 | | | 10 | 49221566 | 3.00E−04 |
| RS2590386 | 980 | | | 10 | 49255436 | 3.00E−04 |
| RS10508901 | 981 | MAPK8 | 5599 | 10 | 49297404 | 3.00E−04 |
| RS10493456 | 23 | | | 1 | 69591130 | 4.00E−04 |
| RS1749541 | 57 | ABCD3 | 5825 | 1 | 94670399 | 4.00E−04 |
| RS756203 | 166 | CRIM1 | 51232 | 2 | 36598324 | 4.00E−04 |
| RS1433171 | 873 | ANGPT1 | 284 | 8 | 108561416 | 4.00E−04 |
| RS4770403 | 1173 | SGCG | 6445 | 13 | 22653127 | 4.00E−04 |
| RS1014938 | 718 | | | 7 | 37714392 | 5.00E−04 |
| RS10503573 | 780 | MSR1 | 4481 | 8 | 16047855 | 5.00E−04 |
| RS10503586 | 803 | | | 8 | 16792998 | 5.00E−04 |
| RS974279 | 858 | CNGB3 | 54714 | 8 | 87657635 | 5.00E−04 |
| RS7850478 | 946 | | | 9 | 117366392 | 5.00E−04 |
| RS10491855 | 947 | | | 9 | 117375274 | 5.00E−04 |
| RS727081 | 1172 | | | 13 | 22630199 | 5.00E−04 |
| RS787494 | 13 | | | 1 | 67797607 | 6.00E−04 |
| RS848504 | 164 | CRIM1 | 51232 | 2 | 36567045 | 6.00E−04 |
| RS9312601 | 467 | | | 4 | 177742191 | 6.00E−04 |
| RS1383162 | 795 | | | 8 | 16181469 | 6.00E−04 |
| RS1480145 | 796 | | | 8 | 16181494 | 6.00E−04 |
| RS970633 | 862 | CNGB3 | 54714 | 8 | 87759864 | 6.00E−04 |
| RS10505108 | 874 | ANGPT1 | 284 | 8 | 108564233 | 6.00E−04 |
| RS2435307 | 1371 | NPC1 | 4864 | 18 | 19381908 | 6.00E−04 |
| RS976000 | 485 | | | 4 | 188603100 | 7.00E−04 |
| RS10520904 | 503 | | | 5 | 24966531 | 7.00E−04 |
| RS1493479 | 504 | | | 5 | 25023525 | 7.00E−04 |
| RS10498717 | 624 | | | 6 | 23833783 | 7.00E−04 |
| RS1484751 | 781 | MSR1 | 4481 | 8 | 16064328 | 7.00E−04 |
| RS10503576 | 794 | | | 8 | 16179617 | 7.00E−04 |
| RS1112268 | 978 | | | 10 | 49217160 | 7.00E−04 |
| RS2896587 | 1044 | | | 11 | 11726440 | 7.00E−04 |
| RS10491185 | 1348 | | | 17 | 58614495 | 7.00E−04 |
| RS7523565 | 12 | | | 1 | 67783013 | 8.00E−04 |
| RS243965 | 399 | | | 4 | 111647482 | 8.00E−04 |
| RS2333467 | 465 | | | 4 | 177731837 | 8.00E−04 |
| RS921691 | 466 | | | 4 | 177735818 | 8.00E−04 |
| RS1158815 | 482 | | | 4 | 188232341 | 8.00E−04 |
| RS1547641 | 623 | | | 6 | 23833200 | 8.00E−04 |
| RS2182068 | 625 | | | 6 | 23845925 | 8.00E−04 |
| RS6938470 | 640 | | | 6 | 47446050 | 8.00E−04 |
| RS351572 | 782 | MSR1 | 4481 | 8 | 16065839 | 8.00E−04 |
| RS665889 | 915 | | | 9 | 85011765 | 8.00E−04 |
| RS10500747 | 1045 | | | 11 | 11728465 | 8.00E−04 |
| RS2447455 | 1349 | TANC2 | 26115 | 17 | 58623570 | 8.00E−04 |
| RS10502440 | 1372 | LAMA3 | 3909 | 18 | 19583575 | 8.00E−04 |
| RS696619 | 58 | F3 | 2152 | 1 | 94717241 | 9.00E−04 |
| RS3770899 | 163 | CRIM1 | 51232 | 2 | 36566423 | 9.00E−04 |
| RS3772781 | 300 | KALRN | 8997 | 3 | 125870129 | 9.00E−04 |
| RS10518393 | 407 | | | 4 | 122870458 | 9.00E−04 |
| RS1021019 | 408 | | | 4 | 122888607 | 9.00E−04 |
| RS1462069 | 486 | | | 4 | 188639576 | 9.00E−04 |
| RS2873911 | 505 | | | 5 | 25065918 | 9.00E−04 |
| RS1052981 | 719 | SFRP4 | 6424 | 7 | 37719741 | 9.00E−04 |
| RS1915546 | 841 | | | 8 | 80202606 | 9.00E−04 |
| RS10491186 | 1347 | | | 17 | 58558080 | 9.00E−04 |
| RS2460110 | 1350 | TANC2 | 26115 | 17 | 58672929 | 9.00E−04 |
| RS948435 | 1373 | LAMA3 | 3909 | 18 | 19637770 | 9.00E−04 |
| RS787493 | 14 | | | 1 | 67797740 | 1.00E−03 |
| RS1418005 | 26 | | | 1 | 69659645 | 1.00E−03 |
| RS891251 | 131 | | | 2 | 5958628 | 1.00E−03 |
| RS2291079 | 301 | ITGB5 | 3693 | 3 | 125965779 | 1.00E−03 |
| RS309753 | 468 | | | 4 | 177749792 | 1.00E−03 |
| RS2118763 | 513 | | | 5 | 44823303 | 1.00E−03 |
| RS35943 | 520 | ARL15 | 54622 | 5 | 53638314 | 1.00E−03 |
| RS7701690 | 521 | | | 5 | 53655784 | 1.00E−03 |
| RS224938 | 538 | | | 5 | 81752932 | 1.00E−03 |
| RS10491243 | 539 | | | 5 | 81770568 | 1.00E−03 |
| RS2461055 | 851 | | | 8 | 80832082 | 1.00E−03 |
| RS10513299 | 941 | ASTN2 | 23245 | 9 | 117184531 | 1.00E−03 |
| RS10508902 | 982 | MAPK8 | 5599 | 10 | 49311187 | 1.00E−03 |
| RS397618 | 1063 | TMEM16C | 63982 | 11 | 26546754 | 1.00E−03 |
| RS293970 | 1064 | TMEM16C | 63982 | 11 | 26549918 | 1.00E−03 |
| RS2278016 | 1308 | | | 16 | 50674510 | 1.00E−03 |
| RS1526211 | 219 | | | 2 | 184836469 | 1.10E−03 |
| RS9290552 | 311 | NAALADL2 | 254827 | 3 | 176588518 | 1.10E−03 |
| RS4611835 | 313 | NAALADL2 | 254827 | 3 | 176610777 | 1.10E−03 |
| RS1961547 | 368 | | | 4 | 83623512 | 1.10E−03 |
| RS10512865 | 514 | | | 5 | 44859124 | 1.10E−03 |
| RS2410410 | 797 | LOC646444 | 646444 | 8 | 16370342 | 1.10E−03 |
| RS10504694 | 842 | | | 8 | 80203155 | 1.10E−03 |
| RS10491854 | 948 | | | 9 | 117454034 | 1.10E−03 |
| RS1926256 | 22 | | | 1 | 69591095 | 1.20E−03 |
| RS1358091 | 220 | | | 2 | 184853176 | 1.20E−03 |
| RS10512629 | 299 | KALRN | 8997 | 3 | 125870100 | 1.20E−03 |
| RS725882 | 409 | | | 4 | 122925927 | 1.20E−03 |
| RS1351633 | 512 | | | 5 | 44579608 | 1.20E−03 |
| RS725819 | 626 | | | 6 | 23849815 | 1.20E−03 |
| RS534196 | 914 | | | 9 | 84907936 | 1.20E−03 |
| RS1621212 | 1033 | ADAM12 | 8038 | 10 | 127831272 | 1.20E−03 |
| RS2193849 | 312 | NAALADL2 | 254827 | 3 | 176601367 | 1.30E−03 |
| RS997720 | 373 | HNRPDL | 9987 | 4 | 83702325 | 1.30E−03 |
| RS250388 | 519 | ARL15 | 54622 | 5 | 53621019 | 1.30E−03 |
| RS997933 | 555 | | | 5 | 113212110 | 1.30E−03 |
| RS1480147 | 793 | | | 8 | 16170358 | 1.30E−03 |
| RS10510150 | 1031 | ADAM12 | 8038 | 10 | 127778924 | 1.30E−03 |
| RS293974 | 1062 | TMEM16C | 63982 | 11 | 26546702 | 1.30E−03 |
| RS7945560 | 1065 | TMEM16C | 63982 | 11 | 26565073 | 1.30E−03 |
| RS1526212 | 218 | | | 2 | 184836363 | 1.40E−03 |
| RS3772812 | 302 | ITGB5 | 3693 | 3 | 125966523 | 1.40E−03 |
| RS2348205 | 401 | | | 4 | 111671174 | 1.40E−03 |
| RS224939 | 537 | | | 5 | 81752858 | 1.40E−03 |
| RS10519364 | 553 | | | 5 | 113207846 | 1.40E−03 |
| RS1351608 | 554 | | | 5 | 113208536 | 1.40E−03 |
| RS4712495 | 612 | | | 6 | 10379610 | 1.40E−03 |
| RS1851006 | 1278 | UNC13C | 440279 | 15 | 52691684 | 1.40E−03 |
| RS10493872 | 55 | ABCD3 | 5825 | 1 | 94602439 | 1.50E−03 |
| RS257342 | 597 | | | 5 | 170997873 | 1.50E−03 |
| RS4510935 | 945 | | | 9 | 117364905 | 1.50E−03 |
| RS577379 | 1174 | SGCG | 6445 | 13 | 22653788 | 1.50E−03 |
| RS787534 | 11 | | | 1 | 67758546 | 1.60E−03 |
| RS6748937 | 221 | | | 2 | 184855451 | 1.60E−03 |
| RS243969 | 398 | | | 4 | 111646074 | 1.60E−03 |
| RS9307353 | 402 | | | 4 | 111671460 | 1.60E−03 |
| RS1027045 | 403 | | | 4 | 111677367 | 1.60E−03 |
| RS2037724 | 798 | LOC646444 | 646444 | 8 | 16370653 | 1.60E−03 |
| RS10504825 | 857 | CPNE3 | 8895 | 8 | 87636964 | 1.60E−03 |
| RS10513297 | 940 | ASTN2 | 23245 | 9 | 117130017 | 1.60E−03 |
| RS1278305 | 1032 | ADAM12 | 8038 | 10 | 127801415 | 1.60E−03 |
| RS959988 | 1164 | SLC15A4 | 121260 | 12 | 127817474 | 1.60E−03 |
| RS9304979 | 1408 | | | 19 | 20949974 | 1.60E−03 |
| RS10494858 | 101 | LOC388730 | 388730 | 1 | 201784380 | 1.70E−03 |
| RS3738154 | 102 | RBBP5 | 5929 | 1 | 201795606 | 1.70E−03 |
| RS726128 | 406 | | | 4 | 122870136 | 1.70E−03 |
| RS224940 | 536 | | | 5 | 81752818 | 1.70E−03 |
| RS7734201 | 552 | | | 5 | 113206989 | 1.70E−03 |
| RS899557 | 595 | | | 5 | 170994216 | 1.70E−03 |
| RS1324673 | 622 | | | 6 | 23820609 | 1.70E−03 |
| RS370067 | 783 | MSR1 | 4481 | 8 | 16088284 | 1.70E−03 |
| RS2220486 | 1162 | DKFZp761O2018 | 92293 | 12 | 127617021 | 1.70E−03 |
| RS1591678 | 31 | | | 1 | 80888871 | 1.80E−03 |
| RS10495528 | 132 | | | 2 | 6008445 | 1.80E−03 |
| RS657715 | 298 | KALRN | 8997 | 3 | 125845712 | 1.80E−03 |
| RS10520349 | 469 | | | 4 | 177765746 | 1.80E−03 |
| RS1905155 | 556 | | | 5 | 113301097 | 1.80E−03 |
| RS10488617 | 717 | TXNDC3 | 51314 | 7 | 37710273 | 1.80E−03 |
| RS2170116 | 791 | | | 8 | 16157007 | 1.80E−03 |
| RS2369693 | 839 | | | 8 | 80179994 | 1.80E−03 |
| RS10504693 | 840 | | | 8 | 80180116 | 1.80E−03 |
| RS6478298 | 939 | ASTN2 | 23245 | 9 | 117074245 | 1.80E−03 |
| RS10512498 | 1346 | | | 17 | 58507111 | 1.80E−03 |
| RS10493813 | 47 | | | 1 | 88583281 | 1.90E−03 |
| RS1026947 | 328 | | | 4 | 27588429 | 1.90E−03 |
| RS1462070 | 487 | | | 4 | 188639972 | 1.90E−03 |
| RS243768 | 650 | | | 6 | 54445089 | 1.90E−03 |
| RS10504710 | 847 | | | 8 | 80781801 | 1.90E−03 |
| RS10494860 | 103 | RIPK5 | 25778 | 1 | 201899456 | 2.00E−03 |
| RS10512920 | 494 | ADCY2 | 108 | 5 | 7455561 | 2.00E−03 |
| RS9325782 | 790 | | | 8 | 16134844 | 2.00E−03 |
| RS3909730 | 838 | | | 8 | 80175151 | 2.00E−03 |
| RS2398417 | 1163 | DKFZp761O2018 | 92293 | 12 | 127678424 | 2.00E−03 |
| RS10491088 | 1330 | GAS7 | 8522 | 17 | 9808904 | 2.00E−03 |
| RS10495529 | 133 | | | 2 | 6032408 | 2.10E−03 |
| RS10497645 | 222 | | | 2 | 184855798 | 2.10E−03 |
| RS4673011 | 243 | | | 2 | 222728693 | 2.10E−03 |
| RS1354083 | 297 | KALRN | 8997 | 3 | 125842553 | 2.10E−03 |
| RS1035093 | 327 | | | 4 | 27588012 | 2.10E−03 |
| RS10512855 | 511 | | | 5 | 44546969 | 2.10E−03 |
| RS1445829 | 522 | | | 5 | 53663936 | 2.10E−03 |
| RS1343496 | 665 | | | 6 | 78284325 | 2.10E−03 |
| RS1545442 | 720 | EPDR1 | 54749 | 7 | 37761531 | 2.10E−03 |
| RS2163120 | 792 | | | 8 | 16165538 | 2.10E−03 |
| RS7032150 | 928 | SVEP1 | 79987 | 9 | 110320280 | 2.10E−03 |
| RS10508903 | 983 | MAPK8 | 5599 | 10 | 49311338 | 2.10E−03 |
| RS1676741 | 1034 | ADAM12 | 8038 | 10 | 127841520 | 2.10E−03 |
| RS10500748 | 1046 | | | 11 | 11728555 | 2.10E−03 |
| RS10502470 | 1378 | KCTD1 | 284252 | 18 | 22452475 | 2.10E−03 |
| RS960202 | 48 | | | 1 | 88605059 | 2.20E−03 |
| RS10497651 | 217 | | | 2 | 184818615 | 2.20E−03 |
| RS10512921 | 495 | ADCY2 | 108 | 5 | 7455656 | 2.20E−03 |
| RS10520900 | 506 | | | 5 | 25102703 | 2.20E−03 |
| RS1317538 | 660 | KCNQ5 | 56479 | 6 | 73706132 | 2.20E−03 |
| RS420931 | 784 | MSR1 | 4481 | 8 | 16088316 | 2.20E−03 |
| RS1672168 | 875 | | | 8 | 108590185 | 2.20E−03 |
| RS1929496 | 921 | | | 9 | 101346508 | 2.20E−03 |
| RS33350 | 1307 | | | 16 | 50608803 | 2.20E−03 |
| RS1942578 | 1402 | | | 18 | 73322001 | 2.20E−03 |
| RS6692241 | 108 | USH2A | 7399 | 1 | 212598067 | 2.30E−03 |
| RS243972 | 397 | | | 4 | 111644685 | 2.30E−03 |
| RS631807 | 613 | LOC442161 | 442161 | 6 | 10475686 | 2.30E−03 |
| RS1586681 | 912 | | | 9 | 84876988 | 2.30E−03 |
| RS1169429 | 913 | | | 9 | 84893406 | 2.30E−03 |
| RS1013985 | 949 | | | 9 | 117454462 | 2.30E−03 |
| RS7983559 | 1170 | | | 13 | 22616580 | 2.30E−03 |
| RS1805082 | 1370 | NPC1 | 4864 | 18 | 19374442 | 2.30E−03 |
| RS632301 | 1380 | | | 18 | 22616756 | 2.30E−03 |
| RS4131672 | 1403 | | | 18 | 73380972 | 2.30E−03 |
| RS1926289 | 16 | | | 1 | 68177953 | 2.40E−03 |
| RS10489000 | 326 | | | 4 | 27586912 | 2.40E−03 |
| RS243976 | 396 | | | 4 | 111641384 | 2.40E−03 |
| RS1485870 | 453 | LOC442117 | 442117 | 4 | 173125585 | 2.40E−03 |
| RS10519689 | 569 | SRFBP1 | 153443 | 5 | 121347888 | 2.40E−03 |
| RS4397191 | 661 | KCNQ5 | 56479 | 6 | 73789192 | 2.40E−03 |
| RS10503574 | 779 | MSR1 | 4481 | 8 | 16028961 | 2.40E−03 |
| RS1565741 | 804 | | | 8 | 16799856 | 2.40E−03 |
| RS57740 | 1409 | | | 19 | 21223021 | 2.40E−03 |
| RS4650488 | 38 | | | 1 | 80995749 | 2.50E−03 |
| RS4704408 | 532 | PDE8B | 8622 | 5 | 76670405 | 2.50E−03 |
| RS10498887 | 662 | KCNQ5 | 56479 | 6 | 73797585 | 2.50E−03 |
| RS10512405 | 927 | SVEP1 | 79987 | 9 | 110316352 | 2.50E−03 |
| RS871120 | 938 | ASTN2 | 23245 | 9 | 117073775 | 2.50E−03 |
| RS1837993 | 1300 | | | 15 | 96618609 | 2.50E−03 |
| RS10491089 | 1331 | GAS7 | 8522 | 17 | 9828100 | 2.50E−03 |
| RS2077811 | 1379 | | | 18 | 22558197 | 2.50E−03 |
| RS1607664 | 1410 | ZNF429 | 353088 | 19 | 21497820 | 2.50E−03 |
| RS10495015 | 110 | USH2A | 7399 | 1 | 212647859 | 2.60E−03 |
| RS992454 | 244 | | | 2 | 222730366 | 2.60E−03 |
| RS1484258 | 325 | | | 4 | 27585846 | 2.60E−03 |
| RS4555709 | 347 | SCFD2 | 152579 | 4 | 53867889 | 2.60E−03 |
| RS171785 | 596 | | | 5 | 170994432 | 2.60E−03 |
| RS1721062 | 805 | | | 8 | 16857802 | 2.60E−03 |
| RS10489547 | 27 | | | 1 | 69681098 | 2.70E−03 |
| RS6540927 | 107 | USH2A | 7399 | 1 | 212581393 | 2.70E−03 |
| RS10512866 | 515 | | | 5 | 44859710 | 2.70E−03 |
| RS250387 | 518 | ARL15 | 54622 | 5 | 53620394 | 2.70E−03 |
| RS9327238 | 570 | SRFBP1 | 153443 | 5 | 121347997 | 2.70E−03 |
| RS1213382 | 666 | | | 6 | 78284528 | 2.70E−03 |
| RS1025926 | 829 | TRPA1 | 8989 | 8 | 73115712 | 2.70E−03 |
| RS4908087 | 71 | | | 1 | 100800615 | 2.80E−03 |
| RS10490876 | 314 | NAALADL2 | 254827 | 3 | 176634491 | 2.80E−03 |
| RS10517214 | 343 | | | 4 | 48101753 | 2.80E−03 |
| RS10512922 | 496 | ADCY2 | 108 | 5 | 7457278 | 2.80E−03 |
| RS631269 | 649 | | | 6 | 54417196 | 2.80E−03 |
| RS10503575 | 778 | MSR1 | 4481 | 8 | 16026250 | 2.80E−03 |
| RS1379742 | 863 | CNGB3 | 54714 | 8 | 87780661 | 2.80E−03 |
| RS722833 | 1332 | GAS7 | 8522 | 17 | 9876974 | 2.80E−03 |
| RS540970 | 1377 | KCTD1 | 284252 | 18 | 22430481 | 2.80E−03 |
| RS4715005 | 641 | | | 6 | 47472916 | 2.90E−03 |
| RS10504524 | 830 | TRPA1 | 8989 | 8 | 73118445 | 2.90E−03 |
| RS1025927 | 831 | TRPA1 | 8989 | 8 | 73125689 | 2.90E−03 |
| RS10491092 | 1333 | GAS7 | 8522 | 17 | 9945918 | 2.90E−03 |
| RS2822668 | 1433 | | | 21 | 14752268 | 2.90E−03 |
| RS841337 | 59 | | | 1 | 94853548 | 3.00E−03 |
| RS1033017 | 188 | MALL | 7851 | 2 | 110210596 | 3.00E−03 |
| RS696236 | 279 | PRICKLE2 | 166336 | 3 | 64184258 | 3.00E−03 |
| RS10519692 | 571 | | | 5 | 121423648 | 3.00E−03 |
| RS666336 | 802 | | | 8 | 16758689 | 3.00E−03 |
| RS1025928 | 832 | TRPA1 | 8989 | 8 | 73125812 | 3.00E−03 |
| RS2297355 | 920 | C9orf125 | 84302 | 9 | 101323312 | 3.00E−03 |
| RS1931321 | 926 | SVEP1 | 79987 | 9 | 110285257 | 3.00E−03 |
| RS1556082 | 1215 | GPC6 | 10082 | 13 | 93502465 | 3.00E−03 |
| RS185266 | 1305 | | | 16 | 50595102 | 3.00E−03 |
| RS2059232 | 1306 | | | 16 | 50596704 | 3.00E−03 |
| RS2822665 | 1432 | | | 21 | 14744278 | 3.00E−03 |
| RS10495014 | 109 | USH2A | 7399 | 1 | 212609915 | 3.10E−03 |
| RS7564203 | 134 | | | 2 | 6032465 | 3.10E−03 |
| RS553346 | 471 | | | 4 | 177769311 | 3.10E−03 |
| RS715428 | 1365 | KIAA1772 | 80000 | 18 | 17131449 | 3.10E−03 |
| RS9284616 | 360 | FRAS1 | 80144 | 4 | 79349710 | 3.20E−03 |
| RS10520899 | 507 | | | 5 | 25103129 | 3.20E−03 |
| RS251422 | 531 | PDE8B | 8622 | 5 | 76609190 | 3.20E−03 |
| RS10514096 | 533 | PDE8B | 8622 | 5 | 76700940 | 3.20E−03 |
| RS2150738 | 919 | C9orf125 | 84302 | 9 | 101320263 | 3.20E−03 |
| RS1033702 | 1255 | KIAA1622 | 57718 | 14 | 93717052 | 3.20E−03 |
| RS888405 | 278 | PRICKLE2 | 166336 | 3 | 64176742 | 3.30E−03 |
| RS495497 | 451 | LOC442117 | 442117 | 4 | 173117269 | 3.30E−03 |
| RS2956538 | 572 | | | 5 | 121427436 | 3.30E−03 |
| RS942662 | 627 | | | 6 | 23850282 | 3.30E−03 |
| RS9296538 | 637 | | | 6 | 47302408 | 3.30E−03 |
| RS4706652 | 667 | | | 6 | 78303823 | 3.30E−03 |
| RS2044271 | 828 | TRPA1 | 8989 | 8 | 73114699 | 3.30E−03 |
| RS4773773 | 1216 | GPC6 | 10082 | 13 | 93509501 | 3.30E−03 |
| RS1362400 | 1315 | AYTL1 | 54947 | 16 | 54129326 | 3.30E−03 |
| RS1981134 | 72 | | | 1 | 100823226 | 3.40E−03 |
| RS3770857 | 167 | CRIM1 | 51232 | 2 | 36608196 | 3.40E−03 |
| RS10517279 | 345 | SCFD2 | 152579 | 4 | 53831522 | 3.40E−03 |
| RS9312637 | 346 | SCFD2 | 152579 | 4 | 53867664 | 3.40E−03 |
| RS1390165 | 450 | | | 4 | 173099567 | 3.40E−03 |
| RS33375 | 598 | | | 5 | 170998718 | 3.40E−03 |
| RS10503572 | 789 | | | 8 | 16131101 | 3.40E−03 |
| RS2278667 | 852 | | | 8 | 80877490 | 3.40E−03 |
| RS2986671 | 925 | SVEP1 | 79987 | 9 | 110272210 | 3.40E−03 |
| RS1797376 | 1133 | LGR5 | 8549 | 12 | 70247964 | 3.40E−03 |
| RS2854946 | 1259 | SERPINA5 | 5104 | 14 | 94118132 | 3.40E−03 |
| RS2681962 | 1276 | UNC13C | 440279 | 15 | 52685879 | 3.40E−03 |
| RS10521320 | 1316 | AYTL1 | 54947 | 16 | 54133662 | 3.40E−03 |
| RS9284617 | 361 | FRAS1 | 80144 | 4 | 79349747 | 3.50E−03 |
| RS10520350 | 472 | | | 4 | 177769342 | 3.50E−03 |
| RS10520903 | 502 | | | 5 | 24966468 | 3.50E−03 |
| RS1283712 | 869 | ANGPT1 | 284 | 8 | 108535430 | 3.50E−03 |
| RS763889 | 1297 | | | 15 | 96543742 | 3.50E−03 |
| RS10497642 | 216 | | | 2 | 184653938 | 3.60E−03 |
| RS714206 | 535 | | | 5 | 81752090 | 3.60E−03 |
| RS4512212 | 611 | | | 6 | 10379387 | 3.60E−03 |
| RS1458402 | 669 | | | 6 | 78319510 | 3.60E−03 |
| RS479600 | 918 | | | 9 | 101282358 | 3.60E−03 |
| RS719223 | 1217 | GPC6 | 10082 | 13 | 93532937 | 3.60E−03 |
| RS8182127 | 1313 | AYTL1 | 54947 | 16 | 54127098 | 3.60E−03 |
| RS10518184 | 359 | FRAS1 | 80144 | 4 | 79341261 | 3.70E−03 |
| RS2348213 | 404 | | | 4 | 111679063 | 3.70E−03 |
| RS432164 | 470 | | | 4 | 177766242 | 3.70E−03 |
| RS10520352 | 473 | | | 4 | 177805099 | 3.70E−03 |
| RS251421 | 530 | PDE8B | 8622 | 5 | 76608726 | 3.70E−03 |
| RS1795462 | 1132 | LGR5 | 8549 | 12 | 70247867 | 3.70E−03 |
| RS10521321 | 1314 | AYTL1 | 54947 | 16 | 54129064 | 3.70E−03 |
| RS2411290 | 344 | SCFD2 | 152579 | 4 | 53780438 | 3.80E−03 |
| RS494770 | 452 | LOC442117 | 442117 | 4 | 173117307 | 3.80E−03 |
| RS990763 | 481 | | | 4 | 188204165 | 3.80E−03 |
| RS1343493 | 668 | | | 6 | 78313564 | 3.80E−03 |
| RS293975 | 1061 | TMEM16C | 63982 | 11 | 26546076 | 3.80E−03 |
| RS959987 | 1165 | SLC15A4 | 121260 | 12 | 127817680 | 3.80E−03 |
| RS1442799 | 1298 | | | 15 | 96589992 | 3.80E−03 |
| RS10502436 | 1366 | KIAA1772 | 80000 | 18 | 17219520 | 3.80E−03 |
| RS9288572 | 245 | | | 2 | 222787868 | 3.90E−03 |
| RS1964670 | 362 | FRAS1 | 80144 | 4 | 79351645 | 3.90E−03 |
| RS10507325 | 1169 | | | 13 | 22615653 | 3.90E−03 |
| RS920690 | 1299 | | | 15 | 96590158 | 3.90E−03 |
| RS243840 | 1312 | MMP2 | 4313 | 16 | 54085660 | 3.90E−03 |
| RS1893290 | 1374 | LAMA3 | 3909 | 18 | 19696619 | 3.90E−03 |
| RS978313 | 17 | | | 1 | 68188495 | 4.00E−03 |
| RS548825 | 21 | | | 1 | 69563639 | 4.00E−03 |
| RS841338 | 60 | | | 1 | 94854749 | 4.00E−03 |
| RS6662930 | 100 | CNTN2 | 6900 | 1 | 201773122 | 4.00E−03 |
| RS10496435 | 189 | NPHP1 | 4867 | 2 | 110286548 | 4.00E−03 |
| RS1502887 | 358 | FRAS1 | 80144 | 4 | 79340562 | 4.00E−03 |
| RS1797374 | 1131 | LGR5 | 8549 | 12 | 70246800 | 4.00E−03 |
| RS1983657 | 1258 | SERPINA5 | 5104 | 14 | 94117046 | 4.00E−03 |
| RS3755214 | 162 | CRIM1 | 51232 | 2 | 36537729 | 4.10E−03 |
| RS1882233 | 181 | | | 2 | 76710013 | 4.10E−03 |
| RS2347597 | 584 | G3BP | 10146 | 5 | 151161511 | 4.10E−03 |
| RS10505109 | 876 | | | 8 | 108635047 | 4.10E−03 |
| RS1999598 | 929 | SVEP1 | 79987 | 9 | 110322993 | 4.10E−03 |
| RS1471013 | 1043 | | | 11 | 11697539 | 4.10E−03 |
| RS2822669 | 1434 | | | 21 | 14752449 | 4.10E−03 |
| RS766643 | 497 | ADCY2 | 108 | 5 | 7499889 | 4.20E−03 |
| RS1385290 | 777 | | | 8 | 15987084 | 4.20E−03 |
| RS1383160 | 788 | | | 8 | 16131054 | 4.20E−03 |
| RS10509210 | 1003 | | | 10 | 66125316 | 4.20E−03 |
| RS1410747 | 1218 | GPC6 | 10082 | 13 | 93533153 | 4.20E−03 |
| RS9284299 | 1277 | UNC13C | 440279 | 15 | 52685923 | 4.20E−03 |
| RS2010122 | 1296 | | | 15 | 96543545 | 4.20E−03 |
| RS4800741 | 1367 | KIAA1772 | 80000 | 18 | 17327951 | 4.20E−03 |
| RS963552 | 180 | | | 2 | 76708216 | 4.30E−03 |
| RS1675511 | 284 | | | 3 | 99775373 | 4.30E−03 |
| RS2704404 | 342 | | | 4 | 48075472 | 4.30E−03 |
| RS487474 | 646 | | | 6 | 54364173 | 4.30E−03 |
| RS3913991 | 647 | | | 6 | 54402115 | 4.30E−03 |
| RS243818 | 648 | | | 6 | 54404636 | 4.30E−03 |
| RS724201 | 1130 | LGR5 | 8549 | 12 | 70242280 | 4.30E−03 |
| RS10493933 | 73 | | | 1 | 100823303 | 4.40E−03 |
| RS10495526 | 130 | | | 2 | 5957046 | 4.40E−03 |
| RS985343 | 182 | | | 2 | 76823437 | 4.40E−03 |
| RS1549920 | 583 | | | 5 | 151121037 | 4.40E−03 |
| RS1379782 | 1161 | | | 12 | 127583749 | 4.40E−03 |
| RS8042622 | 1280 | UNC13C | 440279 | 15 | 52692250 | 4.40E−03 |
| RS6114144 | 1426 | | | 20 | 23410022 | 4.40E−03 |
| RS1854510 | 63 | PAP2D | 163404 | 1 | 99102265 | 4.50E−03 |
| RS10517281 | 348 | SCFD2 | 152579 | 4 | 53868523 | 4.50E−03 |
| RS997721 | 374 | HNRPDL | 9987 | 4 | 83702471 | 4.50E−03 |
| RS4695953 | 459 | GLRA3 | 8001 | 4 | 176006441 | 4.50E−03 |
| RS1366236 | 586 | GLRA1 | 2741 | 5 | 151264796 | 4.50E−03 |
| RS2614059 | 811 | LOC389644 | 389644 | 8 | 28212815 | 4.50E−03 |
| RS10508900 | 977 | FRMPD2 | 143162 | 10 | 49126059 | 4.50E−03 |
| RS876705 | 1000 | | | 10 | 66057533 | 4.50E−03 |
| RS1389455 | 1066 | TMEM16C | 63982 | 11 | 26604761 | 4.50E−03 |
| RS555210 | 1096 | | | 11 | 94735784 | 4.50E−03 |
| RS10506057 | 1120 | | | 12 | 30159671 | 4.50E−03 |
| RS1280823 | 1129 | LGR5 | 8549 | 12 | 70210415 | 4.50E−03 |
| RS9317535 | 1175 | SGCG | 6445 | 13 | 22653892 | 4.50E−03 |
| RS1818711 | 1279 | UNC13C | 440279 | 15 | 52692074 | 4.50E−03 |
| RS9324714 | 585 | GLRA1 | 2741 | 5 | 151223956 | 4.60E−03 |
| RS10498888 | 663 | KCNQ5 | 56479 | 6 | 73800439 | 4.60E−03 |
| RS4497357 | 1067 | TMEM16C | 63982 | 11 | 26619122 | 4.60E−03 |
| RS10506055 | 1119 | | | 12 | 30097508 | 4.60E−03 |
| RS948720 | 1401 | | | 18 | 73314377 | 4.60E−03 |
| RS1571502 | 65 | PAP2D | 163404 | 1 | 99137091 | 4.70E−03 |
| RS349018 | 246 | | | 2 | 222789893 | 4.70E−03 |
| RS7696949 | 418 | | | 4 | 126771743 | 4.70E−03 |
| RS7826088 | 785 | | | 8 | 16106013 | 4.70E−03 |
| RS1480139 | 787 | | | 8 | 16129067 | 4.70E−03 |
| RS10501826 | 1097 | | | 11 | 94739512 | 4.70E−03 |
| RS1765618 | 54 | | | 1 | 94562596 | 4.80E−03 |
| RS1506363 | 417 | FAT4 | 79633 | 4 | 126759952 | 4.80E−03 |
| RS1382901 | 594 | | | 5 | 170969444 | 4.80E−03 |
| RS1915603 | 1004 | | | 10 | 66125650 | 4.80E−03 |
| RS6493693 | 1275 | UNC13C | 440279 | 15 | 52679875 | 4.80E−03 |
| RS2764536 | 28 | | | 1 | 69759261 | 4.90E−03 |
| RS2141494 | 285 | | | 3 | 99779985 | 4.90E−03 |
| RS999476 | 488 | | | 4 | 188660406 | 4.90E−03 |
| RS6902538 | 610 | | | 6 | 10377786 | 4.90E−03 |
| RS477516 | 800 | | | 8 | 16758362 | 4.90E−03 |
| RS1832331 | 30 | | | 1 | 80873358 | 5.00E−03 |
| RS4694890 | 341 | TEC | 7006 | 4 | 48067195 | 5.00E−03 |
| RS10519693 | 573 | | | 5 | 121427572 | 5.00E−03 |
| RS1459709 | 1035 | ADAM12 | 8038 | 10 | 127862818 | 5.00E−03 |
| RS3911569 | 1095 | | | 11 | 94735683 | 5.00E−03 |
| RS4656049 | 49 | | | 1 | 88656915 | 5.10E−03 |
| RS10510915 | 277 | PRICKLE2 | 166336 | 3 | 64169587 | 5.10E−03 |
| RS7117834 | 1047 | | | 11 | 11733493 | 5.10E−03 |
| RS787533 | 10 | | | 1 | 67758330 | 5.20E−03 |
| RS10489919 | 64 | PAP2D | 163404 | 1 | 99136909 | 5.20E−03 |
| RS10510912 | 275 | PRICKLE2 | 166336 | 3 | 64140589 | 5.20E−03 |
| RS10513288 | 937 | ASTN2 | 23245 | 9 | 117069393 | 5.20E−03 |
| RS10509209 | 1001 | | | 10 | 66120490 | 5.20E−03 |
| RS1915602 | 1002 | | | 10 | 66125009 | 5.20E−03 |
| RS9326628 | 190 | | | 2 | 110324042 | 5.30E−03 |
| RS2110509 | 329 | | | 4 | 27588882 | 5.30E−03 |
| RS393279 | 474 | | | 4 | 177816548 | 5.30E−03 |
| RS3816240 | 856 | CPNE3 | 8895 | 8 | 87636421 | 5.30E−03 |
| RS1062225 | 984 | MAPK8 | 5599 | 10 | 49313232 | 5.30E−03 |
| RS1072769 | 1117 | | | 12 | 30067073 | 5.30E−03 |
| RS723856 | 1435 | | | 21 | 14771576 | 5.30E−03 |
| RS1368080 | 146 | KBTBD9 | 114818 | 2 | 23623079 | 5.40E−03 |
| RS153717 | 276 | PRICKLE2 | 166336 | 3 | 64147033 | 5.40E−03 |
| RS1352058 | 458 | GLRA3 | 8001 | 4 | 175981135 | 5.40E−03 |
| RS10512876 | 516 | | | 5 | 45285361 | 5.40E−03 |
| RS1956456 | 1254 | KIAA1622 | 57718 | 14 | 93712715 | 5.40E−03 |
| RS1544299 | 111 | USH2A | 7399 | 1 | 212652783 | 5.50E−03 |
| RS10495524 | 126 | | | 2 | 5881900 | 5.50E−03 |
| RS10506054 | 1118 | | | 12 | 30096174 | 5.50E−03 |
| RS10518391 | 410 | ANXA5 | 308 | 4 | 122965453 | 5.60E−03 |
| RS4128165 | 457 | GLRA3 | 8001 | 4 | 175948433 | 5.60E−03 |
| RS10512919 | 493 | ADCY2 | 108 | 5 | 7455004 | 5.60E−03 |
| RS10512854 | 510 | | | 5 | 44546854 | 5.60E−03 |
| RS10498889 | 664 | KCNQ5 | 56479 | 6 | 73839097 | 5.60E−03 |
| RS899683 | 786 | | | 8 | 16113895 | 5.60E−03 |
| RS2136886 | 1411 | | | 19 | 21847999 | 5.60E−03 |
| RS7606048 | 145 | KBTBD9 | 114818 | 2 | 23545863 | 5.70E−03 |
| RS4655769 | 18 | | | 1 | 68188999 | 5.80E−03 |
| RS1430745 | 19 | | | 1 | 68258754 | 5.80E−03 |
| RS10495525 | 129 | | | 2 | 5937776 | 5.80E−03 |
| RS10490750 | 147 | KBTBD9 | 114818 | 2 | 23623216 | 5.80E−03 |
| RS1495291 | 449 | | | 4 | 173062483 | 5.80E−03 |
| RS4690697 | 464 | | | 4 | 177690238 | 5.80E−03 |
| RS411244 | 541 | | | 5 | 82242350 | 5.80E−03 |
| RS725937 | 582 | SPARC | 6678 | 5 | 151036904 | 5.80E−03 |
| RS4078145 | 1042 | | | 11 | 11620469 | 5.80E−03 |
| RS533926 | 1094 | | | 11 | 94716912 | 5.80E−03 |
| RS9322320 | 690 | C6orf96 | 55005 | 6 | 151844022 | 5.90E−03 |
| RS823603 | 806 | | | 8 | 16861200 | 5.90E−03 |
| RS2614060 | 812 | LOC389644 | 389644 | 8 | 28212897 | 5.90E−03 |
| RS10506053 | 1116 | | | 12 | 30061626 | 5.90E−03 |
| RS1992099 | 106 | USH2A | 7399 | 1 | 212556039 | 6.00E−03 |
| RS1912163 | 340 | TEC | 7006 | 4 | 48051581 | 6.00E−03 |
| RS3800273 | 691 | C6orf96 | 55005 | 6 | 151851149 | 6.00E−03 |
| RS2983301 | 1427 | | | 20 | 23434589 | 6.00E−03 |
| RS1709329 | 148 | KBTBD9 | 114818 | 2 | 23634850 | 6.10E−03 |
| RS10488616 | 716 | TXNDC3 | 51314 | 7 | 37706299 | 6.10E−03 |
| RS4112759 | 944 | | | 9 | 117313823 | 6.10E−03 |
| RS1877568 | 303 | ITGB5 | 3693 | 3 | 125966877 | 6.20E−03 |
| RS1818704 | 1274 | UNC13C | 440279 | 15 | 52679696 | 6.20E−03 |
| RS10492899 | 1304 | | | 16 | 50583588 | 6.20E−03 |
| RS930528 | 1351 | TANC2 | 26115 | 17 | 58680003 | 6.20E−03 |
| RS10495530 | 135 | | | 2 | 6034027 | 6.30E−03 |
| RS10489920 | 66 | PAP2D | 163404 | 1 | 99146514 | 6.40E−03 |
| RS1346612 | 128 | | | 2 | 5936521 | 6.40E−03 |
| RS6867905 | 560 | | | 5 | 113473289 | 6.40E−03 |
| RS2410373 | 776 | | | 8 | 15968877 | 6.40E−03 |
| RS10502475 | 1376 | KCTD1 | 284252 | 18 | 22427942 | 6.40E−03 |
| RS10518467 | 416 | FAT4 | 79633 | 4 | 126755739 | 6.50E−03 |
| RS10514098 | 534 | PDE8B | 8622 | 5 | 76740837 | 6.50E−03 |
| RS6940110 | 609 | | | 6 | 10377050 | 6.50E−03 |
| RS716329 | 689 | ZBTB2 | 57621 | 6 | 151791227 | 6.50E−03 |
| RS723855 | 1436 | | | 21 | 14771745 | 6.50E−03 |
| RS1841544 | 286 | | | 3 | 99839093 | 6.60E−03 |
| RS2461063 | 846 | | | 8 | 80781668 | 6.60E−03 |
| RS953211 | 414 | FAT4 | 79633 | 4 | 126708654 | 6.70E−03 |
| RS10503585 | 801 | | | 8 | 16758568 | 6.70E−03 |
| RS2614074 | 813 | | | 8 | 28222739 | 6.70E−03 |
| RS10502439 | 1369 | NPC1 | 4864 | 18 | 19366772 | 6.70E−03 |
| RS989901 | 922 | | | 9 | 101346820 | 6.80E−03 |
| RS9284634 | 405 | | | 4 | 122864741 | 7.00E−03 |
| RS433563 | 542 | | | 5 | 82242557 | 7.00E−03 |
| RS2721304 | 559 | | | 5 | 113462331 | 7.00E−03 |
| RS726217 | 1430 | CST9 | 128822 | 20 | 23532116 | 7.00E−03 |
| RS1485871 | 454 | LOC442117 | 442117 | 4 | 173125753 | 7.10E−03 |
| RS10513300 | 942 | ASTN2 | 23245 | 9 | 117209760 | 7.20E−03 |
| RS3844028 | 74 | | | 1 | 100823636 | 7.30E−03 |
| RS1429235 | 127 | | | 2 | 5910112 | 7.30E−03 |
| RS6436292 | 247 | | | 2 | 222798248 | 7.30E−03 |
| RS115931 | 287 | ST3GAL6 | 10402 | 3 | 99952479 | 7.30E−03 |
| RS10518466 | 415 | FAT4 | 79633 | 4 | 126737972 | 7.30E−03 |
| RS1046852 | 20 | C1orf139 | 79971 | 1 | 68303125 | 7.40E−03 |
| RS3753855 | 99 | CNTN2 | 6900 | 1 | 201772383 | 7.40E−03 |
| RS1381115 | 810 | | | 8 | 28202690 | 7.40E−03 |
| RS976891 | 324 | | | 4 | 27585616 | 7.50E−03 |
| RS3800274 | 692 | C6orf96 | 55005 | 6 | 151851306 | 7.50E−03 |
| RS9322321 | 693 | C6orf96 | 55005 | 6 | 151851744 | 7.50E−03 |
| RS4570241 | 950 | | | 9 | 117476532 | 7.50E−03 |
| RS1571500 | 61 | PAP2D | 163404 | 1 | 99098314 | 7.70E−03 |
| RS4695357 | 339 | TEC | 7006 | 4 | 48040123 | 7.70E−03 |
| RS10513301 | 943 | ASTN2 | 23245 | 9 | 117241331 | 7.70E−03 |
| RS3848309 | 1309 | | | 16 | 50690687 | 7.70E−03 |
| RS3821359 | 288 | ST3GAL6 | 10402 | 3 | 99973295 | 7.80E−03 |
| RS27378 | 498 | ADCY2 | 108 | 5 | 7521560 | 7.90E−03 |
| RS2031393 | 621 | | | 6 | 23796368 | 7.90E−03 |
| RS1942598 | 1400 | | | 18 | 73276885 | 7.90E−03 |
| RS3734953 | 721 | EPDR1 | 54749 | 7 | 37763462 | 8.00E−03 |
| RS10484047 | 1260 | SERPINA5 | 5104 | 14 | 94118981 | 8.00E−03 |
| RS10519366 | 557 | | | 5 | 113326892 | 8.10E−03 |
| RS9296547 | 642 | | | 6 | 47474339 | 8.10E−03 |
| RS10491511 | 917 | | | 9 | 101259503 | 8.10E−03 |
| RS9304978 | 1407 | | | 19 | 20949730 | 8.10E−03 |
| RS10516086 | 599 | | | 5 | 171002992 | 8.20E−03 |
| RS2378672 | 911 | NTRK2 | 4915 | 9 | 84860588 | 8.20E−03 |
| RS7921148 | 998 | | | 10 | 66047006 | 8.20E−03 |
| RS2394081 | 999 | | | 10 | 66050572 | 8.20E−03 |
| RS10519377 | 561 | | | 5 | 113535053 | 8.30E−03 |
| RS1003203 | 1428 | LOC128820 | 128820 | 20 | 23477910 | 8.30E−03 |
| RS1700595 | 540 | | | 5 | 82242104 | 8.40E−03 |
| RS10502435 | 1368 | | | 18 | 17361089 | 8.40E−03 |
| RS1158167 | 1429 | | | 20 | 23526189 | 8.40E−03 |
| RS10493679 | 39 | | | 1 | 81046048 | 8.50E−03 |
| RS823600 | 807 | | | 8 | 16861804 | 8.50E−03 |
| RS10504523 | 827 | TRPA1 | 8989 | 8 | 73114044 | 8.50E−03 |
| RS10484886 | 614 | | | 6 | 10477661 | 8.60E−03 |
| RS2460605 | 1281 | UNC13C | 440279 | 15 | 52693011 | 8.60E−03 |
| RS7580778 | 187 | | | 2 | 109757198 | 8.70E−03 |
| RS2974490 | 558 | | | 5 | 113433775 | 8.70E−03 |
| RS1801758 | 1329 | GAS7 | 8522 | 17 | 9756951 | 8.70E−03 |
| RS306322 | 50 | | | 1 | 88673430 | 8.80E−03 |
| RS329204 | 916 | | | 9 | 85028966 | 8.80E−03 |
| RS564038 | 1093 | | | 11 | 94715938 | 8.80E−03 |
| RS10490874 | 310 | NAALADL2 | 254827 | 3 | 176538732 | 8.90E−03 |
| RS288989 | 1364 | ROCK1 | 6093 | 18 | 16836580 | 8.90E−03 |
| RS10495745 | 144 | KBTBD9 | 114818 | 2 | 23543335 | 9.00E−03 |
| RS2396817 | 636 | | | 6 | 47255521 | 9.10E−03 |
| RS470732 | 1166 | | | 12 | 128008175 | 9.10E−03 |
| RS9301934 | 1219 | GPC6 | 10082 | 13 | 93537860 | 9.20E−03 |
| RS10503827 | 814 | | | 8 | 28223645 | 9.30E−03 |
| RS10501054 | 1060 | TMEM16C | 63982 | 11 | 26536121 | 9.40E−03 |
| RS344924 | 15 | | | 1 | 67817474 | 9.80E−03 |
| RS10520158 | 183 | | | 2 | 76851929 | 9.80E−03 |
| RS293966 | 1059 | TMEM16C | 63982 | 11 | 26536069 | 9.80E−03 |
| RS10489918 | 62 | PAP2D | 163404 | 1 | 99101483 | 1.00E−02 |
| RS1956455 | 1253 | KIAA1622 | 57718 | 14 | 93711620 | 1.00E−02 |
| RS10506521 | 1352 | TANC2 | 26115 | 17 | 58695046 | 1.00E−02 |
| RS37551 | 543 | | | 5 | 82256024 | 1.02E−02 |
| RS10493814 | 51 | LOC401954 | 401954 | 1 | 88695848 | 1.06E−02 |
| RS1426935 | 456 | | | 4 | 175905195 | 1.06E−02 |
| RS10514245 | 544 | | | 5 | 82256614 | 1.06E−02 |
| RS10513731 | 315 | NAALADL2 | 254827 | 3 | 176661277 | 1.07E−02 |
| RS7950644 | 1098 | | | 11 | 94739539 | 1.08E−02 |
| RS10519378 | 563 | | | 5 | 113555966 | 1.11E−02 |
| RS10500214 | 1406 | ZNF85 | 7639 | 19 | 20914290 | 1.11E−02 |
| RS10520290 | 460 | GLRA3 | 8001 | 4 | 176006547 | 1.13E−02 |
| RS526715 | 799 | | | 8 | 16754047 | 1.13E−02 |
| RS293969 | 1056 | TMEM16C | 63982 | 11 | 26532067 | 1.13E−02 |
| RS29620 | 593 | | | 5 | 170969416 | 1.16E−02 |
| RS1519991 | 179 | | | 2 | 76698833 | 1.17E−02 |
| RS9326911 | 562 | | | 5 | 113535625 | 1.18E−02 |
| RS10501053 | 1058 | TMEM16C | 63982 | 11 | 26533301 | 1.20E−02 |
| RS10493425 | 9 | | | 1 | 67722148 | 1.22E−02 |
| RS871151 | 75 | | | 1 | 100835758 | 1.23E−02 |
| RS1018912 | 1412 | ZNF208 | 7757 | 19 | 21948425 | 1.24E−02 |
| RS724202 | 1057 | TMEM16C | 63982 | 11 | 26532254 | 1.26E−02 |
| RS8013924 | 1252 | OTUB2 | 78990 | 14 | 93572196 | 1.26E−02 |
| RS1382902 | 592 | | | 5 | 170969171 | 1.28E−02 |
| RS10516085 | 591 | | | 5 | 170949443 | 1.29E−02 |
| RS941490 | 1251 | | | 14 | 93508235 | 1.29E−02 |
| RS6891243 | 492 | | | 5 | 7414980 | 1.30E−02 |
| RS10502476 | 1381 | | | 18 | 22641232 | 1.31E−02 |
| RS10512915 | 491 | | | 5 | 7380172 | 1.35E−02 |
| RS1589273 | 184 | | | 2 | 76872376 | 1.38E−02 |
| RS4128873 | 1041 | GALNTL4 | 374378 | 11 | 11499597 | 1.38E−02 |
| RS896322 | 104 | TMCC2 | 9911 | 1 | 201941402 | 1.41E−02 |
| RS10520348 | 463 | | | 4 | 177636887 | 1.42E−02 |
| RS1552069 | 411 | | | 4 | 122983540 | 1.45E−02 |
| RS7937815 | 1048 | | | 11 | 11738741 | 1.52E−02 |
| RS10484046 | 1256 | | | 14 | 94077768 | 1.55E−02 |
| RS997280 | 1261 | LOC390503 | 390503 | 14 | 94171096 | 1.55E−02 |
| RS1342337 | 659 | KCNQ5 | 56479 | 6 | 73692956 | 1.57E−02 |
| RS3770949 | 161 | CRIM1 | 51232 | 2 | 36515414 | 1.58E−02 |
| RS1429220 | 125 | | | 2 | 5881639 | 1.67E−02 |
| RS2722897 | 815 | PNOC | 5368 | 8 | 28229116 | 1.68E−02 |
| RS10520288 | 455 | | | 4 | 175884923 | 1.69E−02 |
| RS1955649 | 1262 | | | 14 | 94196733 | 1.69E−02 |
| RS2200455 | 1160 | | | 12 | 127317497 | 1.77E−02 |
| RS1541679 | 523 | | | 5 | 53729676 | 1.79E−02 |
| RS9292042 | 517 | ARL15 | 54622 | 5 | 53572755 | 1.81E−02 |
| RS241068 | 1345 | | | 17 | 58441292 | 1.82E−02 |
| RS717806 | 160 | | | 2 | 36487186 | 1.94E−02 |
| RS7156546 | 1257 | | | 14 | 94078129 | 1.96E−02 |
| RS322401 | 480 | | | 4 | 188200452 | 1.99E−02 |
| RS167721 | 479 | | | 4 | 188154690 | 2.11E−02 |

TABLE 5
SNP markers with P-value less than 0.005 for APOE1 from t-test assuming
equal variaces between groups. Number of observations are marked by n1 and n2
and group avarages by avg1 and avg2.

| RS_id | SEQ_ID_no | Gene | Gene_id | Chr | Position | n1 | avg1 | n2 | avg2 | ttest | df | P-value |
|---|---|---|---|---|---|---|---|---|---|---|---|---|
| RS6954679 | 728 | | | 7 | 63134099 | 437 | 1.32 | 41 | 1.51 | 4.79 | 476 | 2.23E−06 |
| RS10502465 | 2490 | | | 18 | 20848719 | 446 | 1.32 | 30 | 1.53 | 4.74 | 474 | 2.83E−06 |
| RS1016579 | 881 | | | 8 | 129269143 | 399 | 1.32 | 25 | 1.54 | 4.61 | 422 | 5.34E−06 |
| RS1867584 | 985 | | | 10 | 49321355 | 190 | 1.4 | 244 | 1.29 | 4.52 | 432 | 8.00E−06 |
| RS295577 | 524 | NDUFA12L | 91942 | 5 | 60457592 | 455 | 1.32 | 13 | 1.62 | 4.51 | 466 | 8.21E−06 |
| RS10503626 | 808 | PSD3 | 23362 | 8 | 18591364 | 459 | 1.32 | 21 | 1.56 | 4.45 | 478 | 1.07E−05 |
| RS3912653 | 1091 | FAT3 | 120114 | 11 | 92135172 | 197 | 1.27 | 279 | 1.38 | 4.45 | 474 | 1.70E−05 |
| RS1792369 | 1090 | FAT3 | 120114 | 11 | 92092081 | 196 | 1.28 | 276 | 1.38 | 4.34 | 470 | 1.75E−05 |
| RS10498673 | 1912 | BMP6 | 654 | 6 | 7770256 | 406 | 1.31 | 68 | 1.45 | 4.28 | 472 | 2.26E−05 |
| RS2889611 | 2213 | | | 10 | 49321754 | 184 | 1.4 | 280 | 1.3 | 4.28 | 462 | 2.27E−05 |
| RS10498293 | 2380 | | | 14 | 27284686 | 417 | 1.31 | 65 | 1.45 | 4.27 | 480 | 2.36E−05 |
| RS2056247 | 1092 | | | 11 | 93315872 | 391 | 1.31 | 91 | 1.43 | 4.2 | 480 | 3.18E−05 |
| RS10507768 | 2359 | | | 13 | 68865849 | 19 | 1.56 | 441 | 1.32 | 4.15 | 458 | 3.96E−05 |
| RS295525 | 525 | | | 5 | 60508623 | 470 | 1.33 | 12 | 1.62 | 4.11 | 480 | 4.65E−05 |
| RS572241 | 2264 | FAT3 | 120114 | 11 | 91942082 | 269 | 1.29 | 199 | 1.38 | 4.04 | 466 | 6.25E−05 |
| RS10517836 | 1747 | | | 4 | 63541480 | 405 | 1.32 | 45 | 1.47 | 4.04 | 448 | 6.29E−05 |
| RS10508901 | 981 | MAPK8 | 5599 | 10 | 49297404 | 185 | 1.39 | 287 | 1.29 | 4.03 | 470 | 6.50E−05 |
| RS2503102 | 679 | LOC442238 | 442238 | 6 | 100650488 | 272 | 1.37 | 142 | 1.27 | 4.01 | 412 | 7.21E−05 |
| RS36617 | 2266 | FAT3 | 120114 | 11 | 91943638 | 278 | 1.29 | 202 | 1.38 | 3.97 | 478 | 8.29E−05 |
| RS2854946 | 1259 | SERPINA5 | 5104 | 14 | 94118132 | 321 | 1.3 | 91 | 1.41 | 3.97 | 410 | 8.49E−05 |
| RS4128928 | 1678 | | | 3 | 119083686 | 288 | 1.29 | 192 | 1.39 | 3.95 | 478 | 8.99E−05 |
| RS10508896 | 2210 | MARCH8 | 220972 | 10 | 45350649 | 451 | 1.34 | 29 | 1.16 | 3.95 | 478 | 8.99E−05 |
| RS10491855 | 947 | | | 9 | 119335541 | 347 | 1.31 | 123 | 1.41 | 3.94 | 468 | 9.39E−05 |
| RS352427 | 816 | | | 8 | 28571996 | 465 | 1.32 | 15 | 1.58 | 3.92 | 478 | 1.01E−04 |
| RS10501797 | 2276 | FAT3 | 120114 | 11 | 92167618 | 182 | 1.28 | 298 | 1.37 | 3.92 | 478 | 1.01E−04 |
| RS901682 | 2208 | MARCH8 | 220972 | 10 | 45285867 | 449 | 1.35 | 25 | 1.15 | 3.88 | 472 | 1.19E−04 |
| RS1893120 | 1788 | NDST4 | 64579 | 4 | 116230217 | 142 | 1.27 | 258 | 1.37 | 3.88 | 398 | 1.22E−04 |
| RS2425570 | 2554 | PTPRT | 11122 | 20 | 41111765 | 347 | 1.31 | 51 | 1.45 | 3.86 | 396 | 1.32E−04 |
| RS10509929 | 1021 | | | 10 | 112482278 | 55 | 1.45 | 405 | 1.32 | 3.83 | 458 | 1.46E−04 |
| RS7412987 | 1532 | LOC128153 | 128153 | 1 | 216013079 | 163 | 1.39 | 277 | 1.3 | 3.83 | 438 | 1.47E−04 |
| RS878016 | 757 | DLGAP2 | 9228 | 8 | 1585693 | 341 | 1.31 | 139 | 1.4 | 3.81 | 478 | 1.57E−04 |
| RS2474263 | 678 | LOC442238 | 442238 | 6 | 100641976 | 302 | 1.36 | 172 | 1.27 | 3.81 | 472 | 1.57E−04 |
| RS2045765 | 1760 | | | 4 | 63775688 | 421 | 1.32 | 57 | 1.45 | 3.8 | 476 | 1.63E−04 |
| RS2296697 | 40 | LPHN2 | 23266 | 1 | 82209807 | 393 | 1.31 | 75 | 1.43 | 3.79 | 466 | 1.70E−04 |
| RS10499436 | 2020 | | | 7 | 13649002 | 123 | 1.4 | 337 | 1.3 | 3.76 | 458 | 1.92E−04 |
| RS9317744 | 1176 | | | 13 | 22963766 | 433 | 1.33 | 11 | 1.61 | 3.74 | 442 | 2.08E−04 |
| RS7850478 | 946 | | | 9 | 119326659 | 356 | 1.31 | 126 | 1.4 | 3.73 | 480 | 2.14E−04 |
| RS2292889 | 2250 | DEPC-1 | 221120 | 11 | 43898353 | 94 | 1.25 | 378 | 1.35 | 3.73 | 470 | 2.15E−04 |
| RS2353296 | 1733 | | | 4 | 47956320 | 423 | 1.32 | 31 | 1.5 | 3.71 | 452 | 2.33E−04 |
| RS1961544 | 2559 | | | 21 | 19839174 | 402 | 1.32 | 24 | 1.52 | 3.71 | 424 | 2.35E−04 |
| RS7841198 | 2096 | PSD3 | 23362 | 8 | 18597648 | 447 | 1.32 | 31 | 1.49 | 3.7 | 476 | 2.41E−04 |
| RS250387 | 518 | ARL15 | 54622 | 5 | 53620394 | 399 | 1.31 | 77 | 1.43 | 3.69 | 474 | 2.50E−04 |
| RS10499437 | 2021 | | | 7 | 13649243 | 130 | 1.4 | 342 | 1.3 | 3.69 | 470 | 2.51E−04 |
| RS870241 | 1654 | FAM19A4 | 151647 | 3 | 68908072 | 324 | 1.31 | 126 | 1.41 | 3.69 | 448 | 2.52E−04 |
| RS10516125 | 1893 | | | 5 | 174413439 | 474 | 1.33 | 8 | 1.65 | 3.68 | 480 | 2.60E−04 |
| RS10488888 | 395 | | | 4 | 111503157 | 472 | 1.33 | 8 | 1.65 | 3.68 | 478 | 2.60E−04 |
| RS7792197 | 2055 | | | 7 | 87899449 | 446 | 1.32 | 32 | 1.49 | 3.68 | 476 | 2.60E−04 |
| RS10509702 | 1009 | | | 10 | 97914086 | 466 | 1.33 | 12 | 1.59 | 3.68 | 476 | 2.60E−04 |
| RS3760626 | 2533 | CLPTM1 | 1209 | 19 | 50148945 | 262 | 1.29 | 216 | 1.38 | 3.68 | 476 | 2.60E−04 |
| RS2072506 | 722 | AMPH | 273 | 7 | 38468920 | 442 | 1.32 | 30 | 1.5 | 3.68 | 470 | 2.60E−04 |
| RS6830598 | 1775 | NDST4 | 64579 | 4 | 116084463 | 161 | 1.39 | 259 | 1.3 | 3.68 | 418 | 2.64E−04 |
| RS10495074 | 1530 | LOC128153 | 128153 | 1 | 215998288 | 296 | 1.3 | 184 | 1.39 | 3.65 | 478 | 2.91E−04 |
| RS967326 | 2094 | PSD3 | 23362 | 8 | 18591541 | 448 | 1.32 | 32 | 1.49 | 3.65 | 478 | 2.91E−04 |
| RS1112809 | 210 | | | 2 | 148197197 | 420 | 1.35 | 50 | 1.21 | 3.65 | 468 | 2.92E−04 |
| RS10498292 | 1229 | | | 14 | 27284462 | 397 | 1.31 | 85 | 1.42 | 3.63 | 480 | 3.14E−04 |
| RS3827702 | 1423 | TASP1 | 55617 | 20 | 13446812 | 300 | 1.31 | 98 | 1.41 | 3.63 | 396 | 3.21E−04 |
| RS1528842 | 209 | | | 2 | 148174046 | 426 | 1.35 | 56 | 1.22 | 3.62 | 480 | 3.26E−04 |
| RS10512980 | 1823 | | | 5 | 51991165 | 319 | 1.36 | 151 | 1.27 | 3.62 | 468 | 3.27E−04 |
| RS3104997 | 2143 | | | 8 | 96630910 | 341 | 1.35 | 123 | 1.26 | 3.62 | 462 | 3.27E−04 |
| RS547008 | 2299 | | | 11 | 121523071 | 424 | 1.32 | 40 | 1.46 | 3.62 | 462 | 3.27E−04 |
| RS10516142 | 601 | FLT4 | 2324 | 5 | 180003769 | 474 | 1.33 | 8 | 1.64 | 3.61 | 480 | 3.38E−04 |
| RS10503573 | 780 | MSR1 | 4481 | 8 | 16047855 | 468 | 1.33 | 14 | 1.57 | 3.61 | 480 | 3.38E−04 |
| RS2793207 | 1900 | CDYL | 9425 | 6 | 4901002 | 447 | 1.33 | 17 | 1.55 | 3.59 | 462 | 3.66E−04 |
| RS1427421 | 1661 | MITF | 4286 | 3 | 69870404 | 410 | 1.32 | 44 | 1.46 | 3.59 | 452 | 3.67E−04 |
| RS10489688 | 1523 | | | 1 | 197054569 | 315 | 1.31 | 163 | 1.39 | 3.58 | 476 | 3.79E−04 |
| RS10518901 | 2441 | C15orf41 | 84529 | 15 | 34807406 | 256 | 1.3 | 192 | 1.38 | 3.58 | 446 | 3.81E−04 |
| RS4131462 | 1972 | | | 6 | 99364568 | 309 | 1.36 | 171 | 1.28 | 3.57 | 478 | 3.93E−04 |
| RS1499976 | 1684 | LOC389142 | 389142 | 3 | 119284020 | 369 | 1.3 | 101 | 1.4 | 3.57 | 468 | 3.94E−04 |
| RS10509448 | 1005 | NRG3 | 10718 | 10 | 83972610 | 432 | 1.32 | 20 | 1.52 | 3.57 | 450 | 3.95E−04 |
| RS758780 | 2036 | PTHB1 | 27241 | 7 | 33444985 | 302 | 1.3 | 180 | 1.39 | 3.56 | 480 | 4.08E−04 |
| RS758779 | 2037 | PTHB1 | 27241 | 7 | 33445083 | 302 | 1.3 | 180 | 1.39 | 3.56 | 480 | 4.08E−04 |
| RS2671762 | 1633 | | | 3 | 5110111 | 431 | 1.32 | 49 | 1.45 | 3.55 | 478 | 4.23E−04 |
| RS10514009 | 2530 | | | 18 | 67250588 | 449 | 1.32 | 31 | 1.49 | 3.55 | 478 | 4.23E−04 |
| RS950085 | 1006 | | | 10 | 95592637 | 139 | 1.37 | 199 | 1.28 | 3.56 | 336 | 4.24E−04 |
| RS1456196 | 293 | | | 3 | 119137243 | 358 | 1.31 | 84 | 1.41 | 3.55 | 440 | 4.27E−04 |
| RS2825673 | 2561 | | | 21 | 19888587 | 415 | 1.32 | 55 | 1.45 | 3.54 | 468 | 4.40E−04 |
| RS10492047 | 1158 | LOC144678 | 144678 | 12 | 125501283 | 457 | 1.32 | 25 | 1.5 | 3.53 | 480 | 4.56E−04 |
| RS4945630 | 1990 | | | 6 | 119472010 | 286 | 1.36 | 186 | 1.28 | 3.53 | 470 | 4.56E−04 |
| RS10484973 | 1966 | RIMS1 | 22999 | 6 | 72677020 | 366 | 1.32 | 98 | 1.42 | 3.52 | 462 | 4.74E−04 |
| RS2217610 | 700 | SDK1 | 221935 | 7 | 3697162 | 348 | 1.31 | 96 | 1.41 | 3.51 | 442 | 4.94E−04 |
| RS7107851 | 2287 | HEPHL1 | 341208 | 11 | 93396703 | 389 | 1.31 | 93 | 1.41 | 3.49 | 480 | 5.28E−04 |
| RS492681 | 2472 | | | 18 | 9853662 | 383 | 1.32 | 89 | 1.42 | 3.49 | 470 | 5.29E−04 |
| RS1945011 | 2298 | GRIK4 | 2900 | 11 | 120126812 | 395 | 1.35 | 83 | 1.25 | 3.48 | 476 | 5.48E−04 |
| RS2833581 | 2567 | HUNK | 30811 | 21 | 32254024 | 181 | 1.39 | 295 | 1.3 | 3.47 | 474 | 5.68E−04 |
| RS4122274 | 1873 | | | 5 | 102998589 | 152 | 1.39 | 320 | 1.31 | 3.47 | 470 | 5.68E−04 |
| RS10508895 | 2209 | MARCH8 | 220972 | 10 | 45287774 | 414 | 1.35 | 24 | 1.17 | 3.47 | 436 | 5.72E−04 |
| RS362865 | 2007 | GRM1 | 2911 | 6 | 146704527 | 414 | 1.35 | 64 | 1.23 | 3.45 | 476 | 6.10E−04 |
| RS4492337 | 2142 | C8orf37 | 157657 | 8 | 96336117 | 313 | 1.31 | 143 | 1.4 | 3.45 | 454 | 6.13E−04 |
| RS10487019 | 2056 | | | 7 | 87915131 | 449 | 1.32 | 31 | 1.48 | 3.44 | 478 | 6.33E−04 |
| RS261861 | 1550 | RGS7 | 6000 | 1 | 239162199 | 279 | 1.36 | 183 | 1.28 | 3.44 | 460 | 6.35E−04 |
| RS283086 | 1986 | SLC35F1 | 222553 | 6 | 118703634 | 187 | 1.3 | 207 | 1.38 | 3.44 | 392 | 6.44E−04 |
| RS2195989 | 872 | ANGPT1 | 284 | 8 | 108555088 | 69 | 1.43 | 393 | 1.32 | 3.43 | 460 | 6.58E−04 |
| RS2357344 | 1242 | SYNE2 | 23224 | 14 | 63578006 | 390 | 1.31 | 34 | 1.46 | 3.43 | 422 | 6.63E−04 |
| RS2362932 | 120 | SMYD3 | 64754 | 1 | 243980907 | 137 | 1.4 | 263 | 1.31 | 3.43 | 398 | 6.67E−04 |
| RS10506681 | 2328 | | | 12 | 73653196 | 308 | 1.3 | 172 | 1.39 | 3.42 | 478 | 6.80E−04 |
| RS1427422 | 1660 | | | 3 | 69866955 | 432 | 1.32 | 46 | 1.45 | 3.42 | 476 | 6.80E−04 |
| RS10485116 | 1946 | LOC653802 | 653802 | 6 | 42430492 | 467 | 1.33 | 9 | 1.61 | 3.41 | 474 | 7.05E−04 |
| RS665889 | 915 | | | 9 | 86972031 | 268 | 1.3 | 204 | 1.38 | 3.4 | 470 | 7.31E−04 |
| RS6109935 | 1424 | TASP1 | 55617 | 20 | 13467837 | 120 | 1.4 | 316 | 1.31 | 3.4 | 434 | 7.36E−04 |
| RS10521007 | 1822 | ADAMTS12 | 81792 | 5 | 33794319 | 383 | 1.35 | 99 | 1.26 | 3.39 | 480 | 7.57E−04 |
| RS7828096 | 2095 | PSD3 | 23362 | 8 | 18597554 | 449 | 1.32 | 31 | 1.48 | 3.38 | 478 | 7.84E−04 |
| RS1406444 | 1595 | | | 2 | 148189717 | 425 | 1.35 | 53 | 1.23 | 3.38 | 476 | 7.84E−04 |
| RS9306468 | 2570 | MTMR3 | 8897 | 22 | 28704281 | 373 | 1.35 | 103 | 1.26 | 3.38 | 474 | 7.85E−04 |
| RS1501622 | 1705 | FGF12 | 2257 | 3 | 193634069 | 187 | 1.28 | 285 | 1.36 | 3.38 | 470 | 7.85E−04 |
| RS1369592 | 2435 | | | 15 | 34185952 | 209 | 1.29 | 231 | 1.37 | 3.38 | 438 | 7.90E−04 |
| RS817504 | 1679 | | | 3 | 119161552 | 344 | 1.3 | 58 | 1.42 | 3.38 | 400 | 7.96E−04 |
| RS10517211 | 1717 | | | 4 | 30253481 | 421 | 1.32 | 61 | 1.43 | 3.37 | 480 | 8.12E−04 |
| RS950206 | 1791 | | | 4 | 116400004 | 179 | 1.28 | 297 | 1.36 | 3.37 | 474 | 8.13E−04 |
| RS10494717 | 1519 | | | 1 | 193195136 | 410 | 1.35 | 46 | 1.22 | 3.37 | 454 | 8.16E−04 |
| RS672489 | 1674 | | | 3 | 111133475 | 290 | 1.3 | 164 | 1.38 | 3.37 | 452 | 8.16E−04 |
| RS10516971 | 1769 | UNC5C | 8633 | 4 | 96560069 | 364 | 1.35 | 112 | 1.26 | 3.36 | 474 | 8.42E−04 |
| RS7874576 | 896 | | | 9 | 12600410 | 470 | 1.33 | 4 | 1.74 | 3.36 | 472 | 8.43E−04 |
| RS6902590 | 2008 | | | 6 | 148672183 | 146 | 1.39 | 326 | 1.31 | 3.36 | 470 | 8.43E−04 |
| RS2414689 | 1285 | RORA | 6095 | 15 | 59051547 | 75 | 1.4 | 189 | 1.29 | 3.37 | 262 | 8.65E−04 |
| RS580758 | 390 | | | 4 | 105790400 | 433 | 1.32 | 47 | 1.45 | 3.35 | 478 | 8.72E−04 |
| RS962592 | 476 | | | 4 | 181229107 | 474 | 1.33 | 6 | 1.67 | 3.35 | 478 | 8.72E−04 |
| RS6990997 | 867 | ZFPM2 | 23414 | 8 | 106881703 | 426 | 1.32 | 50 | 1.45 | 3.35 | 474 | 8.73E−04 |
| RS198278 | 711 | | | 7 | 24214718 | 224 | 1.29 | 236 | 1.37 | 3.35 | 458 | 8.75E−04 |
| RS4331176 | 2327 | | | 12 | 73652799 | 163 | 1.38 | 297 | 1.3 | 3.35 | 458 | 8.75E−04 |
| RS283063 | 1987 | SLC35F1 | 222553 | 6 | 118728007 | 444 | 1.32 | 36 | 1.47 | 3.34 | 478 | 9.03E−04 |
| RS4418368 | 758 | DLGAP2 | 9228 | 8 | 1585785 | 253 | 1.31 | 143 | 1.39 | 3.34 | 394 | 9.18E−04 |
| RS166861 | 2352 | | | 13 | 55557523 | 426 | 1.32 | 54 | 1.44 | 3.33 | 478 | 9.36E−04 |
| RS10510995 | 281 | | | 3 | 70525819 | 14 | 1.55 | 458 | 1.33 | 3.33 | 470 | 9.37E−04 |
| RS737911 | 2571 | MTMR3 | 8897 | 22 | 28729417 | 368 | 1.35 | 96 | 1.26 | 3.33 | 462 | 9.38E−04 |
| RS1851006 | 1278 | UNC13C | 440279 | 15 | 52691684 | 153 | 1.27 | 203 | 1.36 | 3.33 | 354 | 9.60E−04 |
| RS1467708 | 1526 | | | 1 | 203662300 | 289 | 1.33 | 39 | 1.47 | 3.33 | 326 | 9.68E−04 |
| RS1903154 | 2101 | | | 8 | 21140781 | 449 | 1.34 | 29 | 1.19 | 3.32 | 476 | 9.69E−04 |
| RS1828384 | 2335 | | | 12 | 114060113 | 248 | 1.3 | 230 | 1.37 | 3.32 | 476 | 9.69E−04 |
| RS10501811 | 2282 | | | 11 | 93309061 | 390 | 1.31 | 78 | 1.41 | 3.32 | 466 | 9.71E−04 |
| RS1871292 | 1646 | | | 3 | 35101859 | 279 | 1.34 | 39 | 1.21 | 3.33 | 316 | 9.71E−04 |
| RS1410364 | 1915 | | | 6 | 9837105 | 93 | 1.42 | 349 | 1.32 | 3.32 | 440 | 9.75E−04 |
| RS873651 | 2566 | C21orf41 | 54073 | 21 | 29703719 | 263 | 1.37 | 155 | 1.28 | 3.32 | 416 | 9.80E−04 |
| RS4564209 | 1531 | LOC128153 | 128153 | 1 | 215999639 | 312 | 1.3 | 168 | 1.38 | 3.31 | 478 | 1.00E−03 |
| RS2241577 | 1898 | KIAA1191 | 57179 | 5 | 175707471 | 398 | 1.32 | 78 | 1.42 | 3.31 | 474 | 1.00E−03 |
| RS717806 | 160 | | | 2 | 36429039 | 345 | 1.31 | 117 | 1.4 | 3.31 | 460 | 1.01E−03 |
| RS10497650 | 223 | | | 2 | 184931280 | 451 | 1.32 | 5 | 1.69 | 3.31 | 454 | 1.01E−03 |
| RS335251 | 2099 | PSD3 | 23362 | 8 | 18691618 | 146 | 1.39 | 332 | 1.31 | 3.3 | 476 | 1.04E−03 |
| RS10485494 | 2537 | CGI-09 | 51605 | 20 | 5867378 | 461 | 1.32 | 15 | 1.53 | 3.3 | 474 | 1.04E−03 |
| RS723043 | 157 | LTBP1 | 4052 | 2 | 33395291 | 249 | 1.3 | 177 | 1.38 | 3.3 | 424 | 1.05E−03 |
| RS6829390 | 338 | TEC | 7006 | 4 | 47893743 | 466 | 1.33 | 16 | 1.53 | 3.29 | 480 | 1.08E−03 |
| RS1280310 | 1485 | TCTEX1D1 | 200132 | 1 | 66989411 | 248 | 1.3 | 234 | 1.37 | 3.28 | 480 | 1.11E−03 |
| RS6919514 | 688 | UST | 10090 | 6 | 149151922 | 439 | 1.32 | 41 | 1.45 | 3.28 | 478 | 1.11E−03 |
| RS10484865 | 2011 | | | 6 | 153313545 | 452 | 1.34 | 22 | 1.16 | 3.28 | 472 | 1.11E−03 |
| RS4131954 | 2324 | | | 12 | 73597675 | 167 | 1.38 | 303 | 1.3 | 3.28 | 468 | 1.12E−03 |
| RS10521316 | 2461 | | | 16 | 53286727 | 243 | 1.3 | 223 | 1.38 | 3.28 | 464 | 1.12E−03 |
| RS1298362 | 1136 | | | 12 | 74493708 | 216 | 1.29 | 242 | 1.36 | 3.28 | 456 | 1.12E−03 |
| RS32218 | 1876 | FBN2 | 2201 | 5 | 127719132 | 357 | 1.32 | 93 | 1.41 | 3.28 | 448 | 1.12E−03 |
| RS10508158 | 2371 | | | 13 | 105045125 | 309 | 1.35 | 139 | 1.27 | 3.28 | 446 | 1.12E−03 |
| RS2060180 | 2018 | SDK1 | 221935 | 7 | 3696083 | 347 | 1.31 | 93 | 1.41 | 3.28 | 438 | 1.12E−03 |
| RS10508903 | 983 | MAPK8 | 5599 | 10 | 49311338 | 208 | 1.39 | 212 | 1.3 | 3.28 | 418 | 1.13E−03 |
| RS1919447 | 1593 | | | 2 | 148179334 | 420 | 1.35 | 52 | 1.23 | 3.27 | 470 | 1.15E−03 |
| RS2018842 | 1696 | | | 3 | 148362044 | 279 | 1.36 | 193 | 1.29 | 3.27 | 470 | 1.15E−03 |
| RS1519684 | 1983 | | | 6 | 114571663 | 174 | 1.38 | 296 | 1.3 | 3.27 | 468 | 1.15E−03 |
| RS1374028 | 1825 | PDE4D | 5144 | 5 | 58611588 | 398 | 1.34 | 34 | 1.19 | 3.27 | 430 | 1.16E−03 |
| RS637225 | 1517 | | | 1 | 192302414 | 207 | 1.38 | 219 | 1.3 | 3.27 | 424 | 1.16E−03 |
| RS763553 | 2112 | | | 8 | 31565038 | 232 | 1.3 | 116 | 1.39 | 3.27 | 346 | 1.18E−03 |
| RS2291386 | 2013 | SYTL3 | 94120 | 6 | 159104495 | 456 | 1.32 | 26 | 1.49 | 3.26 | 480 | 1.19E−03 |
| RS4350286 | 2217 | | | 10 | 49997106 | 140 | 1.39 | 336 | 1.31 | 3.26 | 474 | 1.19E−03 |
| RS1542731 | 1521 | | | 1 | 193349361 | 45 | 1.22 | 433 | 1.34 | 3.25 | 476 | 1.24E−03 |
| RS4611835 | 313 | NAALADL2 | 254827 | 3 | 176610769 | 444 | 1.33 | 22 | 1.5 | 3.25 | 464 | 1.24E−03 |
| RS10497649 | 1611 | | | 2 | 184930781 | 475 | 1.33 | 7 | 1.63 | 3.24 | 480 | 1.28E−03 |
| RS965158 | 868 | | | 8 | 107564094 | 471 | 1.33 | 11 | 1.57 | 3.24 | 480 | 1.28E−03 |
| RS2916 | 1484 | TCTEX1D1 | 200132 | 1 | 66989285 | 245 | 1.3 | 233 | 1.37 | 3.24 | 476 | 1.28E−03 |
| RS10495390 | 116 | RYR2 | 6262 | 1 | 235379636 | 432 | 1.32 | 44 | 1.45 | 3.24 | 474 | 1.28E−03 |
| RS548987 | 629 | SLC17A3 | 10786 | 6 | 25977350 | 442 | 1.32 | 34 | 1.46 | 3.24 | 474 | 1.28E−03 |
| RS6966318 | 2060 | IFRD1 | 3475 | 7 | 111880093 | 149 | 1.28 | 321 | 1.35 | 3.24 | 468 | 1.28E−03 |
| RS4510935 | 945 | | | 9 | 119325172 | 408 | 1.32 | 72 | 1.42 | 3.23 | 478 | 1.32E−03 |
| RS7838059 | 2083 | MSRA | 4482 | 8 | 10216220 | 387 | 1.32 | 53 | 1.44 | 3.23 | 438 | 1.33E−03 |
| RS2060184 | 2017 | SDK1 | 221935 | 7 | 3695551 | 374 | 1.31 | 108 | 1.4 | 3.22 | 480 | 1.37E−03 |
| RS796707 | 1979 | ATG5 | 9474 | 6 | 106800214 | 440 | 1.32 | 40 | 1.45 | 3.22 | 478 | 1.37E−03 |
| RS1497724 | 1714 | | | 4 | 27838505 | 265 | 1.36 | 213 | 1.29 | 3.22 | 476 | 1.37E−03 |
| RS7809161 | 2023 | DGKB | 1607 | 7 | 14739036 | 64 | 1.24 | 414 | 1.35 | 3.22 | 476 | 1.37E−03 |
| RS1416824 | 1548 | | | 1 | 237505708 | 436 | 1.34 | 38 | 1.21 | 3.22 | 472 | 1.37E−03 |
| RS10518900 | 2440 | C15orf41 | 84529 | 15 | 34807277 | 271 | 1.3 | 203 | 1.38 | 3.22 | 472 | 1.37E−03 |
| RS1905214 | 1902 | | | 6 | 6019201 | 187 | 1.29 | 285 | 1.36 | 3.22 | 470 | 1.37E−03 |
| RS10510809 | 274 | | | 3 | 59414093 | 386 | 1.32 | 78 | 1.42 | 3.22 | 462 | 1.37E−03 |
| RS661592 | 2040 | C7orf25 | 79020 | 7 | 42919011 | 428 | 1.35 | 34 | 1.21 | 3.22 | 460 | 1.37E−03 |
| RS1708370 | 1694 | TMEM108 | 66000 | 3 | 134499004 | 376 | 1.32 | 56 | 1.44 | 3.22 | 430 | 1.38E−03 |
| RS2198683 | 2513 | | | 18 | 56636476 | 196 | 1.29 | 262 | 1.37 | 3.21 | 456 | 1.42E−03 |
| RS989177 | 748 | | | 7 | 124200122 | 66 | 1.42 | 350 | 1.32 | 3.21 | 414 | 1.43E−03 |
| RS7664158 | 1792 | | | 4 | 117482746 | 406 | 1.32 | 74 | 1.42 | 3.2 | 478 | 1.47E−03 |
| RS1211435 | 2460 | | | 16 | 53138814 | 326 | 1.31 | 148 | 1.39 | 3.2 | 472 | 1.47E−03 |
| RS507575 | 1995 | TPD52L1 | 7164 | 6 | 125559665 | 424 | 1.32 | 42 | 1.45 | 3.2 | 464 | 1.47E−03 |
| RS7845961 | 2100 | | | 8 | 19783125 | 208 | 1.37 | 274 | 1.3 | 3.19 | 480 | 1.52E−03 |
| RS4692297 | 1713 | | | 4 | 27837884 | 266 | 1.37 | 214 | 1.29 | 3.19 | 478 | 1.52E−03 |
| RS3218493 | 2069 | XRCC2 | 7516 | 7 | 151984596 | 470 | 1.33 | 10 | 1.58 | 3.19 | 478 | 1.52E−03 |
| RS2294311 | 1457 | HSPC117 | 51493 | 22 | 31123269 | 357 | 1.31 | 121 | 1.39 | 3.19 | 476 | 1.52E−03 |
| RS6987277 | 766 | | | 8 | 8977241 | 436 | 1.32 | 38 | 1.46 | 3.19 | 472 | 1.52E−03 |
| RS7775656 | 1913 | TXNDC5 | 81567 | 6 | 7954759 | 388 | 1.35 | 76 | 1.25 | 3.19 | 462 | 1.52E−03 |
| RS918092 | 2306 | GRIN2B | 2904 | 12 | 13632070 | 372 | 1.31 | 86 | 1.4 | 3.19 | 456 | 1.52E−03 |
| RS1252225 | 854 | LOC138046 | 138046 | 8 | 85677664 | 284 | 1.31 | 98 | 1.4 | 3.19 | 380 | 1.54E−03 |
| RS10509173 | 2220 | | | 10 | 64281895 | 179 | 1.38 | 185 | 1.3 | 3.19 | 362 | 1.55E−03 |
| RS10514280 | 1854 | | | 5 | 85744155 | 455 | 1.34 | 23 | 1.17 | 3.18 | 476 | 1.57E−03 |
| RS1810113 | 2153 | TM7SF4 | 81501 | 8 | 105433770 | 323 | 1.31 | 155 | 1.39 | 3.18 | 476 | 1.57E−03 |
| RS1290030 | 2330 | | | 12 | 74498851 | 215 | 1.37 | 261 | 1.3 | 3.18 | 474 | 1.57E−03 |
| RS1861373 | 1150 | | | 12 | 96296999 | 204 | 1.29 | 272 | 1.37 | 3.18 | 474 | 1.57E−03 |
| RS9296009 | 1938 | | | 6 | 32222493 | 331 | 1.36 | 143 | 1.28 | 3.18 | 472 | 1.57E−03 |
| RS773013 | 2188 | | | 9 | 76973237 | 459 | 1.33 | 9 | 1.59 | 3.18 | 466 | 1.57E−03 |
| RS4703141 | 1855 | | | 5 | 97943067 | 289 | 1.3 | 141 | 1.38 | 3.18 | 428 | 1.58E−03 |
| RS1121922 | 2093 | PSD3 | 23362 | 8 | 18563312 | 432 | 1.32 | 50 | 1.44 | 3.17 | 480 | 1.62E−03 |
| RS10509698 | 1008 | C10orf130 | 387707 | 10 | 97782342 | 466 | 1.33 | 14 | 1.54 | 3.17 | 478 | 1.62E−03 |
| RS178462 | 2421 | | | 14 | 79520688 | 48 | 1.44 | 430 | 1.32 | 3.17 | 476 | 1.62E−03 |
| RS1046852 | 20 | C1orf139 | 79971 | 1 | 68363692 | 287 | 1.3 | 185 | 1.38 | 3.17 | 470 | 1.62E−03 |
| RS972432 | 2098 | PSD3 | 23362 | 8 | 18599317 | 444 | 1.32 | 28 | 1.46 | 3.17 | 470 | 1.62E−03 |
| RS2111368 | 2341 | | | 12 | 126098472 | 401 | 1.31 | 67 | 1.42 | 3.17 | 466 | 1.62E−03 |
| RS10503998 | 2113 | | | 8 | 37046107 | 410 | 1.32 | 46 | 1.44 | 3.17 | 454 | 1.63E−03 |
| RS1901439 | 204 | | | 2 | 134153941 | 425 | 1.32 | 55 | 1.43 | 3.16 | 478 | 1.68E−03 |
| RS6699989 | 44 | | | 1 | 87844970 | 75 | 1.42 | 399 | 1.32 | 3.16 | 472 | 1.68E−03 |
| RS10488861 | 1771 | | | 4 | 105845763 | 430 | 1.32 | 44 | 1.44 | 3.16 | 472 | 1.68E−03 |
| RS10504143 | 2121 | RB1CC1 | 9821 | 8 | 53714280 | 446 | 1.33 | 20 | 1.51 | 3.16 | 464 | 1.68E−03 |
| RS3874273 | 2434 | | | 15 | 34183438 | 290 | 1.31 | 172 | 1.38 | 3.16 | 460 | 1.68E−03 |
| RS10490347 | 1566 | FLJ10379 | 55133 | 2 | 45548444 | 434 | 1.34 | 48 | 1.23 | 3.15 | 480 | 1.73E−03 |
| RS10502475 | 1376 | KCTD1 | 284252 | 18 | 22427942 | 174 | 1.38 | 308 | 1.31 | 3.15 | 480 | 1.73E−03 |
| RS10517210 | 1716 | | | 4 | 30253178 | 423 | 1.32 | 57 | 1.43 | 3.15 | 478 | 1.74E−03 |
| RS10498944 | 1968 | | | 6 | 85414678 | 438 | 1.32 | 42 | 1.45 | 3.15 | 478 | 1.74E−03 |
| RS10483457 | 1232 | GARNL1 | 253959 | 14 | 35211683 | 447 | 1.32 | 33 | 1.47 | 3.15 | 478 | 1.74E−03 |
| RS10518178 | 1762 | | | 4 | 78638707 | 417 | 1.32 | 59 | 1.43 | 3.15 | 474 | 1.74E−03 |
| RS543465 | 1980 | ATG5 | 9474 | 6 | 106855297 | 438 | 1.33 | 38 | 1.46 | 3.15 | 474 | 1.74E−03 |
| RS10517562 | 436 | | | 4 | 153255193 | 160 | 1.28 | 314 | 1.36 | 3.15 | 472 | 1.74E−03 |
| RS8089476 | 1392 | | | 18 | 46107418 | 175 | 1.29 | 297 | 1.36 | 3.15 | 470 | 1.74E−03 |
| RS1473869 | 1699 | CCDC50 | 152137 | 3 | 192542016 | 446 | 1.34 | 24 | 1.18 | 3.15 | 468 | 1.74E−03 |
| RS10494722 | 1520 | | | 1 | 193329773 | 37 | 1.21 | 429 | 1.34 | 3.15 | 464 | 1.74E−03 |
| RS223128 | 1337 | | | 17 | 26933541 | 309 | 1.31 | 157 | 1.39 | 3.15 | 464 | 1.74E−03 |
| RS10486853 | 2058 | FLJ36166 | 349152 | 7 | 102642309 | 419 | 1.32 | 45 | 1.44 | 3.15 | 462 | 1.74E−03 |
| RS2353193 | 1607 | | | 2 | 170142309 | 393 | 1.32 | 87 | 1.41 | 3.14 | 478 | 1.79E−03 |
| RS6945746 | 2016 | SDK1 | 221935 | 7 | 3548845 | 411 | 1.32 | 65 | 1.42 | 3.14 | 474 | 1.79E−03 |
| RS10494716 | 1518 | | | 1 | 193182462 | 53 | 1.23 | 421 | 1.34 | 3.14 | 472 | 1.80E−03 |
| RS1025302 | 1875 | | | 5 | 120843420 | 384 | 1.31 | 90 | 1.41 | 3.14 | 472 | 1.80E−03 |
| RS2248815 | 1453 | ITSN1 | 6453 | 21 | 34060196 | 377 | 1.35 | 55 | 1.24 | 3.14 | 430 | 1.81E−03 |
| RS10493274 | 1481 | | | 1 | 60204577 | 414 | 1.32 | 16 | 1.52 | 3.14 | 428 | 1.81E−03 |
| RS10504433 | 824 | | | 8 | 70021434 | 430 | 1.32 | 52 | 1.43 | 3.13 | 480 | 1.85E−03 |
| RS7706518 | 576 | | | 5 | 127308921 | 432 | 1.32 | 48 | 1.44 | 3.13 | 478 | 1.86E−03 |
| RS10510271 | 1629 | | | 3 | 3546367 | 326 | 1.31 | 152 | 1.38 | 3.13 | 476 | 1.86E−03 |
| RS717357 | 1709 | | | 4 | 13994312 | 341 | 1.31 | 137 | 1.39 | 3.13 | 476 | 1.86E−03 |
| RS4673999 | 1612 | | | 2 | 216058272 | 222 | 1.29 | 252 | 1.36 | 3.13 | 472 | 1.86E−03 |
| RS1290939 | 2418 | RAD51L1 | 5890 | 14 | 67759566 | 399 | 1.32 | 55 | 1.43 | 3.13 | 452 | 1.86E−03 |
| RS2662258 | 1859 | RGMB | 285704 | 5 | 98153430 | 294 | 1.31 | 138 | 1.39 | 3.13 | 430 | 1.87E−03 |
| RS723981 | 1967 | SH3BGRL2 | 83699 | 6 | 80421994 | 420 | 1.32 | 6 | 1.65 | 3.13 | 424 | 1.87E−03 |
| RS10503344 | 2075 | | | 8 | 5804694 | 341 | 1.33 | 49 | 1.45 | 3.13 | 388 | 1.88E−03 |
| RS7701735 | 1853 | | | 5 | 85719306 | 457 | 1.34 | 25 | 1.18 | 3.12 | 480 | 1.92E−03 |
| RS958425 | 2204 | C10orf115 | 387642 | 10 | 23551932 | 418 | 1.32 | 62 | 1.43 | 3.12 | 478 | 1.92E−03 |
| RS1578975 | 1805 | | | 4 | 170533173 | 444 | 1.34 | 32 | 1.2 | 3.12 | 474 | 1.92E−03 |
| RS4631686 | 3 | | | 1 | 14450061 | 464 | 1.33 | 8 | 1.61 | 3.12 | 470 | 1.92E−03 |
| RS7798413 | 2039 | | | 7 | 40879048 | 398 | 1.31 | 72 | 1.41 | 3.12 | 468 | 1.92E−03 |
| RS10504166 | 2123 | LOC392222 | 392222 | 8 | 55556364 | 399 | 1.32 | 65 | 1.42 | 3.12 | 462 | 1.92E−03 |
| RS2409037 | 577 | | | 5 | 127314089 | 430 | 1.32 | 48 | 1.44 | 3.11 | 476 | 1.98E−03 |
| RS785039 | 1574 | | | 2 | 115087031 | 435 | 1.32 | 41 | 1.45 | 3.11 | 474 | 1.98E−03 |
| RS1314913 | 2419 | RAD51L1 | 5890 | 14 | 67769347 | 415 | 1.32 | 57 | 1.43 | 3.11 | 470 | 1.98E−03 |
| RS2769261 | 1504 | | | 1 | 113368430 | 340 | 1.32 | 80 | 1.42 | 3.11 | 418 | 2.00E−03 |
| RS10518896 | 2439 | C15orf41 | 84529 | 15 | 34731373 | 467 | 1.33 | 13 | 1.54 | 3.1 | 478 | 2.05E−03 |
| RS10510986 | 1655 | | | 3 | 69746452 | 430 | 1.32 | 48 | 1.44 | 3.1 | 476 | 2.05E−03 |
| RS7691071 | 1798 | | | 4 | 126835433 | 194 | 1.37 | 284 | 1.3 | 3.1 | 476 | 2.05E−03 |
| RS889053 | 1888 | | | 5 | 159028256 | 377 | 1.35 | 99 | 1.26 | 3.1 | 474 | 2.05E−03 |
| RS3218491 | 753 | XRCC2 | 7516 | 7 | 151984681 | 466 | 1.33 | 6 | 1.64 | 3.1 | 470 | 2.05E−03 |
| RS2034531 | 1799 | | | 4 | 137364060 | 23 | 1.49 | 447 | 1.33 | 3.1 | 468 | 2.05E−03 |
| RS1154307 | 550 | | | 5 | 111049411 | 409 | 1.33 | 41 | 1.45 | 3.1 | 448 | 2.06E−03 |
| RS10516627 | 1793 | | | 4 | 117487171 | 56 | 1.42 | 392 | 1.32 | 3.1 | 446 | 2.06E−03 |
| RS10487544 | 2068 | | | 7 | 143266616 | 284 | 1.35 | 164 | 1.28 | 3.1 | 446 | 2.06E−03 |
| RS1471531 | 2120 | | | 8 | 43226199 | 380 | 1.35 | 62 | 1.24 | 3.1 | 440 | 2.06E−03 |
| RS10494231 | 1505 | | | 1 | 120125842 | 220 | 1.29 | 184 | 1.37 | 3.1 | 402 | 2.07E−03 |
| RS1480145 | 796 | | | 8 | 16181494 | 470 | 1.33 | 12 | 1.55 | 3.09 | 480 | 2.12E−03 |
| RS370480 | 2353 | | | 13 | 55575987 | 154 | 1.38 | 328 | 1.31 | 3.09 | 480 | 2.12E−03 |
| RS10521210 | 1335 | | | 17 | 12966928 | 226 | 1.37 | 250 | 1.3 | 3.09 | 474 | 2.12E−03 |
| RS757668 | 744 | | | 7 | 122368820 | 180 | 1.38 | 292 | 1.31 | 3.09 | 470 | 2.12E−03 |
| RS2195875 | 2022 | | | 7 | 13707083 | 414 | 1.34 | 44 | 1.22 | 3.09 | 456 | 2.12E−03 |
| RS10508705 | 2205 | | | 10 | 26117594 | 413 | 1.32 | 43 | 1.44 | 3.09 | 454 | 2.12E−03 |
| RS10484171 | 2412 | | | 14 | 44213271 | 222 | 1.36 | 94 | 1.26 | 3.09 | 314 | 2.18E−03 |
| RS7002137 | 769 | | | 8 | 10653610 | 413 | 1.32 | 69 | 1.42 | 3.08 | 480 | 2.19E−03 |
| RS10491068 | 1011 | BLNK | 29760 | 10 | 97946892 | 467 | 1.33 | 15 | 1.53 | 3.08 | 480 | 2.19E−03 |
| RS10514614 | 2464 | | | 16 | 84837699 | 442 | 1.32 | 36 | 1.46 | 3.08 | 476 | 2.19E−03 |
| RS1110581 | 1568 | | | 2 | 66930615 | 401 | 1.32 | 75 | 1.41 | 3.08 | 474 | 2.19E−03 |
| RS998359 | 1777 | NDST4 | 64579 | 4 | 116090480 | 345 | 1.31 | 131 | 1.39 | 3.08 | 474 | 2.19E−03 |
| RS1889465 | 1985 | SLC35F1 | 222553 | 6 | 118649503 | 444 | 1.32 | 32 | 1.47 | 3.08 | 474 | 2.19E−03 |
| RS2448361 | 2219 | CDC2 | 983 | 10 | 62223133 | 402 | 1.32 | 72 | 1.42 | 3.08 | 472 | 2.19E−03 |
| RS2123769 | 1557 | LTBP1 | 4052 | 2 | 33378911 | 313 | 1.35 | 155 | 1.28 | 3.08 | 466 | 2.19E−03 |
| RS183628 | 1856 | | | 5 | 98007391 | 325 | 1.31 | 143 | 1.39 | 3.08 | 466 | 2.19E−03 |
| RS2485488 | 2345 | | | 13 | 37441039 | 101 | 1.26 | 365 | 1.35 | 3.08 | 464 | 2.19E−03 |
| RS10486529 | 2034 | PTHB1 | 27241 | 7 | 33363277 | 385 | 1.35 | 75 | 1.25 | 3.08 | 458 | 2.19E−03 |
| RS6837953 | 437 | | | 4 | 154150413 | 415 | 1.32 | 35 | 1.45 | 3.08 | 448 | 2.20E−03 |
| RS3749216 | 1650 | FAM19A1 | 407738 | 3 | 68548925 | 478 | 1.33 | 4 | 1.71 | 3.07 | 480 | 2.26E−03 |
| RS1883736 | 2248 | ABTB2 | 25841 | 11 | 34330019 | 466 | 1.33 | 14 | 1.53 | 3.07 | 478 | 2.26E−03 |
| RS2167871 | 2044 | | | 7 | 61715567 | 427 | 1.32 | 51 | 1.43 | 3.07 | 476 | 2.26E−03 |
| RS10505768 | 1112 | | | 12 | 12790297 | 107 | 1.27 | 371 | 1.35 | 3.07 | 476 | 2.26E−03 |
| RS1433450 | 1293 | | | 15 | 85342066 | 433 | 1.34 | 45 | 1.23 | 3.07 | 476 | 2.26E−03 |
| RS1979980 | 1858 | RGMB | 285704 | 5 | 98134967 | 339 | 1.31 | 135 | 1.39 | 3.07 | 472 | 2.26E−03 |
| RS6955211 | 2050 | | | 7 | 63509775 | 175 | 1.38 | 291 | 1.31 | 3.07 | 464 | 2.27E−03 |
| RS7765062 | 2012 | | | 6 | 153316119 | 26 | 1.19 | 436 | 1.34 | 3.07 | 460 | 2.27E−03 |
| RS10226709 | 708 | DGKB | 1607 | 7 | 14764159 | 364 | 1.35 | 68 | 1.25 | 3.07 | 430 | 2.28E−03 |
| RS745708 | 976 | FRMPD2 | 143162 | 10 | 49115681 | 351 | 1.31 | 75 | 1.41 | 3.07 | 424 | 2.28E−03 |
| RS2138537 | 1701 | | | 3 | 193034173 | 221 | 1.37 | 261 | 1.3 | 3.06 | 480 | 2.34E−03 |
| RS10500720 | 2237 | SBF2 | 81846 | 11 | 10159532 | 467 | 1.34 | 15 | 1.14 | 3.06 | 480 | 2.34E−03 |
| RS10500723 | 2238 | SBF2 | 81846 | 11 | 10212811 | 467 | 1.34 | 15 | 1.14 | 3.06 | 480 | 2.34E−03 |
| RS10497472 | 1609 | | | 2 | 177492847 | 450 | 1.33 | 30 | 1.47 | 3.06 | 478 | 2.34E−03 |
| RS9325124 | 1887 | | | 5 | 148229011 | 296 | 1.31 | 184 | 1.38 | 3.06 | 478 | 2.34E−03 |
| RS3823290 | 1982 | FLJ10159 | 55084 | 6 | 108026930 | 438 | 1.32 | 42 | 1.45 | 3.06 | 478 | 2.34E−03 |
| RS10509759 | 2226 | CNNM2 | 54805 | 10 | 104679655 | 453 | 1.32 | 27 | 1.48 | 3.06 | 478 | 2.34E−03 |
| RS10514844 | 1182 | | | 13 | 37808480 | 471 | 1.33 | 7 | 1.62 | 3.06 | 476 | 2.34E−03 |
| RS346421 | 527 | | | 5 | 62565739 | 429 | 1.32 | 47 | 1.44 | 3.06 | 474 | 2.34E−03 |
| RS7339969 | 1516 | C1orf27 | 54953 | 1 | 184648393 | 156 | 1.39 | 318 | 1.31 | 3.06 | 472 | 2.34E−03 |
| RS6427753 | 1524 | | | 1 | 197069037 | 322 | 1.31 | 152 | 1.38 | 3.06 | 472 | 2.34E−03 |
| RS10510987 | 1656 | | | 3 | 69785447 | 430 | 1.32 | 42 | 1.44 | 3.06 | 470 | 2.34E−03 |
| RS10518211 | 365 | | | 4 | 80156089 | 253 | 1.36 | 219 | 1.29 | 3.06 | 470 | 2.34E−03 |
| RS2163721 | 2333 | PPFIA2 | 8499 | 12 | 80343092 | 463 | 1.34 | 9 | 1.09 | 3.06 | 470 | 2.34E−03 |
| RS10486530 | 2035 | PTHB1 | 27241 | 7 | 33364558 | 385 | 1.35 | 77 | 1.25 | 3.06 | 460 | 2.34E−03 |
| RS10501990 | 2290 | | | 11 | 101172368 | 290 | 1.31 | 140 | 1.39 | 3.06 | 428 | 2.35E−03 |
| RS1465135 | 697 | | | 6 | 164115617 | 314 | 1.3 | 114 | 1.38 | 3.06 | 426 | 2.35E−03 |
| RS1905015 | 1668 | | | 3 | 100210226 | 180 | 1.38 | 298 | 1.31 | 3.05 | 476 | 2.42E−03 |
| RS947271 | 1507 | REG4 | 83998 | 1 | 120138926 | 258 | 1.3 | 218 | 1.37 | 3.05 | 474 | 2.42E−03 |
| RS10484926 | 2006 | | | 6 | 146336371 | 470 | 1.33 | 4 | 1.71 | 3.05 | 472 | 2.42E−03 |
| RS1805781 | 1111 | PHC1 | 1911 | 12 | 8957155 | 468 | 1.33 | 6 | 1.64 | 3.05 | 472 | 2.42E−03 |
| RS7234279 | 2509 | | | 18 | 55200765 | 428 | 1.34 | 46 | 1.23 | 3.05 | 472 | 2.42E−03 |
| RS6895637 | 1821 | ADAMTS12 | 81792 | 5 | 33785569 | 319 | 1.35 | 141 | 1.28 | 3.05 | 458 | 2.42E−03 |
| RS10499134 | 2000 | | | 6 | 126479722 | 426 | 1.34 | 30 | 1.2 | 3.05 | 454 | 2.42E−03 |
| RS2785949 | 1195 | | | 13 | 53806676 | 316 | 1.31 | 138 | 1.39 | 3.05 | 452 | 2.42E−03 |
| RS26207 | 1877 | ADAMTS19 | 171019 | 5 | 129063995 | 260 | 1.36 | 138 | 1.28 | 3.05 | 396 | 2.44E−03 |
| RS10510104 | 1030 | ATE1 | 11101 | 10 | 123521812 | 394 | 1.32 | 88 | 1.41 | 3.04 | 480 | 2.50E−03 |
| RS10500719 | 2236 | SBF2 | 81846 | 11 | 10146418 | 465 | 1.34 | 15 | 1.14 | 3.04 | 478 | 2.50E−03 |
| RS260933 | 1715 | | | 4 | 28982993 | 261 | 1.36 | 215 | 1.29 | 3.04 | 474 | 2.50E−03 |
| RS7447076 | 1872 | | | 5 | 100585396 | 134 | 1.28 | 342 | 1.36 | 3.04 | 474 | 2.50E−03 |
| RS6079097 | 2542 | TASP1 | 55617 | 20 | 13474577 | 139 | 1.38 | 337 | 1.31 | 3.04 | 474 | 2.50E−03 |
| RS6601146 | 1899 | LOC646210 | 646210 | 5 | 180226111 | 127 | 1.28 | 345 | 1.35 | 3.04 | 470 | 2.50E−03 |
| RS9307159 | 1770 | UNC5C | 8633 | 4 | 96560909 | 391 | 1.35 | 79 | 1.26 | 3.04 | 468 | 2.50E−03 |
| RS2527729 | 2074 | | | 8 | 5376923 | 130 | 1.27 | 334 | 1.35 | 3.04 | 462 | 2.50E−03 |
| RS1268444 | 1937 | | | 6 | 30519525 | 277 | 1.31 | 157 | 1.38 | 3.04 | 432 | 2.51E−03 |
| RS7573989 | 1569 | | | 2 | 66974299 | 414 | 1.32 | 68 | 1.42 | 3.03 | 480 | 2.58E−03 |
| RS10509705 | 1010 | | | 10 | 97914784 | 468 | 1.33 | 14 | 1.53 | 3.03 | 480 | 2.58E−03 |
| RS1079634 | 2462 | WWOX | 51741 | 16 | 77468635 | 363 | 1.31 | 119 | 1.39 | 3.03 | 480 | 2.58E−03 |
| RS10494583 | 1514 | TPR | 7175 | 1 | 184557118 | 385 | 1.32 | 95 | 1.4 | 3.03 | 478 | 2.58E−03 |
| RS2291385 | 2014 | SYTL3 | 94120 | 6 | 159104665 | 455 | 1.32 | 25 | 1.48 | 3.03 | 478 | 2.58E−03 |
| RS2743562 | 1978 | ATG5 | 9474 | 6 | 106799841 | 437 | 1.32 | 37 | 1.45 | 3.03 | 472 | 2.58E−03 |
| RS2043835 | 1874 | | | 5 | 111010485 | 434 | 1.32 | 48 | 1.44 | 3.02 | 480 | 2.66E−03 |
| RS4504494 | 2001 | ARHGAP18 | 93663 | 6 | 129954780 | 469 | 1.33 | 13 | 1.54 | 3.02 | 480 | 2.66E−03 |
| RS6949530 | 2062 | | | 7 | 122406461 | 411 | 1.32 | 71 | 1.42 | 3.02 | 480 | 2.66E−03 |
| RS10512000 | 2186 | | | 9 | 73345993 | 37 | 1.22 | 445 | 1.34 | 3.02 | 480 | 2.66E−03 |
| RS10492135 | 1114 | GRIN2B | 2904 | 12 | 13777535 | 105 | 1.4 | 375 | 1.31 | 3.02 | 478 | 2.66E−03 |
| RS1443120 | 2296 | | | 11 | 109749438 | 441 | 1.32 | 37 | 1.45 | 3.02 | 476 | 2.66E−03 |
| RS854404 | 2373 | | | 14 | 24273746 | 309 | 1.31 | 169 | 1.38 | 3.02 | 476 | 2.66E−03 |
| RS2825681 | 2562 | | | 21 | 19910552 | 415 | 1.32 | 61 | 1.43 | 3.02 | 474 | 2.66E−03 |
| RS10493409 | 1483 | SGIP1 | 84251 | 1 | 66778534 | 408 | 1.32 | 64 | 1.42 | 3.02 | 470 | 2.67E−03 |
| RS9306281 | 2573 | LARGE | 9215 | 22 | 32343807 | 127 | 1.39 | 343 | 1.31 | 3.02 | 468 | 2.67E−03 |
| RS10484230 | 1236 | | | 14 | 43700947 | 365 | 1.31 | 101 | 1.4 | 3.02 | 464 | 2.67E−03 |
| RS1993306 | 1555 | LTBP1 | 4052 | 2 | 33299839 | 361 | 1.35 | 99 | 1.26 | 3.02 | 458 | 2.67E−03 |
| RS7752672 | 1999 | | | 6 | 125629122 | 185 | 1.38 | 273 | 1.31 | 3.02 | 456 | 2.67E−03 |
| RS547595 | 1503 | SLC25A24 | 29957 | 1 | 108527709 | 173 | 1.29 | 277 | 1.36 | 3.02 | 448 | 2.67E−03 |
| RS1526595 | 1648 | CACNA2D3 | 55799 | 3 | 54903536 | 320 | 1.31 | 126 | 1.39 | 3.02 | 444 | 2.67E−03 |
| RS2458349 | 1105 | | | 11 | 105805096 | 204 | 1.38 | 240 | 1.31 | 3.02 | 442 | 2.67E−03 |
| RS2405708 | 428 | | | 4 | 137201172 | 161 | 1.38 | 223 | 1.3 | 3.02 | 382 | 2.70E−03 |
| RS9285429 | 1988 | | | 6 | 118811259 | 307 | 1.36 | 175 | 1.29 | 3.01 | 480 | 2.75E−03 |
| RS1324088 | 628 | | | 6 | 25949101 | 444 | 1.32 | 36 | 1.45 | 3.01 | 478 | 2.75E−03 |
| RS1914542 | 2233 | | | 10 | 125651321 | 174 | 1.38 | 306 | 1.31 | 3.01 | 478 | 2.75E−03 |
| RS1945627 | 2245 | LOC387755 | 387755 | 11 | 15182763 | 453 | 1.32 | 27 | 1.47 | 3.01 | 478 | 2.75E−03 |
| RS10503572 | 789 | | | 8 | 16131101 | 473 | 1.33 | 5 | 1.67 | 3.01 | 476 | 2.75E−03 |
| RS539111 | 1961 | BAI3 | 577 | 6 | 69638536 | 269 | 1.36 | 207 | 1.3 | 3.01 | 474 | 2.75E−03 |
| RS717997 | 2334 | | | 12 | 82658105 | 292 | 1.31 | 178 | 1.38 | 3.01 | 468 | 2.75E−03 |
| RS787433 | 1590 | | | 2 | 145580328 | 260 | 1.31 | 200 | 1.38 | 3.01 | 458 | 2.76E−03 |
| RS2124726 | 1549 | FMN2 | 56776 | 1 | 238431700 | 301 | 1.36 | 157 | 1.29 | 3.01 | 456 | 2.76E−03 |
| RS10514136 | 1849 | AP3B1 | 8546 | 5 | 77467642 | 376 | 1.34 | 48 | 1.23 | 3.01 | 422 | 2.77E−03 |
| RS2470549 | 1635 | ANKRD28 | 23243 | 3 | 15712702 | 206 | 1.34 | 134 | 1.27 | 3.01 | 338 | 2.81E−03 |
| RS2591651 | 1826 | PDE4D | 5144 | 5 | 58615479 | 477 | 1.33 | 5 | 1.66 | 3 | 480 | 2.84E−03 |
| RS10519569 | 2430 | LOC653227 | 653227 | 15 | 24902890 | 412 | 1.32 | 70 | 1.42 | 3 | 480 | 2.84E−03 |
| RS10519571 | 2429 | LOC653227 | 653227 | 15 | 24899931 | 409 | 1.32 | 71 | 1.41 | 3 | 478 | 2.84E−03 |
| RS10495466 | 118 | FMN2 | 56776 | 1 | 238575190 | 458 | 1.33 | 20 | 1.5 | 3 | 476 | 2.84E−03 |
| RS7688920 | 1800 | | | 4 | 146812432 | 447 | 1.34 | 31 | 1.2 | 3 | 476 | 2.84E−03 |
| RS3924594 | 2329 | CAPS2 | 84698 | 12 | 73969063 | 190 | 1.37 | 284 | 1.3 | 3 | 472 | 2.84E−03 |
| RS2817738 | 1927 | | | 6 | 24899079 | 51 | 1.44 | 411 | 1.32 | 3 | 460 | 2.85E−03 |
| RS726789 | 2532 | EMR3 | 84658 | 19 | 14604003 | 292 | 1.36 | 170 | 1.28 | 3 | 460 | 2.85E−03 |
| RS6601438 | 2084 | MSRA | 4482 | 8 | 10217077 | 385 | 1.32 | 73 | 1.41 | 3 | 456 | 2.85E−03 |
| RS2301448 | 2574 | | | 22 | 44417456 | 144 | 1.39 | 274 | 1.31 | 3 | 416 | 2.86E−03 |
| RS9323682 | 2422 | | | 14 | 79536316 | 436 | 1.32 | 40 | 1.45 | 2.99 | 474 | 2.93E−03 |
| RS8099275 | 2511 | | | 18 | 56551281 | 214 | 1.3 | 262 | 1.36 | 2.99 | 474 | 2.93E−03 |
| RS4695371 | 1736 | | | 4 | 48018575 | 408 | 1.31 | 62 | 1.41 | 2.99 | 468 | 2.94E−03 |
| RS10515792 | 1889 | | | 5 | 159028460 | 369 | 1.35 | 101 | 1.27 | 2.99 | 468 | 2.94E−03 |
| RS1604613 | 1197 | | | 13 | 69021604 | 210 | 1.37 | 246 | 1.3 | 2.99 | 454 | 2.94E−03 |
| RS1579274 | 2119 | | | 8 | 41778080 | 349 | 1.31 | 129 | 1.39 | 2.98 | 476 | 3.03E−03 |
| RS10506626 | 1128 | TSPAN8 | 7103 | 12 | 69815758 | 328 | 1.31 | 148 | 1.39 | 2.98 | 474 | 3.03E−03 |
| RS779320 | 1702 | | | 3 | 193065627 | 333 | 1.35 | 141 | 1.28 | 2.98 | 472 | 3.03E−03 |
| RS1063964 | 2053 | SRI | 6717 | 7 | 87673405 | 307 | 1.36 | 167 | 1.29 | 2.98 | 472 | 3.03E−03 |
| RS10516028 | 1892 | | | 5 | 165975170 | 58 | 1.24 | 414 | 1.35 | 2.98 | 470 | 3.03E−03 |
| RS4146127 | 350 | | | 4 | 59114124 | 392 | 1.32 | 78 | 1.41 | 2.98 | 468 | 3.03E−03 |
| RS7678343 | 1801 | | | 4 | 161555665 | 91 | 1.4 | 379 | 1.32 | 2.98 | 468 | 3.03E−03 |
| RS32967 | 1852 | | | 5 | 80214012 | 118 | 1.27 | 352 | 1.35 | 2.98 | 468 | 3.03E−03 |
| RS2292250 | 1634 | SRGAP3 | 9901 | 3 | 9064178 | 445 | 1.33 | 19 | 1.5 | 2.98 | 462 | 3.03E−03 |
| RS10495787 | 1561 | LTBP1 | 4052 | 2 | 33453813 | 114 | 1.39 | 348 | 1.31 | 2.98 | 460 | 3.04E−03 |
| RS1527420 | 196 | | | 2 | 119267761 | 395 | 1.32 | 53 | 1.43 | 2.98 | 446 | 3.04E−03 |
| RS1372184 | 2227 | | | 10 | 109230619 | 365 | 1.32 | 79 | 1.41 | 2.98 | 442 | 3.04E−03 |
| RS490680 | 1501 | SLC25A24 | 29957 | 1 | 108526041 | 188 | 1.29 | 292 | 1.36 | 2.97 | 478 | 3.13E−03 |
| RS2825652 | 1441 | | | 21 | 19865219 | 470 | 1.33 | 10 | 1.56 | 2.97 | 478 | 3.13E−03 |
| RS579588 | 1963 | BAI3 | 577 | 6 | 69639537 | 270 | 1.36 | 208 | 1.29 | 2.97 | 476 | 3.13E−03 |
| RS10510175 | 1631 | | | 3 | 3747717 | 417 | 1.32 | 55 | 1.42 | 2.97 | 470 | 3.13E−03 |
| RS566537 | 1962 | BAI3 | 577 | 6 | 69639246 | 266 | 1.36 | 206 | 1.3 | 2.97 | 470 | 3.13E−03 |
| RS2608030 | 2163 | | | 8 | 129234261 | 373 | 1.32 | 99 | 1.4 | 2.97 | 470 | 3.13E−03 |
| RS9287674 | 1552 | | | 2 | 6411955 | 313 | 1.36 | 153 | 1.28 | 2.97 | 464 | 3.13E−03 |
| RS491785 | 1502 | SLC25A24 | 29957 | 1 | 108526216 | 180 | 1.29 | 284 | 1.36 | 2.97 | 462 | 3.13E−03 |
| RS6077461 | 2539 | | | 20 | 8888587 | 192 | 1.38 | 250 | 1.31 | 2.97 | 440 | 3.14E−03 |
| RS333856 | 1576 | | | 2 | 117692216 | 475 | 1.33 | 7 | 1.61 | 2.96 | 480 | 3.23E−03 |
| RS279511 | 1781 | NDST4 | 64579 | 4 | 116125572 | 343 | 1.31 | 139 | 1.39 | 2.96 | 480 | 3.23E−03 |
| RS285406 | 855 | | | 8 | 87094129 | 460 | 1.33 | 22 | 1.49 | 2.96 | 480 | 3.23E−03 |
| RS9314803 | 2187 | | | 9 | 75072811 | 455 | 1.32 | 27 | 1.47 | 2.96 | 480 | 3.23E−03 |
| RS6544711 | 1565 | DYNC2LI1 | 51626 | 2 | 43853999 | 335 | 1.36 | 145 | 1.28 | 2.96 | 478 | 3.23E−03 |
| RS1480974 | 1719 | | | 4 | 34852928 | 146 | 1.28 | 334 | 1.36 | 2.96 | 478 | 3.23E−03 |
| RS245340 | 1851 | MSH3 | 4437 | 5 | 80197256 | 124 | 1.28 | 356 | 1.35 | 2.96 | 478 | 3.23E−03 |
| RS4509682 | 2216 | C10orf72 | 196740 | 10 | 49983627 | 136 | 1.39 | 342 | 1.31 | 2.96 | 476 | 3.23E−03 |
| RS3951463 | 2305 | | | 12 | 5284520 | 118 | 1.39 | 360 | 1.31 | 2.96 | 476 | 3.23E−03 |
| RS2133675 | 2321 | | | 12 | 33086481 | 435 | 1.34 | 41 | 1.22 | 2.96 | 474 | 3.23E−03 |
| RS10483512 | 2402 | | | 14 | 39587177 | 264 | 1.31 | 208 | 1.38 | 2.96 | 470 | 3.23E−03 |
| RS1506575 | 2366 | | | 13 | 70452523 | 392 | 1.32 | 76 | 1.41 | 2.96 | 466 | 3.23E−03 |
| RS540970 | 1377 | KCTD1 | 284252 | 18 | 22430481 | 161 | 1.37 | 303 | 1.3 | 2.96 | 462 | 3.23E−03 |
| RS4723970 | 2038 | C7orf10 | 79783 | 7 | 40542997 | 410 | 1.35 | 52 | 1.24 | 2.96 | 460 | 3.24E−03 |
| RS935056 | 1506 | | | 1 | 120134921 | 250 | 1.3 | 210 | 1.37 | 2.96 | 458 | 3.24E−03 |
| RS10517293 | 1737 | | | 4 | 54284572 | 426 | 1.32 | 30 | 1.46 | 2.96 | 454 | 3.24E−03 |
| RS6937642 | 1989 | | | 6 | 118812481 | 162 | 1.29 | 292 | 1.36 | 2.96 | 452 | 3.24E−03 |
| RS1404838 | 2063 | | | 7 | 122483459 | 167 | 1.38 | 287 | 1.31 | 2.96 | 452 | 3.24E−03 |
| RS339906 | 2481 | RAC1P1 | 387612 | 18 | 20521263 | 307 | 1.3 | 143 | 1.37 | 2.96 | 448 | 3.24E−03 |
| RS243965 | 399 | | | 4 | 111509327 | 313 | 1.36 | 133 | 1.28 | 2.96 | 444 | 3.24E−03 |
| RS2131774 | 1468 | CSMD2 | 114784 | 1 | 33841310 | 69 | 1.26 | 303 | 1.36 | 2.96 | 370 | 3.27E−03 |
| RS10493351 | 1482 | | | 1 | 63946731 | 472 | 1.33 | 10 | 1.56 | 2.95 | 480 | 3.33E−03 |
| RS2971828 | 1567 | | | 2 | 66408091 | 386 | 1.32 | 94 | 1.4 | 2.95 | 478 | 3.33E−03 |
| RS978876 | 2263 | | | 11 | 91525144 | 406 | 1.35 | 72 | 1.25 | 2.95 | 476 | 3.33E−03 |
| RS9317885 | 2365 | | | 13 | 69628755 | 226 | 1.3 | 252 | 1.36 | 2.95 | 476 | 3.33E−03 |
| RS8004187 | 2400 | PAX9 | 5083 | 14 | 36210255 | 256 | 1.36 | 220 | 1.3 | 2.95 | 474 | 3.33E−03 |
| RS4866602 | 1807 | | | 5 | 3742363 | 387 | 1.35 | 77 | 1.26 | 2.95 | 462 | 3.34E−03 |
| RS1526813 | 2326 | | | 12 | 73643812 | 175 | 1.37 | 259 | 1.3 | 2.95 | 432 | 3.35E−03 |
| RS2288338 | 369 | HNRPD | 3184 | 4 | 83493658 | 324 | 1.36 | 108 | 1.28 | 2.95 | 430 | 3.35E−03 |
| RS2222654 | 430 | | | 4 | 137225511 | 190 | 1.38 | 236 | 1.31 | 2.95 | 424 | 3.35E−03 |
| RS10510248 | 1625 | CNTN4 | 152330 | 3 | 3023519 | 281 | 1.3 | 139 | 1.37 | 2.95 | 418 | 3.36E−03 |
| RS7009724 | 2078 | | | 8 | 8976535 | 233 | 1.3 | 185 | 1.37 | 2.95 | 416 | 3.36E−03 |
| RS10515526 | 1886 | | | 5 | 143157421 | 475 | 1.33 | 7 | 1.61 | 2.94 | 480 | 3.44E−03 |
| RS6954168 | 755 | LOC653748 | 653748 | 7 | 153727085 | 473 | 1.33 | 7 | 1.6 | 2.94 | 478 | 3.44E−03 |
| RS10513319 | 952 | | | 9 | 119741270 | 464 | 1.33 | 16 | 1.51 | 2.94 | 478 | 3.44E−03 |
| RS7148403 | 1234 | | | 14 | 38248309 | 426 | 1.32 | 52 | 1.43 | 2.94 | 476 | 3.44E−03 |
| RS1948617 | 2516 | | | 18 | 56819944 | 199 | 1.29 | 277 | 1.36 | 2.94 | 474 | 3.44E−03 |
| RS10485441 | 2557 | BCAS1 | 8537 | 20 | 51997775 | 44 | 1.44 | 432 | 1.32 | 2.94 | 474 | 3.44E−03 |
| RS10484317 | 604 | FARS2 | 10667 | 6 | 5614669 | 452 | 1.32 | 22 | 1.48 | 2.94 | 472 | 3.44E−03 |
| RS1219739 | 2232 | CPXM2 | 119587 | 10 | 125515459 | 460 | 1.34 | 14 | 1.14 | 2.94 | 472 | 3.44E−03 |
| RS168142 | 2482 | | | 18 | 20523065 | 321 | 1.3 | 153 | 1.38 | 2.94 | 472 | 3.44E−03 |
| RS6798797 | 271 | | | 3 | 40741966 | 18 | 1.5 | 454 | 1.32 | 2.94 | 470 | 3.44E−03 |
| RS868691 | 971 | | | 10 | 25945882 | 434 | 1.32 | 32 | 1.46 | 2.94 | 464 | 3.45E−03 |
| RS10499231 | 2004 | UTRN | 7402 | 6 | 144903639 | 307 | 1.31 | 155 | 1.38 | 2.94 | 460 | 3.45E−03 |
| RS10489168 | 1476 | PRNPIP | 79033 | 1 | 44500588 | 441 | 1.32 | 19 | 1.49 | 2.94 | 458 | 3.45E−03 |
| RS2832298 | 2565 | C21orf41 | 54073 | 21 | 29665734 | 283 | 1.36 | 173 | 1.29 | 2.94 | 454 | 3.45E−03 |
| RS10521202 | 2470 | KIAA0672 | 9912 | 17 | 12755289 | 234 | 1.3 | 198 | 1.37 | 2.94 | 430 | 3.46E−03 |
| RS10508244 | 2200 | PFKP | 5214 | 10 | 3143203 | 454 | 1.32 | 28 | 1.47 | 2.93 | 480 | 3.55E−03 |
| RS1441638 | 1778 | NDST4 | 64579 | 4 | 116094415 | 363 | 1.31 | 117 | 1.39 | 2.93 | 478 | 3.55E−03 |
| RS2529588 | 2061 | IFRD1 | 3475 | 7 | 111890291 | 152 | 1.28 | 324 | 1.35 | 2.93 | 474 | 3.55E−03 |
| RS763836 | 1553 | ALK | 238 | 2 | 29758808 | 100 | 1.4 | 372 | 1.31 | 2.93 | 470 | 3.55E−03 |
| RS10499432 | 2019 | | | 7 | 13646983 | 228 | 1.3 | 244 | 1.36 | 2.93 | 470 | 3.55E−03 |
| RS6807699 | 1689 | LOC389142 | 389142 | 3 | 119736159 | 126 | 1.28 | 342 | 1.35 | 2.93 | 466 | 3.56E−03 |
| RS285866 | 2154 | | | 8 | 106132633 | 301 | 1.36 | 133 | 1.28 | 2.93 | 432 | 3.57E−03 |
| RS581277 | 2247 | MPPED2 | 744 | 11 | 30436710 | 421 | 1.32 | 59 | 1.42 | 2.92 | 478 | 3.67E−03 |
| RS10518660 | 1492 | LPHN2 | 23266 | 1 | 82154655 | 449 | 1.32 | 29 | 1.46 | 2.92 | 476 | 3.67E−03 |
| RS4970642 | 1498 | | | 1 | 82238319 | 426 | 1.32 | 52 | 1.43 | 2.92 | 476 | 3.67E−03 |
| RS10495058 | 1529 | GPATC2 | 55105 | 1 | 215697001 | 461 | 1.33 | 7 | 1.61 | 2.92 | 466 | 3.67E−03 |
| RS9299445 | 993 | LOC644954 | 644954 | 10 | 63244794 | 431 | 1.32 | 37 | 1.44 | 2.92 | 466 | 3.67E−03 |
| RS3844148 | 1981 | | | 6 | 106923377 | 164 | 1.29 | 302 | 1.36 | 2.92 | 464 | 3.67E−03 |
| RS4714203 | 1942 | DNAH8 | 1769 | 6 | 39048774 | 401 | 1.33 | 53 | 1.43 | 2.92 | 452 | 3.68E−03 |
| RS2200038 | 2447 | | | 15 | 37262421 | 207 | 1.37 | 247 | 1.3 | 2.92 | 452 | 3.68E−03 |
| RS744319 | 2448 | | | 15 | 55668992 | 315 | 1.31 | 109 | 1.4 | 2.92 | 422 | 3.69E−03 |
| RS722708 | 1363 | SPIRE1 | 56907 | 18 | 12535581 | 175 | 1.37 | 203 | 1.29 | 2.92 | 376 | 3.71E−03 |
| RS3904322 | 2343 | LOC646184 | 646184 | 13 | 22266506 | 290 | 1.34 | 86 | 1.25 | 2.92 | 374 | 3.71E−03 |
| RS514644 | 2246 | MPPED2 | 744 | 11 | 30408757 | 423 | 1.32 | 59 | 1.42 | 2.91 | 480 | 3.78E−03 |
| RS4901706 | 2414 | C14orf101 | 54916 | 14 | 56184138 | 421 | 1.32 | 59 | 1.42 | 2.91 | 478 | 3.78E−03 |
| RS10518442 | 1797 | ANKRD50 | 57182 | 4 | 125813718 | 110 | 1.39 | 366 | 1.32 | 2.91 | 474 | 3.78E−03 |
| RS2825672 | 2560 | | | 21 | 19887865 | 386 | 1.32 | 90 | 1.4 | 2.91 | 474 | 3.78E−03 |
| RS7718000 | 551 | | | 5 | 112998258 | 469 | 1.33 | 5 | 1.65 | 2.91 | 472 | 3.79E−03 |
| RS3924016 | 2002 | | | 6 | 139665701 | 328 | 1.31 | 146 | 1.38 | 2.91 | 472 | 3.79E−03 |
| RS4131953 | 2325 | | | 12 | 73597990 | 291 | 1.31 | 181 | 1.38 | 2.91 | 470 | 3.79E−03 |
| RS622345 | 1467 | | | 1 | 24513043 | 371 | 1.33 | 67 | 1.42 | 2.91 | 436 | 3.80E−03 |
| RS10500985 | 1052 | LUZP2 | 338645 | 11 | 24590527 | 83 | 1.4 | 353 | 1.31 | 2.91 | 434 | 3.80E−03 |
| RS578499 | 1958 | | | 6 | 54509226 | 316 | 1.36 | 166 | 1.29 | 2.9 | 480 | 3.90E−03 |
| RS173036 | 2449 | | | 15 | 58145519 | 472 | 1.33 | 10 | 1.56 | 2.9 | 480 | 3.90E−03 |
| RS737769 | 2569 | RUTBC2 | 129049 | 22 | 23605393 | 37 | 1.45 | 445 | 1.32 | 2.9 | 480 | 3.90E−03 |
| RS953177 | 823 | DNAJC5B | 85479 | 8 | 67096174 | 452 | 1.33 | 26 | 1.47 | 2.9 | 476 | 3.90E−03 |
| RS966430 | 1817 | | | 5 | 13736184 | 242 | 1.36 | 232 | 1.3 | 2.9 | 472 | 3.91E−03 |
| RS6838661 | 1763 | FRAS1 | 80144 | 4 | 79296422 | 112 | 1.27 | 360 | 1.35 | 2.9 | 470 | 3.91E−03 |
| RS10487729 | 2030 | LOC223075 | 223075 | 7 | 31538809 | 383 | 1.32 | 89 | 1.4 | 2.9 | 470 | 3.91E−03 |
| RS250388 | 519 | ARL15 | 54622 | 5 | 53621019 | 409 | 1.32 | 61 | 1.42 | 2.9 | 468 | 3.91E−03 |
| RS10510444 | 1636 | | | 3 | 15985480 | 250 | 1.31 | 218 | 1.37 | 2.9 | 466 | 3.91E−03 |
| RS10513765 | 316 | PEX5L | 51555 | 3 | 181027564 | 171 | 1.38 | 295 | 1.31 | 2.9 | 464 | 3.91E−03 |
| RS10508204 | 2199 | C10orf110 | 55853 | 10 | 1074360 | 228 | 1.3 | 238 | 1.37 | 2.9 | 464 | 3.91E−03 |
| RS7556543 | 1512 | PRG4 | 10216 | 1 | 184537042 | 81 | 1.41 | 375 | 1.32 | 2.9 | 454 | 3.91E−03 |
| RS2304376 | 727 | GBAS | 2631 | 7 | 56016513 | 376 | 1.35 | 76 | 1.26 | 2.9 | 450 | 3.91E−03 |
| RS2832296 | 2564 | C21orf41 | 54073 | 21 | 59663325 | 280 | 1.36 | 170 | 1.29 | 2.9 | 448 | 3.92E−03 |
| RS998291 | 1667 | EPHA6 | 285220 | 3 | 98257090 | 386 | 1.33 | 56 | 1.43 | 2.9 | 440 | 3.92E−03 |
| RS10510128 | 2231 | | | 10 | 125050938 | 374 | 1.31 | 56 | 1.42 | 2.9 | 428 | 3.92E−03 |
| RS1169429 | 913 | | | 9 | 86853672 | 361 | 1.35 | 67 | 1.25 | 2.9 | 426 | 3.92E−03 |
| RS2861754 | 1571 | | | 2 | 67931211 | 246 | 1.36 | 162 | 1.29 | 2.9 | 406 | 3.93E−03 |
| RS10490838 | 263 | | | 3 | 24574382 | 467 | 1.33 | 15 | 1.51 | 2.89 | 480 | 4.03E−03 |
| RS1938327 | 1551 | | | 1 | 240789859 | 462 | 1.33 | 18 | 1.5 | 2.89 | 478 | 4.03E−03 |
| RS10484575 | 1977 | ATG5 | 9474 | 6 | 106738553 | 441 | 1.32 | 39 | 1.44 | 2.89 | 478 | 4.03E−03 |
| RS4131643 | 2234 | | | 10 | 125827901 | 225 | 1.37 | 251 | 1.3 | 2.89 | 474 | 4.03E−03 |
| RS1564223 | 2512 | | | 18 | 56563874 | 254 | 1.36 | 222 | 1.3 | 2.89 | 474 | 4.03E−03 |
| RS2672734 | 1806 | EXOC3 | 11336 | 5 | 508696 | 229 | 1.3 | 245 | 1.36 | 2.89 | 472 | 4.03E−03 |
| RS4785953 | 2452 | ADCY9 | 115 | 16 | 4094852 | 415 | 1.32 | 59 | 1.42 | 2.89 | 472 | 4.03E−03 |
| RS10483942 | 2423 | | | 14 | 79548170 | 431 | 1.32 | 39 | 1.44 | 2.89 | 468 | 4.03E−03 |
| RS2001097 | 1939 | | | 6 | 32491836 | 389 | 1.32 | 69 | 1.42 | 2.89 | 456 | 4.04E−03 |
| RS10497352 | 1608 | | | 2 | 170664017 | 91 | 1.41 | 361 | 1.32 | 2.89 | 450 | 4.04E−03 |
| RS1680339 | 2483 | | | 18 | 20555981 | 152 | 1.38 | 268 | 1.31 | 2.89 | 418 | 4.05E−03 |
| RS1480972 | 1718 | | | 4 | 34852796 | 103 | 1.28 | 297 | 1.37 | 2.89 | 398 | 4.06E−03 |

TABLE 6
SNP markers with P-value less than 0.005 for APOA1 from F-test (one-way ANOVA).
Alleles are codes as 1 = A, 2 = C, 3 = G, and 4 = T. For each group number
of observations ‘n’ and average value ‘avg’ are presented.

| RS_id | SEQ_ID_no | Gene | Gene_id | Chr | Position | g1 | n1 |
|---|---|---|---|---|---|---|---|
| RS6954679 | 728 | | | 7 | 63134099 | 1/1 | 201 |
| RS2854946 | 1259 | SERPINA5 | 5104 | 14 | 94118132 | 2/2 | 13 |
| RS10504433 | 824 | | | 8 | 70021434 | 2/2 | 190 |
| RS1399503 | 1159 | | | 12 | 127204934 | 3/3 | 197 |
| RS10517155 | 330 | | | 4 | 28812651 | 2/2 | 1 |
| RS10503626 | 808 | PSD3 | 23362 | 8 | 18591364 | 1/1 | 2 |
| RS1442799 | 1298 | | | 15 | 96589992 | 2/2 | 210 |
| RS3886738 | 992 | LOC644954 | 644954 | 10 | 63234254 | 1/1 | 1 |
| RS10509160 | 995 | LOC644954 | 644954 | 10 | 63245218 | 2/2 | 1 |
| RS10509161 | 996 | LOC644954 | 644954 | 10 | 63245347 | 1/1 | 1 |
| RS10486467 | 712 | | | 7 | 26621039 | 1/1 | 1 |
| RS2170398 | 773 | | | 8 | 13592708 | 2/2 | 1 |
| RS10510995 | 281 | | | 3 | 70525819 | 1/1 | 1 |
| RS718759 | 575 | | | 5 | 125467810 | 2/2 | 1 |
| RS10498260 | 253 | MGC42174 | 129563 | 2 | 232764865 | 1/1 | 1 |
| RS744832 | 191 | | | 2 | 117565111 | 1/1 | 1 |
| RS10498321 | 1231 | NPAS3 | 64067 | 14 | 33263982 | 1/1 | 181 |
| RS2120650 | 1292 | | | 15 | 84419700 | 1/1 | 216 |
| RS4102401 | 866 | RRM2B | 50484 | 8 | 103288416 | 2/2 | 1 |
| RS352766 | 775 | | | 8 | 15688930 | 2/2 | 1 |
| RS641445 | 1398 | | | 18 | 72182254 | 3/3 | 1 |
| RS10488888 | 395 | | | 4 | 111503157 | 2/2 | 1 |
| RS1467596 | 605 | FLJ33708 | 285780 | 6 | 6360933 | 1/1 | 1 |
| RS2230133 | 580 | TAF7 | 6879 | 5 | 140679241 | 2/2 | 197 |
| RS10485909 | 738 | MAGI2 | 9863 | 7 | 78077684 | 2/2 | 219 |
| RS4960221 | 606 | LY86 | 9450 | 6 | 6547432 | 2/2 | 179 |
| RS10516418 | 384 | | | 4 | 97967932 | 1/1 | 206 |
| RS10521381 | 2582 | | | X | 85978186 | 3/3 | 60 |
| RS470559 | 1399 | MBP | 4155 | 18 | 72887390 | 2/2 | 3 |
| RS10511194 | 1670 | | | 3 | 103457196 | 1/1 | 14 |
| RS547008 | 2299 | | | 11 | 121523071 | 1/1 | 2 |
| RS10507768 | 2359 | | | 13 | 68865849 | 1/3 | 19 |
| RS10509929 | 1021 | | | 10 | 112482278 | 2/2 | 3 |
| RS7413158 | 53 | GBP2 | 2634 | 1 | 89353284 | 1/1 | 6 |
| RS1016579 | 881 | | | 8 | 129269143 | 1/1 | 1 |
| RS3912653 | 1091 | FAT3 | 120114 | 11 | 92135172 | 1/1 | 42 |
| RS2056247 | 1092 | | | 11 | 93315872 | 3/3 | 7 |
| RS10514614 | 2464 | | | 16 | 84837699 | 1/1 | 205 |
| RS10483457 | 1232 | GARNL1 | 253959 | 14 | 35211683 | 1/1 | 209 |
| RS387661 | 587 | GABRG2 | 2566 | 5 | 161476289 | 2/2 | 2 |
| RS5923642 | 2580 | DACH2 | 117154 | X | 85930864 | 1/1 | 69 |
| RS6811055 | 336 | | | 4 | 46586667 | 2/2 | 71 |
| RS10513673 | 1698 | MDS1 | 4197 | 3 | 170736860 | 1/1 | 2 |
| RS10498673 | 1912 | BMP6 | 654 | 6 | 7770256 | 2/2 | 174 |
| RS1867584 | 985 | | | 10 | 49321355 | 2/2 | 72 |
| RS10502465 | 2490 | | | 18 | 20848719 | 1/1 | 4 |
| RS9299445 | 993 | LOC644954 | 644954 | 10 | 63244794 | 2/2 | 199 |
| RS1792369 | 1090 | FAT3 | 120114 | 11 | 92092081 | 1/1 | 42 |
| RS2889611 | 2213 | | | 10 | 49321754 | 1/1 | 83 |
| RS2101435 | 254 | MGC42174 | 129563 | 2 | 232823402 | 2/2 | 184 |
| RS1112809 | 210 | | | 2 | 148197197 | 2/2 | 185 |
| RS1528842 | 209 | | | 2 | 148174046 | 1/1 | 185 |
| RS10493822 | 52 | GBP1 | 2633 | 1 | 89302268 | 2/2 | 5 |
| RS10508901 | 981 | MAPK8 | 5599 | 10 | 49297404 | 3/3 | 83 |
| RS10491954 | 2303 | RAB6IP2 | 23085 | 12 | 1224606 | 1/1 | 17 |
| RS9299446 | 994 | LOC644954 | 644954 | 10 | 63244886 | 1/1 | 2 |
| RS1073939 | 2302 | RAB6IP2 | 23085 | 12 | 1156438 | 1/1 | 143 |
| RS4352250 | 1600 | | | 2 | 167249624 | 2/2 | 165 |
| RS1961544 | 2559 | | | 21 | 19839174 | 2/2 | 189 |
| RS1810113 | 2153 | TM7SF4 | 81501 | 8 | 105433770 | 2/2 | 118 |
| RS548987 | 629 | SLC17A3 | 10786 | 6 | 25977350 | 2/2 | 205 |
| RS2072506 | 722 | AMPH | 273 | 7 | 38468920 | 1/1 | 206 |
| RS2899901 | 2403 | | | 14 | 39602916 | 1/1 | 74 |
| RS4341948 | 1601 | | | 2 | 167249871 | 2/2 | 5 |
| RS9317744 | 1176 | | | 13 | 22963766 | 2/4 | 11 |
| RS1410126 | 687 | C6orf103 | 79747 | 6 | 147125370 | 1/1 | 5 |
| RS10498293 | 2380 | | | 14 | 27284686 | 1/1 | 7 |
| RS10485110 | 682 | | | 6 | 116309557 | 3/3 | 6 |
| RS2235216 | 683 | | | 6 | 116310188 | 1/1 | 6 |
| RS10485864 | 702 | SDK1 | 221935 | 7 | 3890182 | 2/2 | 181 |
| RS10509119 | 988 | ANK3 | 288 | 10 | 61482646 | 1/1 | 194 |
| RS4128928 | 1678 | | | 3 | 119083686 | 2/2 | 36 |
| RS2383024 | 898 | C9orf39 | 54875 | 9 | 17477945 | 1/1 | 101 |
| RS10497298 | 1598 | | | 2 | 167245545 | 1/1 | 5 |
| RS166861 | 2352 | | | 13 | 55557523 | 1/1 | 189 |
| RS1324088 | 628 | | | 6 | 25949101 | 2/2 | 205 |
| RS10516125 | 1893 | | | 5 | 174413439 | 2/4 | 8 |
| RS10497295 | 1597 | | | 2 | 167235217 | 3/3 | 5 |
| RS295577 | 524 | mimitin | 91942 | 5 | 60457592 | 2/2 | 2 |
| RS10493425 | 9 | | | 1 | 67782715 | 1/1 | 207 |
| RS10501797 | 2276 | FAT3 | 120114 | 11 | 92167618 | 2/2 | 32 |
| RS6702277 | 1528 | ESRRG | 2104 | 1 | 215023308 | 1/1 | 19 |
| RS4368350 | 211 | | | 2 | 167249829 | 2/2 | 4 |
| RS10514009 | 2530 | | | 18 | 67250588 | 2/4 | 31 |
| RS1282646 | 2529 | | | 18 | 66590397 | 1/1 | 204 |
| RS1902049 | 154 | LTBP1 | 4052 | 2 | 33223086 | 3/3 | 129 |
| RS1380835 | 1390 | | | 18 | 39747045 | 3/3 | 176 |
| RS2255565 | 194 | | | 2 | 118114972 | 1/1 | 184 |
| RS2793207 | 1900 | CDYL | 9425 | 6 | 4901002 | 1/1 | 215 |
| RS10503573 | 780 | MSR1 | 4481 | 8 | 16047855 | 1/1 | 227 |
| RS10521382 | 2581 | DACH2 | 117154 | X | 85931510 | 1/1 | 62 |
| RS2113515 | 195 | | | 2 | 118115259 | 2/2 | 180 |
| RS692419 | 1819 | | | 5 | 18024601 | 2/2 | 64 |
| RS10484066 | 2428 | | | 14 | 98519148 | 1/1 | 133 |
| RS1329120 | 1499 | GBP2 | 2634 | 1 | 89357153 | 1/1 | 7 |
| RS1406444 | 1595 | | | 2 | 148189717 | 1/1 | 186 |
| RS10504166 | 2123 | LOC392222 | 392222 | 8 | 55556364 | 1/1 | 4 |
| RS572241 | 2264 | FAT3 | 120114 | 11 | 91942082 | 2/2 | 78 |
| RS1945011 | 2298 | GRIK4 | 2900 | 11 | 120126812 | 1/1 | 4 |
| RS536617 | 2266 | FAT3 | 120114 | 11 | 91943638 | 2/2 | 80 |
| RS8068574 | 1343 | | | 17 | 50327246 | 1/1 | 146 |
| RS10487012 | 2054 | | | 7 | 87854386 | 2/2 | 154 |
| RS866651 | 262 | THRB | 7068 | 3 | 24237159 | 2/2 | 161 |
| RS3760626 | 2533 | CLPTM1 | 1209 | 19 | 50148945 | 1/1 | 66 |
| RS10491855 | 947 | | | 9 | 119335541 | 2/2 | 17 |
| RS2099803 | 2228 | | | 10 | 109365959 | 1/1 | 48 |
| RS1509649 | 1599 | | | 2 | 167247754 | 2/2 | 5 |
| RS10489249 | 88 | | | 1 | 169981144 | 2/2 | 175 |
| RS877345 | 958 | TAF3 | 83860 | 10 | 8093782 | 1/1 | 180 |
| RS3116231 | 252 | MGC42174 | 129563 | 2 | 232600527 | 2/2 | 2 |
| RS950206 | 1791 | | | 4 | 116400004 | 2/2 | 34 |
| RS1828384 | 2335 | | | 12 | 114060113 | 2/2 | 51 |
| RS10495074 | 1530 | LOC128153 | 128153 | 1 | 215998288 | 2/2 | 87 |
| RS10483507 | 2405 | | | 14 | 39614861 | 1/1 | 53 |
| RS7792197 | 2055 | | | 7 | 87899449 | 1/1 | 4 |
| RS7412987 | 1532 | LOC128153 | 128153 | 1 | 216013079 | 2/2 | 31 |
| RS6838 | 2230 | ACADSB | 36 | 10 | 124807738 | 1/1 | 36 |
| RS950085 | 1006 | | | 10 | 95592637 | 1/1 | 24 |
| RS1919447 | 1593 | | | 2 | 148179334 | 2/2 | 184 |
| RS12026256 | 1511 | | | 1 | 175700199 | 1/1 | 154 |
| RS283063 | 1987 | SLC35F1 | 222553 | 6 | 118728007 | 3/4 | 36 |
| RS1456196 | 293 | | | 3 | 119137243 | 1/1 | 4 |
| RS4516081 | 2367 | | | 13 | 89219195 | 2/2 | 105 |
| RS10498032 | 239 | ABCA12 | 26154 | 2 | 215662730 | 1/1 | 29 |
| RS8049647 | 1310 | | | 16 | 50695297 | 1/1 | 163 |
| RS10485435 | 1964 | BAI3 | 577 | 6 | 69672336 | 1/1 | 21 |
| RS10503419 | 771 | | | 8 | 11164210 | 1/1 | 150 |
| RS10484065 | 2427 | | | 14 | 98518521 | 1/1 | 134 |
| RS10495783 | 155 | LTBP1 | 4052 | 2 | 33255136 | 2/2 | 48 |
| RS6919514 | 688 | UST | 10090 | 6 | 149151922 | 3/4 | 41 |
| RS10489251 | 89 | | | 1 | 169984157 | 3/3 | 5 |
| RS1372184 | 2227 | | | 10 | 109230619 | 3/3 | 147 |
| RS10485116 | 1946 | LOC653802 | 653802 | 6 | 42430492 | 2/4 | 9 |
| RS250387 | 518 | ARL15 | 54622 | 5 | 53620394 | 1/1 | 168 |
| RS817504 | 1679 | | | 3 | 119161552 | 3/3 | 152 |
| RS1407390 | 931 | C9orf84 | 158401 | 9 | 113504308 | 2/2 | 165 |
| RS9308931 | 156 | LTBP1 | 4052 | 2 | 33275201 | 1/1 | 61 |
| RS1884772 | 2404 | | | 14 | 39609074 | 2/2 | 54 |
| RS227521 | 1510 | ASTN | 460 | 1 | 175355785 | 1/1 | 191 |
| RS2353296 | 1733 | | | 4 | 47956320 | 1/1 | 1 |
| RS10241173 | 2065 | | | 7 | 124240494 | 1/1 | 162 |
| RS10508896 | 2210 | MARCH8 | 220972 | 10 | 45350649 | 1/1 | 213 |
| RS2292889 | 2250 | DEPC-1 | 221120 | 11 | 43898353 | 2/2 | 8 |
| RS10486853 | 2058 | FLJ36166 | 349152 | 7 | 102642309 | 2/2 | 189 |
| RS1559751 | 1525 | | | 1 | 198148785 | 1/1 | 105 |
| RS10484317 | 604 | FARS2 | 10667 | 6 | 5614669 | 2/2 | 217 |
| RS896990 | 251 | MGC42174 | 129563 | 2 | 232597100 | 1/1 | 2 |
| RS965193 | 1577 | | | 2 | 118106053 | 3/3 | 161 |
| RS7850478 | 946 | | | 9 | 119326659 | 1/1 | 17 |
| RS6492611 | 2369 | GPC5 | 2262 | 13 | 91940707 | 1/1 | 14 |
| RS492681 | 2472 | | | 18 | 9853662 | 2/2 | 12 |
| RS1029233 | 1452 | | | 21 | 32327571 | 1/1 | 23 |
| RS487707 | 2297 | FAM55D | 54827 | 11 | 113955918 | 1/1 | 116 |
| RS1427421 | 1661 | MITF | 4286 | 3 | 69870404 | 1/1 | 3 |
| RS10520892 | 1820 | | | 5 | 23623415 | 2/2 | 197 |
| RS10484921 | 2009 | | | 6 | 152083953 | 1/1 | 22 |
| RS1880438 | 2256 | LOC283247 | 283247 | 11 | 49327274 | 2/2 | 107 |
| RS945849 | 933 | C9orf84 | 158401 | 9 | 113545740 | 1/1 | 161 |
| RS10518821 | 1289 | | | 15 | 67669717 | 1/1 | 154 |
| RS352427 | 816 | | | 8 | 28571996 | 1/1 | 226 |
| RS768747 | 2300 | | | 11 | 123133551 | 2/2 | 167 |
| RS295525 | 525 | | | 5 | 60508623 | 2/2 | 2 |
| RS10517836 | 1747 | | | 4 | 63541480 | 2/2 | 186 |
| RS6477845 | 930 | C9orf84 | 158401 | 9 | 113502143 | 1/1 | 3 |
| RS7874576 | 896 | | | 9 | 12600410 | 1/3 | 4 |
| RS823638 | 934 | C9orf84 | 158401 | 9 | 113582582 | 1/1 | 3 |
| RS1387551 | 2135 | | | 8 | 82842822 | 2/2 | 1 |
| RS962592 | 476 | | | 4 | 181229107 | 2/2 | 234 |
| RS10500985 | 1052 | LUZP2 | 338645 | 11 | 24590527 | 1/1 | 6 |
| RS7470491 | 932 | C9orf84 | 158401 | 9 | 113530129 | 2/2 | 161 |
| RS9294812 | 1965 | BAI3 | 577 | 6 | 69674845 | 2/2 | 115 |
| RS7719212 | 501 | | | 5 | 23658681 | 2/2 | 222 |
| RS2291386 | 2013 | SYTL3 | 94120 | 6 | 159104495 | 2/2 | 215 |
| RS10498292 | 1229 | | | 14 | 27284462 | 1/1 | 165 |
| RS10494066 | 81 | NTNG1 | 22854 | 1 | 107591031 | 2/2 | 6 |
| RS9303347 | 1342 | | | 17 | 49585177 | 1/1 | 1 |
| RS10521202 | 2470 | KIAA0672 | 9912 | 17 | 12755289 | 2/2 | 52 |
| RS700049 | 2192 | | | 9 | 113622470 | 1/1 | 3 |
| RS9304301 | 1391 | | | 18 | 40017390 | 2/2 | 217 |
| RS718153 | 1554 | | | 2 | 30117122 | 1/1 | 26 |
| RS1893120 | 1788 | NDST4 | 64579 | 4 | 116230217 | 2/2 | 31 |
| RS10500668 | 1039 | | | 11 | 6285001 | 2/2 | 1 |
| RS10497650 | 223 | | | 2 | 184931280 | 2/4 | 5 |
| RS878016 | 757 | DLGAP2 | 9228 | 8 | 1585693 | 1/1 | 130 |
| RS2474263 | 678 | LOC442238 | 442238 | 6 | 100641976 | 3/3 | 33 |
| RS10518533 | 420 | | | 4 | 129677708 | 1/1 | 4 |
| RS1566728 | 1075 | PTPRJ | 5795 | 11 | 48121423 | 1/1 | 132 |
| RS7841198 | 2096 | PSD3 | 23362 | 8 | 18597648 | 2/2 | 1 |
| RS3827702 | 1423 | TASP1 | 55617 | 20 | 13446812 | 2/2 | 120 |
| RS901682 | 2208 | MARCH8 | 220972 | 10 | 45285867 | 1/1 | 2 |
| RS10490353 | 172 | UNQ6975 | 400952 | 2 | 45270098 | 2/2 | 196 |
| RS246558 | 1848 | | | 5 | 71859074 | 1/1 | 173 |
| RS10513993 | 2528 | | | 18 | 66588976 | 3/3 | 1 |
| RS8027192 | 2450 | | | 15 | 69793594 | 2/2 | 6 |
| RS9327673 | 1885 | | | 5 | 133230970 | 1/1 | 21 |
| RS7107851 | 2287 | HEPHL1 | 341208 | 11 | 93396703 | 2/2 | 154 |
| RS6798797 | 271 | | | 3 | 40741966 | 1/1 | 220 |
| RS2369669 | 2301 | RAB6IP2 | 23085 | 12 | 1080279 | 1/1 | 113 |
| RS967326 | 2094 | PSD3 | 23362 | 8 | 18591541 | 2/2 | 209 |
| RS10484518 | 698 | | | 6 | 164182068 | 3/3 | 23 |
| RS10521979 | 1461 | DMD | 1756 | X | 31697540 | 3/3 | 41 |
| RS965158 | 868 | | | 8 | 107564094 | 2/4 | 11 |
| RS2294311 | 1457 | HSPC117 | 51493 | 22 | 3112369 | 1/1 | 16 |
| RS10497649 | 1611 | | | 2 | 184930781 | 1/3 | 7 |
| RS1217654 | 761 | CSMD1 | 64478 | 8 | 4294769 | 2/2 | 194 |
| RS1773409 | 1535 | | | 1 | 234905895 | 1/1 | 6 |
| RS9323682 | 2422 | | | 14 | 79536316 | 1/1 | 2 |
| RS2503102 | 679 | LOC442238 | 442238 | 6 | 100650488 | 1/1 | 99 |
| RS3745718 | 1414 | CARD8 | 22900 | 19 | 53406965 | 1/1 | 74 |
| RS370480 | 2353 | | | 13 | 55575987 | 3/3 | 22 |
| RS1217656 | 759 | CSMD1 | 64478 | 8 | 4293171 | 1/1 | 1 |
| RS10510715 | 269 | | | 3 | 40714716 | 2/2 | 2 |
| RS4510935 | 945 | | | 9 | 119325172 | 2/2 | 6 |
| RS2064572 | 1969 | | | 6 | 92111778 | 1/1 | 66 |
| RS10486848 | 2052 | SEMA3D | 223117 | 7 | 84575619 | 2/2 | 165 |
| RS10515869 | 1891 | | | 5 | 163444804 | 1/1 | 49 |
| RS10499436 | 2020 | | | 7 | 13649002 | 1/1 | 19 |
| RS10510714 | 270 | | | 3 | 40736947 | 1/1 | 224 |
| RS724620 | 2579 | DACH2 | 117154 | X | 85825740 | 2/2 | 72 |
| RS6109935 | 1424 | TASP1 | 55617 | 20 | 13467837 | 2/2 | 19 |
| RS10521613 | 2576 | MID1 | 4281 | X | 10555454 | 2/2 | 12 |
| RS10505439 | 2162 | C8ORFK36 | 340359 | 8 | 124731285 | 1/1 | 1 |
| RS2045765 | 1760 | | | 4 | 63775688 | 2/2 | 189 |
| RS10512041 | 2189 | | | 9 | 77255358 | 2/2 | 1 |
| RS10504143 | 2121 | RB1CC1 | 9821 | 8 | 53714280 | 2/4 | 20 |
| RS6079097 | 2542 | TASP1 | 55617 | 20 | 13474577 | 1/1 | 18 |
| RS10494583 | 1514 | TPR | 7175 | 1 | 184557118 | 2/2 | 152 |
| RS1527420 | 196 | | | 2 | 119267761 | 1/1 | 176 |
| RS3218493 | 2069 | XRCC2 | 7516 | 7 | 151984596 | 2/3 | 10 |
| RS10499437 | 2021 | | | 7 | 13649243 | 1/1 | 19 |
| RS10512980 | 1823 | | | 5 | 51991165 | 1/1 | 26 |
| RS10485441 | 2557 | BCAS1 | 8537 | 20 | 51997775 | 1/1 | 2 |
| RS10509448 | 1005 | NRG3 | 10718 | 10 | 83972610 | 2/2 | 208 |
| RS32967 | 1852 | | | 5 | 80214012 | 2/2 | 15 |
| RS2371319 | 268 | | | 3 | 40701948 | 2/2 | 2 |
| RS10496649 | 203 | | | 2 | 126293862 | 2/2 | 200 |
| RS1462478 | 1708 | | | 3 | 194331608 | 2/2 | 138 |
| RS773013 | 2188 | | | 9 | 76973237 | 2/4 | 9 |
| RS6830598 | 1775 | NDST4 | 64579 | 4 | 116084463 | 2/2 | 35 |
| RS1959715 | 2426 | | | 14 | 87234963 | 1/1 | 1 |
| RS2111368 | 2341 | | | 12 | 126098472 | 1/1 | 168 |
| RS965788 | 2203 | PLXDC2 | 84898 | 10 | 20241557 | 1/1 | 11 |
| RS4564209 | 1531 | LOC128153 | 128153 | 1 | 215999639 | 2/2 | 97 |
| RS1889465 | 1985 | SLC35F1 | 222553 | 6 | 118649503 | 1/1 | 206 |
| RS10485490 | 2535 | | | 20 | 5376494 | 2/2 | 219 |
| RS6735220 | 229 | HECW2 | 57520 | 2 | 196963731 | 2/2 | 200 |
| RS1427422 | 1660 | | | 3 | 69866955 | 2/2 | 3 |
| RS245340 | 1851 | MSH3 | 4437 | 5 | 80197256 | 3/3 | 15 |
| RS4131462 | 1972 | | | 6 | 99364568 | 2/2 | 101 |
| RS207616 | 1564 | | | 2 | 40952801 | 1/1 | 97 |
| RS672489 | 1674 | | | 3 | 111133475 | 2/2 | 93 |
| RS7829473 | 2111 | | | 8 | 30297511 | 2/2 | 138 |
| RS1459914 | 280 | | | 3 | 67849940 | 3/3 | 195 |
| RS10501811 | 2282 | | | 11 | 93309061 | 1/1 | 2 |
| RS890783 | 1845 | | | 5 | 71031294 | 1/1 | 197 |
| RS10497472 | 1609 | | | 2 | 177492847 | 2/4 | 30 |
| RS709087 | 1700 | | | 3 | 192938993 | 2/2 | 123 |
| RS3104997 | 2143 | | | 8 | 96630910 | 2/2 | 17 |
| RS1235012 | 760 | CSMD1 | 64478 | 8 | 4294524 | 2/2 | 201 |
| RS1589629 | 2258 | | | 11 | 50431035 | 1/1 | 17 |
| RS10483456 | 2396 | GARNL1 | 253959 | 14 | 35105918 | 2/2 | 179 |
| RS10521240 | 2459 | | | 16 | 49914823 | 1/1 | 6 |
| RS4739647 | 2140 | | | 8 | 83563963 | 1/1 | 6 |
| RS1121640 | 1099 | | | 11 | 96694636 | 2/2 | 210 |
| RS10512000 | 2186 | | | 9 | 73345993 | 2/4 | 37 |
| RS10486470 | 2028 | | | 7 | 26636375 | 1/1 | 11 |
| RS1362406 | 2458 | DNAJA2 | 10294 | 16 | 45554058 | 3/3 | 214 |
| RS8054578 | 1323 | | | 16 | 77874316 | 1/1 | 3 |
| RS1228685 | 2015 | | | 6 | 165388456 | 3/3 | 181 |
| RS10508903 | 983 | MAPK8 | 5599 | 10 | 49311338 | 1/1 | 45 |
| RS6068024 | 2556 | | | 20 | 50001018 | 2/2 | 57 |
| RS4945630 | 1990 | | | 6 | 119472010 | 1/1 | 86 |
| RS2425570 | 2554 | PTPRT | 11122 | 20 | 41111765 | 2/2 | 158 |
| RS758780 | 2036 | PTHB1 | 27241 | 7 | 33444985 | 2/2 | 94 |
| RS758779 | 2037 | PTHB1 | 27241 | 7 | 33445083 | 1/1 | 94 |
| RS798101 | 2431 | LOC643811 | 643811 | 15 | 28890187 | 2/2 | 67 |
| RS6895637 | 1821 | ADAMTS12 | 81792 | 5 | 33785569 | 3/3 | 112 |
| RS6777583 | 1671 | | | 3 | 107546384 | 2/2 | 75 |
| RS10518896 | 2439 | C15orf41 | 84529 | 15 | 34731373 | 2/4 | 13 |
| RS10498944 | 1968 | | | 6 | 85414678 | 1/1 | 2 |
| RS4631686 | 3 | | | 1 | 14450061 | 1/3 | 8 |
| RS10503057 | 2520 | PIGN | 23556 | 18 | 57905398 | 1/1 | 152 |
| RS246580 | 1846 | | | 5 | 71851370 | 2/2 | 173 |
| RS719530 | 2059 | | | 7 | 109678201 | 2/2 | 54 |
| RS10483942 | 2423 | | | 14 | 79548170 | 2/2 | 2 |
| RS1499976 | 1684 | LOC389142 | 389142 | 3 | 119284020 | 2/2 | 148 |
| RS2769261 | 1504 | | | 1 | 113368430 | 1/1 | 10 |
| RS752355 | 213 | | | 2 | 176428271 | 1/1 | 15 |
| RS10485492 | 2536 | | | 20 | 5376989 | 2/2 | 1 |
| RS2296697 | 40 | LPHN2 | 23266 | 1 | 82209807 | 2/2 | 169 |
| RS717806 | 160 | | | 2 | 36429039 | 1/1 | 11 |
| RS1887545 | 1508 | POGZ | 23126 | 1 | 149651335 | 1/1 | 3 |
| RS1506672 | 2128 | XKR4 | 114786 | 8 | 56438061 | 2/2 | 42 |
| RS10494584 | 1515 | TPR | 7175 | 1 | 184557144 | 2/2 | 153 |
| RS1480145 | 796 | | | 8 | 16181494 | 2/2 | 229 |
| RS3218491 | 753 | XRCC2 | 7516 | 7 | 151984681 | 2/4 | 6 |
| RS10510104 | 1030 | ATE1 | 11101 | 10 | 123521812 | 2/2 | 5 |
| RS1465135 | 697 | | | 6 | 164115617 | 1/1 | 120 |
| RS1010504 | 2136 | | | 8 | 82922818 | 1/1 | 1 |
| RS956408 | 2304 | RAB6IP2 | 23085 | 12 | 1376941 | 2/2 | 112 |
| RS10497568 | 1610 | | | 2 | 181147675 | 2/2 | 44 |
| RS10510986 | 1655 | | | 3 | 69746452 | 1/1 | 194 |
| RS929332 | 2043 | | | 7 | 45300667 | 2/2 | 129 |
| RS6772743 | 1657 | | | 3 | 69804679 | 2/2 | 9 |
| RS10521297 | 2471 | | | 17 | 14999716 | 1/1 | 191 |
| RS7775656 | 1913 | TXNDC5 | 81567 | 6 | 7954759 | 1/1 | 164 |
| RS2291385 | 2014 | SYTL3 | 94120 | 6 | 159104665 | 1/3 | 25 |
| RS10490656 | 1562 | KIAA1414 | 54497 | 2 | 37085212 | 1/1 | 140 |
| RS3820182 | 1513 | TPR | 7175 | 1 | 184556956 | 1/1 | 7 |
| RS6990997 | 867 | ZFPM2 | 23414 | 8 | 106881703 | 2/2 | 6 |
| RS1883736 | 2248 | ABTB2 | 25841 | 11 | 34330019 | 1/3 | 14 |
| RS228563 | 1491 | | | 1 | 79418807 | 1/1 | 45 |
| RS2353193 | 1607 | | | 2 | 170142309 | 2/2 | 157 |
| RS10483887 | 2420 | ADCK1 | 57143 | 14 | 77355667 | 2/2 | 1 |
| RS10492047 | 1158 | LOC144678 | 144678 | 12 | 125501283 | 3/3 | 2 |
| RS6955049 | 2051 | CALN1 | 83698 | 7 | 71135644 | 1/1 | 1 |
| RS7181421 | 1291 | | | 15 | 77626966 | 2/2 | 91 |
| RS246575 | 1847 | | | 5 | 71855573 | 1/1 | 172 |
| RS10510809 | 274 | | | 3 | 59414093 | 2/2 | 164 |
| RS10517211 | 1717 | | | 4 | 30253481 | 2/2 | 184 |
| RS10517954 | 1802 | | | 4 | 168847633 | 1/1 | 6 |
| RS7529360 | 1489 | C1orf139 | 79971 | 1 | 68382555 | 2/2 | 94 |
| RS1046852 | 20 | C1orf139 | 79971 | 1 | 68363692 | 1/1 | 37 |
| RS10509702 | 1009 | | | 10 | 97914086 | 1/1 | 228 |
| RS958425 | 2204 | C10orf115 | 387642 | 10 | 23551932 | 1/1 | 180 |
| RS4673999 | 1612 | | | 2 | 216058272 | 1/1 | 64 |
| RS4888946 | 1324 | | | 16 | 77874931 | 1/1 | 171 |
| RS2163721 | 2333 | PPFIA2 | 8499 | 12 | 80343092 | 2/2 | 227 |
| RS7782588 | 2027 | | | 7 | 26635712 | 1/1 | 157 |
| RS10489688 | 1523 | | | 1 | 197054569 | 1/1 | 108 |
| RS10514844 | 1182 | | | 13 | 37808480 | 2/3 | 7 |
| RS779320 | 1702 | | | 3 | 193065627 | 2/2 | 120 |
| RS3749216 | 1650 | FAM19A1 | 407738 | 3 | 68548925 | 1/1 | 237 |
| RS10496466 | 1573 | | | 2 | 115053720 | 2/2 | 127 |
| RS650298 | 1466 | FBXO42 | 54455 | 1 | 16538195 | 2/2 | 75 |
| RS2671762 | 1633 | | | 3 | 5110111 | 2/2 | 194 |
| RS10517293 | 1737 | | | 4 | 54284572 | 1/3 | 30 |
| RS1851006 | 1278 | UNC13C | 440279 | 15 | 52691684 | 2/2 | 45 |
| RS2761089 | 2005 | | | 6 | 145473596 | 2/2 | 80 |
| RS10510987 | 1656 | | | 3 | 69785447 | 2/2 | 3 |
| RS362865 | 2007 | GRM1 | 2911 | 6 | 146704527 | 2/2 | 180 |
| RS8089476 | 1392 | | | 18 | 46107418 | 2/2 | 33 |
| RS1805781 | 1111 | PHC1 | 1911 | 12 | 8957155 | 1/3 | 6 |
| RS6837953 | 437 | | | 4 | 154150413 | 1/1 | 1 |
| RS4504494 | 2001 | ARHGAP18 | 93663 | 6 | 129954780 | 1/3 | 13 |
| RS7556543 | 1512 | PRG4 | 10216 | 1 | 184537042 | 1/1 | 7 |
| RS2001097 | 1939 | | | 6 | 32491836 | 1/1 | 5 |
| RS286300 | 1404 | LOC401898 | 401898 | 19 | 11658308 | 2/2 | 213 |
| RS10484926 | 2006 | | | 6 | 146336371 | 2/2 | 233 |
| RS2290547 | 272 | HYPB | 29072 | 3 | 47036187 | 1/1 | 13 |
| RS543465 | 1980 | ATG5 | 9474 | 6 | 106855297 | 2/2 | 203 |
| RS320694 | 2067 | | | 7 | 136695249 | 2/2 | 49 |
| RS9329911 | 2141 | | | 8 | 86594772 | 1/1 | 121 |
| RS10486471 | 2029 | | | 7 | 26636424 | 2/2 | 11 |
| RS6938470 | 640 | | | 6 | 47446050 | 1/1 | 3 |
| RS1501622 | 1705 | FGF12 | 2257 | 3 | 193634069 | 2/2 | 45 |
| RS4611835 | 313 | NAALADL2 | 254827 | 3 | 176610769 | 1/1 | 2 |
| RS10483426 | 2393 | NPAS3 | 64067 | 14 | 32670589 | 2/2 | 63 |
| RS2292250 | 1634 | SRGAP3 | 9901 | 3 | 9064178 | 1/1 | 213 |
| RS4952605 | 1563 | SLC8A1 | 6546 | 2 | 40376993 | 1/1 | 21 |
| RS1957425 | 2417 | | | 14 | 64428329 | 1/1 | 102 |
| RS3773636 | 1640 | TGFBR2 | 7048 | 3 | 30675445 | 1/1 | 13 |
| RS4405428 | 1213 | GPC5 | 2262 | 13 | 91916519 | 2/2 | 183 |
| RS10498096 | 1613 | | | 2 | 221469394 | 2/2 | 1 |
| RS2316853 | 2575 | | | X | 2575296 | 2/2 | 27 |
| RS723043 | 157 | LTBP1 | 4052 | 2 | 33395291 | 3/3 | 68 |
| RS2217610 | 700 | SDK1 | 221935 | 7 | 3697162 | 1/1 | 137 |
| RS1878144 | 193 | | | 2 | 117895659 | 3/3 | 181 |
| RS10496527 | 1575 | | | 2 | 117242961 | 2/2 | 42 |
| RS2971828 | 1567 | | | 2 | 66408091 | 1/1 | 156 |
| RS2669892 | 290 | ZBTB20 | 26137 | 3 | 115817713 | 1/1 | 159 |
| RS1915546 | 841 | | | 8 | 80202606 | 2/2 | 77 |
| RS823692 | 1907 | | | 6 | 7435616 | 1/1 | 1 |
| RS1334722 | 2201 | ITIH5 | 80760 | 10 | 7715013 | 1/1 | 107 |
| RS580758 | 390 | | | 4 | 105790400 | 1/1 | 4 |
| RS285406 | 855 | | | 8 | 87094129 | 2/2 | 219 |
| RS10500683 | 2235 | | | 11 | 7145579 | 2/2 | 93 |
| RS1413191 | 2370 | GPC5 | 2262 | 13 | 92011477 | 1/1 | 5 |
| RS600524 | 1570 | | | 2 | 67527284 | 2/2 | 100 |
| RS1506326 | 2525 | KIAA1468 | 57614 | 18 | 58099371 | 3/3 | 11 |
| RS10495774 | 150 | | | 2 | 30360180 | 2/2 | 10 |
| RS10497783 | 230 | HECW2 | 57520 | 2 | 196963769 | 1/1 | 2 |
| RS1579274 | 2119 | | | 8 | 41778080 | 1/1 | 19 |
| RS10503572 | 789 | | | 8 | 16131101 | 1/3 | 5 |
| RS2195989 | 872 | ANGPT1 | 284 | 8 | 108555088 | 2/2 | 9 |
| RS2223753 | 2538 | | | 20 | 6615135 | 1/1 | 76 |
| RS737769 | 2569 | RUTBC2 | 129049 | 22 | 23605393 | 2/4 | 37 |
| RS39626 | 1850 | MSH3 | 4437 | 5 | 80154550 | 2/2 | 130 |
| RS7828096 | 2095 | PSD3 | 23362 | 8 | 18597554 | 1/1 | 1 |
| RS10518660 | 1492 | LPHN2 | 23266 | 1 | 82154655 | 1/1 | 210 |
| RS958978 | 1614 | SP110 | 3431 | 2 | 230758230 | 1/1 | 145 |
| RS717897 | 2255 | OR4C4P | 79550 | 11 | 48332180 | 1/1 | 11 |
| RS10517210 | 1716 | | | 4 | 30253178 | 2/2 | 1 |
| RS720614 | 2064 | | | 7 | 124240026 | 2/2 | 9 |
| RS6949530 | 2062 | | | 7 | 122406461 | 1/1 | 174 |
| RS989177 | 748 | | | 7 | 124200122 | 2/2 | 13 |
| RS2591651 | 1826 | PDE4D | 5144 | 5 | 58615479 | 1/3 | 5 |
| RS962354 | 1738 | | | 4 | 56214479 | 1/1 | 172 |
| RS590599 | 2010 | | | 6 | 153161845 | 2/2 | 7 |
| RS198278 | 711 | | | 7 | 24214718 | 1/1 | 53 |
| RS870241 | 1654 | FAM19A4 | 151647 | 3 | 68908072 | 2/2 | 123 |
| RS727505 | 2066 | | | 7 | 124249317 | 2/2 | 157 |
| RS10503068 | 2526 | KIAA1468 | 57614 | 18 | 58102113 | 2/2 | 10 |
| RS1356216 | 192 | | | 2 | 117879031 | 3/3 | 187 |
| RS10483988 | 1250 | GALC | 2581 | 14 | 87504310 | 2/2 | 173 |
| RS2040868 | 2042 | | | 7 | 45293528 | 2/2 | 128 |
| RS694795 | 1666 | | | 3 | 85016728 | 1/1 | 53 |
| RS4073474 | 294 | IQCB1 | 9657 | 3 | 122990105 | 1/1 | 100 |
| RS10513166 | 1818 | | | 5 | 14046495 | 2/2 | 2 |
| RS9306468 | 2570 | MTMR3 | 8897 | 22 | 28704281 | 1/1 | 146 |
| RS2825652 | 1441 | | | 21 | 19865219 | 2/2 | 230 |
| RS9325559 | 2229 | EIF3S10 | 8661 | 10 | 120811615 | 1/1 | 6 |
| RS1519684 | 1983 | | | 6 | 114571663 | 2/2 | 28 |
| RS4533975 | 1959 | | | 6 | 68537723 | 2/2 | 156 |
| RS2973234 | 1725 | | | 4 | 37143568 | 2/2 | 115 |
| RS10513319 | 952 | | | 9 | 119741270 | 1/1 | 224 |
| RS847852 | 1940 | ANKS1 | 23294 | 6 | 35012070 | 1/1 | 39 |
| RS9319997 | 2522 | PIGN | 23556 | 18 | 57925017 | 2/2 | 151 |
| RS953177 | 823 | DNAJC5B | 85479 | 8 | 67096174 | 2/2 | 213 |
| RS10489474 | 1509 | PAPPA2 | 60676 | 1 | 174809772 | 2/2 | 159 |
| RS10490347 | 1566 | FLJ10379 | 55133 | 2 | 45548444 | 2/2 | 1 |
| RS947271 | 1507 | REG4 | 83998 | 1 | 120138926 | 1/1 | 56 |
| RS1608272 | 2259 | | | 11 | 50446832 | 1/1 | 129 |
| RS9320780 | 1991 | | | 6 | 121303614 | 1/1 | 13 |
| RS10516971 | 1769 | UNC5C | 8633 | 4 | 96560069 | 1/1 | 139 |
| RS4597125 | 2331 | | | 12 | 74694839 | 1/1 | 6 |
| RS10493351 | 1482 | | | 1 | 63946731 | 2/2 | 231 |
| RS756458 | 2463 | CDH13 | 1012 | 16 | 81846617 | 2/2 | 18 |
| RS333856 | 1576 | | | 2 | 117692216 | 1/2 | 7 |
| RS6844368 | 1764 | FRAS1 | 80144 | 4 | 79658959 | 3/3 | 38 |
| RS4146127 | 350 | | | 4 | 59114124 | 1/1 | 161 |
| RS1254275 | 2415 | | | 14 | 59915736 | 3/3 | 6 |
| RS2804861 | 2206 | | | 10 | 33815896 | 1/1 | 169 |
| RS1268444 | 1937 | | | 6 | 30519525 | 2/2 | 87 |
| RS176157 | 2568 | CABIN1 | 23523 | 22 | 22833987 | 1/1 | 2 |
| RS10506681 | 2328 | | | 12 | 73653196 | 2/2 | 99 |
| RS1581412 | 1697 | RSRC1 | 51319 | 3 | 159374881 | 2/2 | 32 |
| RS2111826 | 309 | CLDN11 | 5010 | 3 | 171623540 | 1/3 | 37 |
| RS2018842 | 1696 | | | 3 | 148362044 | 1/1 | 80 |
| RS1298362 | 1136 | | | 12 | 74493708 | 1/1 | 52 |
| RS4492337 | 2142 | C8orf37 | 157657 | 8 | 96336117 | 1/1 | 109 |
| RS2246219 | 1672 | GUCA1C | 9626 | 3 | 110141846 | 1/1 | 17 |
| RS9285977 | 1914 | | | 6 | 8517284 | 2/2 | 68 |
| RS9322914 | 2392 | NPAS3 | 64067 | 14 | 32571950 | 2/2 | 178 |
| RS4721923 | 2024 | LOC646385 | 646385 | 7 | 20604246 | 3/3 | 21 |
| RS4140855 | 2155 | | | 8 | 117023770 | 1/1 | 8 |
| RS10518014 | 1761 | | | 4 | 67282503 | 1/1 | 20 |
| RS1979739 | 2433 | RYR3 | 6263 | 15 | 31737586 | 1/1 | 34 |
| RS10515526 | 1886 | | | 5 | 143157421 | 1/3 | 7 |
| RS2887919 | 1647 | | | 3 | 39791844 | 1/1 | 21 |
| RS6954168 | 755 | LOC653748 | 653748 | 7 | 153727085 | 2/2 | 233 |
| RS2825673 | 2561 | | | 21 | 19888587 | 2/2 | 6 |
| RS4608876 | 1794 | | | 4 | 117668911 | 1/1 | 104 |
| RS7241109 | 2473 | C18orf1 | 753 | 18 | 13303114 | 1/1 | 112 |
| RS2042651 | 1795 | | | 4 | 117669326 | 1/1 | 18 |
| RS4661451 | 1465 | | | 1 | 14133149 | 1/1 | 53 |
| RS6708913 | 1596 | | | 2 | 165583661 | 1/1 | 3 |
| RS211587 | 1695 | NMNAT3 | 349565 | 3 | 140763843 | 2/2 | 2 |
| RS2409113 | 2076 | LOC645960 | 645960 | 8 | 8849712 | 2/2 | 152 |
| RS7926211 | 2257 | TYRL | 7300 | 11 | 49394908 | 1/1 | 20 |
| RS11232919 | 1087 | LOC646176 | 646176 | 11 | 81284714 | 2/2 | 2 |
| RS720613 | 749 | | | 7 | 124239906 | 2/2 | 142 |
| RS10498518 | 2416 | | | 14 | 64427295 | 1/1 | 30 |
| RS2670607 | 201 | | | 2 | 124114105 | 1/1 | 3 |
| RS10506700 | 2323 | | | 12 | 73340061 | 2/2 | 150 |
| RS10496626 | 1578 | CNTNAP5 | 129648 | 2 | 124593042 | 2/4 | 19 |
| RS10514450 | 1322 | | | 16 | 77874213 | 2/2 | 3 |
| RS2288272 | 1669 | ZBTB11 | 27107 | 3 | 102861166 | 2/2 | 113 |
| RS1938327 | 1551 | | | 1 | 240789859 | 1/1 | 222 |
| RS1296210 | 1522 | | | 1 | 197008567 | 2/2 | 57 |
| RS10495058 | 1529 | GPATC2 | 55105 | 1 | 215697001 | 1/3 | 7 |
| RS10507605 | 2350 | | | 13 | 55506479 | 1/1 | 211 |
| RS1944989 | 2510 | CCBE1 | 147372 | 18 | 55353222 | 1/1 | 14 |
| RS10490838 | 263 | | | 3 | 24574382 | 1/1 | 226 |
| RS2257086 | 1984 | | | 6 | 116925713 | 2/2 | 134 |
| RS6601146 | 1899 | LOC646210 | 646210 | 5 | 180226111 | 3/3 | 23 |
| RS25931 | 1816 | CTNND2 | 1501 | 5 | 11560083 | 2/3 | 38 |
| RS10516613 | 1796 | | | 4 | 123193063 | 2/2 | 61 |
| RS1931991 | 2003 | | | 6 | 142639061 | 3/3 | 7 |
| RS261861 | 1550 | RGS7 | 6000 | 1 | 239162199 | 1/1 | 38 |
| RS1460176 | 2134 | | | 8 | 80409177 | 1/1 | 105 |
| RS10504744 | 2138 | | | 8 | 82952758 | 2/2 | 99 |
| RS10484407 | 1957 | | | 6 | 53086362 | 1/1 | 2 |
| RS4722207 | 2025 | DRCTNNB1A | 84668 | 7 | 22959312 | 1/1 | 27 |
| RS2009373 | 1415 | CARD8 | 22900 | 19 | 53411226 | 2/2 | 41 |
| RS10518901 | 2441 | C15orf41 | 84529 | 15 | 34807406 | 1/1 | 80 |
| RS173036 | 2449 | | | 15 | 58145519 | 2/2 | 231 |
| RS7718000 | 551 | | | 5 | 112998258 | 1/3 | 5 |
| RS10496647 | 202 | | | 2 | 126205406 | 1/1 | 1 |
| RS2374031 | 2368 | GPC5 | 2262 | 13 | 91939563 | 1/1 | 143 |
| RS10503066 | 2523 | PIGN | 23556 | 18 | 57976538 | 1/1 | 153 |
| RS4764277 | 2314 | | | 12 | 16577657 | 1/1 | 73 |
| RS6977649 | 2057 | | | 7 | 88161552 | 2/2 | 2 |
| RS1119006 | 2354 | | | 13 | 68718453 | 3/3 | 216 |
| RS2827082 | 1444 | | | 21 | 22119060 | 2/2 | 5 |
| RS185926 | 2531 | LOC401898 | 401898 | 19 | 11658252 | 1/1 | 1 |
| RS1434817 | 1732 | | | 4 | 43312247 | 1/1 | 1 |
| RS9313999 | 1890 | | | 5 | 163444569 | 2/2 | 63 |
| RS1011439 | 1339 | | | 17 | 49582464 | 1/1 | 1 |
| RS7706518 | 576 | | | 5 | 127308921 | 1/1 | 195 |
| RS2157768 | 1340 | | | 17 | 49582892 | 1/1 | 226 |
| RS718883 | 1490 | SLC44A5 | 204962 | 1 | 75837778 | 1/1 | 140 |
| RS6673693 | 1527 | | | 1 | 207790043 | 1/1 | 7 |
| RS889053 | 1888 | | | 5 | 159028256 | 1/1 | 11 |
| RS10498602 | 2424 | | | 14 | 85663057 | 2/4 | 16 |
| RS10483512 | 2402 | | | 14 | 39587177 | 2/2 | 80 |
| RS2362932 | 120 | SMYD3 | 64754 | 1 | 243980907 | 1/1 | 34 |
| RS1497504 | 1637 | | | 3 | 21018495 | 1/1 | 7 |
| RS10521241 | 1303 | | | 16 | 49914787 | 1/1 | 4 |
| RS1025748 | 438 | | | 4 | 154783347 | 1/1 | 148 |
| RS10484973 | 1966 | RIMS1 | 22999 | 6 | 72677020 | 2/2 | 14 |
| RS1374028 | 1825 | PDE4D | 5144 | 5 | 58611588 | 1/1 | 183 |
| RS2842527 | 1941 | ZFAND3 | 60685 | 6 | 38083679 | 1/1 | 86 |
| RS5979345 | 2577 | MID1 | 4281 | X | 10625012 | 1/1 | 227 |
| RS9312013 | 260 | | | 3 | 8299589 | 3/3 | 214 |
| RS10507607 | 2351 | | | 13 | 55507498 | 2/2 | 211 |
| RS10494826 | 98 | LOC647219 | 647219 | 1 | 199352619 | 2/2 | 11 |
| RS634785 | 1572 | SEC15L2 | 23233 | 2 | 72442508 | 2/2 | 208 |
| RS737911 | 2571 | MTMR3 | 8897 | 22 | 28729417 | 1/1 | 11 |
| RS4684321 | 256 | | | 3 | 1842614 | 1/1 | 218 |
| RS2175935 | 2139 | | | 8 | 83561779 | 3/3 | 175 |
| RS25937 | 1815 | CTNND2 | 1501 | 5 | 11556189 | 1/3 | 37 |
| RS10507118 | 1151 | TMEM16D | 121601 | 12 | 99744565 | 2/2 | 4 |
| RS2833581 | 2567 | HUNK | 30811 | 21 | 32254024 | 2/2 | 41 |
| RS10482871 | 1439 | | | 21 | 15700259 | 2/3 | 5 |
| RS4122274 | 1873 | | | 5 | 102998589 | 1/1 | 28 |
| RS10521316 | 2461 | | | 16 | 53286727 | 2/2 | 64 |
| RS6673237 | 1534 | LYST | 1130 | 1 | 234107752 | 1/1 | 167 |
| RS2409037 | 577 | | | 5 | 127314089 | 3/3 | 194 |
| RS10503069 | 2527 | KIAA1468 | 57614 | 18 | 58122956 | 1/1 | 152 |
| RS9784951 | 2041 | | | 7 | 45293270 | 1/1 | 129 |
| RS10501171 | 2249 | | | 11 | 37100583 | 2/2 | 88 |
| RS660274 | 2262 | | | 11 | 84482949 | 1/1 | 10 |
| RS10508378 | 961 | | | 10 | 10085297 | 2/2 | 1 |
| RS1386971 | 1632 | LRRN1 | 57633 | 3 | 3814711 | 2/2 | 234 |
| RS1153606 | 1639 | RARB | 5915 | 3 | 25549190 | 1/1 | 158 |
| RS1905015 | 1668 | | | 3 | 100210226 | 1/1 | 31 |
| RS10506168 | 2322 | MUC19 | 283463 | 12 | 39222063 | 1/1 | 153 |
| RS1409993 | 2578 | DGAT2L4 | 158835 | X | 69182937 | 2/2 | 113 |
| RS2131774 | 1468 | CSMD2 | 114784 | 1 | 33841310 | 1/1 | 6 |
| RS2831853 | 1449 | | | 21 | 28781867 | 2/2 | 227 |
| RS10515792 | 1889 | | | 5 | 159028460 | 2/2 | 145 |
| RS6902590 | 2008 | | | 6 | 148672183 | 1/1 | 114 |
| RS1904165 | 1824 | ITGA1 | 3672 | 5 | 52271559 | 1/1 | 3 |
| RS10487019 | 2056 | | | 7 | 87915131 | 2/2 | 2 |
| RS9310715 | 1638 | UBE2E2 | 7325 | 3 | 23228345 | 1/1 | 134 |
| RS1410364 | 1915 | | | 6 | 9837105 | 2/2 | 140 |
| RS1044045 | 1167 | EFHA1 | 221154 | 13 | 20965072 | 1/1 | 158 |
| RS5754499 | 2572 | | | 22 | 31978717 | 2/4 | 22 |
| RS3770899 | 163 | CRIM1 | 51232 | 2 | 36508276 | 1/1 | 1 |
| RS2241577 | 1898 | KIAA1191 | 57179 | 5 | 175707471 | 1/1 | 6 |
| RS10502543 | 2507 | | | 18 | 26284782 | 1/1 | 22 |
| RS10508405 | 2202 | | | 10 | 10608856 | 1/1 | 112 |
| RS6776797 | 1675 | LOC653584 | 653584 | 3 | 116136262 | 2/2 | 228 |
| RS7241410 | 2474 | C18orf1 | 753 | 18 | 13303263 | 2/2 | 22 |
| RS896043 | 2239 | | | 11 | 10303570 | 3/3 | 21 |
| RS397405 | 2413 | | | 14 | 45388652 | 2/4 | 22 |
| RS2054915 | 1089 | FLJ22104 | 65084 | 11 | 86668250 | 1/1 | 61 |
| RS32218 | 1876 | FBN2 | 2201 | 5 | 127719132 | 2/2 | 13 |
| RS10485828 | 2558 | TUBB1 | 81027 | 20 | 57034050 | 2/2 | 7 |
| RS10495390 | 116 | RYR2 | 6262 | 1 | 235379636 | 2/2 | 197 |
| RS7161975 | 2432 | SGNE1 | 6447 | 15 | 30755131 | 2/2 | 190 |
| RS10510156 | 1036 | | | 10 | 128294406 | 1/1 | 5 |
| RS615696 | 2508 | MBD2 | 8932 | 18 | 49946910 | 2/2 | 6 |
| RS10492136 | 2313 | GRIN2B | 2904 | 12 | 13803044 | 2/2 | 2 |
| RS1318822 | 1804 | PALLD | 23022 | 4 | 170001064 | 1/1 | 87 |
| RS823986 | 579 | FSTL4 | 23105 | 5 | 132894139 | 1/1 | 21 |
| RS924159 | 2583 | | | X | 122041921 | 2/2 | 164 |
| RS10508876 | 2207 | | | 10 | 43774192 | 2/2 | 205 |
| RS4935712 | 2218 | LOC653480 | 653480 | 10 | 51929419 | 2/2 | 65 |
| RS10497723 | 227 | TMEFF2 | 23671 | 2 | 192700568 | 1/1 | 207 |
| RS1063964 | 2053 | SRI | 6717 | 7 | 87673405 | 2/2 | 103 |
| RS2169141 | 2563 | NCAM2 | 4685 | 21 | 21756991 | 1/1 | 64 |
| RS10484846 | 1901 | | | 6 | 5048614 | 2/2 | 5 |
| RS10483541 | 2401 | TRAPPC6B | 122553 | 14 | 38708680 | 2/4 | 10 |
| RS9304675 | 2534 | CARD8 | 22900 | 19 | 53429261 | 2/2 | 96 |
| RS10508895 | 2209 | MARCH8 | 220972 | 10 | 45287774 | 1/1 | 2 |
| RS10494231 | 1505 | | | 1 | 120125842 | 2/2 | 54 |
| RS1944487 | 2261 | | | 11 | 79493829 | 1/1 | 63 |
| RS10503056 | 2521 | PIGN | 23556 | 18 | 57916617 | 2/2 | 151 |
| RS4578215 | 1475 | HIVEP3 | 59269 | 1 | 41836179 | 2/4 | 7 |
| RS1429130 | 432 | | | 4 | 148443213 | 1/1 | 202 |
| RS1915552 | 844 | | | 8 | 80237554 | 1/1 | 33 |
| RS724023 | 2372 | | | 14 | 24201300 | 1/1 | 208 |
| RS1993306 | 1555 | LTBP1 | 4052 | 2 | 33299839 | 1/1 | 11 |
| RS2593963 | 1673 | MORC1 | 27136 | 3 | 110188056 | 2/2 | 28 |
| RS10516142 | 601 | FLT4 | 2324 | 5 | 180003769 | 2/2 | 234 |
| RS8076457 | 1328 | NTN1 | 9423 | 17 | 8884654 | 2/2 | 163 |
| RS522085 | 1803 | | | 4 | 169481301 | 1/1 | 150 |
| RS7794555 | 754 | LOC653748 | 653748 | 7 | 153675164 | 2/2 | 207 |
| RS2014617 | 2102 | | | 8 | 27633311 | 2/2 | 137 |
| RS10495099 | 1533 | | | 1 | 216829548 | 2/2 | 3 |
| RS10516865 | 379 | | | 4 | 91324324 | 3/3 | 236 |
| RS935056 | 1506 | | | 1 | 120134921 | 2/2 | 54 |
| RS1376917 | 1649 | CADPS | 8618 | 3 | 62473418 | 2/4 | 18 |
| RS10498603 | 2425 | | | 14 | 85680709 | 2/3 | 17 |
| RS10511729 | 901 | | | 9 | 23547227 | 1/1 | 86 |
| RS9323026 | 2406 | | | 14 | 40050143 | 1/1 | 41 |
| RS2346593 | 1756 | | | 4 | 63606400 | 1/1 | 2 |
| RS10515985 | 2524 | KIAA1468 | 57614 | 18 | 58097923 | 2/2 | 143 |
| RS6966318 | 2060 | IFRD1 | 3475 | 7 | 111880093 | 2/2 | 110 |
| RS987479 | 714 | | | 7 | 34922949 | 1/1 | 147 |
| RS10506835 | 2332 | | | 12 | 79667966 | 2/2 | 22 |
| RS10504742 | 2137 | | | 8 | 82950922 | 1/1 | 99 |
| RS6134885 | 2540 | TASP1 | 55617 | 20 | 13421323 | 3/3 | 140 |
| RS4131870 | 1960 | | | 6 | 68549169 | 2/2 | 16 |
| RS4785953 | 2452 | ADCY9 | 115 | 16 | 4094852 | 1/1 | 184 |
| RS3847222 | 2184 | | | 9 | 1779931 | 1/1 | 128 |
| RS3874273 | 2434 | | | 15 | 34183438 | 1/1 | 96 |
| RS570008 | 2260 | | | 11 | 79479756 | 3/3 | 63 |
| RS745708 | 976 | FRMPD2 | 143162 | 10 | 49115681 | 1/1 | 146 |
| RS4984442 | 2451 | | | 15 | 93183231 | 1/1 | 127 |
| RS2973237 | 334 | | | 4 | 37148085 | 2/2 | 26 |
| RS10521007 | 1822 | ADAMTS12 | 81792 | 5 | 33794319 | 2/2 | 156 |
| RS10489117 | 1712 | | | 4 | 16754666 | 1/1 | 4 |
| RS581277 | 2247 | MPPED2 | 744 | 11 | 30436710 | 2/2 | 3 |
| RS10489115 | 1710 | | | 4 | 16736425 | 2/2 | 4 |
| RS162676 | 1857 | | | 5 | 98019403 | 2/2 | 17 |
| RS3789343 | 2541 | TASP1 | 55617 | 20 | 13446547 | 1/1 | 141 |
| RS918092 | 2306 | GRIN2B | 2904 | 12 | 13632070 | 3/3 | 152 |
| RS9303878 | 1360 | | | 18 | 1564764 | 1/1 | 193 |
| RS2078536 | 2026 | DRCTNNB1A | 84668 | 7 | 22983172 | 2/2 | 23 |
| RS2347061 | 1731 | | | 4 | 43232434 | 1/1 | 235 |
| RS9325124 | 1887 | | | 5 | 148229011 | 1/1 | 33 |
| RS972432 | 2098 | PSD3 | 23362 | 8 | 18599317 | 2/2 | 1 |
| RS2209426 | 2185 | SH3GL2 | 6456 | 9 | 17756315 | 1/1 | 145 |
| RS1526723 | 1665 | | | 3 | 74113598 | 2/2 | 31 |
| RS2041442 | 1711 | | | 4 | 16746993 | 2/2 | 183 |
| RS2357344 | 1242 | SYNE2 | 23224 | 14 | 63578006 | 2/2 | 5 |
| RS10514136 | 1849 | AP3B1 | 8546 | 5 | 77467642 | 1/1 | 170 |
| RS637225 | 1517 | | | 1 | 192302414 | 2/2 | 55 |
| RS3859990 | 2584 | FLJ30058 | 158763 | X | 130050135 | 1/1 | 209 |

| RS_id | avg1 | g2 | n2 | avg2 | g3 | n3 | avg3 | F | dft | dfe | P-value |
|---|---|---|---|---|---|---|---|---|---|---|---|
| RS6954679 | 1.31 | 1/3 | 35 | 1.42 | 3/3 | 3 | 2.03 | 16.76 | 2 | 236 | 1.56E−07 |
| RS2854946 | 1.67 | 2/3 | 64 | 1.31 | 3/3 | 128 | 1.3 | 15.76 | 2 | 202 | 4.36E−07 |
| RS10504433 | 1.31 | 2/4 | 50 | 1.39 | 4/4 | 1 | 2.5 | 14.53 | 2 | 238 | 1.11E−06 |
| RS1399503 | 1.32 | 3/4 | 39 | 1.32 | 4/4 | 2 | 2.21 | 14.04 | 2 | 235 | 1.74E−06 |
| RS10517155 | 2.5 | 2/4 | 49 | 1.28 | 4/4 | 191 | 1.34 | 13.57 | 2 | 238 | 2.62E−06 |
| RS10503626 | 2.15 | 1/3 | 17 | 1.43 | 3/3 | 221 | 1.32 | 13.54 | 2 | 237 | 2.70E−06 |
| RS1442799 | 1.32 | 2/4 | 30 | 1.38 | 4/4 | 1 | 2.5 | 12.89 | 2 | 238 | 4.84E−06 |
| RS3886738 | 2.5 | 1/3 | 34 | 1.37 | 3/3 | 201 | 1.32 | 12.85 | 2 | 233 | 5.08E−06 |
| RS10509160 | 2.5 | 2/3 | 38 | 1.37 | 3/3 | 202 | 1.32 | 12.79 | 2 | 238 | 5.30E−06 |
| RS10509161 | 2.5 | 1/3 | 33 | 1.38 | 3/3 | 203 | 1.32 | 12.67 | 2 | 234 | 5.96E−06 |
| RS10486467 | 2.5 | 1/3 | 9 | 1.25 | 3/3 | 228 | 1.33 | 12.6 | 2 | 235 | 6.34E−06 |
| RS2170398 | 2.5 | 2/4 | 40 | 1.36 | 4/4 | 199 | 1.32 | 12.53 | 2 | 237 | 6.72E−06 |
| RS10510995 | 2.5 | 1/3 | 12 | 1.4 | 3/3 | 223 | 1.33 | 12.37 | 2 | 233 | 7.84E−06 |
| RS718759 | 2.5 | 2/3 | 25 | 1.31 | 3/3 | 199 | 1.33 | 12.27 | 2 | 222 | 8.82E−06 |
| RS10498260 | 2.5 | 1/3 | 37 | 1.35 | 3/3 | 187 | 1.32 | 12.25 | 2 | 222 | 8.98E−06 |
| RS744832 | 2.5 | 1/3 | 51 | 1.33 | 3/3 | 186 | 1.32 | 12.16 | 2 | 235 | 9.44E−06 |
| RS10498321 | 1.33 | 1/2 | 56 | 1.34 | 2/2 | 1 | 2.5 | 12.16 | 2 | 235 | 9.44E−06 |
| RS2120650 | 1.33 | 1/3 | 24 | 1.35 | 3/3 | 1 | 2.5 | 12.15 | 2 | 238 | 9.45E−06 |
| RS4102401 | 2.5 | 2/4 | 18 | 1.36 | 4/4 | 221 | 1.33 | 12.13 | 2 | 237 | 9.65E−06 |
| RS352766 | 2.5 | 2/4 | 28 | 1.31 | 4/4 | 211 | 1.33 | 12.1 | 2 | 237 | 9.92E−06 |
| RS641445 | 2.5 | 3/4 | 21 | 1.31 | 4/4 | 191 | 1.33 | 12.16 | 2 | 210 | 1.01E−05 |
| RS10488888 | 2.5 | 2/4 | 6 | 1.37 | 4/4 | 233 | 1.33 | 12.08 | 2 | 237 | 1.01E−05 |
| RS1467596 | 2.5 | 1/3 | 12 | 1.35 | 3/3 | 226 | 1.33 | 12.07 | 2 | 236 | 1.02E−05 |
| RS2230133 | 1.33 | 2/3 | 42 | 1.32 | 3/3 | 1 | 2.5 | 12.06 | 2 | 237 | 1.03E−05 |
| RS10485909 | 1.33 | 2/4 | 19 | 1.32 | 4/4 | 1 | 2.5 | 11.99 | 2 | 236 | 1.10E−05 |
| RS4960221 | 1.33 | 2/4 | 59 | 1.33 | 4/4 | 1 | 2.5 | 11.98 | 2 | 236 | 1.11E−05 |
| RS10516418 | 1.33 | 1/3 | 26 | 1.33 | 3/3 | 1 | 2.5 | 11.91 | 2 | 230 | 1.20E−05 |
| RS10521381 | 1.44 | 4/4 | 142 | 1.27 | 0/0 | 0 | 0 | 19.94 | 1 | 200 | 1.33E−05 |
| RS470559 | 1.91 | 2/3 | 51 | 1.26 | 3/3 | 187 | 1.34 | 11.25 | 2 | 238 | 2.15E−05 |
| RS10511194 | 1.45 | 1/3 | 78 | 1.23 | 3/3 | 132 | 1.38 | 11.28 | 2 | 221 | 2.16E−05 |
| RS547008 | 2.04 | 1/3 | 36 | 1.4 | 3/3 | 194 | 1.31 | 11.26 | 2 | 229 | 2.17E−05 |
| RS10507768 | 1.56 | 3/3 | 211 | 1.31 | 0/0 | 0 | 0 | 18.68 | 1 | 228 | 2.31E−05 |
| RS10509929 | 1.9 | 2/3 | 49 | 1.4 | 3/3 | 178 | 1.31 | 11.18 | 2 | 227 | 2.34E−05 |
| RS7413158 | 1.78 | 1/2 | 61 | 1.3 | 2/2 | 174 | 1.33 | 10.97 | 2 | 238 | 2.77E−05 |
| RS1016579 | 1.79 | 1/3 | 23 | 1.52 | 3/3 | 188 | 1.31 | 10.85 | 2 | 209 | 3.29E−05 |
| RS3912653 | 1.24 | 1/3 | 113 | 1.3 | 3/3 | 83 | 1.43 | 10.59 | 2 | 235 | 3.95E−05 |
| RS2056247 | 1.64 | 3/4 | 77 | 1.39 | 4/4 | 157 | 1.29 | 10.47 | 2 | 238 | 4.38E−05 |
| RS10514614 | 1.31 | 1/3 | 32 | 1.5 | 3/3 | 2 | 1.06 | 10.46 | 2 | 236 | 4.44E−05 |
| RS10483457 | 1.32 | 1/3 | 29 | 1.38 | 3/3 | 2 | 2.07 | 10.32 | 2 | 237 | 5.04E−05 |
| RS387661 | 2.08 | 2/4 | 36 | 1.29 | 4/4 | 202 | 1.33 | 10.16 | 2 | 237 | 5.84E−05 |
| RS5923642 | 1.43 | 3/3 | 167 | 1.29 | 0/0 | 0 | 0 | 16.7 | 1 | 234 | 6.02E−05 |
| RS6811055 | 1.3 | 2/4 | 100 | 1.42 | 4/4 | 52 | 1.25 | 10.04 | 2 | 220 | 6.72E−05 |
| RS10513673 | 2.05 | 1/4 | 16 | 1.24 | 4/4 | 221 | 1.33 | 9.96 | 2 | 236 | 7.04E−05 |
| RS10498673 | 1.29 | 2/4 | 58 | 1.44 | 4/4 | 5 | 1.47 | 9.93 | 2 | 234 | 7.26E−05 |
| RS1867584 | 1.25 | 2/4 | 100 | 1.35 | 4/4 | 45 | 1.46 | 9.96 | 2 | 214 | 7.31E−05 |
| RS10502465 | 1.75 | 1/3 | 22 | 1.46 | 3/3 | 212 | 1.31 | 9.89 | 2 | 235 | 7.52E−05 |
| RS9299445 | 1.31 | 2/4 | 33 | 1.37 | 4/4 | 2 | 2.05 | 9.89 | 2 | 231 | 7.57E−05 |
| RS1792369 | 1.24 | 1/4 | 112 | 1.3 | 4/4 | 82 | 1.43 | 9.88 | 2 | 233 | 7.61E−05 |
| RS2889611 | 1.26 | 1/4 | 114 | 1.35 | 4/4 | 35 | 1.47 | 9.85 | 2 | 229 | 7.88E−05 |
| RS2101435 | 1.32 | 2/3 | 32 | 1.34 | 3/3 | 2 | 2.08 | 9.71 | 2 | 215 | 9.18E−05 |
| RS1112809 | 1.37 | 2/4 | 50 | 1.21 | 0/0 | 0 | 0 | 15.63 | 1 | 233 | 1.02E−04 |
| RS1528842 | 1.37 | 1/3 | 56 | 1.22 | 0/0 | 0 | 0 | 15.56 | 1 | 239 | 1.05E−04 |
| RS10493822 | 1.79 | 2/4 | 55 | 1.31 | 4/4 | 181 | 1.33 | 9.49 | 2 | 238 | 1.08E−04 |
| RS10508901 | 1.26 | 3/4 | 121 | 1.34 | 4/4 | 32 | 1.47 | 9.32 | 2 | 233 | 1.28E−04 |
| RS10491954 | 1.54 | 1/3 | 78 | 1.27 | 3/3 | 143 | 1.34 | 9.31 | 2 | 235 | 1.29E−04 |
| RS9299446 | 2.05 | 1/2 | 36 | 1.36 | 2/2 | 203 | 1.32 | 9.27 | 2 | 238 | 1.33E−04 |
| RS1073939 | 1.34 | 1/3 | 80 | 1.27 | 3/3 | 16 | 1.56 | 9.27 | 2 | 236 | 1.33E−04 |
| RS4352250 | 1.35 | 2/4 | 53 | 1.24 | 4/4 | 5 | 1.68 | 9.26 | 2 | 220 | 1.38E−04 |
| RS1961544 | 1.31 | 2/3 | 24 | 1.52 | 0/0 | 0 | 0 | 15.08 | 1 | 211 | 1.38E−04 |
| RS1810113 | 1.27 | 2/4 | 87 | 1.42 | 4/4 | 34 | 1.35 | 9.22 | 2 | 236 | 1.39E−04 |
| RS548987 | 1.31 | 2/3 | 32 | 1.42 | 3/3 | 1 | 2.21 | 9.21 | 2 | 235 | 1.41E−04 |
| RS2072506 | 1.31 | 1/4 | 30 | 1.5 | 0/0 | 0 | 0 | 14.97 | 1 | 234 | 1.42E−04 |
| RS2899901 | 1.34 | 1/3 | 113 | 1.38 | 3/3 | 51 | 1.21 | 9.2 | 2 | 235 | 1.42E−04 |
| RS4341948 | 1.68 | 2/4 | 64 | 1.25 | 4/4 | 171 | 1.35 | 9.08 | 2 | 237 | 1.59E−04 |
| RS9317744 | 1.61 | 4/4 | 211 | 1.32 | 0/0 | 0 | 0 | 14.77 | 1 | 220 | 1.59E−04 |
| RS1410126 | 1.74 | 1/2 | 58 | 1.27 | 2/2 | 174 | 1.34 | 9.01 | 2 | 234 | 1.70E−04 |
| RS10498293 | 1.62 | 1/3 | 51 | 1.41 | 3/3 | 183 | 1.3 | 8.99 | 2 | 238 | 1.72E−04 |
| RS10485110 | 1.74 | 3/4 | 69 | 1.33 | 4/4 | 163 | 1.32 | 8.98 | 2 | 235 | 1.75E−04 |
| RS2235216 | 1.74 | 1/3 | 71 | 1.33 | 3/3 | 142 | 1.32 | 8.97 | 2 | 216 | 1.81E−04 |
| RS10485864 | 1.32 | 2/3 | 49 | 1.35 | 3/3 | 4 | 1.83 | 8.94 | 2 | 231 | 1.82E−04 |
| RS10509119 | 1.34 | 1/3 | 45 | 1.28 | 3/3 | 2 | 2.01 | 8.87 | 2 | 238 | 1.93E−04 |
| RS4128928 | 1.47 | 2/3 | 120 | 1.34 | 3/3 | 84 | 1.27 | 8.84 | 2 | 237 | 1.98E−04 |
| RS2383024 | 1.27 | 1/2 | 99 | 1.41 | 2/2 | 35 | 1.29 | 8.83 | 2 | 232 | 2.01E−04 |
| RS10497298 | 1.68 | 1/3 | 63 | 1.26 | 3/3 | 163 | 1.35 | 8.83 | 2 | 228 | 2.03E−04 |
| RS166861 | 1.31 | 1/3 | 48 | 1.39 | 3/3 | 3 | 1.85 | 8.81 | 2 | 237 | 2.04E−04 |
| RS1324088 | 1.31 | 2/4 | 34 | 1.41 | 4/4 | 1 | 2.21 | 8.77 | 2 | 237 | 2.12E−04 |
| RS10516125 | 1.65 | 4/4 | 233 | 1.32 | 0/0 | 0 | 0 | 14.13 | 1 | 239 | 2.15E−04 |
| RS10497295 | 1.68 | 3/4 | 64 | 1.26 | 4/4 | 171 | 1.35 | 8.73 | 2 | 237 | 2.20E−04 |
| RS295577 | 1.93 | 2/3 | 9 | 1.49 | 3/3 | 223 | 1.32 | 8.73 | 2 | 231 | 2.21E−04 |
| RS10493425 | 1.33 | 1/2 | 13 | 1.23 | 2/2 | 2 | 1.98 | 8.74 | 2 | 219 | 2.23E−04 |
| RS10501797 | 1.24 | 2/3 | 118 | 1.3 | 3/3 | 90 | 1.41 | 8.7 | 2 | 237 | 2.26E−04 |
| RS6702277 | 1.5 | 1/3 | 103 | 1.27 | 3/3 | 114 | 1.35 | 8.7 | 2 | 233 | 2.27E−04 |
| RS4368350 | 1.79 | 2/4 | 36 | 1.26 | 4/4 | 173 | 1.35 | 8.73 | 2 | 210 | 2.28E−04 |
| RS10514009 | 1.49 | 4/4 | 209 | 1.31 | 0/0 | 0 | 0 | 13.93 | 1 | 238 | 2.37E−04 |
| RS1282646 | 1.34 | 1/4 | 33 | 1.24 | 4/4 | 2 | 1.94 | 8.63 | 2 | 236 | 2.41E−04 |
| RS1902049 | 1.31 | 3/4 | 90 | 1.3 | 4/4 | 18 | 1.55 | 8.62 | 2 | 234 | 2.44E−04 |
| RS1380835 | 1.34 | 3/4 | 62 | 1.28 | 4/4 | 3 | 1.85 | 8.59 | 2 | 238 | 2.50E−04 |
| RS2255565 | 1.33 | 1/2 | 53 | 1.31 | 2/2 | 4 | 1.83 | 8.57 | 2 | 238 | 2.55E−04 |
| RS2793207 | 1.32 | 1/3 | 17 | 1.55 | 0/0 | 0 | 0 | 13.76 | 1 | 230 | 2.60E−04 |
| RS10503573 | 1.32 | 1/2 | 14 | 1.57 | 0/0 | 0 | 0 | 13.74 | 1 | 239 | 2.61E−04 |
| RS10521382 | 1.44 | 2/2 | 149 | 1.3 | 0/0 | 0 | 0 | 13.75 | 1 | 209 | 2.68E−04 |
| RS2113515 | 1.33 | 2/4 | 55 | 1.32 | 4/4 | 4 | 1.83 | 8.46 | 2 | 236 | 2.83E−04 |
| RS692419 | 1.3 | 2/4 | 120 | 1.39 | 4/4 | 54 | 1.24 | 8.45 | 2 | 235 | 2.86E−04 |
| RS10484066 | 1.35 | 1/2 | 76 | 1.26 | 2/2 | 22 | 1.49 | 8.43 | 2 | 228 | 2.94E−04 |
| RS1329120 | 1.7 | 1/3 | 60 | 1.3 | 3/3 | 172 | 1.33 | 8.4 | 2 | 236 | 2.99E−04 |
| RS1406444 | 1.36 | 1/2 | 53 | 1.23 | 0/0 | 0 | 0 | 13.38 | 1 | 237 | 3.13E−04 |
| RS10504166 | 1.78 | 1/2 | 57 | 1.37 | 2/2 | 171 | 1.31 | 8.35 | 2 | 229 | 3.16E−04 |
| RS572241 | 1.25 | 2/4 | 113 | 1.35 | 4/4 | 43 | 1.43 | 8.29 | 2 | 231 | 3.33E−04 |
| RS1945011 | 1.32 | 1/3 | 75 | 1.24 | 3/3 | 160 | 1.37 | 8.23 | 2 | 236 | 3.51E−04 |
| RS536617 | 1.25 | 2/4 | 118 | 1.35 | 4/4 | 42 | 1.43 | 8.22 | 2 | 237 | 3.54E−04 |
| RS8068574 | 1.33 | 1/3 | 78 | 1.31 | 3/3 | 13 | 1.6 | 8.21 | 2 | 234 | 3.58E−04 |
| RS10487012 | 1.3 | 2/4 | 74 | 1.41 | 4/4 | 6 | 1.07 | 8.21 | 2 | 231 | 3.59E−04 |
| RS866651 | 1.32 | 2/3 | 69 | 1.31 | 3/3 | 10 | 1.64 | 8.2 | 2 | 237 | 3.60E−04 |
| RS3760626 | 1.26 | 1/3 | 130 | 1.33 | 3/3 | 43 | 1.45 | 8.18 | 2 | 236 | 3.67E−04 |
| RS10491855 | 1.51 | 2/3 | 89 | 1.37 | 3/3 | 129 | 1.29 | 8.15 | 2 | 232 | 3.80E−04 |
| RS2099803 | 1.31 | 1/4 | 124 | 1.39 | 4/4 | 66 | 1.24 | 8.11 | 2 | 235 | 3.93E−04 |
| RS1509649 | 1.68 | 2/4 | 58 | 1.26 | 4/4 | 169 | 1.35 | 8.1 | 2 | 229 | 3.99E−04 |
| RS10489249 | 1.33 | 2/3 | 61 | 1.3 | 3/3 | 5 | 1.75 | 8.05 | 2 | 238 | 4.14E−04 |
| RS877345 | 1.32 | 1/2 | 39 | 1.35 | 2/2 | 3 | 1.9 | 8.06 | 2 | 219 | 4.19E−04 |
| RS3116231 | 1.96 | 2/3 | 48 | 1.38 | 3/3 | 189 | 1.31 | 8.01 | 2 | 236 | 4.31E−04 |
| RS950206 | 1.29 | 2/4 | 111 | 1.28 | 4/4 | 93 | 1.41 | 7.97 | 2 | 235 | 4.48E−04 |
| RS1828384 | 1.38 | 2/4 | 128 | 1.37 | 4/4 | 60 | 1.23 | 7.95 | 2 | 236 | 4.56E−04 |
| RS10495074 | 1.27 | 2/4 | 122 | 1.34 | 4/4 | 31 | 1.48 | 7.94 | 2 | 237 | 4.60E−04 |
| RS10483507 | 1.22 | 1/4 | 112 | 1.38 | 4/4 | 74 | 1.34 | 7.94 | 2 | 236 | 4.60E−04 |
| RS7792197 | 1.78 | 1/3 | 24 | 1.39 | 3/3 | 211 | 1.32 | 7.91 | 2 | 236 | 4.73E−04 |
| RS7412987 | 1.48 | 2/4 | 101 | 1.34 | 4/4 | 88 | 1.27 | 7.93 | 2 | 217 | 4.74E−04 |
| RS6838 | 1.48 | 1/4 | 122 | 1.29 | 4/4 | 75 | 1.33 | 7.91 | 2 | 230 | 4.76E−04 |
| RS950085 | 1.47 | 1/4 | 91 | 1.32 | 4/4 | 54 | 1.25 | 8 | 2 | 166 | 4.82E−04 |
| RS1919447 | 1.37 | 2/4 | 52 | 1.23 | 0/0 | 0 | 0 | 12.5 | 1 | 234 | 4.90E−04 |
| RS12026256 | 1.34 | 1/3 | 38 | 1.27 | 3/3 | 1 | 2.21 | 7.91 | 2 | 190 | 5.02E−04 |
| RS283063 | 1.47 | 4/4 | 204 | 1.31 | 0/0 | 0 | 0 | 12.43 | 1 | 238 | 5.07E−04 |
| RS1456196 | 1.61 | 1/2 | 76 | 1.39 | 2/2 | 141 | 1.29 | 7.85 | 2 | 218 | 5.10E−04 |
| RS4516081 | 1.29 | 2/4 | 68 | 1.43 | 4/4 | 33 | 1.28 | 7.87 | 2 | 203 | 5.11E−04 |
| RS10498032 | 1.17 | 1/4 | 100 | 1.37 | 4/4 | 80 | 1.34 | 7.83 | 2 | 206 | 5.28E−04 |
| RS8049647 | 1.32 | 1/3 | 71 | 1.33 | 3/3 | 6 | 1.72 | 7.76 | 2 | 237 | 5.44E−04 |
| RS10485435 | 1.53 | 1/2 | 102 | 1.3 | 2/2 | 113 | 1.32 | 7.74 | 2 | 233 | 5.57E−04 |
| RS10503419 | 1.31 | 1/2 | 68 | 1.36 | 2/2 | 4 | 1.77 | 7.73 | 2 | 219 | 5.70E−04 |
| RS10484065 | 1.35 | 1/2 | 74 | 1.26 | 2/2 | 22 | 1.49 | 7.71 | 2 | 227 | 5.76E−04 |
| RS10495783 | 1.45 | 2/4 | 128 | 1.29 | 4/4 | 62 | 1.33 | 7.7 | 2 | 235 | 5.77E−04 |
| RS6919514 | 1.45 | 4/4 | 199 | 1.31 | 0/0 | 0 | 0 | 12.17 | 1 | 238 | 5.78E−04 |
| RS10489251 | 1.75 | 3/4 | 57 | 1.31 | 4/4 | 175 | 1.33 | 7.67 | 2 | 234 | 5.94E−04 |
| RS1372184 | 1.3 | 3/4 | 71 | 1.43 | 4/4 | 4 | 1.26 | 7.68 | 2 | 219 | 5.98E−04 |
| RS10485116 | 1.61 | 4/4 | 229 | 1.32 | 0/0 | 0 | 0 | 12.09 | 1 | 236 | 6.03E−04 |
| RS250387 | 1.29 | 1/3 | 63 | 1.43 | 3/3 | 7 | 1.42 | 7.65 | 2 | 235 | 6.04E−04 |
| RS817504 | 1.29 | 3/4 | 40 | 1.45 | 4/4 | 9 | 1.35 | 7.69 | 2 | 198 | 6.08E−04 |
| RS1407390 | 1.32 | 2/4 | 72 | 1.32 | 4/4 | 3 | 1.87 | 7.64 | 2 | 237 | 6.09E−04 |
| RS9308931 | 1.35 | 1/2 | 105 | 1.28 | 2/2 | 48 | 1.45 | 7.66 | 2 | 211 | 6.15E−04 |
| RS1884772 | 1.22 | 2/4 | 112 | 1.38 | 4/4 | 72 | 1.34 | 7.62 | 2 | 235 | 6.22E−04 |
| RS227521 | 1.34 | 1/3 | 43 | 1.28 | 3/3 | 1 | 2.21 | 7.62 | 2 | 232 | 6.23E−04 |
| RS2353296 | 1.89 | 1/2 | 29 | 1.47 | 2/2 | 197 | 1.31 | 7.6 | 2 | 224 | 6.41E−04 |
| RS10241173 | 1.32 | 1/3 | 54 | 1.35 | 3/3 | 1 | 2.21 | 7.61 | 2 | 214 | 6.42E−04 |
| RS10508896 | 1.35 | 1/3 | 25 | 1.17 | 3/3 | 2 | 1.11 | 7.58 | 2 | 237 | 6.44E−04 |
| RS2292889 | 1.2 | 2/3 | 78 | 1.26 | 3/3 | 150 | 1.38 | 7.57 | 2 | 233 | 6.53E−04 |
| RS10486853 | 1.31 | 2/4 | 41 | 1.4 | 4/4 | 2 | 1.9 | 7.57 | 2 | 229 | 6.55E−04 |
| RS1559751 | 1.33 | 1/3 | 105 | 1.29 | 3/3 | 31 | 1.49 | 7.56 | 2 | 238 | 6.56E−04 |
| RS10484317 | 1.32 | 2/3 | 18 | 1.37 | 3/3 | 2 | 1.98 | 7.56 | 2 | 234 | 6.58E−04 |
| RS896990 | 1.96 | 1/2 | 41 | 1.38 | 2/2 | 191 | 1.32 | 7.56 | 2 | 231 | 6.60E−04 |
| RS965193 | 1.34 | 3/4 | 74 | 1.29 | 4/4 | 6 | 1.69 | 7.54 | 2 | 238 | 6.68E−04 |
| RS7850478 | 1.51 | 1/3 | 92 | 1.36 | 3/3 | 132 | 1.29 | 7.54 | 2 | 238 | 6.68E−04 |
| RS6492611 | 1.58 | 1/3 | 75 | 1.33 | 3/3 | 142 | 1.32 | 7.55 | 2 | 228 | 6.68E−04 |
| RS492681 | 1.39 | 2/4 | 65 | 1.43 | 4/4 | 159 | 1.29 | 7.53 | 2 | 233 | 6.78E−04 |
| RS1029233 | 1.24 | 1/2 | 84 | 1.41 | 2/2 | 131 | 1.3 | 7.51 | 2 | 235 | 6.89E−04 |
| RS487707 | 1.29 | 1/3 | 102 | 1.4 | 3/3 | 21 | 1.24 | 7.5 | 2 | 236 | 6.95E−04 |
| RS1427421 | 1.39 | 1/3 | 38 | 1.47 | 3/3 | 186 | 1.3 | 7.51 | 2 | 224 | 6.97E−04 |
| RS10520892 | 1.32 | 2/4 | 39 | 1.33 | 4/4 | 3 | 1.87 | 7.48 | 2 | 236 | 7.08E−04 |
| RS10484921 | 1.4 | 1/2 | 83 | 1.25 | 2/2 | 115 | 1.38 | 7.5 | 2 | 217 | 7.09E−04 |
| RS1880438 | 1.3 | 2/4 | 94 | 1.41 | 4/4 | 30 | 1.24 | 7.48 | 2 | 228 | 7.14E−04 |
| RS945849 | 1.33 | 1/3 | 76 | 1.32 | 3/3 | 3 | 1.87 | 7.47 | 2 | 237 | 7.14E−04 |
| RS10518821 | 1.34 | 1/3 | 80 | 1.3 | 3/3 | 6 | 1.69 | 7.47 | 2 | 237 | 7.14E−04 |
| RS352427 | 1.32 | 1/3 | 13 | 1.57 | 3/3 | 1 | 1.6 | 7.44 | 2 | 237 | 7.35E−04 |
| RS768747 | 1.3 | 2/4 | 63 | 1.43 | 4/4 | 8 | 1.18 | 7.43 | 2 | 235 | 7.43E−04 |
| RS295525 | 1.93 | 2/4 | 8 | 1.47 | 4/4 | 231 | 1.32 | 7.42 | 2 | 238 | 7.48E−04 |
| RS10517836 | 1.31 | 2/4 | 33 | 1.41 | 4/4 | 6 | 1.64 | 7.43 | 2 | 222 | 7.53E−04 |
| RS6477845 | 1.87 | 1/3 | 71 | 1.32 | 3/3 | 165 | 1.32 | 7.41 | 2 | 236 | 7.57E−04 |
| RS7874576 | 1.74 | 3/3 | 233 | 1.33 | 0/0 | 0 | 0 | 11.64 | 1 | 235 | 7.60E−04 |
| RS823638 | 1.87 | 1/3 | 57 | 1.34 | 3/3 | 155 | 1.32 | 7.43 | 2 | 212 | 7.61E−04 |
| RS1387551 | 2.21 | 2/4 | 27 | 1.39 | 4/4 | 206 | 1.32 | 7.39 | 2 | 231 | 7.75E−04 |
| RS962592 | 1.32 | 2/4 | 6 | 1.67 | 0/0 | 0 | 0 | 11.57 | 1 | 238 | 7.86E−04 |
| RS10500985 | 1.66 | 1/2 | 71 | 1.35 | 2/2 | 141 | 1.3 | 7.39 | 2 | 215 | 7.87E−04 |
| RS7470491 | 1.33 | 2/4 | 74 | 1.32 | 4/4 | 3 | 1.87 | 7.36 | 2 | 235 | 7.94E−04 |
| RS9294812 | 1.32 | 2/3 | 90 | 1.31 | 3/3 | 21 | 1.53 | 7.36 | 2 | 223 | 8.03E−04 |
| RS7719212 | 1.32 | 2/3 | 16 | 1.41 | 3/3 | 1 | 2.21 | 7.34 | 2 | 236 | 8.08E−04 |
| RS2291386 | 1.31 | 2/4 | 26 | 1.49 | 0/0 | 0 | 0 | 11.5 | 1 | 239 | 8.14E−04 |
| RS10498292 | 1.29 | 1/3 | 67 | 1.42 | 3/3 | 9 | 1.42 | 7.3 | 2 | 238 | 8.38E−04 |
| RS10494066 | 1.71 | 2/4 | 82 | 1.32 | 4/4 | 150 | 1.33 | 7.3 | 2 | 235 | 8.40E−04 |
| RS9303347 | 1 | 1/3 | 13 | 1.57 | 3/3 | 221 | 1.32 | 7.28 | 2 | 232 | 8.58E−04 |
| RS10521202 | 1.45 | 2/4 | 94 | 1.29 | 4/4 | 70 | 1.31 | 7.3 | 2 | 213 | 8.58E−04 |
| RS700049 | 1.87 | 1/2 | 74 | 1.33 | 2/2 | 157 | 1.33 | 7.26 | 2 | 231 | 8.75E−04 |
| RS9304301 | 1.33 | 2/4 | 19 | 1.26 | 4/4 | 1 | 2.21 | 7.25 | 2 | 234 | 8.81E−04 |
| RS718153 | 1.5 | 1/3 | 110 | 1.31 | 3/3 | 100 | 1.31 | 7.25 | 2 | 233 | 8.82E−04 |
| RS1893120 | 1.24 | 2/4 | 80 | 1.29 | 4/4 | 89 | 1.4 | 7.29 | 2 | 197 | 8.82E−04 |
| RS10500668 | 2.21 | 2/4 | 27 | 1.28 | 4/4 | 213 | 1.34 | 7.23 | 2 | 238 | 8.95E−04 |
| RS10497650 | 1.69 | 4/4 | 223 | 1.32 | 0/0 | 0 | 0 | 11.33 | 1 | 226 | 8.96E−04 |
| RS878016 | 1.3 | 1/2 | 81 | 1.34 | 2/2 | 29 | 1.49 | 7.21 | 2 | 237 | 9.13E−04 |
| RS2474263 | 1.23 | 3/4 | 106 | 1.3 | 4/4 | 98 | 1.4 | 7.2 | 2 | 234 | 9.24E−04 |
| RS10518533 | 1.78 | 1/2 | 100 | 1.31 | 2/2 | 122 | 1.33 | 7.2 | 2 | 233 | 9.33E−04 |
| RS1566728 | 1.29 | 1/3 | 94 | 1.41 | 3/3 | 14 | 1.25 | 7.18 | 2 | 237 | 9.39E−04 |
| RS7841198 | 1.79 | 2/3 | 29 | 1.47 | 3/3 | 209 | 1.31 | 7.18 | 2 | 236 | 9.40E−04 |
| RS3827702 | 1.3 | 2/4 | 60 | 1.34 | 4/4 | 19 | 1.53 | 7.22 | 2 | 196 | 9.43E−04 |
| RS901682 | 1.11 | 1/3 | 21 | 1.16 | 3/3 | 214 | 1.35 | 7.15 | 2 | 234 | 9.68E−04 |
| RS10490353 | 1.33 | 2/3 | 43 | 1.3 | 3/3 | 2 | 1.96 | 7.11 | 2 | 238 | 1.00E−03 |
| RS246558 | 1.31 | 1/3 | 61 | 1.36 | 3/3 | 5 | 1.72 | 7.11 | 2 | 236 | 1.00E−03 |
| RS10513993 | 2.21 | 3/4 | 26 | 1.28 | 4/4 | 212 | 1.34 | 7.11 | 2 | 236 | 1.00E−03 |
| RS8027192 | 1.7 | 2/4 | 64 | 1.3 | 4/4 | 170 | 1.33 | 7.1 | 2 | 237 | 1.01E−03 |
| RS9327673 | 1.52 | 1/3 | 95 | 1.31 | 3/3 | 125 | 1.32 | 7.09 | 2 | 238 | 1.02E−03 |
| RS7107851 | 1.29 | 2/3 | 81 | 1.39 | 3/3 | 6 | 1.57 | 7.08 | 2 | 238 | 1.03E−03 |
| RS6798797 | 1.32 | 1/3 | 14 | 1.36 | 3/3 | 2 | 1.97 | 7.08 | 2 | 233 | 1.04E−04 |
| RS2369669 | 1.33 | 1/3 | 65 | 1.28 | 3/3 | 33 | 1.48 | 7.09 | 2 | 208 | 1.05E−03 |
| RS967326 | 1.31 | 2/4 | 30 | 1.47 | 4/4 | 1 | 1.79 | 7.06 | 2 | 237 | 1.05E−03 |
| RS10484518 | 1.34 | 3/4 | 100 | 1.4 | 4/4 | 111 | 1.27 | 7.05 | 2 | 231 | 1.07E−03 |
| RS10521979 | 1.45 | 4/4 | 198 | 1.31 | 0/0 | 0 | 0 | 10.97 | 1 | 237 | 1.07E−03 |
| RS965158 | 1.57 | 4/4 | 230 | 1.32 | 0/0 | 0 | 0 | 10.95 | 1 | 239 | 1.08E−03 |
| RS2294311 | 1.37 | 1/3 | 89 | 1.4 | 3/3 | 134 | 1.28 | 7 | 2 | 236 | 1.11E−03 |
| RS10497649 | 1.63 | 3/3 | 234 | 1.32 | 0/0 | 0 | 0 | 10.88 | 1 | 239 | 1.12E−03 |
| RS1217654 | 1.34 | 2/4 | 36 | 1.3 | 4/4 | 1 | 2.21 | 7 | 2 | 228 | 1.12E−03 |
| RS1773409 | 1.65 | 1/3 | 47 | 1.26 | 3/3 | 188 | 1.34 | 6.99 | 2 | 238 | 1.12E−03 |
| RS9323682 | 1.21 | 1/3 | 36 | 1.47 | 3/3 | 200 | 1.31 | 6.99 | 2 | 235 | 1.12E−03 |
| RS2503102 | 1.4 | 1/3 | 74 | 1.3 | 3/3 | 34 | 1.24 | 7.02 | 2 | 204 | 1.13E−03 |
| RS3745718 | 1.4 | 1/3 | 122 | 1.27 | 3/3 | 45 | 1.39 | 6.98 | 2 | 238 | 1.13E−03 |
| RS370480 | 1.51 | 3/4 | 110 | 1.33 | 4/4 | 109 | 1.3 | 6.97 | 2 | 238 | 1.14E−03 |
| RS1217656 | 2.21 | 1/2 | 38 | 1.29 | 2/2 | 200 | 1.34 | 6.97 | 2 | 236 | 1.15E−03 |
| RS10510715 | 1.97 | 2/4 | 8 | 1.28 | 4/4 | 230 | 1.33 | 6.93 | 2 | 237 | 1.19E−03 |
| RS4510935 | 1.66 | 2/4 | 60 | 1.37 | 4/4 | 174 | 1.31 | 6.93 | 2 | 237 | 1.19E−03 |
| RS2064572 | 1.3 | 1/3 | 100 | 1.41 | 3/3 | 50 | 1.26 | 6.95 | 2 | 213 | 1.19E−03 |
| RS10486848 | 1.32 | 2/4 | 61 | 1.32 | 4/4 | 11 | 1.6 | 6.93 | 2 | 234 | 1.19E−03 |
| RS10515869 | 1.44 | 1/3 | 119 | 1.29 | 3/3 | 69 | 1.33 | 6.91 | 2 | 234 | 1.21E−03 |
| RS10499436 | 1.45 | 1/3 | 85 | 1.38 | 3/3 | 126 | 1.28 | 6.91 | 2 | 227 | 1.22E−03 |
| RS10510714 | 1.33 | 1/3 | 15 | 1.35 | 3/3 | 2 | 1.97 | 6.9 | 2 | 238 | 1.22E−03 |
| RS724620 | 1.25 | 4/4 | 148 | 1.37 | 0/0 | 0 | 0 | 10.72 | 1 | 218 | 1.23E−03 |
| RS6109935 | 1.52 | 2/4 | 82 | 1.34 | 4/4 | 117 | 1.3 | 6.9 | 2 | 215 | 1.25E−03 |
| RS10521613 | 1.56 | 4/4 | 228 | 1.32 | 0/0 | 0 | 0 | 10.66 | 1 | 238 | 1.26E−03 |
| RS10505439 | 2.21 | 1/3 | 28 | 1.36 | 3/3 | 212 | 1.32 | 6.87 | 2 | 238 | 1.26E−03 |
| RS2045765 | 1.31 | 2/4 | 43 | 1.4 | 4/4 | 7 | 1.6 | 6.86 | 2 | 236 | 1.27E−03 |
| RS10512041 | 2.21 | 2/4 | 53 | 1.3 | 4/4 | 185 | 1.34 | 6.86 | 2 | 236 | 1.27E−03 |
| RS10504143 | 1.51 | 4/4 | 213 | 1.32 | 0/0 | 0 | 0 | 10.64 | 1 | 231 | 1.27E−03 |
| RS6079097 | 1.52 | 1/3 | 103 | 1.33 | 3/3 | 117 | 1.3 | 6.85 | 2 | 235 | 1.28E−03 |
| RS10494583 | 1.3 | 2/3 | 81 | 1.36 | 3/3 | 7 | 1.63 | 6.84 | 2 | 237 | 1.29E−03 |
| RS1527420 | 1.31 | 1/2 | 43 | 1.36 | 2/2 | 5 | 1.71 | 6.85 | 2 | 221 | 1.30E−03 |
| RS3218493 | 1.58 | 3/3 | 230 | 1.32 | 0/0 | 0 | 0 | 10.58 | 1 | 238 | 1.31E−03 |
| RS10499437 | 1.45 | 1/3 | 92 | 1.37 | 3/3 | 125 | 1.28 | 6.83 | 2 | 233 | 1.31E−03 |
| RS10512980 | 1.25 | 1/3 | 99 | 1.29 | 3/3 | 110 | 1.4 | 6.83 | 2 | 232 | 1.31E−03 |
| RS10485441 | 1.88 | 1/3 | 40 | 1.39 | 3/3 | 196 | 1.31 | 6.82 | 2 | 235 | 1.32E−04 |
| RS10509448 | 1.31 | 2/4 | 15 | 1.55 | 4/4 | 2 | 1.5 | 6.83 | 2 | 222 | 1.32E−03 |
| RS32967 | 1.31 | 2/3 | 88 | 1.26 | 3/3 | 132 | 1.38 | 6.82 | 2 | 232 | 1.32E−03 |
| RS2371319 | 1.97 | 2/4 | 18 | 1.33 | 4/4 | 221 | 1.33 | 6.8 | 2 | 238 | 1.34E−03 |
| RS10496649 | 1.35 | 2/4 | 38 | 1.24 | 4/4 | 1 | 1.96 | 6.8 | 2 | 236 | 1.35E−03 |
| RS1462478 | 1.31 | 2/4 | 15 | 1.3 | 4/4 | 3 | 1.82 | 6.9 | 2 | 153 | 1.35E−03 |
| RS773013 | 1.59 | 4/4 | 225 | 1.32 | 0/0 | 0 | 0 | 10.52 | 1 | 232 | 1.35E−03 |
| RS6830598 | 1.46 | 2/3 | 91 | 1.34 | 3/3 | 84 | 1.28 | 6.81 | 2 | 207 | 1.37E−03 |
| RS1959715 | 2.21 | 1/3 | 7 | 1.28 | 3/3 | 228 | 1.33 | 6.78 | 2 | 233 | 1.37E−03 |
| RS2111368 | 1.3 | 1/4 | 65 | 1.4 | 4/4 | 1 | 1.83 | 6.77 | 2 | 231 | 1.39E−03 |
| RS965788 | 1.24 | 1/3 | 47 | 1.45 | 3/3 | 175 | 1.31 | 6.77 | 2 | 230 | 1.39E−03 |
| RS4564209 | 1.28 | 2/4 | 118 | 1.34 | 4/4 | 25 | 1.48 | 6.75 | 2 | 237 | 1.41E−03 |
| RS1889465 | 1.31 | 1/3 | 32 | 1.47 | 0/0 | 0 | 0 | 10.42 | 1 | 236 | 1.42E−03 |
| RS10485490 | 1.33 | 2/4 | 17 | 1.29 | 4/4 | 1 | 2.21 | 6.72 | 2 | 234 | 1.45E−03 |
| RS6735220 | 1.33 | 2/4 | 39 | 1.29 | 4/4 | 2 | 1.94 | 6.71 | 2 | 238 | 1.46E−03 |
| RS1427422 | 1.39 | 2/4 | 40 | 1.46 | 4/4 | 196 | 1.31 | 6.71 | 2 | 236 | 1.46E−03 |
| RS245340 | 1.31 | 3/4 | 94 | 1.26 | 4/4 | 131 | 1.39 | 6.7 | 2 | 237 | 1.48E−03 |
| RS4131462 | 1.39 | 2/4 | 107 | 1.32 | 4/4 | 32 | 1.21 | 6.7 | 2 | 237 | 1.48E−03 |
| RS207616 | 1.38 | 1/2 | 110 | 1.27 | 2/2 | 10 | 1.45 | 6.72 | 2 | 214 | 1.48E−03 |
| RS672489 | 1.26 | 2/4 | 104 | 1.38 | 4/4 | 30 | 1.4 | 6.7 | 2 | 224 | 1.49E−03 |
| RS7829473 | 1.33 | 2/4 | 80 | 1.3 | 4/4 | 17 | 1.54 | 6.68 | 2 | 232 | 1.51E−03 |
| RS1459914 | 1.33 | 3/4 | 43 | 1.31 | 4/4 | 1 | 2.21 | 6.66 | 2 | 236 | 1.54E−03 |
| RS10501811 | 1.58 | 1/2 | 74 | 1.4 | 2/2 | 158 | 1.29 | 6.66 | 2 | 231 | 1.54E−03 |
| RS890783 | 1.33 | 1/3 | 27 | 1.49 | 3/3 | 6 | 1.17 | 6.65 | 2 | 227 | 1.56E−03 |
| RS10497472 | 1.47 | 4/4 | 210 | 1.31 | 0/0 | 0 | 0 | 10.22 | 1 | 238 | 1.58E−03 |
| RS709087 | 1.37 | 2/4 | 74 | 1.28 | 4/4 | 5 | 1.64 | 6.66 | 2 | 199 | 1.59E−03 |
| RS3104997 | 1.18 | 2/3 | 89 | 1.29 | 3/3 | 126 | 1.38 | 6.63 | 2 | 229 | 1.59E−03 |
| RS1235012 | 1.34 | 2/4 | 34 | 1.31 | 4/4 | 1 | 2.21 | 6.62 | 2 | 233 | 1.60E−03 |
| RS1589629 | 1.22 | 1/3 | 88 | 1.41 | 3/3 | 107 | 1.3 | 6.63 | 2 | 209 | 1.62E−03 |
| RS10483456 | 1.32 | 2/4 | 22 | 1.34 | 4/4 | 1 | 2.21 | 6.64 | 2 | 199 | 1.62E−03 |
| RS10521240 | 1.56 | 1/2 | 63 | 1.25 | 2/2 | 163 | 1.36 | 6.61 | 2 | 229 | 1.62−03 |
| RS4739647 | 1.13 | 1/3 | 51 | 1.43 | 3/3 | 174 | 1.31 | 6.61 | 2 | 228 | 1.62E−03 |
| RS1121640 | 1.32 | 2/4 | 27 | 1.39 | 4/4 | 2 | 1.92 | 6.6 | 2 | 236 | 1.63E−03 |
| RS10512000 | 1.22 | 4/4 | 204 | 1.35 | 0/0 | 0 | 0 | 10.15 | 1 | 239 | 1.64E−03 |
| RS10486470 | 1.59 | 1/3 | 68 | 1.31 | 3/3 | 159 | 1.32 | 6.58 | 2 | 235 | 1.66E−03 |
| RS1362406 | 1.33 | 3/4 | 22 | 1.33 | 4/4 | 1 | 2.21 | 6.58 | 2 | 234 | 1.66E−03 |
| RS8054578 | 1.81 | 1/3 | 62 | 1.36 | 3/3 | 170 | 1.31 | 6.58 | 2 | 232 | 1.66E−03 |
| RS1228685 | 1.35 | 3/4 | 37 | 1.22 | 4/4 | 4 | 1.59 | 6.59 | 2 | 219 | 1.66E−03 |
| RS10508903 | 1.45 | 1/2 | 118 | 1.33 | 2/2 | 47 | 1.27 | 6.6 | 2 | 207 | 1.66E−03 |
| RS6068024 | 1.31 | 2/4 | 104 | 1.39 | 4/4 | 65 | 1.25 | 6.58 | 2 | 223 | 1.67E−03 |
| RS4945630 | 1.4 | 1/3 | 114 | 1.31 | 3/3 | 36 | 1.24 | 6.56 | 2 | 233 | 1.69E−03 |
| RS2425570 | 1.3 | 2/4 | 31 | 1.36 | 4/4 | 10 | 1.58 | 6.59 | 2 | 196 | 1.70E−03 |
| RS758780 | 1.27 | 2/4 | 114 | 1.35 | 4/4 | 33 | 1.44 | 6.55 | 2 | 238 | 1.70E−03 |
| RS758779 | 1.27 | 1/4 | 114 | 1.35 | 4/4 | 33 | 1.44 | 6.55 | 2 | 238 | 1.70E−03 |
| RS798101 | 1.32 | 2/4 | 121 | 1.29 | 4/4 | 46 | 1.45 | 6.55 | 2 | 231 | 1.71E−03 |
| RS6895637 | 1.39 | 3/4 | 94 | 1.27 | 4/4 | 23 | 1.3 | 6.55 | 2 | 226 | 1.72E−03 |
| RS6777583 | 1.34 | 2/4 | 114 | 1.37 | 4/4 | 47 | 1.22 | 6.54 | 2 | 233 | 1.72E−03 |
| RS10518896 | 1.54 | 4/4 | 227 | 1.32 | 0/0 | 0 | 0 | 10.04 | 1 | 238 | 1.73E−03 |
| RS10498944 | 1.83 | 1/4 | 38 | 1.41 | 4/4 | 200 | 1.31 | 6.53 | 2 | 237 | 1.74E−03 |
| RS4631686 | 1.61 | 3/3 | 228 | 1.33 | 0/0 | 0 | 0 | 10.04 | 1 | 234 | 1.74E−03 |
| RS10503057 | 1.37 | 1/4 | 77 | 1.25 | 4/4 | 9 | 1.45 | 6.53 | 2 | 235 | 1.74E−03 |
| RS246580 | 1.32 | 2/4 | 58 | 1.35 | 4/4 | 6 | 1.68 | 6.53 | 2 | 234 | 1.74E−03 |
| RS719530 | 1.26 | 2/4 | 93 | 1.4 | 4/4 | 65 | 1.31 | 6.55 | 2 | 209 | 1.74E−03 |
| RS10483942 | 1.21 | 2/4 | 35 | 1.46 | 4/4 | 198 | 1.3 | 6.53 | 2 | 232 | 1.74E−03 |
| RS1499976 | 1.29 | 2/4 | 73 | 1.36 | 4/4 | 14 | 1.51 | 6.52 | 2 | 232 | 1.76E−03 |
| RS2769261 | 1.37 | 1/2 | 60 | 1.44 | 2/2 | 140 | 1.3 | 6.54 | 2 | 207 | 1.76E−03 |
| RS752355 | 1.56 | 1/2 | 101 | 1.32 | 2/2 | 110 | 1.33 | 6.52 | 2 | 223 | 1.77E−03 |
| RS10485492 | 2.21 | 2/4 | 16 | 1.32 | 4/4 | 221 | 1.33 | 6.51 | 2 | 235 | 1.77E−03 |
| RS2296697 | 1.3 | 2/4 | 55 | 1.4 | 4/4 | 10 | 1.52 | 6.51 | 2 | 231 | 1.78E−03 |
| RS717806 | 1.52 | 1/4 | 95 | 1.36 | 4/4 | 125 | 1.28 | 6.5 | 2 | 228 | 1.80E−03 |
| RS1887545 | 0.96 | 1/3 | 37 | 1.42 | 3/3 | 178 | 1.32 | 6.5 | 2 | 215 | 1.82E−03 |
| RS1506672 | 1.43 | 2/4 | 95 | 1.27 | 4/4 | 78 | 1.35 | 6.5 | 2 | 212 | 1.82E−03 |
| RS10494584 | 1.31 | 2/4 | 78 | 1.36 | 4/4 | 7 | 1.63 | 6.48 | 2 | 235 | 1.82E−03 |
| RS1480145 | 1.32 | 2/3 | 12 | 1.55 | 0/0 | 0 | 0 | 9.94 | 1 | 239 | 1.82E−03 |
| RS3218491 | 1.64 | 4/4 | 230 | 1.32 | 0/0 | 0 | 0 | 9.94 | 1 | 234 | 1.83E−03 |
| RS10510104 | 1.65 | 2/4 | 78 | 1.37 | 4/4 | 158 | 1.3 | 6.47 | 2 | 238 | 1.84E−03 |
| RS1465135 | 1.27 | 1/3 | 74 | 1.4 | 3/3 | 20 | 1.35 | 6.49 | 2 | 211 | 1.84E−03 |
| RS1010504 | 2.21 | 1/3 | 27 | 1.34 | 3/3 | 210 | 1.33 | 6.47 | 2 | 235 | 1.84E−03 |
| RS956408 | 1.31 | 2/4 | 92 | 1.3 | 4/4 | 33 | 1.47 | 6.47 | 2 | 234 | 1.84E−03 |
| RS10497568 | 1.23 | 2/3 | 112 | 1.39 | 3/3 | 73 | 1.32 | 6.47 | 2 | 226 | 1.85E−03 |
| RS10510986 | 1.31 | 1/3 | 42 | 1.46 | 3/3 | 3 | 1.31 | 6.46 | 2 | 236 | 1.86E−03 |
| RS929332 | 1.28 | 2/4 | 93 | 1.4 | 4/4 | 17 | 1.34 | 6.46 | 2 | 236 | 1.86E−03 |
| RS6772743 | 1.61 | 2/3 | 60 | 1.34 | 3/3 | 166 | 1.31 | 6.46 | 2 | 232 | 1.86E−03 |
| RS10521297 | 1.33 | 1/3 | 43 | 1.34 | 3/3 | 1 | 2.21 | 6.46 | 2 | 232 | 1.86E−03 |
| RS7775656 | 1.37 | 1/3 | 60 | 1.24 | 3/3 | 8 | 1.3 | 6.46 | 2 | 229 | 1.87E−03 |
| RS2291385 | 1.48 | 3/3 | 215 | 1.31 | 0/0 | 0 | 0 | 9.88 | 1 | 238 | 1.88E−03 |
| RS10490656 | 1.37 | 1/3 | 84 | 1.26 | 3/3 | 17 | 1.41 | 6.44 | 2 | 238 | 1.89E−03 |
| RS3820182 | 1.63 | 1/3 | 79 | 1.35 | 3/3 | 153 | 1.31 | 6.44 | 2 | 236 | 1.89E−03 |
| RS6990997 | 1.4 | 2/4 | 38 | 1.46 | 4/4 | 194 | 1.31 | 6.44 | 2 | 235 | 1.89E−03 |
| RS1883736 | 1.53 | 3/3 | 226 | 1.32 | 0/0 | 0 | 0 | 9.86 | 1 | 238 | 1.90E−03 |
| RS228563 | 1.22 | 1/2 | 90 | 1.38 | 2/2 | 83 | 1.35 | 6.45 | 2 | 215 | 1.90E−03 |
| RS2353193 | 1.29 | 2/3 | 79 | 1.41 | 3/3 | 4 | 1.36 | 6.43 | 2 | 237 | 1.91E−03 |
| RS10483887 | 2.21 | 2/4 | 19 | 1.33 | 4/4 | 215 | 1.33 | 6.43 | 2 | 232 | 1.91E−03 |
| RS10492047 | 1.46 | 3/4 | 21 | 1.51 | 4/4 | 218 | 1.31 | 6.42 | 2 | 238 | 1.93E−03 |
| RS6955049 | 2.21 | 1/3 | 48 | 1.33 | 3/3 | 177 | 1.33 | 6.42 | 2 | 223 | 1.95E−03 |
| RS7181421 | 1.29 | 2/4 | 116 | 1.39 | 4/4 | 30 | 1.24 | 6.41 | 2 | 234 | 1.95E−03 |
| RS246575 | 1.31 | 1/2 | 60 | 1.36 | 2/2 | 5 | 1.69 | 6.4 | 2 | 234 | 1.97E−03 |
| RS10510809 | 1.3 | 2/4 | 58 | 1.43 | 4/4 | 10 | 1.38 | 6.4 | 2 | 229 | 1.97E−03 |
| RS10517211 | 1.31 | 2/4 | 53 | 1.4 | 4/4 | 4 | 1.65 | 6.39 | 2 | 238 | 1.98E−03 |
| RS10517954 | 1.1 | 1/3 | 44 | 1.43 | 3/3 | 177 | 1.32 | 6.4 | 2 | 224 | 1.98E−03 |
| RS7529360 | 1.31 | 2/4 | 107 | 1.31 | 4/4 | 35 | 1.47 | 6.39 | 2 | 233 | 1.99E−03 |
| RS1046852 | 1.46 | 1/3 | 111 | 1.32 | 3/3 | 88 | 1.29 | 6.38 | 2 | 233 | 2.01E−03 |
| RS10509702 | 1.32 | 1/3 | 10 | 1.52 | 3/3 | 1 | 1.96 | 6.37 | 2 | 236 | 2.02E−03 |
| RS958425 | 1.31 | 1/2 | 58 | 1.4 | 2/2 | 2 | 1.75 | 6.36 | 2 | 237 | 2.04E−03 |
| RS4673999 | 1.42 | 1/4 | 124 | 1.3 | 4/4 | 49 | 1.28 | 6.36 | 2 | 234 | 2.04E−03 |
| RS4888946 | 1.31 | 1/4 | 59 | 1.35 | 4/4 | 3 | 1.81 | 6.36 | 2 | 230 | 2.05E−03 |
| RS2163721 | 1.34 | 2/4 | 9 | 1.09 | 0/0 | 0 | 0 | 9.71 | 1 | 234 | 2.06E−03 |
| RS7782588 | 1.32 | 1/3 | 46 | 1.28 | 3/3 | 13 | 1.55 | 6.36 | 2 | 213 | 2.08E−03 |
| RS10489688 | 1.29 | 1/3 | 99 | 1.35 | 3/3 | 32 | 1.46 | 6.34 | 2 | 236 | 2.08E−03 |
| RS10514844 | 1.62 | 3/3 | 232 | 1.33 | 0/0 | 0 | 0 | 9.69 | 1 | 237 | 2.08E−03 |
| RS779320 | 1.39 | 2/4 | 92 | 1.27 | 4/4 | 24 | 1.3 | 6.33 | 2 | 233 | 2.10E−03 |
| RS3749216 | 1.33 | 1/3 | 4 | 1.71 | 0/0 | 0 | 0 | 9.65 | 1 | 239 | 2.12E−03 |
| RS10496466 | 1.38 | 2/4 | 73 | 1.27 | 4/4 | 8 | 1.5 | 6.34 | 2 | 205 | 2.13E−03 |
| RS650298 | 1.4 | 2/4 | 110 | 1.28 | 4/4 | 53 | 1.36 | 6.31 | 2 | 235 | 2.14E−03 |
| RS2671762 | 1.3 | 2/4 | 43 | 1.44 | 4/4 | 3 | 1.53 | 6.29 | 2 | 237 | 2.18E−03 |
| RS10517293 | 1.46 | 3/3 | 198 | 1.31 | 0/0 | 0 | 0 | 9.61 | 1 | 226 | 2.18E−03 |
| RS1851006 | 1.28 | 2/4 | 63 | 1.26 | 4/4 | 70 | 1.4 | 6.34 | 2 | 175 | 2.20E−03 |
| RS2761089 | 1.3 | 2/4 | 105 | 1.38 | 4/4 | 31 | 1.22 | 6.3 | 2 | 213 | 2.20E−03 |
| RS10510987 | 1.31 | 2/4 | 36 | 1.46 | 4/4 | 197 | 1.31 | 6.28 | 2 | 233 | 2.21E−03 |
| RS362865 | 1.36 | 2/3 | 54 | 1.26 | 3/3 | 5 | 1.06 | 6.27 | 2 | 236 | 2.22E−03 |
| RS8089476 | 1.28 | 2/4 | 109 | 1.29 | 4/4 | 94 | 1.4 | 6.27 | 2 | 233 | 2.23E−03 |
| RS1805781 | 1.64 | 3/3 | 231 | 1.33 | 0/0 | 0 | 0 | 9.55 | 1 | 235 | 2.24E−03 |
| RS6837953 | 1.93 | 1/3 | 33 | 1.42 | 3/3 | 191 | 1.31 | 6.27 | 2 | 222 | 2.24E−03 |
| RS4504494 | 1.54 | 3/3 | 228 | 1.32 | 0/0 | 0 | 0 | 9.54 | 1 | 239 | 2.25E−03 |
| RS7556543 | 1.63 | 1/3 | 67 | 1.36 | 3/3 | 154 | 1.31 | 6.26 | 2 | 225 | 2.26E−03 |
| RS2001097 | 1.66 | 1/2 | 58 | 1.39 | 2/2 | 165 | 1.31 | 6.26 | 2 | 225 | 2.26E−03 |
| RS286300 | 1.31 | 2/4 | 26 | 1.48 | 4/4 | 1 | 1.04 | 6.25 | 2 | 237 | 2.26E−03 |
| RS10484926 | 1.33 | 2/4 | 4 | 1.71 | 0/0 | 0 | 0 | 9.53 | 1 | 235 | 2.26E−03 |
| RS2290547 | 1.27 | 1/3 | 87 | 1.4 | 3/3 | 138 | 1.29 | 6.25 | 2 | 235 | 2.27E−03 |
| RS543465 | 1.31 | 2/4 | 32 | 1.48 | 4/4 | 3 | 1.35 | 6.25 | 2 | 235 | 2.27E−03 |
| RS320694 | 1.28 | 2/3 | 101 | 1.4 | 3/3 | 78 | 1.28 | 6.25 | 2 | 225 | 2.28E−03 |
| RS9329911 | 1.35 | 1/3 | 107 | 1.34 | 3/3 | 12 | 1.09 | 6.24 | 2 | 237 | 2.29E−03 |
| RS10486471 | 1.59 | 2/3 | 72 | 1.32 | 3/3 | 155 | 1.32 | 6.24 | 2 | 235 | 2.29E−03 |
| RS6938470 | 1.76 | 1/3 | 52 | 1.37 | 3/3 | 173 | 1.31 | 6.24 | 2 | 225 | 2.30E−03 |
| RS1501622 | 1.28 | 2/4 | 97 | 1.29 | 4/4 | 94 | 1.4 | 6.23 | 2 | 233 | 2.31E−03 |
| RS4611835 | 1.88 | 1/2 | 18 | 1.42 | 2/2 | 213 | 1.32 | 6.22 | 2 | 230 | 2.34E−03 |
| RS10483426 | 1.37 | 2/4 | 122 | 1.28 | 4/4 | 55 | 1.41 | 6.21 | 2 | 237 | 2.35E−03 |
| RS2292250 | 1.33 | 1/3 | 19 | 1.5 | 0/0 | 0 | 0 | 9.46 | 1 | 230 | 2.35E−03 |
| RS4952605 | 1.18 | 1/4 | 101 | 1.38 | 4/4 | 92 | 1.3 | 6.22 | 2 | 211 | 2.37E−03 |
| RS1957425 | 1.36 | 1/3 | 108 | 1.28 | 3/3 | 30 | 1.45 | 6.2 | 2 | 237 | 2.37E−03 |
| RS3773636 | 1.52 | 1/3 | 72 | 1.27 | 3/3 | 139 | 1.35 | 6.21 | 2 | 221 | 2.38E−03 |
| RS4405428 | 1.33 | 2/4 | 52 | 1.32 | 4/4 | 2 | 1.93 | 6.2 | 2 | 234 | 2.38E−03 |
| RS10498096 | 1.07 | 2/4 | 25 | 1.49 | 4/4 | 210 | 1.32 | 6.2 | 2 | 233 | 2.38E−03 |
| RS2316853 | 1.47 | 3/3 | 102 | 1.29 | 0/0 | 0 | 0 | 9.61 | 1 | 127 | 2.38E−03 |
| RS723043 | 1.26 | 3/4 | 113 | 1.34 | 4/4 | 32 | 1.45 | 6.21 | 2 | 210 | 2.40E−03 |
| RS2217610 | 1.29 | 1/3 | 74 | 1.38 | 3/3 | 11 | 1.49 | 6.2 | 2 | 219 | 2.40E−03 |
| RS1878144 | 1.33 | 3/4 | 47 | 1.31 | 4/4 | 5 | 1.71 | 6.19 | 2 | 230 | 2.41E−03 |
| RS10496527 | 1.29 | 2/4 | 94 | 1.26 | 4/4 | 57 | 1.41 | 6.22 | 2 | 190 | 2.42E−03 |
| RS2971828 | 1.31 | 1/3 | 74 | 1.35 | 3/3 | 10 | 1.58 | 6.18 | 2 | 237 | 2.42E−03 |
| RS2669892 | 1.29 | 1/3 | 70 | 1.42 | 3/3 | 9 | 1.33 | 6.18 | 2 | 235 | 2.24E−03 |
| RS1915546 | 1.26 | 2/4 | 117 | 1.39 | 4/4 | 35 | 1.3 | 6.18 | 2 | 226 | 2.44E−03 |
| RS823692 | 1.78 | 1/2 | 31 | 1.21 | 2/2 | 209 | 1.35 | 6.17 | 2 | 238 | 2.44E−03 |
| RS1334722 | 1.3 | 1/3 | 110 | 1.39 | 3/3 | 24 | 1.22 | 6.17 | 2 | 238 | 2.44E−03 |
| RS580758 | 1.68 | 1/2 | 39 | 1.4 | 2/2 | 197 | 1.31 | 6.17 | 2 | 237 | 2.44E−03 |
| RS285406 | 1.32 | 2/4 | 22 | 1.49 | 0/0 | 0 | 0 | 9.38 | 1 | 239 | 2.44E−03 |
| RS10500683 | 1.32 | 2/4 | 110 | 1.3 | 4/4 | 33 | 1.47 | 6.17 | 2 | 233 | 2.45E−03 |
| RS1413191 | 1.7 | 1/3 | 64 | 1.35 | 3/3 | 160 | 1.32 | 6.17 | 2 | 226 | 2.46E−03 |
| RS600524 | 1.33 | 2/4 | 94 | 1.3 | 4/4 | 32 | 1.47 | 6.17 | 2 | 223 | 2.47E−03 |
| RS1506326 | 1.39 | 3/4 | 63 | 1.24 | 4/4 | 152 | 1.37 | 6.17 | 2 | 223 | 2.47E−03 |
| RS10495774 | 1.6 | 2/4 | 75 | 1.32 | 4/4 | 154 | 1.32 | 6.16 | 2 | 236 | 2.47E−03 |
| RS10497783 | 1.94 | 1/4 | 33 | 1.31 | 4/4 | 202 | 1.33 | 6.16 | 2 | 234 | 2.47E−03 |
| RS1579274 | 1.51 | 1/2 | 91 | 1.33 | 2/2 | 129 | 1.3 | 6.14 | 2 | 236 | 2.51E−03 |
| RS10503572 | 1.67 | 3/3 | 234 | 1.33 | 0/0 | 0 | 0 | 9.32 | 1 | 237 | 2.53E−03 |
| RS2195989 | 1.58 | 2/4 | 51 | 1.38 | 4/4 | 171 | 1.31 | 6.14 | 2 | 228 | 2.53E−03 |
| RS2223753 | 1.32 | 1/4 | 121 | 1.3 | 4/4 | 44 | 1.45 | 6.13 | 2 | 238 | 2.54E−03 |
| RS737769 | 1.45 | 4/4 | 204 | 1.31 | 0/0 | 0 | 0 | 9.3 | 1 | 239 | 2.55E−03 |
| RS39626 | 1.38 | 2/4 | 95 | 1.27 | 4/4 | 15 | 1.31 | 6.12 | 2 | 237 | 2.56E−03 |
| RS7828096 | 1.79 | 1/3 | 29 | 1.45 | 3/3 | 210 | 1.31 | 6.11 | 2 | 237 | 2.59E−03 |
| RS10518660 | 1.31 | 1/3 | 29 | 1.46 | 0/0 | 0 | 0 | 9.27 | 1 | 237 | 2.59E−03 |
| RS958978 | 1.31 | 1/3 | 81 | 1.34 | 3/3 | 9 | 1.6 | 6.11 | 2 | 232 | 2.59E−03 |
| RS717897 | 1.26 | 1/3 | 87 | 1.4 | 3/3 | 132 | 1.29 | 6.11 | 2 | 227 | 2.60E−03 |
| RS10517210 | 1.78 | 2/3 | 55 | 1.42 | 3/3 | 184 | 1.31 | 6.1 | 2 | 237 | 2.61E−03 |
| RS720614 | 1.6 | 2/4 | 70 | 1.34 | 4/4 | 160 | 1.31 | 6.1 | 2 | 236 | 2.61E−03 |
| RS6949530 | 1.31 | 1/4 | 63 | 1.38 | 4/4 | 4 | 1.67 | 6.09 | 2 | 238 | 2.63E−03 |
| RS989177 | 1.56 | 2/4 | 40 | 1.33 | 4/4 | 155 | 1.31 | 6.11 | 2 | 205 | 2.65E−03 |
| RS2591651 | 1.66 | 3/3 | 236 | 1.33 | 0/0 | 0 | 0 | 9.21 | 1 | 239 | 2.67E−03 |
| RS962354 | 1.32 | 1/4 | 49 | 1.3 | 4/4 | 8 | 1.61 | 6.08 | 2 | 226 | 2.68E−03 |
| RS590599 | 1.33 | 2/3 | 53 | 1.44 | 3/3 | 169 | 1.3 | 6.08 | 2 | 226 | 2.68E−03 |
| RS198278 | 1.24 | 1/3 | 118 | 1.34 | 3/3 | 59 | 1.39 | 6.07 | 2 | 227 | 2.70E−03 |
| RS870241 | 1.29 | 2/4 | 78 | 1.37 | 4/4 | 24 | 1.46 | 6.07 | 2 | 222 | 2.71E−03 |
| RS727505 | 1.31 | 2/4 | 71 | 1.34 | 4/4 | 9 | 1.6 | 6.06 | 2 | 234 | 2.72E−03 |
| RS10503068 | 1.42 | 2/4 | 75 | 1.26 | 4/4 | 137 | 1.37 | 6.07 | 2 | 219 | 2.72E−03 |
| RS1356216 | 1.33 | 3/4 | 47 | 1.31 | 4/4 | 5 | 1.71 | 6.05 | 2 | 236 | 2.74E−03 |
| RS10483988 | 1.34 | 2/4 | 62 | 1.3 | 4/4 | 4 | 1.73 | 6.05 | 2 | 236 | 2.74E−03 |
| RS2040868 | 1.28 | 2/4 | 81 | 1.4 | 4/4 | 18 | 1.34 | 6.05 | 2 | 224 | 2.76E−03 |
| RS694795 | 1.36 | 1/4 | 115 | 1.29 | 4/4 | 58 | 1.42 | 6.05 | 2 | 223 | 2.76E−03 |
| RS4073474 | 1.35 | 1/3 | 107 | 1.28 | 3/3 | 33 | 1.45 | 6.04 | 2 | 237 | 2.76E−03 |
| RS10513166 | 1.91 | 2/3 | 43 | 1.29 | 3/3 | 195 | 1.34 | 6.04 | 2 | 237 | 2.76E−03 |
| RS9306468 | 1.37 | 1/3 | 81 | 1.29 | 3/3 | 11 | 1.15 | 6.04 | 2 | 235 | 2.77E−03 |
| RS2825652 | 1.32 | 2/3 | 10 | 1.56 | 0/0 | 0 | 0 | 9.14 | 1 | 238 | 2.77E−03 |
| RS9325559 | 1.67 | 1/3 | 67 | 1.31 | 3/3 | 119 | 1.32 | 6.07 | 2 | 189 | 2.79E−03 |
| RS1519684 | 1.42 | 2/4 | 118 | 1.36 | 4/4 | 89 | 1.26 | 6.03 | 2 | 232 | 2.80E−03 |
| RS4533975 | 1.33 | 2/4 | 60 | 1.28 | 4/4 | 16 | 1.52 | 6.03 | 2 | 229 | 2.80E−03 |
| RS2973234 | 1.33 | 2/4 | 78 | 1.3 | 4/4 | 22 | 1.51 | 6.04 | 2 | 212 | 2.81E−03 |
| RS10513319 | 1.32 | 1/3 | 16 | 1.51 | 0/0 | 0 | 0 | 9.11 | 1 | 238 | 2.82E−03 |
| RS847852 | 1.26 | 1/3 | 110 | 1.39 | 3/3 | 89 | 1.29 | 6.01 | 2 | 235 | 2.85E−03 |
| RS9319997 | 1.37 | 2/4 | 76 | 1.26 | 4/4 | 7 | 1.46 | 6.01 | 2 | 231 | 2.85E−03 |
| RS953177 | 1.32 | 2/4 | 26 | 1.47 | 0/0 | 0 | 0 | 9.08 | 1 | 237 | 2.86E−03 |
| RS10489474 | 1.33 | 2/4 | 72 | 1.29 | 4/4 | 10 | 1.58 | 6 | 2 | 238 | 2.87E−03 |
| RS10490347 | 1.36 | 2/4 | 46 | 1.22 | 4/4 | 194 | 1.36 | 6 | 2 | 238 | 2.87E−03 |
| RS947271 | 1.43 | 1/3 | 106 | 1.31 | 3/3 | 76 | 1.3 | 6 | 2 | 235 | 2.87E−03 |
| RS1608272 | 1.3 | 1/4 | 93 | 1.4 | 4/4 | 18 | 1.24 | 5.99 | 2 | 237 | 2.90E−03 |
| RS9320780 | 1.17 | 1/3 | 74 | 1.4 | 3/3 | 132 | 1.32 | 6 | 2 | 216 | 2.91E−03 |
| RS10516971 | 1.38 | 1/3 | 86 | 1.28 | 3/3 | 13 | 1.23 | 5.98 | 2 | 235 | 2.93E−03 |
| RS4597125 | 1.63 | 1/3 | 65 | 1.36 | 3/3 | 149 | 1.3 | 5.99 | 2 | 217 | 2.94E−03 |
| RS10493351 | 1.32 | 2/4 | 10 | 1.56 | 0/0 | 0 | 0 | 9.03 | 1 | 239 | 2.94E−03 |
| RS756458 | 1.28 | 2/3 | 79 | 1.41 | 3/3 | 144 | 1.3 | 5.97 | 2 | 238 | 2.95E−03 |
| RS333856 | 1.61 | 2/2 | 234 | 1.32 | 0/0 | 0 | 0 | 9.02 | 1 | 239 | 2.95E−03 |
| RS6844368 | 1.31 | 3/4 | 120 | 1.29 | 4/4 | 81 | 1.41 | 5.97 | 2 | 236 | 2.96E−03 |
| RS4146127 | 1.31 | 1/3 | 70 | 1.39 | 3/3 | 4 | 1.66 | 5.97 | 2 | 232 | 2.96E−03 |
| RS1254275 | 1.66 | 3/4 | 58 | 1.3 | 4/4 | 169 | 1.33 | 5.97 | 2 | 230 | 2.97E−03 |
| RS2804861 | 1.32 | 1/3 | 55 | 1.42 | 3/3 | 8 | 1.16 | 5.97 | 2 | 229 | 2.97E−03 |
| RS1268444 | 1.26 | 2/4 | 103 | 1.38 | 4/4 | 27 | 1.39 | 5.98 | 2 | 214 | 2.97E−03 |
| RS176157 | 1.89 | 1/3 | 34 | 1.38 | 3/3 | 205 | 1.32 | 5.96 | 2 | 238 | 2.98E−03 |
| RS10506681 | 1.27 | 2/4 | 110 | 1.36 | 4/4 | 31 | 1.43 | 5.96 | 2 | 237 | 2.98E−03 |
| RS1581412 | 1.24 | 2/4 | 98 | 1.39 | 4/4 | 109 | 1.3 | 5.96 | 2 | 236 | 2.98E−03 |
| RS2111826 | 1.22 | 3/3 | 201 | 1.35 | 0/0 | 0 | 0 | 9 | 1 | 236 | 2.99E−03 |
| RS2018842 | 1.39 | 1/3 | 119 | 1.33 | 3/3 | 37 | 1.22 | 5.96 | 2 | 233 | 2.99E−03 |
| RS1298362 | 1.23 | 1/3 | 112 | 1.34 | 3/3 | 65 | 1.38 | 5.96 | 2 | 226 | 3.00E−03 |
| RS4492337 | 1.28 | 1/3 | 95 | 1.38 | 3/3 | 24 | 1.44 | 5.96 | 2 | 225 | 3.00E−03 |
| RS2246219 | 1.14 | 1/3 | 95 | 1.35 | 3/3 | 127 | 1.34 | 5.95 | 2 | 236 | 3.01E−03 |
| RS9285977 | 1.3 | 2/4 | 110 | 1.39 | 4/4 | 55 | 1.26 | 5.95 | 2 | 230 | 3.02E−03 |
| RS9322914 | 1.33 | 2/4 | 48 | 1.31 | 4/4 | 5 | 1.7 | 5.95 | 2 | 228 | 3.03E−03 |
| RS4721923 | 1.28 | 3/4 | 89 | 1.41 | 4/4 | 129 | 1.3 | 5.94 | 2 | 236 | 3.04E−03 |
| RS4140855 | 1.5 | 1/3 | 36 | 1.23 | 3/3 | 155 | 1.36 | 5.97 | 2 | 196 | 3.04E−03 |
| RS10518014 | 1.29 | 1/3 | 104 | 1.28 | 3/3 | 110 | 1.39 | 5.93 | 2 | 231 | 3.08E−03 |
| RS1979739 | 1.25 | 1/3 | 60 | 1.42 | 3/3 | 100 | 1.31 | 5.96 | 2 | 191 | 3.08E−03 |
| RS10515526 | 1.61 | 3/3 | 234 | 1.32 | 0/0 | 0 | 0 | 8.93 | 1 | 239 | 3.10E−03 |
| RS2887919 | 1.42 | 1/4 | 97 | 1.27 | 4/4 | 120 | 1.36 | 5.92 | 2 | 235 | 3.10E−03 |
| RS6954168 | 1.32 | 2/4 | 7 | 1.6 | 0/0 | 0 | 0 | 8.91 | 1 | 238 | 3.13E−03 |
| RS2825673 | 1.48 | 2/4 | 43 | 1.44 | 4/4 | 186 | 1.31 | 5.91 | 2 | 232 | 3.14E−03 |
| RS4608876 | 1.29 | 1/2 | 105 | 1.39 | 2/2 | 32 | 1.27 | 5.9 | 2 | 238 | 3.16E−03 |
| RS7241109 | 1.27 | 1/3 | 100 | 1.39 | 3/3 | 23 | 1.35 | 5.9 | 2 | 232 | 3.17E−03 |
| RS2042651 | 1.27 | 1/3 | 104 | 1.4 | 3/3 | 98 | 1.29 | 5.91 | 2 | 217 | 3.17E−03 |
| RS4661451 | 1.28 | 1/3 | 127 | 1.39 | 3/3 | 59 | 1.28 | 5.89 | 2 | 236 | 3.19E−03 |
| RS6708913 | 1.79 | 1/3 | 38 | 1.29 | 3/3 | 180 | 1.33 | 5.9 | 2 | 218 | 3.20E−03 |
| RS211587 | 1.89 | 2/4 | 52 | 1.29 | 4/4 | 186 | 1.34 | 5.88 | 2 | 237 | 3.22E−03 |
| RS2409113 | 1.29 | 2/4 | 75 | 1.41 | 4/4 | 11 | 1.34 | 5.88 | 2 | 235 | 3.22E−03 |
| RS7926211 | 1.24 | 1/3 | 82 | 1.41 | 3/3 | 138 | 1.3 | 5.87 | 2 | 237 | 3.25E−03 |
| RS11232919 | 1.92 | 2/4 | 61 | 1.32 | 4/4 | 177 | 1.33 | 5.87 | 2 | 237 | 3.25E−03 |
| RS720613 | 1.31 | 2/4 | 71 | 1.34 | 4/4 | 10 | 1.59 | 5.88 | 2 | 220 | 3.25E−03 |
| RS10498518 | 1.45 | 1/3 | 106 | 1.28 | 3/3 | 102 | 1.36 | 5.87 | 2 | 235 | 3.25E−03 |
| RS2670607 | 1.82 | 1/4 | 44 | 1.33 | 4/4 | 190 | 1.33 | 5.87 | 2 | 234 | 3.26E−03 |
| RS10506700 | 1.36 | 2/4 | 76 | 1.3 | 4/4 | 6 | 1.03 | 5.87 | 2 | 229 | 3.27E−03 |
| RS10496626 | 1.49 | 4/4 | 216 | 1.32 | 0/0 | 0 | 0 | 8.83 | 1 | 233 | 3.27E−03 |
| RS10514450 | 1.81 | 2/4 | 53 | 1.34 | 4/4 | 168 | 1.32 | 5.87 | 2 | 221 | 3.28E−03 |
| RS2288272 | 1.33 | 2/4 | 83 | 1.28 | 4/4 | 34 | 1.45 | 5.86 | 2 | 227 | 3.30E−03 |
| RS1938327 | 1.32 | 1/3 | 18 | 1.5 | 0/0 | 0 | 0 | 8.81 | 1 | 238 | 3.30E−03 |
| RS1296210 | 1.43 | 2/3 | 112 | 1.29 | 3/3 | 71 | 1.32 | 5.85 | 2 | 237 | 3.31E−03 |
| RS10495058 | 1.61 | 3/3 | 227 | 1.32 | 0/0 | 0 | 0 | 8.79 | 1 | 232 | 3.34E−03 |
| RS10507605 | 1.32 | 1/4 | 25 | 1.39 | 4/4 | 2 | 1.88 | 5.84 | 2 | 235 | 3.35E−03 |
| RS1944989 | 1.47 | 1/3 | 96 | 1.27 | 3/3 | 126 | 1.36 | 5.84 | 2 | 233 | 3.35E−03 |
| RS10490838 | 1.32 | 1/3 | 15 | 1.51 | 0/0 | 0 | 0 | 8.78 | 1 | 239 | 3.35E−03 |
| RS2257086 | 1.3 | 2/4 | 78 | 1.42 | 4/4 | 15 | 1.35 | 5.84 | 2 | 224 | 3.37E−03 |
| RS6601146 | 1.29 | 3/4 | 81 | 1.27 | 4/4 | 132 | 1.38 | 5.83 | 2 | 233 | 3.38E−03 |
| RS25931 | 1.44 | 3/3 | 203 | 1.31 | 0/0 | 0 | 0 | 8.76 | 1 | 239 | 3.39E−03 |
| RS10516613 | 1.29 | 2/3 | 89 | 1.42 | 3/3 | 43 | 1.3 | 5.86 | 2 | 190 | 3.39E−03 |
| RS1931991 | 1.08 | 3/4 | 34 | 1.42 | 4/4 | 200 | 1.33 | 5.82 | 2 | 238 | 3.41E−03 |
| RS261861 | 1.24 | 1/3 | 107 | 1.31 | 3/3 | 86 | 1.39 | 5.82 | 2 | 228 | 3.43E−03 |
| RS1460176 | 1.28 | 1/2 | 107 | 1.4 | 2/2 | 27 | 1.29 | 5.81 | 2 | 236 | 3.44E−03 |
| RS10504744 | 1.31 | 2/3 | 108 | 1.31 | 3/3 | 32 | 1.47 | 5.81 | 2 | 236 | 3.44E−03 |
| RS10484407 | 1.06 | 1/3 | 11 | 1.55 | 3/3 | 223 | 1.32 | 5.81 | 2 | 233 | 3.45E−03 |
| RS4722207 | 1.47 | 1/2 | 106 | 1.29 | 2/2 | 103 | 1.34 | 5.81 | 2 | 233 | 3.45E−03 |
| RS2009373 | 1.38 | 2/4 | 122 | 1.28 | 4/4 | 71 | 1.39 | 5.81 | 2 | 231 | 3.45E−03 |
| RS10518901 | 1.27 | 1/3 | 96 | 1.34 | 3/3 | 48 | 1.42 | 5.81 | 2 | 221 | 3.47E−03 |
| RS173036 | 1.32 | 2/4 | 10 | 1.56 | 0/0 | 0 | 0 | 8.71 | 1 | 239 | 3.48E−03 |
| RS7718000 | 1.65 | 3/3 | 232 | 1.32 | 0/0 | 0 | 0 | 8.69 | 1 | 235 | 3.52E−03 |
| RS10496647 | 1.96 | 1/3 | 31 | 1.24 | 3/3 | 209 | 1.34 | 5.78 | 2 | 238 | 3.54E−03 |
| RS2374031 | 1.32 | 1/3 | 82 | 1.32 | 3/3 | 16 | 1.53 | 5.78 | 2 | 238 | 3.54E−03 |
| RS10503066 | 1.37 | 1/3 | 76 | 1.26 | 3/3 | 11 | 1.39 | 5.78 | 2 | 237 | 3.54E−03 |
| RS4764277 | 1.26 | 1/3 | 107 | 1.39 | 3/3 | 44 | 1.32 | 5.79 | 2 | 221 | 3.54E−03 |
| RS6977649 | 1.92 | 2/3 | 32 | 1.31 | 3/3 | 202 | 1.33 | 5.78 | 2 | 233 | 3.55E−03 |
| RS1119006 | 1.31 | 3/4 | 23 | 1.46 | 0/0 | 0 | 0 | 8.67 | 1 | 237 | 3.56E−03 |
| RS2827082 | 1.69 | 2/4 | 78 | 1.33 | 4/4 | 149 | 1.31 | 5.78 | 2 | 229 | 3.56E−03 |
| RS185926 | 1.04 | 1/4 | 16 | 1.52 | 4/4 | 213 | 1.32 | 5.78 | 2 | 227 | 3.56E−03 |
| RS1434817 | 1.86 | 1/3 | 4 | 1.01 | 3/3 | 234 | 1.34 | 5.77 | 2 | 236 | 3.58E−03 |
| RS9313999 | 1.42 | 2/4 | 124 | 1.29 | 4/4 | 51 | 1.35 | 5.77 | 2 | 235 | 3.58E−03 |
| RS1011439 | 1 | 1/3 | 13 | 1.54 | 3/3 | 227 | 1.32 | 5.76 | 2 | 238 | 3.61E−03 |
| RS7706518 | 1.31 | 1/3 | 42 | 1.45 | 3/3 | 3 | 1.37 | 5.76 | 2 | 237 | 3.61E−03 |
| RS2157768 | 1.32 | 1/3 | 13 | 1.54 | 3/3 | 1 | 1 | 5.76 | 2 | 237 | 3.61E−03 |
| RS718883 | 1.32 | 1/3 | 80 | 1.32 | 3/3 | 15 | 1.54 | 5.76 | 2 | 232 | 3.62E−03 |
| RS6673693 | 1.64 | 1/2 | 64 | 1.33 | 2/2 | 156 | 1.32 | 5.76 | 2 | 224 | 3.64E−03 |
| RS889053 | 1.12 | 1/3 | 77 | 1.3 | 3/3 | 150 | 1.36 | 5.75 | 2 | 235 | 3.65E−03 |
| RS10498602 | 1.16 | 4/4 | 224 | 1.34 | 0/0 | 0 | 0 | 8.62 | 1 | 238 | 3.65E−03 |
| RS10483512 | 1.26 | 2/4 | 104 | 1.38 | 4/4 | 52 | 1.37 | 5.75 | 2 | 233 | 3.65E−03 |
| RS2362932 | 1.47 | 1/3 | 69 | 1.33 | 3/3 | 97 | 1.3 | 5.77 | 2 | 197 | 3.67E−03 |
| RS1497504 | 1.62 | 1/3 | 66 | 1.35 | 3/3 | 166 | 1.31 | 5.74 | 2 | 236 | 3.68E−03 |
| RS10521241 | 1.58 | 1/3 | 70 | 1.26 | 3/3 | 162 | 1.35 | 5.74 | 2 | 233 | 3.69E−03 |
| RS1025748 | 1.33 | 1/2 | 70 | 1.29 | 2/2 | 15 | 1.53 | 5.74 | 2 | 230 | 3.69E−03 |
| RS10484973 | 1.48 | 2/4 | 70 | 1.39 | 4/4 | 148 | 1.3 | 5.74 | 2 | 229 | 3.70E−03 |
| RS1374028 | 1.35 | 1/3 | 31 | 1.19 | 3/3 | 1 | 1.19 | 5.75 | 2 | 212 | 3.70E−03 |
| RS2842527 | 1.39 | 1/3 | 104 | 1.27 | 3/3 | 39 | 1.37 | 5.74 | 2 | 226 | 3.70E−03 |
| RS5979345 | 1.32 | 3/3 | 13 | 1.53 | 0/0 | 0 | 0 | 8.59 | 1 | 238 | 3.71E−03 |
| RS9312013 | 1.32 | 3/4 | 11 | 1.54 | 0/0 | 0 | 0 | 8.6 | 1 | 223 | 3.71E−03 |
| RS10507607 | 1.32 | 2/3 | 27 | 1.38 | 3/3 | 2 | 1.88 | 5.73 | 2 | 237 | 3.71E−03 |
| RS10494826 | 1.35 | 2/4 | 71 | 1.41 | 4/4 | 156 | 1.29 | 5.73 | 2 | 235 | 3.72E−03 |
| RS634785 | 1.34 | 2/4 | 23 | 1.19 | 0/0 | 0 | 0 | 8.59 | 1 | 229 | 3.72E−03 |
| RS737911 | 1.15 | 1/3 | 74 | 1.29 | 3/3 | 147 | 1.37 | 5.73 | 2 | 229 | 3.73E−03 |
| RS4684321 | 1.32 | 1/3 | 23 | 1.48 | 0/0 | 0 | 0 | 8.55 | 1 | 239 | 3.79E−03 |
| RS2175935 | 1.32 | 3/4 | 54 | 1.42 | 4/4 | 6 | 1.13 | 5.71 | 2 | 232 | 3.80E−03 |
| RS25937 | 1.44 | 3/3 | 203 | 1.31 | 0/0 | 0 | 0 | 8.54 | 1 | 238 | 3.81E−03 |
| RS10507118 | 1.51 | 2/3 | 39 | 1.23 | 3/3 | 136 | 1.37 | 5.72 | 2 | 176 | 3.81E−03 |
| RS2833581 | 1.41 | 2/3 | 99 | 1.36 | 3/3 | 98 | 1.27 | 5.7 | 2 | 235 | 3.83E−03 |
| RS10482871 | 1.65 | 3/3 | 236 | 1.33 | 0/0 | 0 | 0 | 8.53 | 1 | 239 | 3.83E−03 |
| RS4122274 | 1.44 | 1/3 | 96 | 1.36 | 3/3 | 112 | 1.28 | 5.7 | 2 | 233 | 3.83E−03 |
| RS10521316 | 1.25 | 2/3 | 115 | 1.35 | 3/3 | 54 | 1.4 | 5.7 | 2 | 230 | 3.84E−03 |
| RS6673237 | 1.31 | 1/3 | 67 | 1.41 | 3/3 | 6 | 1.14 | 5.69 | 2 | 237 | 3.86E−03 |
| RS2409037 | 1.31 | 3/4 | 42 | 1.45 | 4/4 | 3 | 1.37 | 5.69 | 2 | 236 | 3.86E−03 |
| RS10503069 | 1.37 | 1/3 | 76 | 1.26 | 3/3 | 11 | 1.39 | 5.69 | 2 | 236 | 3.86E−03 |
| RS9784951 | 1.28 | 1/3 | 88 | 1.39 | 3/3 | 18 | 1.34 | 5.69 | 2 | 232 | 3.87E−03 |
| RS10501171 | 1.37 | 2/4 | 124 | 1.28 | 4/4 | 27 | 1.43 | 5.68 | 2 | 236 | 3.90E−03 |
| RS660274 | 1.32 | 1/3 | 49 | 1.42 | 3/3 | 114 | 1.29 | 5.73 | 2 | 170 | 3.91E−03 |
| RS10508378 | 1.96 | 2/4 | 16 | 1.2 | 4/4 | 224 | 1.34 | 5.67 | 2 | 238 | 3.93E−03 |
| RS1386971 | 1.32 | 2/4 | 6 | 1.62 | 0/0 | 0 | 0 | 8.48 | 1 | 238 | 3.93E−03 |
| RS1153606 | 1.33 | 1/3 | 76 | 1.32 | 3/3 | 5 | 1.7 | 5.67 | 2 | 236 | 3.93E−03 |
| RS1905015 | 1.46 | 1/3 | 118 | 1.33 | 3/3 | 90 | 1.29 | 5.67 | 2 | 236 | 3.93E−03 |
| RS10506168 | 1.35 | 1/2 | 72 | 1.33 | 2/2 | 14 | 1.12 | 5.67 | 2 | 236 | 3.93E−03 |
| RS1409993 | 1.28 | 4/4 | 125 | 1.38 | 0/0 | 0 | 0 | 8.48 | 1 | 236 | 3.93E−03 |
| RS2131774 | 1.31 | 1/3 | 56 | 1.25 | 3/3 | 123 | 1.39 | 5.7 | 2 | 182 | 3.97E−03 |
| RS2831853 | 1.34 | 2/3 | 10 | 1.11 | 0/0 | 0 | 0 | 8.46 | 1 | 235 | 3.98E−03 |
| RS10515792 | 1.36 | 2/4 | 79 | 1.31 | 4/4 | 11 | 1.12 | 5.66 | 2 | 232 | 3.98E−03 |
| RS6902590 | 1.28 | 1/4 | 97 | 1.37 | 4/4 | 24 | 1.44 | 5.66 | 2 | 232 | 3.98E−03 |
| RS1904165 | 1.22 | 1/2 | 30 | 1.47 | 2/2 | 185 | 1.32 | 5.67 | 2 | 215 | 3.98E−03 |
| RS10487019 | 1.66 | 2/4 | 27 | 1.45 | 4/4 | 211 | 1.31 | 5.64 | 2 | 237 | 4.05E−03 |
| RS9310715 | 1.33 | 1/3 | 79 | 1.3 | 3/3 | 10 | 1.57 | 5.65 | 2 | 220 | 4.05E−03 |
| RS1410364 | 1.31 | 2/4 | 69 | 1.38 | 4/4 | 12 | 1.53 | 5.65 | 2 | 218 | 4.05E−03 |
| RS1044045 | 1.37 | 1/3 | 66 | 1.25 | 3/3 | 9 | 1.34 | 5.64 | 2 | 230 | 4.06E−03 |
| RS5754499 | 1.19 | 4/4 | 218 | 1.35 | 0/0 | 0 | 0 | 8.41 | 1 | 238 | 4.08E−03 |
| RS3770899 | 1.05 | 1/3 | 31 | 1.46 | 3/3 | 205 | 1.31 | 5.63 | 2 | 234 | 4.09E−03 |
| RS2241577 | 1.5 | 1/2 | 66 | 1.4 | 2/2 | 166 | 1.3 | 5.62 | 2 | 235 | 4.13E−03 |
| RS10502543 | 1.37 | 1/2 | 98 | 1.39 | 2/2 | 117 | 1.28 | 5.62 | 2 | 234 | 4.13E−03 |
| RS10508405 | 1.32 | 1/3 | 82 | 1.39 | 3/3 | 24 | 1.21 | 5.63 | 2 | 215 | 4.14E−03 |
| RS6776797 | 1.32 | 2/3 | 13 | 1.52 | 0/0 | 0 | 0 | 8.38 | 1 | 239 | 4.14E−03 |
| RS7241410 | 1.36 | 2/3 | 100 | 1.39 | 3/3 | 102 | 1.27 | 5.62 | 2 | 221 | 4.16E−03 |
| RS896043 | 1.5 | 3/4 | 93 | 1.31 | 4/4 | 126 | 1.32 | 5.61 | 2 | 237 | 4.16E−03 |
| RS397405 | 1.47 | 4/4 | 210 | 1.31 | 0/0 | 0 | 0 | 8.37 | 1 | 230 | 4.18E−03 |
| RS2054915 | 1.31 | 1/2 | 125 | 1.38 | 2/2 | 45 | 1.24 | 5.61 | 2 | 228 | 4.18E−03 |
| RS32218 | 1.42 | 2/4 | 67 | 1.41 | 4/4 | 145 | 1.29 | 5.61 | 2 | 222 | 4.20E−03 |
| RS10485828 | 1.06 | 2/3 | 37 | 1.4 | 3/3 | 180 | 1.33 | 5.61 | 2 | 221 | 4.20E−03 |
| RS10495390 | 1.31 | 2/4 | 38 | 1.45 | 4/4 | 3 | 1.43 | 5.6 | 2 | 235 | 4.21E−03 |
| RS7161975 | 1.32 | 2/4 | 36 | 1.45 | 0/0 | 0 | 0 | 8.36 | 1 | 224 | 4.21E−03 |
| RS10510156 | 1.34 | 1/4 | 48 | 1.44 | 4/4 | 174 | 1.3 | 5.6 | 2 | 224 | 4.23E−03 |
| RS615696 | 1.64 | 2/4 | 63 | 1.32 | 4/4 | 155 | 1.32 | 5.6 | 2 | 221 | 4.24E−03 |
| RS10492136 | 1.9 | 2/4 | 33 | 1.3 | 4/4 | 205 | 1.33 | 5.59 | 2 | 237 | 4.24E−03 |
| RS1318822 | 1.26 | 1/3 | 114 | 1.37 | 3/3 | 37 | 1.37 | 5.59 | 2 | 235 | 4.25E−03 |
| RS823986 | 1.39 | 1/3 | 128 | 1.29 | 3/3 | 78 | 1.4 | 5.59 | 2 | 224 | 4.27E−03 |
| RS924159 | 1.3 | 4/4 | 76 | 1.4 | 0/0 | 0 | 0 | 8.32 | 1 | 238 | 4.28E−03 |
| RS10508876 | 1.32 | 2/4 | 27 | 1.48 | 4/4 | 5 | 1.2 | 5.58 | 2 | 234 | 4.29E−03 |
| RS4935712 | 1.31 | 2/4 | 121 | 1.38 | 4/4 | 48 | 1.25 | 5.58 | 2 | 231 | 4.30E−03 |
| RS10497723 | 1.35 | 1/3 | 32 | 1.22 | 0/0 | 0 | 0 | 8.3 | 1 | 237 | 4.33E−03 |
| RS1063964 | 1.4 | 2/4 | 101 | 1.29 | 4/4 | 33 | 1.29 | 5.57 | 2 | 234 | 4.33E−03 |
| RS2169141 | 1.25 | 1/3 | 113 | 1.37 | 3/3 | 60 | 1.35 | 5.57 | 2 | 234 | 4.33E−03 |
| RS10484846 | 1.68 | 2/3 | 50 | 1.34 | 3/3 | 159 | 1.31 | 5.58 | 2 | 211 | 4.35E−03 |
| RS10483541 | 1.55 | 4/4 | 229 | 1.32 | 0/0 | 0 | 0 | 8.29 | 1 | 237 | 4.35E−03 |
| RS9304675 | 1.33 | 2/3 | 111 | 1.29 | 3/3 | 32 | 1.46 | 5.56 | 2 | 236 | 4.37E−03 |
| RS10508895 | 1.11 | 1/3 | 20 | 1.19 | 3/3 | 197 | 1.36 | 5.57 | 2 | 216 | 4.38E−03 |
| RS10494231 | 1.43 | 2/4 | 76 | 1.29 | 4/4 | 72 | 1.29 | 5.58 | 2 | 199 | 4.39E−03 |
| RS1944487 | 1.39 | 1/3 | 119 | 1.28 | 3/3 | 59 | 1.37 | 5.55 | 2 | 238 | 4.41E−03 |
| RS10503056 | 1.37 | 2/4 | 76 | 1.26 | 4/4 | 11 | 1.4 | 5.55 | 2 | 235 | 4.41E−03 |
| RS4578215 | 1.6 | 4/4 | 234 | 1.32 | 0/0 | 0 | 0 | 8.26 | 1 | 239 | 4.42E−03 |
| RS1429130 | 1.32 | 1/3 | 23 | 1.47 | 0/0 | 0 | 0 | 8.25 | 1 | 223 | 4.47E−03 |
| RS1915552 | 1.31 | 1/4 | 128 | 1.38 | 4/4 | 72 | 1.26 | 5.54 | 2 | 230 | 4.47E−03 |
| RS724023 | 1.35 | 1/3 | 28 | 1.2 | 0/0 | 0 | 0 | 8.24 | 1 | 234 | 4.47E−03 |
| RS1993306 | 1.12 | 1/4 | 77 | 1.31 | 4/4 | 142 | 1.36 | 5.54 | 2 | 227 | 4.48E−03 |
| RS2593963 | 1.2 | 2/4 | 100 | 1.37 | 4/4 | 101 | 1.35 | 5.54 | 2 | 226 | 4.48E−03 |
| RS10516142 | 1.32 | 2/4 | 6 | 1.54 | 4/4 | 1 | 1.96 | 5.53 | 2 | 238 | 4.49E−03 |
| RS8076457 | 1.32 | 2/4 | 68 | 1.32 | 4/4 | 8 | 1.61 | 5.53 | 2 | 236 | 4.50E−03 |
| RS522085 | 1.35 | 1/3 | 75 | 1.27 | 3/3 | 11 | 1.5 | 5.53 | 2 | 233 | 4.50E−03 |
| RS7794555 | 1.31 | 2/4 | 32 | 1.45 | 0/0 | 0 | 0 | 8.22 | 1 | 237 | 4.52E−03 |
| RS2014617 | 1.32 | 2/4 | 92 | 1.38 | 4/4 | 12 | 1.15 | 5.52 | 2 | 238 | 4.54E−03 |
| RS10495099 | 1.31 | 2/4 | 39 | 1.45 | 4/4 | 197 | 1.31 | 5.52 | 2 | 236 | 4.54E−03 |
| RS10516865 | 1.33 | 3/4 | 5 | 1.64 | 0/0 | 0 | 0 | 8.2 | 1 | 239 | 4.56E−03 |
| RS935056 | 1.43 | 2/4 | 102 | 1.3 | 4/4 | 74 | 1.29 | 5.52 | 2 | 227 | 4.56E−03 |
| RS1376917 | 1.17 | 4/4 | 221 | 1.34 | 0/0 | 0 | 0 | 8.2 | 1 | 237 | 4.56E−03 |
| RS10498603 | 1.17 | 3/3 | 222 | 1.34 | 0/0 | 0 | 0 | 8.2 | 1 | 237 | 4.56E−03 |
| RS10511729 | 1.33 | 1/2 | 117 | 1.3 | 2/2 | 37 | 1.45 | 5.51 | 2 | 237 | 4.58E−03 |
| RS9323026 | 1.45 | 1/3 | 97 | 1.3 | 3/3 | 84 | 1.32 | 5.52 | 2 | 219 | 4.58E−03 |
| RS2346593 | 1.91 | 1/2 | 14 | 1.31 | 2/2 | 223 | 1.33 | 5.51 | 2 | 236 | 4.58E−03 |
| RS10515985 | 1.37 | 2/4 | 66 | 1.25 | 4/4 | 11 | 1.39 | 5.52 | 2 | 217 | 4.59E−03 |
| RS6966318 | 1.37 | 2/4 | 101 | 1.31 | 4/4 | 24 | 1.2 | 5.51 | 2 | 232 | 4.59E−03 |
| RS987479 | 1.32 | 1/3 | 74 | 1.32 | 3/3 | 11 | 1.56 | 5.51 | 2 | 229 | 4.60E−03 |
| RS10506835 | 1.43 | 2/3 | 85 | 1.27 | 3/3 | 116 | 1.36 | 5.51 | 2 | 220 | 4.62E−03 |
| RS10504742 | 1.31 | 1/4 | 108 | 1.31 | 4/4 | 33 | 1.46 | 5.5 | 2 | 237 | 4.63E−03 |
| RS6134885 | 1.37 | 3/4 | 73 | 1.26 | 4/4 | 8 | 1.49 | 5.51 | 2 | 218 | 4.63E−03 |
| RS4131870 | 1.52 | 2/4 | 74 | 1.29 | 4/4 | 148 | 1.33 | 5.5 | 2 | 235 | 4.63E−03 |
| RS4785953 | 1.31 | 1/3 | 47 | 1.36 | 3/3 | 6 | 1.64 | 5.5 | 2 | 234 | 4.63E−03 |
| RS3847222 | 1.31 | 1/4 | 96 | 1.33 | 4/4 | 16 | 1.52 | 5.49 | 2 | 237 | 4.67E−03 |
| RS3874273 | 1.27 | 1/3 | 98 | 1.37 | 3/3 | 37 | 1.39 | 5.49 | 2 | 228 | 4.69E−03 |
| RS570008 | 1.37 | 3/4 | 116 | 1.28 | 4/4 | 61 | 1.39 | 5.48 | 2 | 237 | 4.71E−03 |
| RS745708 | 1.29 | 1/3 | 59 | 1.41 | 3/3 | 8 | 1.39 | 5.49 | 2 | 210 | 4.74E−03 |
| RS4984442 | 1.29 | 1/3 | 84 | 1.4 | 3/3 | 17 | 1.35 | 5.48 | 2 | 225 | 4.74E−03 |
| RS2973237 | 1.48 | 2/4 | 87 | 1.31 | 4/4 | 112 | 1.32 | 5.48 | 2 | 222 | 4.75E−03 |
| RS10521007 | 1.37 | 2/4 | 71 | 1.27 | 4/4 | 14 | 1.23 | 5.47 | 2 | 238 | 4.76E−03 |
| RS10489117 | 1.73 | 1/2 | 52 | 1.33 | 2/2 | 184 | 1.32 | 5.47 | 2 | 237 | 4.76E−03 |
| RS581277 | 1.69 | 2/4 | 53 | 1.39 | 4/4 | 184 | 1.31 | 5.47 | 2 | 237 | 4.76E−03 |
| RS10489115 | 1.73 | 2/3 | 46 | 1.32 | 3/3 | 189 | 1.32 | 5.47 | 2 | 236 | 4.76E−03 |
| RS162676 | 1.36 | 2/4 | 99 | 1.39 | 4/4 | 123 | 1.28 | 5.47 | 2 | 236 | 4.76E−03 |
| RS3789343 | 1.37 | 1/3 | 78 | 1.25 | 3/3 | 16 | 1.33 | 5.47 | 2 | 232 | 4.77E−03 |
| RS918092 | 1.29 | 3/4 | 68 | 1.4 | 4/4 | 9 | 1.41 | 5.47 | 2 | 226 | 4.79E−03 |
| RS9303878 | 1.34 | 1/4 | 34 | 1.25 | 4/4 | 2 | 1.78 | 5.47 | 2 | 226 | 4.79E−03 |
| RS2078536 | 1.5 | 2/4 | 88 | 1.3 | 4/4 | 101 | 1.34 | 5.48 | 2 | 209 | 4.79E−03 |
| RS2347061 | 1.34 | 1/3 | 5 | 1.06 | 3/3 | 1 | 1.86 | 5.46 | 2 | 238 | 4.80E−03 |
| RS9325124 | 1.4 | 1/3 | 118 | 1.37 | 3/3 | 89 | 1.27 | 5.46 | 2 | 237 | 4.81E−03 |
| RS972432 | 1.79 | 2/3 | 26 | 1.44 | 3/3 | 209 | 1.31 | 5.46 | 2 | 233 | 4.82E−03 |
| RS2209426 | 1.3 | 1/3 | 84 | 1.4 | 3/3 | 11 | 1.26 | 5.45 | 2 | 237 | 4.85E−03 |
| RS1526723 | 1.24 | 2/4 | 115 | 1.39 | 4/4 | 91 | 1.31 | 5.45 | 2 | 234 | 4.86E−03 |
| RS2041442 | 1.32 | 2/4 | 48 | 1.34 | 4/4 | 4 | 1.73 | 5.45 | 2 | 232 | 4.86E−03 |
| RS2357344 | 1.64 | 2/4 | 24 | 1.39 | 4/4 | 183 | 1.31 | 5.46 | 2 | 209 | 4.88E−03 |
| RS10514136 | 1.36 | 1/3 | 36 | 1.21 | 3/3 | 6 | 1.28 | 5.45 | 2 | 209 | 4.93E−03 |
| RS637225 | 1.43 | 2/4 | 97 | 1.32 | 4/4 | 61 | 1.28 | 5.44 | 2 | 210 | 4.97E−03 |
| RS3859990 | 1.31 | 4/4 | 29 | 1.45 | 0/0 | 0 | 0 | 7.8 | 1 | 236 | 5.65E−03 |

TABLE 7
The most significant haplotype regions for APOA1 based
on HaploRec + HPM analysis.

| RS_id | SEQ_ID_no | Gene | Gene_id | Chr | Position | P-value |
|---|---|---|---|---|---|---|
| RS1016579 | 881 | | | 8 | 129269143 | 1.00E−04 |
| RS10502465 | 2490 | | | 18 | 20848719 | 1.00E−04 |
| RS10508903 | 983 | MAPK8 | 5599 | 10 | 49311338 | 1.00E−04 |
| RS10517832 | 1745 | | | 4 | 63462068 | 1.00E−04 |
| RS1395734 | 1746 | | | 4 | 63540641 | 1.00E−04 |
| RS159375 | 1839 | NDUFA12L | 91942 | 5 | 60469024 | 1.00E−04 |
| RS1941915 | 2499 | | | 18 | 21596530 | 1.00E−04 |
| RS2220486 | 1162 | DKFZp761O2018 | 92293 | 12 | 127658094 | 1.00E−04 |
| RS295525 | 525 | | | 5 | 60508623 | 1.00E−04 |
| RS295577 | 524 | NDUFA12L | 91942 | 5 | 60457592 | 1.00E−04 |
| RS39661 | 1840 | NDUFA12L | 91942 | 5 | 60477844 | 1.00E−04 |
| RS536617 | 2266 | FAT3 | 120114 | 11 | 91943638 | 1.00E−04 |
| RS540248 | 2265 | FAT3 | 120114 | 11 | 91943248 | 1.00E−04 |
| RS665889 | 915 | | | 9 | 86972031 | 1.00E−04 |
| RS7407453 | 2498 | | | 18 | 21567347 | 1.00E−04 |
| RS9297772 | 2167 | | | 8 | 129264644 | 1.00E−04 |
| RS9317818 | 2361 | | | 13 | 68921367 | 1.00E−04 |
| RS10502466 | 2491 | | | 18 | 20853692 | 2.00E−04 |
| RS10507767 | 2358 | | | 13 | 68846283 | 2.00E−04 |
| RS1322177 | 1911 | BMP6 | 654 | 6 | 7755920 | 2.00E−04 |
| RS1330538 | 2362 | | | 13 | 68929072 | 2.00E−04 |
| RS1806083 | 1810 | SEMA5A | 9037 | 5 | 9253348 | 2.00E−04 |
| RS2056247 | 1092 | | | 11 | 93315872 | 2.00E−04 |
| RS2398417 | 1163 | DKFZp761O2018 | 92293 | 12 | 127719497 | 2.00E−04 |
| RS2852861 | 2274 | FAT3 | 120114 | 11 | 92092036 | 2.00E−04 |
| RS4131454 | 2492 | ZNF521 | 25925 | 18 | 21035848 | 2.00E−04 |
| RS4838626 | 2214 | ARHGAP22 | 58504 | 10 | 49443676 | 2.00E−04 |
| RS507575 | 1995 | TPD52L1 | 7164 | 6 | 125559665 | 2.00E−04 |
| RS514096 | 1996 | TPD52L1 | 7164 | 6 | 125569006 | 2.00E−04 |
| RS6874699 | 1830 | ELOVL7 | 79993 | 5 | 60109757 | 2.00E−04 |
| RS965986 | 1994 | TPD52L1 | 7164 | 6 | 125544750 | 2.00E−04 |
| RS10492292 | 2168 | | | 8 | 129272373 | 3.00E−04 |
| RS10495390 | 116 | RYR2 | 6262 | 1 | 235379636 | 3.00E−04 |
| RS10498673 | 1912 | BMP6 | 654 | 6 | 7770256 | 3.00E−04 |
| RS10501795 | 2273 | FAT3 | 120114 | 11 | 92087066 | 3.00E−04 |
| RS10503626 | 808 | PSD3 | 23362 | 8 | 18591364 | 3.00E−04 |
| RS10514906 | 1841 | | | 5 | 60510338 | 3.00E−04 |
| RS1806110 | 1811 | SEMA5A | 9037 | 5 | 9253540 | 3.00E−04 |
| RS295561 | 1838 | NDUFA12L | 91942 | 5 | 60435974 | 3.00E−04 |
| RS4235481 | 1831 | ELOVL7 | 79993 | 5 | 60129144 | 3.00E−04 |
| RS4908087 | 71 | | | 1 | 100861182 | 3.00E−04 |
| RS502357 | 2267 | FAT3 | 120114 | 11 | 91953156 | 3.00E−04 |
| RS534196 | 914 | | | 9 | 86868202 | 3.00E−04 |
| RS724313 | 2488 | | | 18 | 20842927 | 3.00E−04 |
| RS787533 | 10 | | | 1 | 67818897 | 3.00E−04 |
| RS787534 | 11 | | | 1 | 67819113 | 3.00E−04 |
| RS8121515 | 2547 | C20orf133 | 140733 | 20 | 15382681 | 3.00E−04 |
| RS9291696 | 1829 | ELOVL7 | 79993 | 5 | 60100142 | 3.00E−04 |
| RS992241 | 2169 | | | 8 | 129272494 | 3.00E−04 |
| RS10492047 | 1158 | LOC144678 | 144678 | 12 | 125501283 | 4.00E−04 |
| RS10498717 | 624 | | | 6 | 23833783 | 4.00E−04 |
| RS1577340 | 2357 | | | 13 | 68840319 | 4.00E−04 |
| RS1902781 | 2170 | | | 8 | 129283194 | 4.00E−04 |
| RS1941616 | 2487 | | | 18 | 20837040 | 4.00E−04 |
| RS2182068 | 625 | | | 6 | 23845925 | 4.00E−04 |
| RS2208137 | 2546 | C20orf133 | 140733 | 20 | 15373454 | 4.00E−04 |
| RS295565 | 1837 | NDUFA12L | 91942 | 5 | 60433914 | 4.00E−04 |
| RS33924 | 1880 | | | 5 | 129264038 | 4.00E−04 |
| RS3847544 | 2278 | FAT3 | 120114 | 11 | 92219842 | 4.00E−04 |
| RS5007492 | 2500 | | | 18 | 21662551 | 4.00E−04 |
| RS530616 | 1786 | NDST4 | 64579 | 4 | 116202568 | 4.00E−04 |
| RS878016 | 757 | DLGAP2 | 9228 | 8 | 1585693 | 4.00E−04 |
| RS927402 | 1910 | BMP6 | 654 | 6 | 7755224 | 4.00E−04 |
| RS9296540 | 638 | | | 6 | 47418964 | 4.00E−04 |
| RS9296545 | 639 | | | 6 | 47433570 | 4.00E−04 |
| RS959988 | 1164 | SLC15A4 | 121260 | 12 | 127858547 | 4.00E−04 |
| RS975830 | 2337 | LOC144678 | 144678 | 12 | 125501837 | 4.00E−04 |
| RS10518315 | 24 | | | 1 | 69652390 | 5.00E−04 |
| RS1805960 | 1812 | SEMA5A | 9037 | 5 | 9268331 | 5.00E−04 |
| RS1902778 | 2166 | | | 8 | 129264032 | 5.00E−04 |
| RS1998846 | 25 | | | 1 | 69674145 | 5.00E−04 |
| RS2683408 | 1748 | | | 4 | 63554481 | 5.00E−04 |
| RS500409 | 1787 | NDST4 | 64579 | 4 | 116203593 | 5.00E−04 |
| RS540138 | 2268 | FAT3 | 120114 | 11 | 91953442 | 5.00E−04 |
| RS572241 | 2264 | FAT3 | 120114 | 11 | 91942082 | 5.00E−04 |
| RS950964 | 1539 | RYR2 | 6262 | 1 | 235487304 | 5.00E−04 |
| RS964906 | 1500 | | | 1 | 100860067 | 5.00E−04 |
| RS10498293 | 2380 | | | 14 | 27284686 | 6.00E−04 |
| RS10507766 | 2356 | | | 13 | 68838026 | 6.00E−04 |
| RS10520061 | 1881 | CSS3 | 337876 | 5 | 129355287 | 6.00E−04 |
| RS1169429 | 913 | | | 9 | 86853672 | 6.00E−04 |
| RS1994592 | 1653 | FAM19A4 | 151647 | 3 | 68907175 | 6.00E−04 |
| RS2168407 | 2160 | COL14A1 | 7373 | 8 | 121425244 | 6.00E−04 |
| RS2909239 | 2165 | | | 8 | 129261531 | 6.00E−04 |
| RS4418368 | 758 | DLGAP2 | 9228 | 8 | 1585785 | 6.00E−04 |
| RS4765395 | 2338 | LOC144678 | 144678 | 12 | 125519464 | 6.00E−04 |
| RS566388 | 1784 | NDST4 | 64579 | 4 | 116200944 | 6.00E−04 |
| RS6470607 | 2164 | | | 8 | 129255251 | 6.00E−04 |
| RS6938784 | 1947 | | | 6 | 42598500 | 6.00E−04 |
| RS9304495 | 2504 | TAF4B | 6875 | 18 | 22176091 | 6.00E−04 |
| RS10485526 | 2548 | C20orf133 | 140733 | 20 | 15383203 | 7.00E−04 |
| RS10501794 | 2272 | FAT3 | 120114 | 11 | 92082107 | 7.00E−04 |
| RS10508901 | 981 | MAPK8 | 5599 | 10 | 49297404 | 7.00E−04 |
| RS10508902 | 982 | MAPK8 | 5599 | 10 | 49311187 | 7.00E−04 |
| RS1445292 | 1832 | ELOVL7 | 79993 | 5 | 60155879 | 7.00E−04 |
| RS1460959 | 1828 | ELOVL7 | 79993 | 5 | 60100094 | 7.00E−04 |
| RS1806111 | 1809 | SEMA5A | 9037 | 5 | 9253329 | 7.00E−04 |
| RS1893120 | 1788 | NDST4 | 64579 | 4 | 116230217 | 7.00E−04 |
| RS1941619 | 2489 | | | 18 | 20848396 | 7.00E−04 |
| RS1972602 | 2476 | LOC390843 | 390843 | 18 | 20367962 | 7.00E−04 |
| RS2139090 | 2284 | | | 11 | 93363251 | 7.00E−04 |
| RS7407701 | 2497 | | | 18 | 21567062 | 7.00E−04 |
| RS7828096 | 2095 | PSD3 | 23362 | 8 | 18597554 | 7.00E−04 |
| RS1006609 | 2477 | LOC390843 | 390843 | 18 | 20368055 | 8.00E−04 |
| RS10491042 | 2215 | ARHGAP22 | 58504 | 10 | 49444215 | 8.00E−04 |
| RS10493425 | 9 | | | 1 | 67782715 | 8.00E−04 |
| RS10517841 | 1755 | | | 4 | 63584709 | 8.00E−04 |
| RS1427422 | 1660 | | | 3 | 69866955 | 8.00E−04 |
| RS1526211 | 219 | | | 2 | 184719208 | 8.00E−04 |
| RS1946424 | 1659 | | | 3 | 69857899 | 8.00E−04 |
| RS2118763 | 513 | | | 5 | 44823303 | 8.00E−04 |
| RS2297355 | 920 | C9orf125 | 84302 | 9 | 103283578 | 8.00E−04 |
| RS352254 | 2363 | | | 13 | 68937642 | 8.00E−04 |
| RS4122086 | 2271 | FAT3 | 120114 | 11 | 92070494 | 8.00E−04 |
| RS4896783 | 1993 | TPD52L1 | 7164 | 6 | 125539447 | 8.00E−04 |
| RS6074891 | 2545 | C20orf133 | 140733 | 20 | 15363912 | 8.00E−04 |
| RS6938470 | 640 | | | 6 | 47446050 | 8.00E−04 |
| RS723276 | 1836 | NDUFA12L | 91942 | 5 | 60412893 | 8.00E−04 |
| RS7841198 | 2096 | PSD3 | 23362 | 8 | 18597648 | 8.00E−04 |
| RS9287218 | 1538 | RYR2 | 6262 | 1 | 235343721 | 8.00E−04 |
| RS10492048 | 2339 | LOC144678 | 144678 | 12 | 125521407 | 9.00E−04 |
| RS10505380 | 2157 | COL14A1 | 7373 | 8 | 121395654 | 9.00E−04 |
| RS10507765 | 2355 | | | 13 | 68805132 | 9.00E−04 |
| RS1351633 | 512 | | | 5 | 44579608 | 9.00E−04 |
| RS1358091 | 220 | | | 2 | 184735915 | 9.00E−04 |
| RS1360156 | 1954 | | | 6 | 47799167 | 9.00E−04 |
| RS1418005 | 26 | | | 1 | 69720212 | 9.00E−04 |
| RS1547641 | 623 | | | 6 | 23833200 | 9.00E−04 |
| RS1929496 | 921 | | | 9 | 103306774 | 9.00E−04 |
| RS4129501 | 2493 | ZNF521 | 25925 | 18 | 21170719 | 9.00E−04 |
| RS6469916 | 2159 | COL14A1 | 7373 | 8 | 121401972 | 9.00E−04 |
| RS7148166 | 2388 | NUBPL | 80224 | 14 | 31303436 | 9.00E−04 |
| RS722833 | 1332 | GAS7 | 8522 | 17 | 9876974 | 9.00E−04 |
| RS763671 | 2126 | SEC11L2 | 157708 | 8 | 55602883 | 9.00E−04 |
| RS9298506 | 2125 | SEC11L2 | 157708 | 8 | 55600077 | 9.00E−04 |
| RS952938 | 1744 | | | 4 | 63436594 | 9.00E−04 |
| RS967326 | 2094 | PSD3 | 23362 | 8 | 18591541 | 9.00E−04 |
| RS10485116 | 1946 | LOC653802 | 653802 | 6 | 42430492 | 1.00E−03 |
| RS10491272 | 1882 | CSS3 | 337876 | 5 | 129423355 | 1.00E−03 |
| RS10492294 | 2175 | | | 8 | 129315597 | 1.00E−03 |
| RS1586681 | 912 | | | 9 | 86837254 | 1.00E−03 |
| RS1622239 | 2478 | | | 18 | 20386505 | 1.00E−03 |
| RS1945148 | 2479 | | | 18 | 20387524 | 1.00E−03 |
| RS325220 | 1862 | | | 5 | 98332464 | 1.00E−03 |
| RS3799751 | 1997 | TPD52L1 | 7164 | 6 | 125569953 | 1.00E−03 |
| RS4871056 | 2158 | COL14A1 | 7373 | 8 | 121395762 | 1.00E−03 |
| RS6079817 | 2544 | C20orf133 | 140733 | 20 | 15358067 | 1.00E−03 |
| RS6916562 | 1953 | GPR115 | 221393 | 6 | 47789453 | 1.00E−03 |
| RS709386 | 1863 | | | 5 | 98357950 | 1.00E−03 |
| RS725819 | 626 | | | 6 | 23849815 | 1.00E−03 |
| RS7523565 | 12 | | | 1 | 67843580 | 1.00E−03 |
| RS927407 | 1909 | BMP6 | 654 | 6 | 7755127 | 1.00E−03 |
| RS9291707 | 1842 | | | 5 | 60511983 | 1.00E−03 |
| RS970488 | 2389 | NUBPL | 80224 | 14 | 31319837 | 1.00E−03 |
| RS719355 | 1473 | | | 1 | 34628978 | 1.10E−03 |
| RS10483457 | 1232 | GARNL1 | 253959 | 14 | 35211683 | 1.10E−03 |
| RS10493933 | 73 | | | 1 | 100883870 | 1.10E−03 |
| RS10504166 | 2123 | LOC392222 | 392222 | 8 | 55556364 | 1.10E−03 |
| RS10512865 | 514 | | | 5 | 44859124 | 1.10E−03 |
| RS1526212 | 218 | | | 2 | 184719102 | 1.10E−03 |
| RS1981134 | 72 | | | 1 | 100883793 | 1.10E−03 |
| RS2939619 | 2283 | | | 11 | 93356828 | 1.10E−03 |
| RS4546275 | 1757 | | | 4 | 63619250 | 1.10E−03 |
| RS4876117 | 2071 | DLGAP2 | 9228 | 8 | 1589804 | 1.10E−03 |
| RS516638 | 1783 | NDST4 | 64579 | 4 | 116198917 | 1.10E−03 |
| RS6830598 | 1775 | NDST4 | 64579 | 4 | 116084463 | 1.10E−03 |
| RS7239405 | 2515 | | | 18 | 56812039 | 1.10E−03 |
| RS10498294 | 2381 | | | 14 | 27285522 | 1.20E−03 |
| RS10501809 | 2280 | | | 11 | 93305911 | 1.20E−03 |
| RS10501810 | 2281 | | | 11 | 93306237 | 1.20E−03 |
| RS10505004 | 2149 | | | 8 | 102157533 | 1.20E−03 |
| RS1343788 | 1616 | MGC42174 | 129563 | 2 | 232759753 | 1.20E−03 |
| RS2346593 | 1756 | | | 4 | 63606400 | 1.20E−03 |
| RS2503102 | 679 | LOC442238 | 442238 | 6 | 100650488 | 1.20E−03 |
| RS2660589 | 1749 | | | 4 | 63555481 | 1.20E−03 |
| RS2863227 | 1879 | | | 5 | 129250974 | 1.20E−03 |
| RS3116174 | 1615 | MGC42174 | 129563 | 2 | 232717833 | 1.20E−03 |
| RS558868 | 1782 | NDST4 | 64579 | 4 | 116198572 | 1.20E−03 |
| RS6748937 | 221 | | | 2 | 184738190 | 1.20E−03 |
| RS7158021 | 2387 | NUBPL | 80224 | 14 | 31301646 | 1.20E−03 |
| RS7758833 | 1955 | | | 6 | 47799521 | 1.20E−03 |
| RS9285766 | 1488 | | | 1 | 67741313 | 1.20E−03 |
| RS1023080 | 1952 | GPR115 | 221393 | 6 | 47778169 | 1.30E−03 |
| RS10491089 | 1331 | GAS7 | 8522 | 17 | 9828100 | 1.30E−03 |
| RS10498260 | 253 | MGC42174 | 129563 | 2 | 232764865 | 1.30E−03 |
| RS10501811 | 2282 | | | 11 | 93309061 | 1.30E−03 |
| RS10503827 | 814 | | | 8 | 28223645 | 1.30E−03 |
| RS10516607 | 1774 | NDST4 | 66579 | 4 | 116047414 | 1.30E−03 |
| RS2094923 | 1920 | | | 6 | 14397061 | 1.30E−03 |
| RS2278016 | 1308 | | | 16 | 50674510 | 1.30E−03 |
| RS32904 | 916 | | | 9 | 86989232 | 1.30E−03 |
| RS3865407 | 2505 | TAF4B | 6875 | 18 | 22191304 | 1.30E−03 |
| RS4349629 | 1758 | | | 4 | 63640765 | 1.30E−03 |
| RS533950 | 1785 | NDST4 | 64579 | 4 | 116200998 | 1.30E−03 |
| RS929780 | 1833 | ERCC8 | 1161 | 5 | 60224783 | 1.30E−03 |
| RS9298505 | 2124 | SEC11L2 | 157708 | 8 | 55599688 | 1.30E−03 |
| RS9312103 | 1754 | | | 4 | 63584684 | 1.30E−03 |
| RS10491092 | 1333 | GAS7 | 8522 | 17 | 9945918 | 1.40E−03 |
| RS10505507 | 2174 | | | 8 | 129311983 | 1.40E−03 |
| RS10508895 | 2209 | MARCH8 | 220972 | 10 | 45287774 | 1.40E−0 |
| RS10516608 | 1776 | NDST4 | 64579 | 4 | 116085298 | 1.40E−03 |
| RS1121922 | 2093 | PSD3 | 23362 | 8 | 18563312 | 1.40E−03 |
| RS1427421 | 1661 | MITF | 4286 | 3 | 69870404 | 1.40E−03 |
| RS1806004 | 1813 | SEMA5A | 9037 | 5 | 9292427 | 1.40E−03 |
| RS1946425 | 1658 | | | 3 | 69857771 | 1.40E−03 |
| RS1948617 | 2516 | | | 18 | 56819944 | 1.40E−03 |
| RS2019441 | 2496 | | | 18 | 21451878 | 1.40E−03 |
| RS2876349 | 1919 | | | 6 | 14354510 | 1.40E−03 |
| RS4981122 | 2386 | NUBPL | 80224 | 14 | 31270802 | 1.40E−03 |
| RS535043 | 1543 | | | 1 | 236762659 | 1.40E−03 |
| RS853392 | 1918 | | | 6 | 14302577 | 1.40E−03 |
| RS861209 | 1917 | | | 6 | 14302443 | 1.40E−03 |
| RS1013997 | 2269 | FAT3 | 120114 | 11 | 91957013 | 1.50E−03 |
| RS10491854 | 948 | | | 9 | 119414301 | 1.50E−03 |
| RS10517411 | 1723 | KIAA1239 | 57495 | 4 | 37068999 | 1.50E−03 |
| RS1324673 | 622 | | | 6 | 23820609 | 1.50E−03 |
| RS2898481 | 2097 | PSD3 | 23362 | 8 | 18598158 | 1.50E−03 |
| RS6079838 | 2549 | C20orf133 | 140733 | 20 | 15383756 | 1.50E−03 |
| RS6601422 | 2082 | MSRA | 4482 | 8 | 10147444 | 1.50E−03 |
| RS6673250 | 1546 | | | 1 | 236842031 | 1.50E−03 |
| RS7823735 | 2150 | | | 8 | 102189451 | 1.50E−03 |
| RS9296387 | 1948 | | | 6 | 42610292 | 1.50E−03 |
| RS998359 | 1777 | NDST4 | 64579 | 4 | 116090480 | 1.50E−03 |
| RS10493456 | 23 | | | 1 | 69651697 | 1.60E−03 |
| RS1919447 | 1593 | | | 2 | 148179334 | 1.60E−03 |
| RS2130437 | 1652 | FAM19A4 | 151647 | 3 | 68906766 | 1.60E−03 |
| RS7001766 | 2131 | | | 8 | 73285827 | 1.60E−03 |
| RS7773311 | 1951 | GPR115 | 221393 | 6 | 47777445 | 1.60E−03 |
| RS7838059 | 2083 | MSRA | 4482 | 8 | 10216220 | 1.60E−03 |
| RS10491855 | 947 | | | 9 | 119335541 | 1.70E−03 |
| RS10498295 | 2382 | | | 14 | 27285584 | 1.70E−03 |
| RS10501813 | 2285 | | | 11 | 93375645 | 1.70E−03 |
| RS10502464 | 2486 | | | 18 | 20821256 | 1.70E−03 |
| RS10502475 | 1376 | KCTD1 | 284252 | 18 | 22427942 | 1.70E−03 |
| RS10512855 | 511 | | | 5 | 44546969 | 1.70E−03 |
| RS1379782 | 1161 | | | 12 | 127624822 | 1.70E−03 |
| RS158935 | 1834 | ERCC8 | 1161 | 5 | 60273741 | 1.70E−03 |
| RS279511 | 1781 | NDST4 | 64579 | 4 | 116125572 | 1.70E−03 |
| RS2949865 | 2286 | HEPHL1 | 341208 | 11 | 93394880 | 1.70E−03 |
| RS3116231 | 252 | MGC42174 | 129563 | 2 | 232600527 | 1.70E−03 |
| RS509423 | 1789 | | | 4 | 116280778 | 1.70E−03 |
| RS10483458 | 2397 | GARNL1 | 253959 | 14 | 35290074 | 1.80E−03 |
| RS10498296 | 2383 | | | 14 | 27308301 | 1.80E−03 |
| RS10503624 | 2092 | PSD3 | 23362 | 8 | 18562990 | 1.80E−03 |
| RS10504534 | 2130 | | | 8 | 73285243 | 1.80E−03 |
| RS10517214 | 343 | | | 4 | 47955582 | 1.80E−03 |
| RS1252144 | 1537 | | | 1 | 235219020 | 1.80E−03 |
| RS2346649 | 1753 | | | 4 | 63584319 | 1.80E−03 |
| RS242687 | 2518 | | | 18 | 56917055 | 1.80E−03 |
| RS2722897 | 815 | PNOC | 5368 | 8 | 28229116 | 1.80E−03 |
| RS2820211 | 1544 | | | 1 | 236767330 | 1.80E−03 |
| RS2874941 | 2104 | FZD3 | 7976 | 8 | 28429267 | 1.80E−03 |
| RS586565 | 1545 | | | 1 | 236825154 | 1.80E−03 |
| RS7835350 | 2176 | | | 8 | 129331257 | 1.80E−03 |
| RS870241 | 1654 | FAM19A4 | 151647 | 3 | 68908072 | 1.80E−03 |
| RS1019384 | 2517 | | | 18 | 56879341 | 1.90E−03 |
| RS10497645 | 222 | | | 2 | 184738537 | 1.90E−03 |
| RS2101435 | 254 | MGC42174 | 129563 | 2 | 232823402 | 1.90E−03 |
| RS2221268 | 1864 | | | 5 | 98385315 | 1.90E−03 |
| RS2326994 | 1908 | BMP6 | 654 | 6 | 7755020 | 1.90E−03 |
| RS2333183 | 2384 | | | 14 | 27367061 | 1.90E−03 |
| RS2346648 | 1752 | | | 4 | 63578452 | 1.90E−03 |
| RS2435307 | 1371 | NPC1 | 4864 | 18 | 19381908 | 1.90E−03 |
| RS3958881 | 2077 | | | 8 | 8939389 | 1.90E−03 |
| RS4981894 | 2385 | NUBPL | 80224 | 14 | 31270542 | 1.90E−03 |
| RS538042 | 1998 | TPD52L1 | 7164 | 6 | 125575909 | 1.90E−03 |
| RS7821424 | 2171 | | | 8 | 129284834 | 1.90E−03 |
| RS9283955 | 2173 | | | 8 | 129288987 | 1.90E−03 |
| RS9298203 | 2129 | | | 8 | 73270276 | 1.90E−03 |
| RS10497651 | 217 | | | 2 | 184701354 | 2.00E−03 |
| RS10503829 | 2103 | FBXO16 | 157574 | 8 | 28389213 | 2.00E−03 |
| RS10516601 | 1773 | NDST4 | 64579 | 4 | 116046280 | 2.00E−03 |
| RS1112268 | 978 | | | 10 | 49217160 | 2.00E−03 |
| RS1460958 | 1827 | ELOVL7 | 79993 | 5 | 60099333 | 2.00E−03 |
| RS1528842 | 209 | | | 2 | 148174046 | 2.00E−03 |
| RS1585920 | 1768 | | | 4 | 80146392 | 2.00E−03 |
| RS1806112 | 1808 | SEMA5A | 9037 | 5 | 9253137 | 2.00E−03 |
| RS276650 | 2132 | | | 8 | 73299514 | 2.00E−03 |
| RS33350 | 1307 | | | 16 | 50608803 | 2.00E−03 |
| RS7009724 | 2078 | | | 8 | 8976535 | 2.00E−03 |
| RS798075 | 1471 | | | 1 | 34709579 | 2.00E−03 |
| RS963291 | 1878 | | | 5 | 129230116 | 2.00E−03 |
| RS10489547 | 27 | | | 1 | 69741665 | 2.10E−03 |
| RS10491274 | 1883 | CSS3 | 337876 | 5 | 129446258 | 2.10E−03 |
| RS10497020 | 1592 | | | 2 | 148173995 | 2.10E−03 |
| RS10502474 | 2506 | TAF4B | 6875 | 18 | 22198825 | 2.10E−03 |
| RS2107191 | 1934 | | | 6 | 29542274 | 2.10E−03 |
| RS2704404 | 342 | | | 4 | 47929301 | 2.10E−03 |
| RS6893752 | 1835 | NDUFA12L | 91942 | 5 | 60410669 | 2.10E−03 |
| RS787494 | 13 | | | 1 | 67858174 | 2.10E−03 |
| RS9287889 | 1606 | STK39 | 27347 | 2 | 168548700 | 2.10E−03 |
| RS10495392 | 1540 | RYR2 | 6262 | 1 | 235489612 | 2.20E−03 |
| RS10510207 | 1620 | CNTN6 | 27255 | 3 | 1309530 | 2.20E−03 |
| RS1480714 | 1767 | | | 4 | 80145855 | 2.20E−03 |
| RS260225 | 1945 | LOC653802 | 563802 | 6 | 42427285 | 2.20E−03 |
| RS6954679 | 728 | | | 7 | 63134099 | 2.20E−03 |
| RS798074 | 1472 | | | 1 | 34709939 | 2.20E−03 |
| RS798076 | 1470 | | | 1 | 34709155 | 2.20E−03 |
| RS10492133 | 2310 | GRIN2B | 2904 | 12 | 13726080 | 2.30E−03 |
| RS10508896 | 2210 | MARCH8 | 220972 | 10 | 45350649 | 2.30E−03 |
| RS10511379 | 1687 | LOC389142 | 389142 | 3 | 119645081 | 2.30E−03 |
| RS411331 | 1621 | CNTN6 | 27255 | 3 | 1320563 | 2.30E−03 |
| RS7349908 | 1932 | | | 6 | 29505500 | 2.30E−03 |
| RS9295805 | 1933 | | | 6 | 29505758 | 2.30E−03 |
| RS1033017 | 188 | MALL | 7851 | 2 | 110210510 | 2.40E−03 |
| RS10484547 | 1935 | | | 6 | 29560753 | 2.40E−03 |
| RS10502440 | 1372 | LAMA3 | 3909 | 18 | 19583575 | 2.40E−03 |
| RS10510270 | 1628 | | | 3 | 3542685 | 2.40E−03 |
| RS10517410 | 1722 | KIAA1239 | 57495 | 4 | 37045599 | 2.40E−03 |
| RS10517842 | 1759 | | | 4 | 63641655 | 2.40E−03 |
| RS1881740 | 979 | | | 10 | 49221566 | 2.40E−03 |
| RS1926256 | 22 | | | 1 | 69651662 | 2.40E−03 |
| RS2059232 | 1306 | | | 16 | 50596704 | 2.40E−03 |
| RS6458297 | 1950 | UBR2 | 23304 | 6 | 42655869 | 2.40E−03 |
| RS723733 | 2151 | | | 8 | 102196496 | 2.40E−03 |
| RS959987 | 1165 | SLC15A4 | 121260 | 12 | 127858753 | 2.40E−03 |
| RS10492049 | 2340 | PGBD3P3 | 267007 | 12 | 125558842 | 2.50E−03 |
| RS10497017 | 208 | | | 2 | 148158282 | 2.50E−03 |
| RS10501793 | 2270 | FAT3 | 120114 | 11 | 91957395 | 2.50E−03 |
| RS10501798 | 2279 | FAT3 | 120114 | 11 | 92224523 | 2.50E−03 |
| RS10518211 | 365 | | | 4 | 80156089 | 2.50E−03 |
| RS185266 | 1305 | | | 16 | 50595102 | 2.50E−03 |
| RS4715005 | 641 | | | 6 | 47472916 | 2.50E−03 |
| RS906648 | 1582 | LRP1B | 53353 | 2 | 142204999 | 2.50E−03 |
| RS9296538 | 637 | | | 6 | 47302408 | 2.50E−03 |
| RS9321873 | 1975 | LOC442238 | 442238 | 6 | 100642690 | 2.50E−03 |
| RS10486529 | 2034 | PTHB1 | 27241 | 7 | 33363277 | 2.60E−03 |
| RS10491088 | 1330 | GAS7 | 8522 | 17 | 9808904 | 2.60E−03 |
| RS10492134 | 2311 | GRIN2B | 2904 | 12 | 13750149 | 2.60E−03 |
| RS10508900 | 977 | FRMPD2 | 143162 | 10 | 49126059 | 2.60E−03 |
| RS10510170 | 1623 | CNTN6 | 27255 | 3 | 1349976 | 2.60E−03 |
| RS10510269 | 1627 | | | 3 | 3542361 | 2.60E−03 |
| RS10510991 | 1662 | MITF | 4286 | 3 | 69924318 | 2.60E−03 |
| RS1233493 | 1936 | LOC442195 | 442195 | 6 | 29566220 | 2.60E−03 |
| RS1785741 | 2501 | | | 18 | 21676577 | 2.60E−03 |
| RS1792624 | 2288 | | | 11 | 93561458 | 2.60E−03 |
| RS6878136 | 1865 | | | 5 | 98386091 | 2.60E−03 |
| RS7107851 | 2287 | HEPHL1 | 341208 | 11 | 93396703 | 2.60E−03 |
| RS779609 | 1686 | LOC389142 | 389142 | 3 | 119539208 | 2.60E−03 |
| RS787493 | 14 | | | 1 | 67858307 | 2.60E−03 |
| RS10493424 | 1487 | | | 1 | 67729796 | 2.70E−03 |
| RS10503167 | 2070 | DLGAP2 | 9228 | 8 | 1585073 | 2.70E−03 |
| RS10512866 | 515 | | | 5 | 44859710 | 2.70E−03 |
| RS1073251 | 1594 | | | 2 | 148179531 | 2.70E−03 |
| RS1499976 | 1684 | LOC389142 | 389142 | 3 | 119284020 | 2.70E−03 |
| RS2150738 | 919 | C9orf125 | 84302 | 9 | 103280529 | 2.70E−03 |
| RS4072525 | 2177 | | | 8 | 129424336 | 2.70E−03 |
| RS7837478 | 2133 | | | 8 | 73323698 | 2.70E−03 |
| RS862527 | 1916 | | | 6 | 14281843 | 2.70E−03 |
| RS901682 | 2208 | MARCH8 | 220972 | 10 | 45285867 | 2.70E−03 |
| RS10496435 | 189 | NPHP1 | 4867 | 2 | 110286462 | 2.80E−03 |
| RS10517407 | 1720 | KIAA1239 | 57495 | 4 | 36977725 | 2.80E−03 |
| RS2165543 | 1780 | NDST4 | 64579 | 4 | 116108687 | 2.80E−03 |
| RS2345475 | 1726 | | | 4 | 42443300 | 2.80E−03 |
| RS2346645 | 1751 | | | 4 | 63565257 | 2.80E−03 |
| RS3772295 | 1622 | CNTN6 | 27255 | 3 | 1349539 | 2.80E−03 |
| RS3844028 | 74 | | | 1 | 100884203 | 2.80E−03 |
| RS723804 | 2437 | | | 15 | 34258962 | 2.80E−03 |
| RS4570241 | 950 | | | 9 | 119436799 | 2.90E−03 |
| RS10511378 | 1688 | LOC389142 | 389142 | 3 | 119645313 | 2.90E−03 |
| RS10512895 | 1693 | TMEM108 | 66000 | 3 | 134498594 | 2.90E−03 |
| RS10518856 | 2438 | | | 15 | 34262252 | 2.90E−03 |
| RS1926289 | 16 | | | 1 | 68238520 | 2.90E−03 |
| RS489743 | 1944 | LOC653802 | 653802 | 6 | 42420978 | 2.90E−03 |
| RS723734 | 2152 | | | 8 | 102196618 | 2.90E−03 |
| RS9312699 | 349 | | | 4 | 59070339 | 2.90E−03 |
| RS10516609 | 1790 | | | 4 | 116303938 | 3.00E−03 |
| RS1360922 | 2348 | | | 13 | 37525010 | 3.00E−03 |
| RS1708370 | 1694 | TMEM108 | 66000 | 3 | 134499004 | 3.00E−03 |
| RS2817738 | 1927 | | | 6 | 24899079 | 3.00E−03 |
| RS4123791 | 2172 | | | 8 | 129288419 | 3.00E−03 |
| RS479600 | 918 | | | 9 | 103242624 | 3.00E−03 |
| RS9289439 | 1690 | TMEM108 | 66000 | 3 | 134343341 | 3.00E−03 |
| RS10484893 | 1931 | | | 6 | 24996530 | 3.10E−03 |
| RS10510271 | 1629 | | | 3 | 3546367 | 3.10E−03 |
| RS1825024 | 1765 | | | 4 | 80126223 | 3.10E−03 |
| RS1851006 | 1278 | UNC13C | 440279 | 15 | 52691684 | 3.10E−03 |
| RS1986511 | 1741 | | | 4 | 59062390 | 3.10E−03 |
| RS2250286 | 2347 | | | 13 | 37480178 | 3.10E−03 |
| RS3790888 | 1495 | LPHN2 | 23266 | 1 | 82185299 | 3.10E−03 |
| RS3790889 | 1494 | LPHN2 | 23266 | 1 | 82185188 | 3.10E−03 |
| RS9312101 | 1750 | | | 4 | 63564480 | 3.10E−03 |
| RS1013985 | 949 | | | 9 | 119414729 | 3.20E−03 |
| RS10493703 | 1493 | LPHN2 | 23266 | 1 | 82158504 | 3.20E−03 |
| RS10510272 | 1630 | | | 3 | 3546420 | 3.20E−03 |
| RS1598052 | 1740 | | | 4 | 59055920 | 3.20E−03 |
| RS407486 | 2543 | C20orf133 | 140733 | 20 | 15355876 | 3.20E−03 |
| RS4538379 | 1651 | | | 3 | 68834680 | 3.20E−03 |
| RS724815 | 2375 | | | 14 | 25666000 | 3.20E−03 |
| RS7599108 | 1617 | MGC42174 | 129563 | 2 | 232845842 | 3.20E−03 |
| RS8094618 | 2485 | | | 18 | 20816188 | 3.20E−03 |
| RS10486527 | 2033 | PTHB1 | 27241 | 7 | 33347198 | 3.30E−03 |
| RS10497642 | 216 | | | 2 | 184536677 | 3.30E−03 |
| RS10517409 | 1721 | KIAA1239 | 57495 | 4 | 37038537 | 3.30E−03 |
| RS10517440 | 1739 | | | 4 | 59051560 | 3.30E−03 |
| RS10517830 | 1743 | | | 4 | 63388819 | 3.30E−03 |
| RS10518659 | 1496 | LPHN2 | 23266 | 1 | 82187284 | 3.30E−03 |
| RS2347027 | 2336 | LOC144678 | 1446768 | 12 | 125501130 | 3.30E−03 |
| RS2378672 | 911 | NTRK2 | 4915 | 9 | 86820854 | 3.30E−03 |
| RS352427 | 816 | | | 8 | 28571996 | 3.30E−03 |
| RS385367 | 1497 | LPHN2 | 23266 | 1 | 82187787 | 3.30E−03 |
| RS809663 | 1685 | LOC389142 | 389142 | 3 | 119537598 | 3.30E−03 |
| RS896990 | 251 | MGC42174 | 129563 | 2 | 232597100 | 3.30E−03 |
| RS10495394 | 1541 | RYR2 | 6262 | 1 | 235498128 | 3.40E−03 |
| RS10504002 | 2118 | | | 8 | 37209534 | 3.40E−03 |
| RS10510268 | 1626 | | | 3 | 3539506 | 3.40E−03 |
| RS1825023 | 1766 | | | 4 | 80126286 | 3.40E−03 |
| RS2670876 | 2182 | DDEF1 | 50807 | 8 | 131339068 | 3.40E−03 |
| RS634403 | 1542 | | | 1 | 236762603 | 3.40E−03 |
| RS745708 | 976 | FRMPD2 | 143162 | 10 | 49115681 | 3.40E−03 |
| RS9297962 | 2145 | | | 8 | 96655791 | 3.40E−03 |
| RS942662 | 627 | | | 6 | 23850282 | 3.40E−03 |
| RS10503389 | 2079 | | | 8 | 8977133 | 3.50E−03 |
| RS10515286 | 1866 | | | 5 | 98464219 | 3.50E−03 |
| RS10518853 | 2436 | | | 15 | 34221604 | 3.50E−03 |
| RS1549455 | 2181 | DDEF1 | 50807 | 8 | 131334202 | 3.50E−03 |
| RS1908691 | 2316 | | | 12 | 29249400 | 3.50E−03 |
| RS2183248 | 1956 | | | 6 | 47807084 | 3.50E−03 |
| RS2590386 | 980 | | | 10 | 49255436 | 3.50E−03 |
| RS4371983 | 2127 | SEC11L2 | 157708 | 8 | 5503190 | 3.50E−03 |
| RS4655769 | 18 | | | 1 | 68249566 | 3.50E−03 |
| RS6900726 | 1949 | UBR2 | 23304 | 6 | 42639628 | 3.50E−03 |
| RS962215 | 2377 | | | 14 | 25707678 | 3.50E−03 |
| RS10483459 | 2398 | GARNL1 | 253959 | 14 | 35290406 | 3.60E−03 |
| RS10505519 | 2178 | | | 8 | 129493979 | 3.60E−03 |
| RS10505548 | 2183 | DDEF1 | 50807 | 8 | 131339636 | 3.60E−03 |
| RS10512891 | 1691 | TMEM108 | 66000 | 3 | 134431549 | 3.60E−03 |
| RS1529585 | 1663 | MITF | 4286 | 3 | 69953285 | 3.60E−03 |
| RS2425570 | 2554 | PTPRT | 11122 | 20 | 41111765 | 3.60E−03 |
| RS2474263 | 678 | LOC442238 | 442238 | 6 | 100641976 | 3.60E−03 |
| RS948435 | 1373 | LAMA3 | 3909 | 18 | 19637770 | 3.60E−03 |
| RS978313 | 17 | | | 1 | 68249062 | 3.60E−03 |
| RS1801758 | 1329 | GAS7 | 8522 | 17 | 9756951 | 3.70E−03 |
| RS10483312 | 2376 | | | 14 | 25681375 | 3.70E−03 |
| RS10517050 | 1728 | | | 4 | 42443702 | 3.70E−03 |
| RS1893290 | 1374 | LAMA3 | 3909 | 18 | 19696619 | 3.70E−03 |
| RS2060639 | 1692 | TMEM108 | 66000 | 3 | 134454348 | 3.70E−03 |
| RS2575517 | 1729 | | | 4 | 42496076 | 3.70E−03 |
| RS3874273 | 2434 | | | 15 | 34183438 | 3.70E−03 |
| RS7970144 | 2308 | GRIN2B | 2904 | 12 | 13724212 | 3.70E−03 |
| RS10504953 | 2144 | | | 8 | 96643015 | 3.80E−03 |
| RS10516125 | 1893 | | | 5 | 174413439 | 3.80E−03 |
| RS10517049 | 1727 | | | 4 | 42443415 | 3.80E−03 |
| RS1369592 | 2435 | | | 15 | 34185952 | 3.80E−03 |
| RS1450917 | 1730 | | | 4 | 42513998 | 3.80E−03 |
| RS4128165 | 457 | GLRA3 | 8001 | 4 | 175810278 | 3.80E−03 |
| RS4695953 | 459 | GLRA3 | 8001 | 4 | 175868286 | 3.80E−03 |
| RS6531533 | 1724 | KIAA1239 | 57495 | 4 | 37071908 | 3.80E−03 |
| RS7037164 | 2198 | | | 9 | 120331565 | 3.80E−03 |
| RS863660 | 2346 | | | 13 | 37470769 | 3.80E−03 |
| RS10518953 | 2443 | LOC390576 | 390576 | 15 | 35569758 | 3.90E−03 |
| RS1430745 | 19 | | | 1 | 68319321 | 3.90E−03 |
| RS171302 | 2108 | | | 8 | 28578345 | 3.90E−03 |
| RS2366659 | 1703 | FGF12 | 2257 | 3 | 193570445 | 3.90E−03 |
| RS273759 | 2495 | | | 18 | 21450194 | 3.90E−03 |
| RS2863171 | 1074 | | | 11 | 45207308 | 3.90E−03 |
| RS484510 | 1992 | TPD52L1 | 7164 | 6 | 125528599 | 3.90E−03 |
| RS548825 | 21 | | | 1 | 69624206 | 3.90E−03 |
| RS817504 | 1679 | | | 3 | 119161552 | 3.90E−03 |
| RS9287324 | 1585 | LRP1B | 53353 | 2 | 142232977 | 3.90E−03 |
| RS10498292 | 1229 | | | 14 | 27284462 | 4.00E−03 |
| RS10518952 | 2442 | | | 15 | 35567312 | 4.00E−03 |
| RS1908690 | 2317 | | | 12 | 29249561 | 4.00E−03 |
| RS2474149 | 1930 | | | 6 | 24993358 | 4.00E−03 |
| RS4694890 | 341 | TEC | 7006 | 4 | 47921024 | 4.00E−03 |
| RS7143770 | 2407 | | | 14 | 43899026 | 4.00E−03 |
| RS797054 | 1642 | GADL1 | 339896 | 3 | 30776179 | 4.00E−03 |
| RS10505005 | 2148 | | | 8 | 102085828 | 4.10E−03 |
| RS2863161 | 2253 | PRDM11 | 56981 | 11 | 45178266 | 4.10E−03 |
| RS6601438 | 2084 | MSRA | 4482 | 8 | 10217077 | 4.10E−03 |
| RS10503511 | 2085 | | | 8 | 14569194 | 4.20E−03 |
| RS10503621 | 2090 | PSD3 | 23362 | 8 | 18512468 | 4.20E−03 |
| RS10512854 | 510 | | | 5 | 44546854 | 4.20E−03 |
| RS1467596 | 605 | FLJ33708 | 285780 | 6 | 6360933 | 4.20E−03 |
| RS1493866 | 2454 | | | 16 | 17822257 | 4.20E−03 |
| RS2894508 | 2180 | DDEF1 | 50807 | 8 | 131314769 | 4.20E−03 |
| RS295546 | 1843 | | | 5 | 60523429 | 4.20E−06 |
| RS4132476 | 2197 | | | 9 | 120318112 | 4.20E−03 |
| RS540970 | 1377 | KCTD1 | 284252 | 18 | 22430481 | 4.20E−03 |
| RS7653241 | 1624 | CNTN6 | 27255 | 3 | 1371414 | 4.20E−03 |
| RS1046852 | 20 | C1orf139 | 79971 | 1 | 68363692 | 4.30E−03 |
| RS10495524 | 126 | | | 2 | 5848753 | 4.30E−03 |
| RS10495525 | 129 | | | 2 | 5904629 | 4.30E−03 |
| RS10496894 | 1584 | LRP1B | 53353 | 2 | 142222836 | 4.30E−03 |
| RS1366236 | 586 | GLRA1 | 2741 | 5 | 151264796 | 4.30E−03 |
| RS1525912 | 1704 | FGF12 | 2257 | 3 | 193571552 | 4.30E−03 |
| RS1874069 | 1742 | | | 4 | 59070504 | 4.30E−03 |
| RS1912163 | 340 | TEC | 7006 | 4 | 47905410 | 4.30E−03 |
| RS2458349 | 1105 | | | 11 | 105805096 | 4.30E−03 |
| RS2887526 | 1868 | | | 5 | 98552712 | 4.30E−03 |
| RS6030634 | 2555 | PTPRT | 11122 | 20 | 41140415 | 4.30E−03 |
| RS6074898 | 2550 | C20orf133 | 140733 | 20 | 15384002 | 4.30E−03 |
| RS9284299 | 1277 | UNC13C | 440279 | 15 | 52685923 | 4.30E−03 |
| RS10496893 | 1583 | LRP1B | 53353 | 2 | 142222200 | 4.40E−03 |
| RS10503622 | 2091 | PSD3 | 23362 | 8 | 18513029 | 4.40E−03 |
| RS10506055 | 1119 | | | 12 | 30097508 | 4.40E−03 |
| RS10510640 | 1644 | GADL1 | 339896 | 3 | 30857035 | 4.40E−03 |
| RS10516126 | 1894 | | | 5 | 174414039 | 4.40E−03 |
| RS1352058 | 458 | GLRA3 | 8001 | 4 | 175842980 | 4.40E−03 |
| RS7588808 | 1589 | | | 2 | 145578357 | 4.40E−03 |
| RS10505379 | 2156 | COL14A1 | 7373 | 8 | 121395608 | 4.50E−03 |
| RS1353447 | 2240 | | | 11 | 14988673 | 4.50E−03 |
| RS1501622 | 1705 | FGF12 | 2257 | 3 | 193634069 | 4.50E−03 |
| RS1818704 | 1274 | UNC13C | 440279 | 15 | 52679696 | 4.50E−03 |
| RS2485488 | 2345 | | | 13 | 37441039 | 4.50E−03 |
| RS3782513 | 2318 | PTX1 | 51290 | 12 | 29397521 | 4.50E−03 |
| RS4660275 | 1479 | | | 1 | 44617545 | 4.50E−03 |
| RS581187 | 2114 | | | 8 | 37119893 | 4.50E−03 |
| RS8009199 | 2408 | | | 14 | 43947540 | 4.50E−03 |
| RS9324714 | 585 | GLRA1 | 2741 | 5 | 151223956 | 4.50E−03 |
| RS949355 | 2480 | | | 18 | 20419693 | 4.50E−03 |
| RS10483313 | 2378 | | | 14 | 25709526 | 4.60E−03 |
| RS10492383 | 2319 | PTX1 | 51290 | 12 | 29419862 | 4.60E−03 |
| RS10503831 | 2107 | | | 8 | 28542929 | 4.60E−03 |
| RS10506057 | 1120 | | | 12 | 30159671 | 4.60E−03 |
| RS1441638 | 1778 | NDST4 | 64579 | 4 | 116094415 | 4.60E−03 |
| RS176379 | 2552 | PTPRT | 11122 | 20 | 40898608 | 4.60E−03 |
| RS1912126 | 1588 | | | 2 | 145568461 | 4.60E−03 |
| RS2274822 | 1921 | | | 6 | 14399068 | 4.60E−03 |
| RS295543 | 1844 | | | 5 | 60524136 | 4.60E−03 |
| RS6493693 | 1275 | UNC13C | 440279 | 15 | 52679875 | 4.60E−03 |
| RS10500396 | 2455 | | | 16 | 17948935 | 4.70E−03 |
| RS2681962 | 1276 | UNC13C | 440279 | 15 | 52685879 | 4.70E−03 |
| RS3758729 | 2252 | PRDM11 | 56981 | 11 | 45165411 | 4.70E−03 |
| RS3790557 | 1486 | SERBP1 | 26135 | 1 | 67657985 | 4.70E−03 |
| RS4583980 | 1929 | C6orf32 | 9750 | 6 | 24954242 | 4.70E−03 |
| RS953816 | 2294 | | | 11 | 105793764 | 4.70E−03 |
| RS1860066 | 1884 | CSS3 | 337876 | 5 | 129475223 | 4.80E−03 |
| RS1993306 | 1555 | LTBP1 | 4052 | 2 | 33299839 | 4.80E−03 |
| RS242695 | 2519 | | | 18 | 56920646 | 4.80E−03 |
| RS4981309 | 2399 | BRMS1L | 84312 | 14 | 35374119 | 4.80E−03 |
| RS532022 | 2116 | | | 8 | 37194096 | 4.80E−03 |
| RS755043 | 2223 | | | 10 | 79780432 | 4.80E−03 |
| RS9317821 | 2364 | | | 13 | 69008405 | 4.80E−03 |
| RS9326628 | 190 | | | 2 | 110323956 | 4.80E−03 |
| RS10484178 | 2409 | | | 14 | 43966379 | 4.90E−03 |
| RS10512876 | 516 | | | 5 | 45285361 | 4.90E−03 |
| RS3866642 | 1480 | C1orf164 | 55182 | 1 | 44658509 | 4.90E−03 |
| RS4695357 | 339 | TEC | 7006 | 4 | 47893952 | 4.90E−03 |
| RS6792186 | 1645 | GADL1 | 339896 | 3 | 30867723 | 4.90E−03 |
| RS7767578 | 1903 | FLJ33708 | 285780 | 6 | 6354844 | 4.90E−03 |
| RS10483456 | 2396 | GARNL1 | 253959 | 14 | 35105918 | 5.00E−03 |
| RS10506054 | 1118 | | | 12 | 30096174 | 5.00E−03 |
| RS1391340 | 2514 | | | 18 | 56778284 | 5.00E−03 |
| RS1541081 | 1905 | FLJ33708 | 285780 | 6 | 6364910 | 5.00E−03 |
| RS2272709 | 2072 | LOC644668 | 644668 | 8 | 1804452 | 5.00E−03 |
| RS2278667 | 852 | | | 8 | 80877490 | 5.00E−03 |
| RS4605931 | 1904 | FLJ33708 | 285780 | 6 | 6360586 | 5.00E−03 |
| RS6992703 | 850 | | | 8 | 80831834 | 5.00E−03 |
| RS7748118 | 1906 | FLJ33708 | 285780 | 6 | 6401019 | 5.00E−03 |
| RS989901 | 922 | | | 9 | 103307086 | 5.00E−03 |
| RS10503512 | 2086 | | | 8 | 14592917 | 5.10E−03 |
| RS1072769 | 1117 | | | 12 | 30067073 | 5.10E−03 |
| RS1342501 | 2196 | | | 9 | 120196645 | 5.10E−03 |
| RS1346612 | 128 | | | 2 | 5903374 | 5.10E−03 |
| RS1393749 | 1643 | GADL1 | 339896 | 3 | 30823369 | 5.10E−03 |
| RS1394615 | 1896 | | | 5 | 174448238 | 5.10E−03 |
| RS2353296 | 1733 | | | 4 | 47956320 | 5.10E−03 |
| RS2979710 | 849 | | | 8 | 80814997 | 5.10E−03 |
| RS10504709 | 848 | | | 8 | 80781919 | 5.20E−03 |
| RS10513331 | 2195 | | | 9 | 120180459 | 5.20E−03 |
| RS1429235 | 127 | | | 2 | 5876965 | 5.20E−03 |
| RS2294591 | 2553 | PTPRT | 11122 | 20 | 41066497 | 5.20E−03 |
| RS4739483 | 2117 | | | 8 | 37195876 | 5.20E−03 |
| RS7580778 | 187 | | | 2 | 109757112 | 5.20E−03 |
| RS7850478 | 946 | | | 9 | 119326659 | 5.20E−03 |
| RS10486526 | 2031 | PTHB1 | 27241 | 7 | 33301917 | 5.30E−03 |
| RS10514773 | 1556 | LTBP1 | 4052 | 2 | 33334527 | 5.30E−03 |
| RS574819 | 1547 | | | 1 | 236844151 | 5.30E−03 |
| RS617689 | 2292 | | | 11 | 105767567 | 5.30E−03 |
| RS9324023 | 2379 | | | 14 | 27261761 | 5.30E−03 |
| RS10506053 | 1116 | | | 12 | 30061626 | 5.40E−03 |
| RS2185950 | 2211 | | | 10 | 46509931 | 5.40E−03 |
| RS2268104 | 2307 | GRIN2B | 2904 | 12 | 13714030 | 5.40E−03 |
| RS9296547 | 642 | | | 6 | 47474339 | 5.40E−03 |
| RS949637 | 1895 | | | 5 | 174447933 | 5.40E−03 |
| RS10486530 | 2035 | PTHB1 | 27241 | 7 | 33364558 | 5.50E−03 |
| RS10492132 | 2309 | GRIN2B | 2904 | 12 | 13724820 | 5.50E−03 |
| RS10492899 | 1304 | | | 16 | 50583588 | 5.50E−03 |
| RS10497332 | 1605 | STK39 | 27347 | 2 | 168548561 | 5.50E−03 |
| RS10502473 | 2503 | TAF4B | 6875 | 18 | 22175498 | 5.50E−03 |
| RS161734 | 1861 | CHD1 | 1105 | 5 | 98243608 | 5.50E−03 |
| RS2347597 | 584 | G3BP | 10146 | 5 | 151161511 | 5.50E−03 |
| RS279538 | 1779 | NDST4 | 64579 | 4 | 116105993 | 5.50E−03 |
| RS723043 | 157 | LTBP1 | 4052 | 2 | 33395291 | 5.50E−03 |
| RS9291321 | 1734 | | | 4 | 47956918 | 5.50E−03 |
| RS10502445 | 2475 | LOC390843 | 390843 | 18 | 20367446 | 5.60E−03 |
| RS10504165 | 2122 | | | 8 | 55476487 | 5.60E−03 |
| RS1149732 | 2221 | | | 10 | 79656358 | 5.60E−03 |
| RS1456196 | 293 | | | 3 | 119137243 | 5.60E−03 |
| RS1785738 | 2502 | | | 18 | 21681642 | 5.60E−03 |
| RS1805082 | 1370 | NPC1 | 4864 | 18 | 19374442 | 5.60E−03 |
| RS2031393 | 621 | | | 6 | 23796368 | 5.60E−03 |
| RS2066387 | 2194 | | | 9 | 120178850 | 5.60E−03 |
| RS2764536 | 28 | | | 1 | 69819828 | 5.60E−03 |
| RS3884537 | 2241 | | | 11 | 15020214 | 5.60E−03 |
| RS9315542 | 2349 | | | 13 | 37529471 | 5.60E−03 |
| RS965017 | 1897 | | | 5 | 174509108 | 5.60E−03 |
| RS10498721 | 1928 | C6orf32 | 9750 | 6 | 24953309 | 5.70E−03 |
| RS10502463 | 2484 | | | 18 | 20815751 | 5.70E−03 |
| RS10503513 | 2087 | | | 8 | 14598632 | 5.70E−03 |
| RS3104997 | 2143 | | | 8 | 96630910 | 5.70E−03 |
| RS6987277 | 766 | | | 8 | 8977241 | 5.70E−03 |
| RS9283566 | 1680 | | | 3 | 119162948 | 5.70E−03 |
| RS1374663 | 1869 | | | 5 | 98552774 | 5.80E−03 |
| RS1401154 | 2115 | | | 8 | 37145702 | 5.80E−03 |
| RS1549920 | 583 | | | 5 | 151121037 | 5.80E−03 |
| RS2102516 | 2088 | | | 8 | 14623080 | 5.80E−03 |
| RS3848309 | 1309 | | | 16 | 50690687 | 5.80E−03 |
| RS10491511 | 917 | | | 9 | 103219769 | 5.90E−03 |
| RS352210 | 2105 | FZD3 | 7976 | 8 | 28438339 | 5.90E−03 |
| RS567992 | 2293 | | | 11 | 105767607 | 5.90E−03 |
| RS716077 | 2179 | DDEF1 | 50807 | 8 | 131194895 | 5.90E−03 |
| RS795446 | 1641 | GADL1 | 339896 | 3 | 30776092 | 5.90E−03 |
| RS10483454 | 2394 | | | 14 | 35046027 | 6.00E−03 |
| RS10492135 | 1114 | GRIN2B | 2904 | 12 | 13777535 | 6.00E−03 |
| RS10516605 | 1772 | NDST4 | 64579 | 4 | 116016924 | 6.00E−03 |
| RS1877591 | 2225 | | | 10 | 79784171 | 6.00E−03 |
| RS2216031 | 2032 | PTHB1 | 27241 | 7 | 33312005 | 6.00E−03 |
| RS2264044 | 2089 | | | 8 | 14623176 | 6.00E−03 |
| RS2282653 | 2242 | CALCB | 797 | 11 | 15055294 | 6.00E−03 |
| RS2461055 | 851 | | | 8 | 80832082 | 6.00E−03 |
| RS10484608 | 1973 | FBXL4 | 26235 | 6 | 99442369 | 6.10E−03 |
| RS10490995 | 2224 | | | 10 | 79780912 | 6.10E−03 |
| RS1493868 | 2453 | | | 16 | 17822090 | 6.10E−03 |
| RS2055420 | 1706 | FGF12 | 2257 | 3 | 193724133 | 6.10E−03 |
| RS2075933 | 1469 | | | 1 | 34693662 | 6.10E−03 |
| RS2123769 | 1557 | LTBP1 | 4052 | 2 | 33378911 | 6.10E−03 |
| RS2163326 | 1664 | MITF | 4286 | 3 | 69959373 | 6.10E−03 |
| RS2284410 | 2312 | GRIN2B | 2904 | 12 | 13751078 | 6.10E−03 |
| RS10483455 | 2395 | LOC122592 | 122592 | 14 | 35053977 | 6.20E−03 |
| RS10492385 | 2315 | | | 12 | 29248257 | 6.20E−03 |
| RS4574886 | 2161 | COL14A1 | 7373 | 8 | 121444326 | 6.20E−03 |
| RS751716 | 2222 | | | 10 | 79720255 | 6.20E−03 |
| RS8032707 | 2444 | LOC390576 | 390576 | 15 | 35572205 | 6.20E−03 |
| RS28181 | 1814 | SEMA5A | 9037 | 5 | 9297526 | 6.30E−03 |
| RS10514762 | 1681 | | | 3 | 119164163 | 6.40E−03 |
| RS1528776 | 1536 | | | 1 | 235218861 | 6.40E−03 |
| RS787433 | 1590 | | | 2 | 145580328 | 6.40E−03 |
| RS9302534 | 2456 | | | 16 | 17956211 | 6.40E−03 |
| RS10500395 | 2457 | | | 16 | 17976966 | 6.50E−03 |
| RS174489 | 1619 | CNTN6 | 27255 | 3 | 1308797 | 6.50E−03 |
| RS3853364 | 1477 | PRNPIP | 79033 | 1 | 44544072 | 6.50E−03 |
| RS1014500 | 2289 | | | 11 | 93574371 | 6.60E−03 |
| RS10496974 | 1591 | | | 2 | 145585112 | 6.60E−03 |
| RS723042 | 1558 | LTBP1 | 4052 | 2 | 33395206 | 6.60E−03 |
| RS9285957 | 1867 | | | 5 | 98528565 | 6.60E−03 |
| RS1592326 | 2212 | LOC643650 | 643650 | 10 | 46542721 | 6.70E−03 |
| RS2662258 | 1859 | RGMB | 285704 | 5 | 98153430 | 6.70E−03 |
| RS10484177 | 2410 | | | 14 | 43966684 | 6.80E−03 |
| RS10496972 | 1587 | | | 2 | 145540029 | 6.80E−03 |
| RS1337647 | 2344 | | | 13 | 37440845 | 6.80E−03 |
| RS161723 | 1478 | PRNPIP | 79033 | 1 | 44555954 | 6.80E−03 |
| RS1073601 | 1870 | | | 5 | 98594496 | 6.90E−03 |
| RS1426935 | 456 | | | 4 | 175767040 | 6.90E−03 |
| RS725937 | 582 | SPARC | 6678 | 5 | 151036904 | 6.90E−03 |
| RS10496895 | 1586 | LRP1B | 53353 | 2 | 142242169 | 7.10E−03 |
| RS1816607 | 1579 | LRP1B | 53353 | 2 | 142194712 | 7.10E−03 |
| RS470732 | 1166 | | | 12 | 128049248 | 7.10E−03 |
| RS896331 | 2254 | | | 11 | 45371611 | 7.10E−03 |
| RS4112759 | 944 | | | 9 | 119274090 | 7.20E−03 |
| RS10491853 | 2193 | | | 9 | 119437440 | 7.30E−03 |
| RS9291322 | 1735 | | | 4 | 47958799 | 7.30E−03 |
| RS10483311 | 2374 | | | 14 | 25665022 | 7.40E−03 |
| RS240943 | 2109 | EXTL3 | 2137 | 8 | 28620759 | 7.40E−03 |
| RS2474265 | 1974 | LOC442238 | 442238 | 6 | 100640983 | 7.40E−03 |
| RS533706 | 1943 | LOC653802 | 653802 | 6 | 42420353 | 7.40E−03 |
| RS10499770 | 2047 | LOC653435 | 653435 | 7 | 63208951 | 7.50E−03 |
| RS10503168 | 2073 | LOC644668 | 644668 | 8 | 1804693 | 7.50E−03 |
| RS1945613 | 2243 | | | 11 | 15057846 | 7.50E−03 |
| RS2200455 | 1160 | | | 12 | 127358570 | 7.50E−03 |
| RS4128928 | 1678 | | | 3 | 119083686 | 7.50E−03 |
| RS4131462 | 1972 | | | 6 | 99364568 | 7.50E−03 |
| RS4510935 | 945 | | | 9 | 119325172 | 7.50E−03 |
| RS10502069 | 2295 | | | 11 | 105821821 | 7.60E−03 |
| RS9323099 | 2411 | | | 14 | 44019800 | 7.70E−03 |
| RS2614074 | 813 | | | 8 | 28222739 | 7.80E−03 |
| RS1436322 | 2320 | | | 12 | 29446072 | 7.90E−03 |
| RS1948697 | 1682 | LOC389142 | 389142 | 3 | 119243756 | 7.90E−03 |
| RS1003194 | 2244 | | | 11 | 15082661 | 8.10E−03 |
| RS2170367 | 1970 | | | 6 | 99201267 | 8.10E−03 |
| RS10492136 | 2313 | GRIN2B | 2904 | 12 | 13803044 | 8.20E−03 |
| RS10511369 | 1677 | | | 3 | 118824534 | 8.20E−03 |
| RS2396817 | 636 | | | 6 | 47255521 | 8.20E−03 |
| RS10496892 | 1581 | LRP1B | 53353 | 2 | 142195142 | 8.30E−03 |
| RS10499023 | 1971 | | | 6 | 99333450 | 8.30E−03 |
| RS10483390 | 2391 | | | 14 | 31411927 | 8.40E−03 |
| RS10503830 | 2106 | FZD3 | 7976 | 8 | 28448462 | 8.60E−03 |
| RS6760896 | 1618 | MGC42174 | 129563 | 2 | 232881167 | 8.60E−03 |
| RS1847340 | 1707 | FGF12 | 2257 | 3 | 193762780 | 8.70E−03 |
| RS331934 | 1860 | | | 5 | 98167620 | 8.70E−03 |
| RS9322878 | 2390 | | | 14 | 31411657 | 8.70E−03 |
| RS6985068 | 2081 | MSRA | 4482 | 8 | 10147230 | 8.80E−03 |
| RS10496891 | 1580 | LRP1B | 53353 | 2 | 142195006 | 9.00E−03 |
| RS10520290 | 460 | GLRA3 | 8001 | 4 | 175868392 | 9.00E−03 |
| RS329205 | 2190 | | | 9 | 8698415 | 9.00E−03 |
| RS4129498 | 2494 | | | 18 | 21255712 | 9.00E−03 |
| RS10484332 | 1922 | | | 6 | 14448309 | 9.10E−03 |
| RS10518956 | 2445 | LOC390576 | 390576 | 15 | 35596072 | 9.10E−03 |
| RS1392265 | 1683 | LOC389142 | 389142 | 3 | 119283235 | 9.10E−03 |
| RS10489168 | 1476 | PRNPIP | 79033 | 1 | 44500588 | 9.60E−03 |
| RS1397853 | 2551 | PTPRT | 11122 | 20 | 40898556 | 9.60E−03 |
| RS1524622 | 2045 | | | 7 | 62473121 | 9.60E−03 |
| RS4840117 | 1976 | | | 6 | 100671550 | 9.70E−03 |
| RS527828 | 2291 | | | 11 | 105756688 | 9.80E−03 |
| RS9295626 | 1923 | KIAA0319 | 9856 | 6 | 24695318 | 9.80E−03 |
| RS10495786 | 1559 | LTBP1 | 4052 | 2 | 33412933 | 9.90E−03 |
| RS2328846 | 1924 | KIAA0319 | 9856 | 6 | 24753174 | 1.01E−02 |
| RS3213942 | 1604 | STK39 | 27347 | 2 | 168529715 | 1.02E−02 |
| RS966905 | 2251 | | | 11 | 45061617 | 1.02E−02 |
| RS1915832 | 2046 | LOC643595 | 643595 | 7 | 57008366 | 1.03E−02 |
| RS10495785 | 158 | LTBP1 | 4052 | 2 | 33414601 | 1.08E−02 |
| RS6543706 | 1560 | LTBP1 | 4052 | 2 | 33413214 | 1.08E−02 |
| RS871151 | 75 | | | 1 | 100896325 | 1.08E−02 |
| RS9989478 | 2468 | GAS7 | 8522 | 17 | 9966550 | 1.10E−02 |
| RS6904345 | 1925 | THEM2 | 55856 | 6 | 24805403 | 1.10E−02 |
| RS10503406 | 2080 | MSRA | 4482 | 8 | 10146382 | 1.12E−02 |
| RS2754777 | 1926 | | | 6 | 24898441 | 1.12E−02 |
| RS4128139 | 2446 | | | 15 | 35710549 | 1.13E−02 |
| RS344924 | 15 | | | 1 | 67878041 | 1.15E−02 |
| RS623892 | 2049 | | | 7 | 63382694 | 1.15E−02 |
| RS9297299 | 2147 | | | 8 | 102085512 | 1.15E−02 |
| RS10495787 | 1561 | LTBP1 | 4052 | 2 | 33453813 | 1.16E−02 |
| RS687547 | 2048 | | | 7 | 63382364 | 1.16E−02 |
| RS10493058 | 1474 | | | 1 | 34761541 | 1.17E−02 |
| RS2227019 | 2110 | | | 8 | 28671025 | 1.18E−02 |
| RS1500251 | 1871 | | | 5 | 98621353 | 1.22E−02 |
| RS632301 | 1380 | | | 18 | 22616756 | 1.22E−02 |
| RS1429220 | 125 | | | 2 | 5848492 | 1.23E−02 |
| RS10520288 | 455 | | | 4 | 175746768 | 1.24E−02 |
| RS1354151 | 1676 | | | 3 | 118818111 | 1.24E−02 |
| RS10491090 | 2469 | GAS7 | 8522 | 17 | 9988337 | 1.25E−02 |
| RS10497331 | 1603 | STK39 | 27347 | 2 | 168527326 | 1.30E−02 |
| RS10502470 | 1378 | KCTD1 | 284252 | 18 | 22452475 | 1.34E−02 |
| RS2077811 | 1379 | | | 18 | 22558197 | 1.41E−02 |
| RS7813443 | 2146 | | | 8 | 102074947 | 1.42E−02 |
| RS10497330 | 1602 | | | 2 | 168508565 | 1.53E−02 |
| RS7030902 | 2191 | | | 9 | 87024911 | 1.61E−02 |
| RS1514897 | 2342 | | | 12 | 127330028 | 2.29E−02 |
| RS7213983 | 2467 | GLP2R | 9340 | 17 | 9732840 | 3.02E−02 |
| RS10521160 | 2465 | USP43 | 124739 | 17 | 9512325 | 3.23E−02 |
| RS1402656 | 2466 | GLP2R | 9340 | 17 | 9681965 | 3.25E−02 |
| RS10501796 | 2275 | FAT3 | 120114 | 11 | 92092804 | <0.0001 |
| RS10501797 | 2276 | FAT3 | 120114 | 11 | 92167618 | <0.0001 |
| RS10507768 | 2359 | | | 13 | 68865849 | <0.0001 |
| RS10517836 | 1747 | | | 4 | 63541480 | <0.0001 |
| RS1062225 | 984 | MAPK8 | 5599 | 10 | 49313232 | <0.0001 |
| RS1792369 | 1090 | FAT3 | 120114 | 11 | 92092081 | <0.0001 |
| RS1867584 | 985 | | | 10 | 49321355 | <0.0001 |
| RS2889611 | 2213 | | | 10 | 49321754 | <0.0001 |
| RS3912653 | 1091 | FAT3 | 120114 | 11 | 92135172 | <0.0001 |
| RS7939598 | 2277 | FAT3 | 120114 | 11 | 92212152 | <0.0001 |
| RS949627 | 2360 | | | 13 | 68893818 | <0.0001 |

TABLE 8
Genes associated with HDL based on t-test,
F-test and HPM results.

| GENE | Gene_ID | Chromosome |
|---|---|---|
| C6orf103 | 79747 | 6 |
| NPAS3 | 64067 | 14 |
| MAGI2 | 9863 | 7 |
| ANGPT1 | 284 | 8 |
| RRM2B | 50484 | 8 |
| TAF7 | 6879 | 5 |
| MGC42174 | 129563 | 2 |
| FLJ33708 | 285780 | 6 |
| LY86 | 9450 | 6 |
| NTNG1 | 22854 | 1 |
| LTBP1 | 4052 | 2 |
| MBP | 4155 | 18 |
| MSR1 | 4481 | 8 |
| SERPINA5 | 5104 | 14 |
| THRB | 7068 | 3 |
| GALC | 2581 | 14 |
| ARL15 | 54622 | 5 |
| GABRG2 | 2566 | 5 |
| TMEM16C | 63982 | 11 |
| UNC13C | 440279 | 15 |
| LOC644954 | 644954 | 10 |
| HNRPD | 3184 | 4 |
| LOC391672 | 391672 | 4 |
| CNGB3 | 54714 | 8 |
| SGCG | 6445 | 13 |
| 13CDNA73 | 10129 | 13 |
| BICC1 | 80114 | 10 |
| ZFPM2 | 23414 | 8 |
| CRIM1 | 51232 | 2 |
| TULP4 | 56995 | 6 |
| EFHA1 | 221154 | 13 |
| GPR110 | 266977 | 6 |
| LUZP2 | 338645 | 11 |
| SYNE2 | 23224 | 14 |
| LOC646176 | 646176 | 11 |
| PYGL | 5836 | 14 |
| FRAS1 | 80144 | 4 |
| CNNM2 | 54805 | 10 |
| HYPB | 29072 | 3 |
| DMD | 1756 | X |
| SVEP1 | 79987 | 9 |
| MAPK8 | 5599 | 10 |
| ABCD3 | 5825 | 1 |
| NTN1 | 9423 | 17 |
| SPATA4 | 132851 | 4 |
| FLJ32942 | 121355 | 12 |
| SNAP25 | 6616 | 20 |
| NAALADL2 | 254827 | 3 |
| CAMTA1 | 23261 | 1 |
| NT5C2 | 22978 | 10 |
| RORA | 6095 | 15 |
| XRCC2 | 7516 | 7 |
| mimitin | 91942 | 5 |
| DOK6 | 220164 | 18 |
| LOC400954 | 400954 | 2 |
| FRMPD2 | 143162 | 10 |
| NPC1 | 4864 | 18 |
| PRKG1 | 5592 | 10 |
| LOC646790 | 646790 | 2 |
| FARS2 | 10667 | 6 |
| GPC5 | 2262 | 13 |
| WRN | 7486 | 8 |
| ZNF429 | 353088 | 19 |
| CACNA1E | 777 | 1 |
| GARNL1 | 253959 | 14 |
| APLP2 | 334 | 11 |
| ASTN2 | 23245 | 9 |
| VGCNL1 | 259232 | 13 |
| TRPA1 | 8989 | 8 |
| DLG2 | 1740 | 11 |
| CARD8 | 22900 | 19 |
| HECW2 | 57520 | 2 |
| LAMA3 | 3909 | 18 |
| TANC2 | 26115 | 17 |
| TAF3 | 83860 | 10 |
| KIAA1843 | 84540 | 2 |
| PTPN12 | 5782 | 7 |
| UNQ6975 | 400952 | 2 |
| NCAM1 | 4684 | 11 |
| FLJ10099 | 55069 | 7 |
| CUBN | 8029 | 10 |
| C10orf130 | 387707 | 10 |
| E2F2 | 1870 | 1 |
| F3 | 2152 | 1 |
| SFRP4 | 6424 | 7 |
| KALRN | 8997 | 3 |
| LOC653748 | 653748 | 7 |
| RIT2 | 6014 | 18 |
| LOC285929 | 285929 | 7 |
| TAS2R16 | 50833 | 7 |
| ITGB5 | 3693 | 3 |
| CCDC26 | 137196 | 8 |
| FLT4 | 2324 | 5 |
| BLNK | 29760 | 10 |
| GLA | 2717 | X |
| LOC644053 | 644053 | 1 |
| PCSK1 | 5122 | 5 |
| CNTN3 | 5067 | 3 |
| LOC646444 | 646444 | 8 |
| C10orf107 | 219621 | 10 |
| LOC138046 | 138046 | 8 |
| ANK3 | 288 | 10 |
| DKFZP566M1046 | 84067 | 11 |
| ADAM12 | 8038 | 10 |
| SMYD3 | 64754 | 1 |
| C10orf112 | 340895 | 10 |
| MGC48628 | 401145 | 4 |
| UFM1 | 51569 | 13 |
| PCDH7 | 5099 | 4 |
| LRP1B | 53353 | 2 |
| CDADC1 | 81602 | 13 |
| NRG3 | 10718 | 10 |
| HNRPDL | 9987 | 4 |
| LOC645492 | 645492 | 6 |
| FMN2 | 56776 | 1 |
| FAT3 | 120114 | 11 |
| FLJ16124 | 440867 | 2 |
| KIAA1797 | 54914 | 9 |
| SLC4A4 | 8671 | 4 |
| C12orf26 | 84190 | 12 |
| AAA1 | 404744 | 7 |
| GABRB1 | 2560 | 4 |
| LOC647219 | 647219 | 1 |
| PGM5 | 5239 | 9 |
| GNAL | 2774 | 18 |
| ADAM29 | 11086 | 4 |
| MAP2K6 | 5608 | 17 |
| PSMA1 | 5682 | 11 |
| TLR4 | 7099 | 9 |
| LOC442161 | 442161 | 6 |
| SLC15A2 | 6565 | 3 |
| AMPH | 273 | 7 |
| UNC5C | 8633 | 4 |
| UBQLN1 | 29979 | 9 |
| LOC388458 | 388458 | 18 |
| CPNE3 | 8895 | 8 |
| SLC15A4 | 121260 | 12 |
| EPHB1 | 2047 | 3 |
| GPR158 | 57512 | 10 |
| GPC6 | 10082 | 13 |
| TRIM24 | 8805 | 7 |
| HLA-DQB2 | 3120 | 6 |
| KIAA1772 | 80000 | 18 |
| VRK1 | 7443 | 14 |
| PTPRT | 11122 | 20 |
| RBBP5 | 5929 | 1 |
| DKFZp761O2018 | 92293 | 12 |
| LOC388730 | 388730 | 1 |
| KCNH5 | 27133 | 14 |
| DGKB | 1607 | 7 |
| COL25A1 | 84570 | 4 |
| BIVM | 54841 | 13 |
| FBXL3 | 26224 | 13 |
| TSPAN8 | 7103 | 12 |
| TXNDC3 | 51314 | 7 |
| GRIN2B | 2904 | 12 |
| LOC283553 | 283553 | 14 |
| PLEKHA7 | 144100 | 11 |
| RAPGEF4 | 11069 | 2 |
| LPHN2 | 23266 | 1 |
| HABP2 | 3026 | 10 |
| SDK1 | 221935 | 7 |
| CNTN5 | 53942 | 11 |
| GBP2 | 2634 | 1 |
| GRIK1 | 2897 | 21 |
| ADCY2 | 108 | 5 |
| GAS7 | 8522 | 17 |
| RIPK5 | 25778 | 1 |
| FAM77D | 286183 | 8 |
| AOX2 | 344454 | 2 |
| BCL2 | 596 | 18 |
| ZBTB10 | 65986 | 8 |
| CSMD1 | 64478 | 8 |
| BACE2 | 25825 | 21 |
| PLEKHH2 | 130271 | 2 |
| EPDR1 | 54749 | 7 |
| KCTD1 | 284252 | 18 |
| LOC442117 | 442117 | 4 |
| C11orf11 | 747 | 11 |
| PAP2D | 163404 | 1 |
| MANBA | 4126 | 4 |
| CRSP8 | 9442 | 9 |
| LOC646697 | 646697 | 4 |
| LOC440261 | 440261 | 15 |
| KCNQ5 | 56479 | 6 |
| GMDS | 2762 | 6 |
| PSD3 | 23362 | 8 |
| CAST1 | 26059 | 3 |
| DLGAP2 | 9228 | 8 |
| USH2A | 7399 | 1 |
| RYR2 | 6262 | 1 |
| MCF2L2 | 23101 | 3 |
| VSNL1 | 7447 | 2 |
| LSAMP | 4045 | 3 |
| FARP1 | 10160 | 13 |
| MCTP2 | 55784 | 15 |
| SRFBP1 | 153443 | 5 |
| EPHA4 | 2043 | 2 |
| CRISP2 | 7180 | 6 |
| LOC442238 | 442238 | 6 |
| POU6F2 | 11281 | 7 |
| LOC144678 | 144678 | 12 |
| KIAA1276 | 27146 | 4 |
| GBAS | 2631 | 7 |
| PDE8B | 8622 | 5 |
| NMNAT2 | 23057 | 1 |
| FLJ34931 | 388939 | 2 |
| PTPRJ | 5795 | 11 |
| OSBPL1A | 114876 | 18 |
| FLJ25323 | 374470 | 12 |
| SCFD2 | 152579 | 4 |
| ABCA12 | 26154 | 2 |
| MAPK10 | 5602 | 4 |
| LOC645939 | 645939 | 1 |
| LOC139060 | 139060 | X |
| USP10 | 9100 | 16 |
| NFIA | 4774 | 1 |
| CLDN11 | 5010 | 3 |
| RGS9 | 8787 | 17 |
| IL6 | 3569 | 7 |
| ITSN1 | 6453 | 21 |
| GALNT14 | 79623 | 2 |
| TASP1 | 55617 | 20 |
| CLPB | 81570 | 11 |
| PTPRR | 5801 | 12 |
| USP24 | 23358 | 1 |
| FLJ39155 | 133584 | 5 |
| MALL | 7851 | 2 |
| C9orf125 | 84302 | 9 |
| PRICKLE2 | 166336 | 3 |
| DNAJC5B | 85479 | 8 |
| DTNA | 1837 | 18 |
| SDHC | 6391 | 1 |
| HSPC117 | 51493 | 22 |
| LCTL | 197021 | 15 |
| MGC9850 | 219404 | 13 |
| LOC119710 | 119710 | 11 |
| LOC645254 | 645254 | 10 |
| UST | 10090 | 6 |
| KIAA1622 | 57718 | 14 |
| MYO1B | 4430 | 2 |
| C9orf39 | 54875 | 9 |
| STAU2 | 27067 | 8 |
| ESRRG | 2104 | 1 |
| AYTL1 | 54947 | 16 |
| ATE1 | 11101 | 10 |
| COMMD10 | 51397 | 5 |
| PHACTR1 | 221692 | 6 |
| YPEL2 | 388403 | 17 |
| KIAA1706 | 80820 | 7 |
| LRRN6C | 158038 | 9 |
| GDF9 | 2661 | 5 |
| LGR5 | 8549 | 12 |
| FLJ45974 | 401337 | 7 |
| VAV3 | 10451 | 1 |
| C20orf133 | 140733 | 20 |
| LOC644802 | 644802 | 22 |
| C10orf80 | 159686 | 10 |
| MEIS2 | 4212 | 15 |
| SLC9A9 | 285195 | 3 |
| GBP1 | 2633 | 1 |
| GPR116 | 221395 | 6 |
| SLC39A12 | 221074 | 10 |
| TEC | 7006 | 4 |
| KIAA0746 | 23231 | 4 |
| FLJ26443 | 400165 | 13 |
| LOC342531 | 342531 | 17 |
| IRS1 | 3667 | 2 |
| FSTL4 | 23105 | 5 |
| ZNF533 | 151126 | 2 |
| CHST11 | 50515 | 12 |
| SLC17A3 | 10786 | 6 |
| MSRB3 | 253827 | 12 |
| DSG4 | 147409 | 18 |
| LOC441900 | 441900 | 1 |
| TMEFF2 | 23671 | 2 |
| ZNF659 | 79750 | 3 |
| RSBN1L | 222194 | 7 |
| PAPPA2 | 60676 | 1 |
| DCAMKL2 | 166614 | 4 |
| LOC645986 | 645986 | 8 |
| GRM7 | 2917 | 3 |
| LOC134541 | 134541 | 5 |
| SPIRE1 | 56907 | 18 |
| EDIL3 | 10085 | 5 |
| ACN9 | 57001 | 7 |
| PEX5L | 51555 | 3 |
| MMP2 | 4313 | 16 |
| ABCC4 | 10257 | 13 |
| NPHP1 | 4867 | 2 |
| CNTN2 | 6900 | 1 |
| GPR73L1 | 128674 | 20 |
| DDAH1 | 23576 | 1 |
| WWOX | 51741 | 16 |
| TCAB1 | 154215 | 6 |
| SYNE1 | 23345 | 6 |
| LOC647154 | 647154 | 13 |
| G3BP | 10146 | 5 |
| COL24A1 | 255631 | 1 |
| SNTG1 | 54212 | 8 |
| IL19 | 29949 | 1 |
| CDH13 | 1012 | 16 |
| FLJ34690 | 284034 | 17 |
| C9orf84 | 158401 | 9 |
| NAV3 | 89795 | 12 |
| SORBS1 | 10580 | 10 |
| SV2V | 22987 | 5 |
| PIK3C3 | 5289 | 18 |
| CNTN4 | 152330 | 3 |
| C6orf96 | 55005 | 6 |
| LOC646169 | 646169 | 11 |
| FBXO33 | 254170 | 14 |
| RGS3 | 5998 | 9 |
| PHC1 | 1911 | 12 |
| SALF | 286749 | 2 |
| GLRA1 | 2741 | 5 |
| GLRA3 | 8001 | 4 |
| LOC389644 | 389644 | 8 |
| PALLD | 23022 | 4 |
| IQCB1 | 9657 | 3 |
| FLJ22104 | 65084 | 11 |
| GRIN2A | 2903 | 16 |
| KIAA0774 | 23281 | 13 |
| DCP1B | 196513 | 12 |
| NUBPL | 80224 | 14 |
| PNLIPRP3 | 119548 | 10 |
| FAT4 | 79633 | 4 |
| LOC401898 | 401898 | 19 |
| COL11A1 | 1301 | 1 |
| GLYAT | 10249 | 11 |
| GLYATL2 | 219970 | 11 |
| GRIN3A | 116443 | 9 |
| FLJ21820 | 60526 | 2 |
| ZBTB20 | 26137 | 3 |
| PTPRD | 5789 | 9 |
| TEX2 | 55852 | 17 |
| SLC6A17 | 388662 | 1 |
| NCAM2 | 4685 | 21 |
| CDK6 | 1021 | 7 |
| ROR1 | 4919 | 1 |
| TMEM16D | 121601 | 12 |
| KBTBD9 | 114818 | 2 |
| ANXA5 | 308 | 4 |
| SPARC | 6678 | 5 |
| ZBTB2 | 57621 | 6 |
| CST9 | 128822 | 20 |
| ST3GAL6 | 10402 | 3 |
| C1orf139 | 79971 | 1 |
| NTRK2 | 4915 | 9 |
| LOC128820 | 128820 | 20 |
| ROCK1 | 6093 | 18 |
| LOC401954 | 401954 | 1 |
| ZNF85 | 7639 | 19 |
| ZNF208 | 7757 | 19 |
| OTUB2 | 78990 | 14 |
| GALNTL4 | 374378 | 11 |
| TMCC2 | 9911 | 1 |
| LOC390503 | 390503 | 14 |
| PNOC | 5368 | 8 |

TABLE 9
Genes associated with APOA1 based on t-test,
F-test and HPM results.

| GENE | Gene_ID | Chromosome |
|---|---|---|
| SERPINA5 | 5104 | 14 |
| PSD3 | 23362 | 8 |
| LOC644954 | 644954 | 10 |
| mimitin | 91942 | 5 |
| MGC42174 | 129563 | 2 |
| NPAS3 | 64067 | 14 |
| RRM2B | 50484 | 8 |
| FLJ33708 | 285780 | 6 |
| TAF7 | 6879 | 5 |
| FAT3 | 120114 | 11 |
| MAGI2 | 9863 | 7 |
| LY86 | 9450 | 6 |
| MBP | 4155 | 18 |
| BMP6 | 654 | 6 |
| GBP2 | 2634 | 1 |
| GARNL1 | 253959 | 14 |
| GABRG2 | 2566 | 5 |
| DACH2 | 117154 | X |
| MAPK8 | 5599 | 10 |
| MDS1 | 4197 | 3 |
| LOC442238 | 442238 | 6 |
| MARCH8 | 220972 | 10 |
| DKFZp761O2018 | 92293 | 12 |
| GBP1 | 2633 | 1 |
| NDST4 | 64579 | 4 |
| RAB6IP2 | 23085 | 12 |
| PTPRT | 11122 | 20 |
| TM7SF4 | 81501 | 8 |
| SLC17A3 | 10786 | 6 |
| AMPH | 273 | 7 |
| LOC128153 | 128153 | 1 |
| DLGAP2 | 9228 | 8 |
| LPHN2 | 23266 | 1 |
| C6orf103 | 79747 | 6 |
| SDK1 | 221935 | 7 |
| ANK3 | 288 | 10 |
| TPD52L1 | 7164 | 6 |
| SEMA5A | 9037 | 5 |
| ZNF521 | 25925 | 18 |
| ARHGAP22 | 58504 | 10 |
| ELOVL7 | 79993 | 5 |
| C9orf39 | 54875 | 9 |
| DEPC-1 | 221120 | 11 |
| ESRRG | 2104 | 1 |
| LTBP1 | 4052 | 2 |
| ARL15 | 54622 | 5 |
| FAM19A4 | 151647 | 3 |
| CLPTM1 | 1209 | 19 |
| CDYL | 9425 | 6 |
| MSR1 | 4481 | 8 |
| RYR2 | 6262 | 1 |
| C20orf133 | 140733 | 20 |
| LOC392222 | 392222 | 8 |
| TASP1 | 55617 | 20 |
| FLT4 | 2324 | 5 |
| GRIK4 | 2900 | 11 |
| THRB | 7068 | 3 |
| MITF | 4286 | 3 |
| C15orf41 | 84529 | 15 |
| LOC389142 | 389142 | 3 |
| NRG3 | 10718 | 10 |
| SLC15A4 | 121260 | 12 |
| LOC144678 | 144678 | 12 |
| PTHB1 | 27241 | 7 |
| TAF3 | 83860 | 10 |
| RIMS1 | 22999 | 6 |
| ACADSB | 36 | 10 |
| SLC35F1 | 222553 | 6 |
| ABCA12 | 26154 | 2 |
| HEPHL1 | 341208 | 11 |
| BAI3 | 577 | 6 |
| HUNK | 30811 | 21 |
| UST | 10090 | 6 |
| TAF4B | 6875 | 18 |
| COL14A1 | 7373 | 8 |
| CSS3 | 337876 | 5 |
| LOC653802 | 653802 | 6 |
| C9orf84 | 158401 | 9 |
| GRM1 | 2911 | 6 |
| C8orf37 | 157657 | 8 |
| ASTN | 460 | 1 |
| RGS7 | 6000 | 1 |
| FLJ36166 | 349152 | 7 |
| ANGPT1 | 284 | 8 |
| FARS2 | 10667 | 6 |
| SYNE2 | 23224 | 14 |
| SMYD3 | 64754 | 1 |
| GPC5 | 2262 | 13 |
| FAM55D | 54827 | 11 |
| LOC390843 | 390843 | 18 |
| LOC283247 | 283247 | 11 |
| ADAMTS12 | 81792 | 5 |
| FGF12 | 2257 | 3 |
| MTMR3 | 8897 | 22 |
| LUZP2 | 338645 | 11 |
| C9orf125 | 84302 | 9 |
| SYTL3 | 94120 | 6 |
| NTNG1 | 22854 | 1 |
| UNC5C | 8633 | 4 |
| KIAA0672 | 9912 | 17 |
| RORA | 6095 | 15 |
| ZFPM2 | 23414 | 8 |
| GAS7 | 8522 | 17 |
| NUBPL | 80224 | 14 |
| SEC11L2 | 157708 | 8 |
| PTPRJ | 5795 | 11 |
| UNC13C | 440279 | 15 |
| C21orf41 | 54073 | 21 |
| KIAA1191 | 57179 | 5 |
| GPR115 | 221393 | 6 |
| UNQ6975 | 400952 | 2 |
| CGI-09 | 51602 | 20 |
| DMD | 1756 | X |
| TEC | 7006 | 4 |
| HSPC117 | 51493 | 22 |
| TCTEX1D1 | 200132 | 1 |
| FBN2 | 2201 | 5 |
| CSMD1 | 64478 | 8 |
| CARD8 | 22900 | 19 |
| PDE4D | 5144 | 5 |
| SEMA3D | 223117 | 7 |
| NAALADL2 | 254827 | 3 |
| MID1 | 4281 | X |
| C8ORFK36 | 340359 | 8 |
| RB1CC1 | 9821 | 8 |
| IFRD1 | 3475 | 7 |
| TPR | 7175 | 1 |
| ERCC8 | 1161 | 5 |
| XRCC2 | 7516 | 7 |
| BCAS1 | 8537 | 20 |
| MSRA | 4482 | 8 |
| DGKB | 1607 | 7 |
| ATG5 | 9474 | 6 |
| C7orf25 | 79020 | 7 |
| TMEM108 | 66000 | 3 |
| PLXDC2 | 84898 | 10 |
| HECW2 | 57520 | 2 |
| MSH3 | 4437 | 5 |
| KIAA1239 | 57495 | 4 |
| GRIN2B | 2904 | 12 |
| TXNDC5 | 81567 | 6 |
| LOC138046 | 138046 | 8 |
| C1orf139 | 79971 | 1 |
| C10orf130 | 387707 | 10 |
| DNAJA2 | 10294 | 16 |
| KCTD1 | 284252 | 18 |
| LOC643811 | 643811 | 15 |
| FLJ10379 | 55133 | 2 |
| PIGN | 23556 | 18 |
| CCDC50 | 152137 | 3 |
| PNOC | 5368 | 8 |
| FZD3 | 7976 | 8 |
| ITSN1 | 6453 | 21 |
| POGZ | 23126 | 1 |
| XKR4 | 114786 | 8 |
| ATE1 | 11101 | 10 |
| RAD51L1 | 5890 | 14 |
| SH3BGRL2 | 83699 | 6 |
| RGMB | 285704 | 5 |
| KIAA1414 | 54497 | 2 |
| NPC1 | 4864 | 18 |
| ABTB2 | 25841 | 11 |
| ADCK1 | 57143 | 14 |
| C10orf115 | 387642 | 10 |
| CALN1 | 83698 | 7 |
| FBXO16 | 157574 | 8 |
| PPFIA2 | 8499 | 12 |
| STK39 | 27347 | 2 |
| FAM19A1 | 407738 | 3 |
| FBXO42 | 54455 | 1 |
| CDC2 | 983 | 10 |
| BLNK | 29760 | 10 |
| CNTN6 | 27255 | 3 |
| PHC1 | 1911 | 12 |
| ARHGAP18 | 93663 | 6 |
| PRG4 | 10216 | 1 |
| LOC401898 | 401898 | 19 |
| HYPB | 29072 | 3 |
| FRMPD2 | 143162 | 10 |
| CNNM2 | 54805 | 10 |
| C1orf27 | 54953 | 1 |
| FLJ10159 | 55084 | 6 |
| SBF2 | 81846 | 11 |
| SRGAP3 | 9901 | 3 |
| SLC8A1 | 6546 | 2 |
| TGFBR2 | 7048 | 3 |
| LAMA3 | 3909 | 18 |
| MALL | 7851 | 2 |
| UBR2 | 23304 | 6 |
| ZBTB20 | 26137 | 3 |
| REG4 | 83998 | 1 |
| ITIH5 | 80760 | 10 |
| ADAMTS19 | 171019 | 5 |
| KIAA1468 | 57614 | 18 |
| LRP1B | 53353 | 2 |
| PGBD3P3 | 267007 | 12 |
| LOC646210 | 646210 | 5 |
| RUTBC2 | 129049 | 22 |
| WWOX | 51741 | 16 |
| SP110 | 3431 | 2 |
| OR4C4P | 79550 | 11 |
| LOC442195 | 442195 | 6 |
| LARGE | 9215 | 22 |
| SLC25A24 | 29957 | 1 |
| CACNA2D3 | 55799 | 3 |
| SGIP1 | 84251 | 1 |
| GALC | 2581 | 14 |
| LOC387755 | 387755 | 11 |
| IQCB1 | 9657 | 3 |
| FMN2 | 56776 | 1 |
| AP3B1 | 8546 | 5 |
| EIF3S10 | 8661 | 10 |
| NPHP1 | 4867 | 2 |
| ANKRD28 | 23243 | 3 |
| CAPS2 | 84698 | 12 |
| LOC653227 | 653227 | 15 |
| ANKS1 | 23294 | 6 |
| EMR3 | 84658 | 19 |
| DNAJC5B | 85479 | 8 |
| PAPPA2 | 60676 | 1 |
| CDH13 | 1012 | 16 |
| FRAS1 | 80144 | 4 |
| CABIN1 | 23523 | 22 |
| RSRC1 | 51319 | 3 |
| CLDN11 | 5010 | 3 |
| GUCA1C | 9626 | 3 |
| SRI | 6717 | 7 |
| TSPAN8 | 7103 | 12 |
| LOC646385 | 646385 | 7 |
| RYR3 | 6263 | 15 |
| LOC653748 | 653748 | 7 |
| C18orf1 | 753 | 18 |
| NMNAT3 | 349565 | 3 |
| LOC645960 | 645960 | 8 |
| DYNC2LI1 | 51626 | 2 |
| C10orf72 | 196740 | 10 |
| C7orf10 | 79783 | 7 |
| RAC1P1 | 387612 | 18 |
| TYRL | 7300 | 11 |
| LOC646176 | 646176 | 11 |
| CSMD2 | 114784 | 1 |
| CNTNAP5 | 129684 | 2 |
| NTRK2 | 4915 | 9 |
| ZBTB11 | 27107 | 3 |
| PAX9 | 5083 | 14 |
| GPATC2 | 55105 | 1 |
| HNRPD | 3184 | 4 |
| CCBE1 | 147372 | 18 |
| CNTN4 | 152330 | 3 |
| CTNND2 | 1501 | 5 |
| DDEF1 | 50807 | 8 |
| CPXM2 | 119587 | 10 |
| UTRN | 7402 | 6 |
| PRNPIP | 79033 | 1 |
| DRCTNNB1A | 84668 | 7 |
| ALK | 238 | 2 |
| PFKP | 5214 | 10 |
| SLC44A5 | 204962 | 1 |
| MPPED2 | 744 | 11 |
| DNAH8 | 1769 | 6 |
| ZFAND3 | 60685 | 6 |
| SPIRE1 | 56907 | 18 |
| LOC646184 | 646184 | 13 |
| SEC15L2 | 23233 | 2 |
| LOC647219 | 647219 | 1 |
| C14orf101 | 54916 | 14 |
| ANKRD50 | 57182 | 4 |
| GLRA3 | 8001 | 4 |
| TMEM16D | 121601 | 12 |
| LYST | 1130 | 1 |
| LOC390576 | 390576 | 15 |
| GBAS | 2631 | 7 |
| PEX5L | 51555 | 3 |
| C10orf110 | 55853 | 10 |
| LOC223075 | 223075 | 7 |
| EPHA6 | 285220 | 3 |
| RARB | 5915 | 3 |
| LRRN1 | 57633 | 3 |
| DGAT2L4 | 158835 | X |
| MUC19 | 283463 | 12 |
| ITGA1 | 3672 | 5 |
| GADL1 | 339896 | 3 |
| ADCY9 | 115 | 16 |
| EXOC3 | 11336 | 5 |
| UBE2E2 | 7325 | 3 |
| EFHA1 | 221154 | 13 |
| CRIM1 | 51232 | 2 |
| PRDM11 | 56981 | 11 |
| LOC653584 | 653584 | 3 |
| FLJ22104 | 65084 | 11 |
| TUBB1 | 81027 | 20 |
| SGNE1 | 6447 | 15 |
| MBD2 | 8932 | 18 |
| PALLD | 23022 | 4 |
| FSTL4 | 23105 | 5 |
| GLRA1 | 2741 | 5 |
| LOC653480 | 653480 | 10 |
| NCAM2 | 4685 | 21 |
| TMEFF2 | 23671 | 2 |
| TRAPPC6B | 122553 | 14 |
| HIVEP3 | 59269 | 1 |
| MORC1 | 27136 | 3 |
| NTN1 | 9423 | 17 |
| PTX1 | 51290 | 12 |
| CADPS | 8618 | 3 |
| C6orf32 | 9750 | 6 |
| SERBP1 | 26135 | 1 |
| BRMS1L | 84312 | 14 |
| SH3GL2 | 6456 | 9 |
| C1orf164 | 55182 | 1 |
| LOC644668 | 644668 | 8 |
| CHD1 | 1105 | 5 |
| G3BP | 10146 | 5 |
| FLJ30058 | 158763 | X |
| CALCB | 797 | 11 |
| FBXL4 | 26235 | 6 |
| LOC122592 | 122592 | 14 |
| LOC643650 | 643650 | 10 |
| SPARC | 6678 | 5 |
| EXTL3 | 2317 | 8 |
| LOC653435 | 653435 | 7 |
| KIAA0319 | 9856 | 6 |
| LOC643595 | 643595 | 7 |
| THEM2 | 55856 | 6 |
| GLP2R | 9340 | 17 |
| USP43 | 124739 | 17 |

TABLE 10
A linear regression model predicting serum HDL-Cholesterol concentration.

| | Sequence | | | | Risk | | Vari- | Un | Std. | | | |
|---|---|---|---|---|---|---|---|---|---|---|---|---|
| dbSNP rs ID | ID | Gene and Gene ID | Chromosome | Position | Allele | Frequency | ants | stand. b | Error | Stand. b | t value | P value |
| RS10485111 | 681 | | 6 | 11629981 | T | 13.36 | C/T | 0.086 | 0.025 | 0.134 | 3.420 | 0.001 |
| RS10497776 | 228 | | 2 | 196678391 | G | 1.31 | A/G | 0.328 | 0.084 | 0.151 | 3.889 | <0.001 |
| RS10506790 | 1141 | | 12 | 7784138 | A | 9.27 | A/G | 0.116 | 0.03 | 0.154 | 3.938 | <0.001 |
| RS1452528 | 670 | | 6 | 79155079 | A | 49.73 | A/G | 0.071 | 0.017 | 0.165 | 4.191 | <0.001 |
| RS1480145 | 796 | | 8 | 16181494 | G | 1.83 | C/G | 0.240 | 0.056 | 0.169 | 4.320 | <0.001 |
| RS1490125 | 651 | | 6 | 66451580 | T | 13.72 | G/T | −0.151 | 0.027 | −0.220 | −5.716 | <0.001 |
| RS1851006 | 1278 | UNC13C 440279 | 15 | 52691684 | C | 46.3 | C/T | −0.068 | 0.017 | −0.151 | −3.921 | <0.001 |
| RS1935229 | 1 | CAMTA1 23261 | 1 | 7683588 | C | 6.84 | C/T | 0.148 | 0.035 | 0.165 | 4.276 | <0.001 |
| RS198278 | 711 | | 7 | 24214718 | A | 47.97 | A/G | −0.083 | 0.017 | −0.185 | −4.803 | <0.001 |
| RS2029259 | 484 | | 4 | 188284137 | C | 11.45 | A/C | 0.094 | 0.026 | 0.142 | 3.639 | <0.001 |
| RS2195989 | 872 | ANGPT1 284 | 8 | 108555088 | C | 13.8 | C/T | 0.089 | 0.023 | 0.153 | 3.842 | <0.001 |
| RS2333821 | 170 | LOC646790 646790 | 2 | 44230030 | T | 26.44 | C/T | 0.085 | 0.019 | 0.174 | 4.556 | <0.001 |
| RS33375 | 598 | | 5 | 170998718 | A | 14.53 | A/G | 0.085 | 0.025 | 0.133 | 3.400 | 0.001 |
| RS4770403 | 1173 | SGCG 6445 | 13 | 22653127 | A | 11.75 | A/G | 0.113 | 0.028 | 0.159 | 4.066 | <0.001 |
| RS723043 | 157 | LTBP1 4052 | 2 | 33395291 | T | 43.22 | G/T | 0.072 | 0.018 | 0.150 | 3.886 | <0.001 |
| RS745708 | 976 | FRMPD2 143162 | 10 | 49115681 | G | 18.04 | A/G | 0.097 | 0.023 | 0.165 | 4.283 | <0.001 |
| RS7794555 | 754 | LOC653748 653748 | 7 | 153675164 | T | 6.84 | C/T | 0.194 | 0.034 | 0.219 | 5.662 | <0.001 |
| RS896990 | 251 | MGC42174 129563 | 2 | 232597100 | A | 8.9 | A/C | 0.139 | 0.028 | 0.192 | 4.973 | <0.001 |

dbSNP_rs_ID: SNP identification number in NCBI dbSNP database build 125
Gene_locus: Gene positioned in the physical position pointed by the SNP according to NCBI Human Genome Build 36

TABLE 11
A linear regression model predicting serum ApoA1 concentration.

| | Sequence | | | | Risk | | Vari- | Un | Std. | | | |
|---|---|---|---|---|---|---|---|---|---|---|---|---|
| dbSNP rs ID | ID | Gene and Gene ID | Chromosome | Position | Allele | Frequency | ants | stand. b | Error | Stand. b | t value | P value |
| RS1016579 | 881 | | 8 | 129269143 | A | 6.41 | A/G | 0.153 | 0.031 | 0.195 | 4.946 | <0.001 |
| RS10498673 | 1912 | BMP6 654 | 6 | 7770256 | T | 14.02 | C/T | 0.146 | 0.020 | 0.288 | 7.437 | <0.001 |
| RS10521381 | 2582 | | X | 85978186 | G | 29.44 | G/T | 0.053 | 0.012 | 0.177 | 4.509 | <0.001 |
| RS1298362 | 1136 | | 12 | 74493708 | A | 47.57 | A/G | −0.057 | 0.014 | −0.158 | −3.983 | <0.001 |
| RS1867584 | 985 | | 10 | 49321355 | T | 41.74 | C/T | 0.053 | 0.014 | 0.146 | 3.687 | <0.001 |
| RS2111826 | 309 | CLDN11 5010 | 3 | 171623540 | A | 9.21 | A/G | −0.102 | 0.027 | −0.147 | −3.809 | <0.001 |
| RS2353296 | 1733 | | 6 | 47956320 | A | 7.08 | A/C | 0.106 | 0.028 | 0.148 | 3.742 | <0.001 |
| RS25937 | 1815 | CTNND2 1501 | 5 | 11556189 | A | 9.55 | A/G | 0.100 | 0.027 | 0.145 | 3.693 | <0.001 |
| RS352427 | 816 | | 8 | 28571996 | G | 3.29 | A/G | 0.152 | 0.038 | 0.158 | 4.064 | <0.001 |
| RS3912653 | 1091 | FAT3 120114 | 11 | 92135172 | A | 39.68 | A/G | −0.056 | 0.014 | −0.157 | −4.027 | <0.001 |
| RS6919514 | 688 | UST 10090 | 6 | 149151922 | G | 9.19 | G/T | 0.108 | 0.026 | 0.164 | 4.205 | <0.001 |
| RS6954679 | 728 | | 7 | 63134099 | G | 8.03 | A/G | 0.127 | 0.024 | 0.208 | 5.272 | <0.001 |
| RS877345 | 958 | TAF3 83860 | 10 | 8093782 | C | 9.5 | A/C | 0.083 | 0.023 | 0.139 | 3.566 | <0.001 |
| RS878016 | 757 | DLGAP2 9228 | 8 | 1585693 | C | 27.11 | A/C | 0.060 | 0.014 | 0.167 | 4.284 | <0.001 |
| RS901682 | 2208 | MARCH8 220972 | 10 | 45285867 | A | 5.67 | A/G | −0.217 | 0.030 | −0.289 | −7.300 | <0.001 |
| RS9323682 | 2422 | | 14 | 79536316 | A | 7.65 | A/G | 0.083 | 0.025 | 0.131 | 3.358 | 0.001 |

dbSNP_rs_ID: SNP identification number in NCBI dbSNP database build 125
Gene_locus: Gene positioned in the physical position pointed by the SNP according to NCBI Human Genome Build 36
Sequence_ID: Sequence identification number
Risk_allele: The allele that is considered as independet variable in the regression model
Frequency: Frequency of the risk-allele in the data
Variants: Variant alleles present in the SNP
Un stand. b: Unstandardized coefficient in the regression model
Std. Error: Standard error of the coefficient
Stand. b: Standardized coefficient
t value: t value for unstandardized coefficient b
P value: Two tailed significance level of t
Coding of SNPs: 0, 1, 2 where code 2 denotes homozygocity of the minor allele code 1 heterozygocity and code 0 homozygocity of non-minor allele.

### REFERENCES

Alberti, K. G. and P. Z. Zimmet, Definition, diagnosis and classification of diabetes mellitus and its complications. Part 1: diagnosis and classification of diabetes melfitus. Provisional report of a WHO consultation. Diabet Med, 1998. 15(7): p. 539-53.
Alberti, K G., Impaired glucose tolerance: what are the clinical implications? Diabetes Res Clin Pract, 1998. 40 Suppl: p. S3-8.
American Heart Association. Heart Disease and Stroke Statistics—2004 Update. http://www.americanheart.org/downloadable/heart/1079736729696HDSStats2O04UpdateR EV3-19-04.pdf (Accessed 14.07.04).
Attie, A-D., J. P. Kastelein, and M. R. Hayden, Pivotal role of ABCA1 in reverse cholesterol transport influencing HDL levels and susceptibility to atherosclerosis. J Lipid Res, 2001. 42(11): p. 1717-26.
Barter, P. J., et al., Antiinflammatory properties of HDL. Circ Res, 2004. 95(8): p. 764-72.
Bierer B, Coligan J E, Margulies D H, Shevach E M, Strober W. 2002. Current Protocols in Immunology. NY:John Wiley & Sons.
Brousseau M E, Goldkamp A L, Collins D, Demissie S, Connolly A C, Cupples L A, Ordovas J M, Bloomfield H E, Robins S J, Schaefer E J. Polymorphisms in the gene encoding lipoprotein lipase in men with low HDL-C and coronary heart disease: the Veterans Affairs HDL Intervention Trial. J Lipid Res. 2004 October;45(10):1885-91.
Cannon, C. P., et al., Intensive versus moderate lipid lowering with statins after acute coronary syndromes. N Engl J Med, 2004. 350(15): p. 1495-504.
Castelli, W. P., et al., Incidence of coronary heart disease and lipoprotein cholesterol levels. The Framingham Study. Jama, 1986. 256(20): p. 2835-8.
Chisolm, G. M. and D. Steinberg, The oxidative modification hypothesis of atherogenesis: an overview. Free Radic Biol Med, 2000. 28(12): p. 1815-26.
Cockerill, G. W., et al., High-density lipoproteins inhibit cytokine-induced expression of endothelial cell adhesion molecules. Arterioscler Thromb Vasc Biol, 1995. 15(11): p. 1987-94.
Cohen J C, Kiss R S, Pertsemlidis A, Marcel Y L, McPherson R, Hobbs H H. Multiple rare alleles contribute to low plasma levels of HDL cholesterol. Science. 2004 Aug. 6;305(5685):869-72.
Erdmann, E., Diabetes and cardiovascular risk markers. Curr Med Res Opin, 2005. 21 Suppl 1: p. S21-8.
Eronen L, Geerts F, Toivonen H. A Markov chain approach to reconstruction of long haplotypes.
Frick, M. H., et al., Helsinki Heart Study: primary-prevention trial with gemfibrozil in middle-aged men with dyslipidemia. Safety of treatment, changes in risk factors, and incidence of coronary heart disease. N Engl J Med, 1987. 317(20): p. 1237-45.
Genest, J. J., et al., Prevalence of risk factors in men with premature coronary artery disease. Am J Cardiol, 1991. 67(15): p. 1185-9.
Griffiths, A. D., et al., Human anti-self antibodies with high specificity from phage display libraries. Embo J, 1993. 12(2): p. 725-34.
Gylling, H., et al., Polymorphisms in the ABCG5 and ABCG8 genes associate with cholesterol absorption and insulin sensitivity. J Lipid Res, 2004. 45(9): p. 1660-5.
Haas, M. J., et al., Suppression of apolipoprotein AI gene expression in HepG2 cells by TNF alpha and IL-1beta. Biochim Biophys Acta, 2003. 1623(2-3): p. 120-8.
Hargrove, G. M., A. Junco, and N.C. Wong, Hormonal regulation of apolipoprotein AI. J Mol Endocrinol, 1999. 22(2): p. 103-11.
Hay, B. N., J. A. Sorge, and B. Shopes, Bacteriophage cloning and Escherichia coli expression of a human IgM Fab. Hum Antibodies Hybridomas, 1992. 3(2): p. 81-5.
Hayashi, N., et al., A single expression system for the display, purification and conjugation of single-chain antibodies. Gene, 1995. 160(1): p. 129-30.
Hering, S., L. Karawajew, and G. Pasternak, Raji-K562 hybrids and their use for trioma production. Biomed Biochim Acta, 1988. 47(2): p. 211-6.
Huse, W. D., et al., Generation of a large combinatorial library of the immunoglobulin repertoire in phage lambda. Science, 1989. 246(4935): p. 1275-81.
Kohier, G. and C. Milstein, Continuous cultures of fused cells secreting antibody of predefined specificity. Nature, 1975. 256(5517): p. 495-7.
Kozbor, D., A. E. Lagarde, and J. C. Roder, Human hybridomas constructed with antigen-specific Epstein-Barr virus-transformed cell lines. Proc Natl Acad Sci USA, 1982. 79(21): p. 6651-5.
Krieger, M., The other side of scavenger receptors: pattern recognition for host defense. Curr Opin Lipidol, 1997. 8(5): p. 275-80.
Kwok, P. Y., Methods for genotyping single nucleotide polymorphisms. Annu Rev Genomics Hum Genet, 2001. 2: p. 235-58.
Laaksonen, D. E., et al., Metabolic syndrome and development of diabetes mellitus: application and validation of recently suggested definitions of the metabolic syndrome in a prospective cohort study. Am J Epidemiol, 2002. 156(11): p. 1070-7.
Lakka, H. M., et al., The metabolic syndrome and total and cardiovascular disease mortality in middle-aged men. Jama, 2002. 288(21): p. 2709-16.
Lewis, G. F. and D. J. Rader, New insights into the regulation of HDL metabolism and reverse cholesterol transport. Circ Res, 2005. 96(12): p. 1221-32.
Levkau, B., et al., High-density lipoprotein stimulates myocardial perfusion in vivo. Circulation, 2004. 110(21): p. 3355-9.
Linsel-Nitschke, P. and A-R. Tall, HDL as a target in the treatment of atherosclerotic cardiovascular disease. Nat Rev Drug Discov, 2005. 4(3): p. 193-205.
Lopez-Candales, A., Metabolic syndrome X: a comprehensive review of the pathophysiology and recommended therapy. J Med, 2001. 32(5-6): p. 283-300.
Mank-Seymour A R, Durham K L, Thompson J F, Seymour A B, Milos P M. Association between single-nucleotide polymorphisms in the endothelial lipase (LIPG) gene and high-density lipoprotein cholesterol levels. Biochim Biophys Acta. 2004 Feb. 27;1636(1):40-6.
Manninen, V., et al., Joint effects of serum triglyceride and LDL cholesterol and HDL cholesterol concentrations on coronary heart disease risk in the Helsinki Heart Study. Implications for treatment. Circulation, 1992. 85(1): p. 37-45.
Miller, N. E., et al., The Tromso heart-study. High-density lipoprotein and coronary heart-disease: a prospective case-control study. Lancet, 1977. 1(8019): p. 965-8.
Miltiadous G et al, Gene polymorphisms affecting HDL-cholesterol levels in the normolipidemic population. Nutr Metab Cardiovascular Dis. 2005 June; 15(3):219-24.
Moestrup, S. K. and R. Kozyraki, Cubilin, a high-density lipoprotein receptor. Curr Opin Lipidol, 2000. 11(2): p. 133-40.
Moestrup, S. K., et al., The intrinsic factor-vitamin B12 receptor and target of teratogenic antibodies is a megalin-binding peripheral membrane protein with homology to developmental proteins. J Biol Chem, 1998. 273(9): p. 5235-42.
Mooradian, A. D., M. J. Haas, and N. C. Wong, Transcriptional control of apolipoprotein A-I gene expression in diabetes. Diabetes, 2004. 53(3): p. 513-20.
Morabia A, Ross B M, Costanza M C, Cayanis E, Flaherty M S, Alvin G B, Das K, James R, Yang A S, Evagrafov O, Gilliam T C. Population-based study of SR-BI genetic variation and lipid profile. Atherosclerosis. 2004 July;175(1):159-68.
Nash, D. T., Cardiovascular risk beyond LDL-C levels. Other lipids are performers in cholesterol story. Postgrad Med, 2004. 116(3): p. 11-5.
National Cholesterol Education Program, Executive Summary of The Third Report of The National Cholesterol Education Program (NCEP) Expert Panel on Detection, Evaluation, And Treatment of High Blood Cholesterol In Adults (Adult Treatment Panel III). Jama, 2001. 285(19): p. 2486-97.
Navab, M., et al., The oxidation hypothesis of atherogenesis: the role of oxidized phospholipids and HDL. J Lipid Res, 2004. 45(6): p. 993-1007.
Nielsen, P. E., et al., Sequence-selective recognition of DNA by strand displacement with a thymine-substituted polyamide. Science, 1991. 254(5037): p. 1497-500.
Peltonen L, Jalanko A, Varilo T. Molecular genetics of the Finnish disease heritage. 1999. Hum Mol Genet. 8: 1913-23.
Pi-Sunyer, F. X., The epidemiology of central fat distribution in relation to disease. Nutr Rev, 2004. 62(7 Pt 2): p. S120-6.
Ross, R., Atherosclerosis—an inflammatory disease. N Engl J Med, 1999. 340(2): p. 115-26.
Rubins, H. B., et al., Gemfibrozil for the secondary prevention of coronary heart disease in men with low levels of high-density lipoprotein cholesterol. Veterans Affairs High-Density Lipoprotein Cholesterol Intervention Trial Study Group. N Engl J Med, 1999. 341(6): p. 410-8.
Salonen, J. T., et al., Autoantibody against oxidised LDL and progression of carotid atherosclerosis. Lancet, 1992. 339(8798): p. 883-7.
Salonen, J. T., et al., Polymorphism in high density lipoprotein paraoxonase gene and risk of acute myocardial infarction in men: prospective nested case-control study. BMJ, 1999. 319(7208): p. 487-9; discussion 490.
Salonen J T, Nyyssonen K, Korpela H, Tuomilehto J, Seppanen R, Salonen R. High stored iron levels are associated with excess risk of myocardial infarction in eastern Finnish men. Circulation. 1992. 86(3): p. 803-11.
Salonen, J. T., Is there a continuing need for longitudinal epidemiologic research? The Kuopio Ischaemic Heart Disease Risk Factor Study. Ann Clin Res, 1988. 20(1-2): p. 46-50.
Salonen J T, Salonen R, Seppanen K, Rauramaa R, Tuomilehto J. HDL, HDL2, and HDL3 subfractions, and the risk of acute myocardial infarction. A prospective populations study in Eastern Finnish men. Circulation 1991. 84: p 129-39.
Sampietro, T., et al., [High-density lipoproteins: the “new” target of cardiovascular medicine]. Ital Heart J Suppl, 2005. 6(6): p. 341-53.
Scheen, A. J., Pathophysiology of type 2 diabetes. Acta Clin Belg, 2003. 58(6): p. 335-41.
Singaraja, R. R., et al., Human ABCA1 BAC transgenic mice show increased high density lipoprotein cholesterol and ApoAI-dependent efflux stimulated by an internal promoter containing liver X receptor response elements in intron 1. J Biol Chem, 2001. 276(36): p. 33969-79.
Stamler J, Greenland P, Neaton J D. 1998. The established major risk factors underlying epidemic coronary and cardiovascular disease. CVD Prevention 1: 82-97.
Stephens, J. W. and S. E. Humphries, The molecular genetics of cardiovascular disease: clinical implications. J Intern Med, 2003. 253(2): p. 120-7.
Syvanen, A. C., Accessing genetic variation: genotyping single nucleotide polymorphisms. Nat Rev Genet, 2001. 2(12): p. 930-42.
Thompson J F, Durham L K, Lira M E, Shear C, Milos P M. CETP polymorphisms associated with HDL cholesterol may differ from those associated with cardiovascular disease. Atherosclerosis. 2005 July;181(1):45-53.
Tietge, U. J., et al., Human secretory phospholipase A2 mediates decreased plasma levels of HDL cholesterol and apoA-I in response to inflammation in human apoA-I transgenic mice. Arterioscler Thromb Vasc Biol, 2002. 22(7): p. 1213-8.
Todorova, B., et al., The G-250A promoter polymorphism of the hepatic lipase gene predicts the conversion from impaired glucose tolerance to type 2 diabetes mellitus: the Finnish Diabetes Prevention Study. J Clin Endocrinol Metab, 2004. 89(5): p. 2019-23.
Toivonen H T, Onkamo P, Vasko K, Ollikainen V, Sevon P, Mannila H, Herr M, Kere J. Data mining applied to linkage disequilibrium mapping. Am J Hum Genet. 2000 July;67(1):133-45.
Tornvall, P., et al., Lipoprotein lipase mass and activity in plasma and their increase after heparin are separate parameters with different relations to plasma lipoproteins. Arterioscler Thromb Vasc Biol, 1995. 15(8): p. 1086-93.
Tremoli, E., et al., Tissue factor in atherosclerosis. Atherosclerosis, 1999. 144(2): p. 273-83.
Tuomainen, T. P., et al., Increased risk of acute myocardial infarction in carriers of the hemochromatosis gene Cys282Tyr mutation: a prospective cohort study in men in eastern Finland. Circulation, 1999. 100(12): p. 1274-9.
Uittenbogaard, A., et al., High density lipoprotein prevents oxidized low density lipoprotein-induced inhibition of endothelial nitric-oxide synthase localization and activation in caveolae. J Biol Chem, 2000. 275(15): p. 11278-83.
Wadham, C., et al., High-density lipoproteins neutralize C-reactive protein proinflammatory activity. Circulation, 2004. 109(17): p. 2116-22.
Vaisman, B. L., et al., ABCA1 overexpression leads to hyperalphalipoproteinemia and increased biliary cholesterol excretion in transgenic mice. J Clin Invest, 2001. 108(2): p. 303-9.
Vajo, Z., J. G. Terry, and E. A. Brinton, Increased intra-abdominal fat may lower HDL levels by increasing the fractional catabolic rate of Lp A-I in postmenopausal women. Atherosclerosis, 2002. 160(2): p. 495-501.
Wang, M. and M. R. Briggs, HDL: the metabolism, function, and therapeutic importance. Chem Rev, 2004. 104(1): p. 119-37.
Wang, N., et al., ATP-binding cassette transporters G1 and G4 mediate cellular cholesterol efflux to high-density lipoproteins. Proc Natl Acad Sci USA, 2004. 101(26): p. 9774-9.
Waters, D. D., et al., Treating to New Targets (TNT) Study: does lowering low-density lipoprotein cholesterol levels below currently recommended guidelines yield incremental clinical benefit? Am J Cardiol, 2004. 93(2): p. 154-8.
World Health Organization.2004. Cardiovascular diseases: prevention and control. Information sheet. http://www.who.int/dietphysicalactivity/media/en/gsfs_cvd.pdf (Accessed 14 Jul. 2004)
Williams C L, Hayman L L, Daniels S R, Robinson T N, Bazzarre T. Cardiovascular health in childhood: A statement for health professionals from the Committee on Atheroscelrosis, Hypertension, and Obesity in the Young (AHOY) of the Council on Cardiovascular Disease in the Young, American Heart Association. Circulation. 2002 Jul. 2; 106(1): 143-60.
Yuhanna, I. S., et al., High-density lipoprotein binding to scavenger receptor-BI activates endothelial nitric oxide synthase. Nat Med, 2001. 7(7): p. 853-7.

1. A method for preventing or treating of a low high density lipoprotein (HDL) condition or trait in a mammalian subject comprising modulation of biological activity, function or concentration of at least one polypeptide encoded by HDL associated genes set forth in tables 1, 8 and 9 in said subject.

2. The method according to claim 1, wherein a low HDL condition or trait is any condition or trait in which the HDL-C level of a subject is below the accepted normal HDL-C levels.

3. The method according to claim 1, wherein the HDL-C level of a subject is below the average HDL-C level of the related population.

4. The method according to claim 1, wherein the HDL-C level of a subject is below 1.3 mmol/l.

5. The method according to claim 1, wherein the subject with normal HDL-C levels has increased risk to a low HDL-C condition or trait.

6. The method according to claim 1, wherein the low HDL condition or trait comprises lipid disorders, inflammation, cancer, Alzheimer disease, oxidative stress, smoking, obesity, cardiovascular disease, type 2 diabetes and the metabolic syndrome.

7. The method according to claim 1, wherein the subject is at elevated risk of a low HDL-C condition or trait because of family history.

8. The method according to claim 6, wherein the lipid disorder comprises low high density lipoprotein, low ApoA1 lipoprotein, elevated VLDL, elevated LDL-C level, elevated triglycerides level or elevated total cholesterol level.

9. The method according to claim 6, wherein the inflammation is characterized by elevated levels of one or several inflammation markers such as but not limited to C-reactive protein, fibrinogen, leukocyte count or amyloid A.

10. The method according to claim 6 wherein oxidative stress is characterized by elevated levels of one or several oxidative stress markers such as oxidatively modified lipids or lipoproteins, autoantibodies against oxidatively modified lipids or lipoproteins, oxidatively modifies bases, nucleotides or DNA or RNA, or oxidatively modified proteins.

11. The method according to claim 1 comprising administering to a mammalian subject in need of such treatment an effective amount of a compound in a pharmaceutically acceptable carrier altering expression of one or more genes set forth in tables 1, 8 and 9.

12. The method according to claim 1 comprising administering to a mammalian subject in need of such treatment an effective amount of a compound in a pharmaceutically acceptable carrier altering biological activity or function of polypeptides encoded by one or more genes set forth in tables 1, 8 and 9.

13. The method according to claim 1 comprising administering to a mammalian subject in need of such treatment an effective amount of a compound in a pharmaceutically acceptable carrier altering activity or function of a biological network or a metabolic pathway related to a gene set forth in table 1, 8 or 9.

14. The method according to claim 1 comprising administering to a mammalian subject in need of such treatment an effective amount of a compound in a pharmaceutically acceptable carrier altering activity of a pathophysiological pathway related to a gene set forth in table 1, 8 or 9.

15. The method according to claim 1 comprising a recombinant polypeptide encoded by a HDL-C associated gene set forth in table 1, 8 or 9, or variants, fragments or derivatives thereof.

16. The method according to claim 1 comprising gene therapy or gene transfer of at least one HDL-C associated gene set forth in table 1, 8 or 9, or variants, fragments or derivatives thereof.

17. The method according to claim 16, wherein said HDL-C associated gene, or its variants, fragments or derivatives thereof are associated with increased levels of HDL-C.

18. The method according to claim 16, wherein said therapy comprises a polynucleotide hybridising under physiological conditions to the regulatory regions and/or to the polypeptide encoding region of a HDL-C associated gene set forth in table 1, 8 or 9, or variants, fragments or derivatives thereof in somatic cells, in stem cells, or in affected tissues of said subject.

19. The method according to claim 16 comprising sequence specific gene silencing agents such as small interfering RNA (siRNA) or small hairpin RNA (shRNA) hybridising to mRNA and/or to hnRNA of a HDL-C associated gene set forth in tables 1, 8 or 9.

20. The method according to claim 16 comprising sequence specific gene silencing agents such as siRNA or shRNA hybridising to mRNA and/or to hnRNA of a gene in a biological network or a metabolic pathway related to a gene set forth in table 1, 8 or 9.

21. The method according to claim 1, wherein said treatment is based on a dietary treatment or a vaccination.

22. A method for identifying a compound for prevention or treatment of a low HDL-C condition or trait comprising determining the effect of a compound on the biological activity or function of at least one polypeptide encoded by the HDL-C associated genes set forth in tables 1, 8 and 9 in living cells, wherein a compound altering biological activity or function of said polypeptide is considered useful in prevention or treatment of low HDL-C condition or trait.

23. The method according to claim 22 comprising determining the effect of a compound on the biological activity or function of a biological network or a metabolic pathway related to a HDL-C associated gene set forth in table 1, 8 or 9, wherein a compound altering biological activity or function of a biological network or metabolic pathway is considered useful in prevention or treatment of low HDL-C condition or trait.

24. The method according to claim 22 comprising non-human transgenic animals, mammalian tissues, organs or organ systems or cultured microbial, insect or mammalian cells expressing one or more of the HDL-C level associated genes set forth in tables 1, 8 and 9.

25. The method according to claim 22 comprising non-human knock-out animals having one or more of the said HDL-C level associated genes inactivated.

26. A pharmaceutical composition for prevention or treatment of a low HDL-C trait or condition comprising one or more compounds in a pharmaceutically acceptable carrier modulating biological activity, function or concentration of a polypeptide encoded by a HDL-C level associated gene set forth in table 1, 8 or 9 in a mammalian subject.

27. The pharmaceutical composition according to claim 26 comprising one or more compounds in a pharmaceutically acceptable carrier modulating the biological activity or function of a biological network or a metabolic pathway related to said HDL-C associated gene set forth in table 1, 8 or 9.

28. A method for manufacturing a medicament for preventing or treating of a low HDL-C condition or trait in a mammalian subject comprising a compound modulating biological activity, function or concentration of at least one polypeptide encoded by HDL-C level associated genes set forth in tables 1, 8 and 9 in said subject.

29. A method for selecting efficient and safe HDL-C level increasing therapy to a subject comprising:
a) providing a biological sample taken from the subject;
b) assessing type and/or level of at least one biomarker in said sample, wherein said biomarkers are associated to one or more of the HDL-C related genes set forth in tables 1, 8 and 9, or said biomarkers are associated to biological networks or metabolic pathways related to said genes; and
c) using the biomarker data to select efficient and safe therapy for the subject.

30. The method according to claim 29, wherein at least one biomarker is selected from polymorphic sites residing in genomic regions containing the genes set forth in tables 1, 8 and 9.

31. The method according to claim 29, wherein at least one biomarker is selected from SNP markers set forth in tables 2 to 7 and 10 to 11.

32. The method according to claim 29, wherein at least one biomarker is selected from polymorphic sites associated with one or more of the SNP markers set forth in tables 2 to 7 and 10 to 11.

33. The method according to claim 29, wherein at least one biomarker is selected from polymorphic sites being in complete linkage disequilibrium with one or more of the SNP markers set forth in tables 2 to 7 and 10 to 11.

34. The method according to claim 29, wherein at least one biomarker is selected from expression products of the genes set forth in tables 1, 8 and 9.

35. The method according to claim 29, wherein at least one biomarker is selected from the polypeptides encoded by the genes set forth in tables 1, 8 and 9.

36. The method according to claim 29, wherein at least one biomarker is selected from the metabolites of the polypeptides encoded by the genes set forth in tables 1, 8 and 9.

37. The method according to claim 29, wherein the low HDL-C condition or trait is any condition or trait in which the HDL-C level of a subject is below the accepted normal HDL-C levels.

38. The method according to claim 29, wherein the HDL-C level of a subject is below the average HDL-C level of the related population.

39. The method according to claim 29, wherein a low HDL-C condition or trait comprises lipid disorders, inflammation, cancer, Alzheimer disease, oxidative stress, smoking, obesity, cardiovascular disease, type 2 diabetes or the metabolic syndrome.

40. The method according to claim 29 further comprising step d) combining personal and clinical information with the biomarker data to make the selection of the therapy.

41. The method according to claim 40, wherein the personal and clinical information, i.e. non-genetic information concerns age, gender, behaviour patterns and habits, biochemical measurements, clinical measurements, obesity, T2D, cardiovascular disease, dyslipoproteinemia, waist-to-hip circumference ratio (cm/cm), socioeconomic status, psychological traits and states, the medical history of the subject and the family history of said conditions.

42. The method according to claim 40, wherein the dyslipoproteinemia comprises high VLDL level and/or high LDL-C level or/and high triglyceride level and/or high total cholesterol level.

43. The method according to claim 40, wherein the clinical information comprises high plasma level of markers of inflammation at any moment of lifespan.

44. The method according to claim 40, wherein the clinical information comprises high plasma level of markers of oxidative stress.

45. The method according to claim 40, wherein the behaviour patterns and habits include tobacco smoking, physical activity, dietary intakes of nutrients, alcohol intake and consumption patterns and coffee consumption and quality.

46. The method according to claim 40, wherein the biochemical measurements include determining blood, serum or plasma VLDL, LDL, HDL or total cholesterol or triglycerides, ApoA1, fibrinogen, ferritin, transferrin receptor, C-reactive protein, glucose or insulin concentration.

47. A test kit based on a method of claim 29 for selecting efficient and safe HDL-C level increasing therapy to a subject comprising:
a) reagents, materials and protocols for assessing type and/or level of one or more biomarkers in a biological sample, wherein said biomarkers are associated to one or more of the HDL-C related genes set forth in tables 1, 8 and 9, or said biomarkers are associated to biological networks or metabolic pathways related to said genes; and
b) instructions and software for using the biomarker data to select efficient and safe therapy for the subject.

48. The test kit according to claim 47 further comprising questionnaire and instructions for collecting personal and clinical information from the subject.
